SEQUENCE LISTING (59 sequences)

SEQ ID NO: 1 - protein, 383 aa, synthetic construct
MDTSSPSPERSAPTPASPVATVSQYACQRRTTLENHNKIFTDAFEILAENYEFNESEGPCLAFMRAASVLKSLPHAISSLKDLEGLPCLGDQTKAVIEDILECGQSSKVQDVLCDERYKAFKLFTSVFGVGLKTAEKWYRRGFRSLEEVKADNSIHFTKMQKAGFLYYDDISRAVSKAEAEAIGQIVEETVHLIVPDAIVTLTGGFRRGKEFGHDVDFLITTPETGKEEGLLHKVINRLKNQGILLYYDIVESTFDKTKLPSRRFEAMDHFQKCFAILKLNKELVEGDGQEQSDERASKDWKAIRVDLVVPPVDNFAFALLGWTGSRQFERDLRRYAHHEKKMLLDNHALYDKTKKVFLKAKTEEDIFAHLGLEYIEPWQRNA

SEQ ID NO: 2 - protein, 389 aa, synthetic construct
MEQQDQSPTLNTPESEVPSFTASKVSQYSCQRKTTLNNYNKKFTDAFEIMAENYEFKENEIFCLEFLRAASVLKFLPFPVVRMKDIQGLPCMGDQVRDVIEEIIEEGESSRAKDVLNDERYKSFKQFTSVFGVGVKTSEKWYRMGLRTLEEVKADKTLKLSKMQKAGFLYYEDLVSCVSKAEADAVSLIVKNTVCTFLPDALVTITGGFRRGKKIGHDIDFLITNPGPREDDELLHKVVDLWKKQGLLLYCDIIESTFVKEQLPSRKVDAMDHFQKCFAILKLYQPRVDNSSYNTSKEFDMAEVKDWKAIRVDLVISPFEQYAYALLGWTGSRQFGRDLRRYATHERKMMLDNHALYDKRKRIFLKAGSEEEIFAHLGLDYVEPWERNA

SEQ ID NO: 3 - protein, 388 aa, synthetic construct
MEQDQSPPLNTPESEVPSFIASKVSQYSCQRKTTLNNYNKKFTDAFEIMAENYEFKENEIFCLEFLRAASVLKFLPFPVTRMKDIQGLPCMGDRVRDVIEEIIEEGESSRAKEVLNDERYKSFKQFTSVFGVGVKTSEKWYRMGLRTLEEVKADKTLKLSKMQKAGFLYYEDLVSCVSKAEADAVSLIVKNTVCTFLPDALVTITGGFRRGKKIGHDIDFLITNPGPREDDELLHKVVDLWKKQGLLLYCDIIESTFVKEQLPSRKVDAMDHFQKCFAILKLYQPRVDNSSYNTSKKFDMAEVKDWKAIRVDLVITPFEQYAYALLGWTGSRQFGRDLRRYATHERKMILDNHALYDRRKRIFLKAGSEEEIFAHLGLDYVEPWERNA

SEQ ID NO: 4 - protein, 35 aa, synthetic construct
SVFGVGLKTAEKWYRRGFRSLEEVKADNSIHFTKM

SEQ ID NO: 5 - protein, 35 aa, synthetic construct
SVFGVGVKTSEKWYRMGLRTLEEVKADKTLKLSKM

SEQ ID NO: 6 - protein, 35 aa, synthetic construct; identical to SEQ ID NO: 5

SEQ ID NO: 7 - protein, 40 aa, synthetic construct
EAEAIGQIVEETVHLIVPDAIVTLTGGFRRGKEFGHDVDF

SEQ ID NO: 8 - protein, 40 aa, synthetic construct
EADAVSLIVKNTVCTFLPDALVTITGGFRRGKKIGHDIDF

SEQ ID NO: 9 - protein, 40 aa, synthetic construct; identical to SEQ ID NO: 8

SEQ ID NO: 10 - protein, 35 aa, synthetic construct
GILLYYDIVESTFDKTKLPSRRFEAMDHFQKCFAI

SEQ ID NO: 11 - protein, 35 aa, synthetic construct
GLLLYCDIIESTFVKEQLPSRKVDAMDHFQKCFAI

SEQ ID NO: 12 - protein, 35 aa, synthetic construct; identical to SEQ ID NO: 11

SEQ ID NO: 13 - protein, 35 aa, synthetic construct
RVDLVVPPVDNFAFALLGWTGSRQFERDLRRYAHH

SEQ ID NO: 14 - protein, 35 aa, synthetic construct
RVDLVISPFEQYAYALLGWTGSRQFGRDLRRYATH

SEQ ID NO: 15 - protein, 35 aa, synthetic construct
RVDLVITPFEQYAYALLGWTGSRQFGRDLRRYATH

SEQ ID NO: 16 - protein, 388 aa, synthetic construct
MEQDQSPPLNTPESEVPSFIASKVSQYSCQRKTTLNNYNKKFTDAFEIMAENYEFKENEIFCLEFLRAASVLKFLPFPVTRMKDIQGLPCMGDRVRDVIEEIIEEGESSRAKEVLNDERYKSFKQFTSVFGVGVKTSEKWYRMGLRTLEEVKADKTLKLSKMQKAGFLYYEDLVSCVSKAEADAVSLIVKNTVCTFLPDALVTITGGFRLGGKIGHDIDFLITNPGPREDDELLHKVVDLWKKQGLLLYCDIIESTFVKEQLPSRKVDAMDHFQKCFAILKLYQPRVDNSSYNTSKKFDMAEVKDWKAIRVDLVITPFEQYAYALLGWTGSRQFGRDLRRYATHERKMILDNHALYDRRKRIFLKAGSEEEIFAHLGLDYVEPWERNA

SEQ ID NO: 17 - protein, 389 aa, synthetic construct
MEQQDQSPTLNTPESEVPSFTASKVSQYSCQRKTTLNNYNKKFTDAFEIMAENYEFKENEIFCLEFLRAASVLKFLPFPVVRMKDIQGLPCMGDQVRDVIEEIIEEGESSRAKDVLNDERYKSFKQFTSVFGVGVKTSEKWYRMGLRTLEEVKADKTLKLSKMQKAGFLYYEDLVSCVSKAEADAVSLIVKNTVCTFLPDALVTITGGFRLGGKIGHDIDFLITNPGPREDDELLHKVVDLWKKQGLLLYCDIIESTFVKEQLPSRKVDAMDHFQKCFAILKLYQPRVDNSSYNTSKEFDMAEVKDWKAIRVDLVISPFEQYAYALLGWTGSRQFGRDLRRYATHERKMMLDNHALYDKRKRIFLKAGSEEEIFAHLGLDYVEPWERNA

SEQ ID NO: 18 - protein, 393 aa, synthetic construct
MESRVASANPDPNAGMLKIQSPATKTISQYACQRRTTLNNHNQRFTDAFEILAKNYEFRENDGTCLTFMRATSVLKCLPFAIVSMKDTEGLPWIGDEVKGIMEEIIEDGESLEVQAVLNDERYQSFKLFTSVFGVGLKTAEKWYRMGFRTLSKIRSDKSLKLTKMQKAGFLYYEDLISCVSKAEADAVSLIVKEAVWTFLPDALVTITGGFRRGKEFGHDVDFLITSPGAEKEQEDQLLQKVTNLWEKQGLLLYYDLIESTFEDLKLPSRKVDALDHFQKCFLILKLYHQRGDKSKWEGPEGSNELEAKNWKAIRVDLVVCPYDRYAYALLGWSGSRQFERDLRRYATHEKKMMLDNHALYDKTKRIFLKAESEEEIFAHLGLEYIEPWERNA

SEQ ID NO: 19 - protein, 393 aa, synthetic construct
MVRRDCSASPNPSTRMTKTPSLATKKISQYACQRRTTLNNCNQKFTDAFEILAENYEFRENEGTCLAFMRAASVLKCLPFTIVSMKDTEGLPWLGDQVKGIIEEIIEDGESSEVKAVLNDERYQSFKLFTSVFGVGLKTSEKWYRMGFRTLSKIRSDKSLKLTKMQKAGFLYYEDLISCVSKAEADAVSLIVKEAVWTFLPDALVTMTGGFRRGKKIGHDVDFLITSPGSGEEEEEQLLHKVINLWEKQGLLLYYDLIESTFEKLKLPSRKVDALDHFQKCFLILKLHHQRVDNSRYEGPEESNEQEAKNWKAIRVDLVMCPYERYAYALLGWTGSRQFERDLRRYATHEKKMMLDNHALYDKTKRIFLKAESEEEIFAHLGLDYIEPWERNA

SEQ ID NO: 20 - protein, 391 aa, synthetic construct
MVQQDCSASSSSSTRPTETPSLAAAKVSQYACQRRTTLNNCNKKFTDAFEILAENYEFRENEGPCLAFMRAASVLKSLPFTIVRMKDIEGLPWLGDQVKGVIEEIIEDGESSKVKDVLNDERYKSFKLFTSVFGVGLKTSEKWYRMGFRTLEEIKADKSLKLTKMQKAGFLYYEDLISCVSKAEADAVSLIVKDTVWTFLPDALVTLTGGFRRGKKIGHDVDFLITSPGPGEEEELLHKVINLWKKQGLLLYYDLIESTFEKTKLPSRKVDALDHFQKCFAILKLHKQRVDNSSSDGPEESDEQEAKNWKAIRVDLVISPFEQYAYALLGWTGSRQFERDLRRYATHEKKMMLDNHALYDKTKRIFLKAESEEEIFAHLGLDYIEPWERNA

SEQ ID NO: 21 - protein, 393 aa, synthetic construct
MVQQDCSASYQSSSSTRPTETSSLAAAKVSQYACQRRTTLNNCNKKFTDAFEILAENYEFRENEGPCLAFMRAASVLKFLPFTIVRMKDIEGLPWMGDQVKGVIEEIIEEGESSRVKDVLNDERYKSFKLFTSVFGVGLKTSEKWYRMGFRTLEEVKADKSLKLTRMQKAGFLYYEDLISCVSKAEADAVSLIVKDTVWTFLPDALVTLTGGFRRGKKIGHDVDFLITSPGPREEEELLHKVIDLWKKQGLLLYYDLIESTFEKTKLPSRKVDALDHFQKCFAILKLHKQRVDNSSSDTSEESDEQEAKDWKAIRVDLVISPFEQYAYALLGWTGSRQFERDLRRYATHEKKMMLDNHALYDKTKRIFLKAESEEEIFAHLGLDYIEPWERNA

SEQ ID NO: 22 - protein, 393 aa, synthetic construct
MVQQDCSASYQSSSSTRPTETSSLAAAKVSQYACQRRTTLNNCNKKFTDAFEILAENYEFRENEGPCLAFMRAASVLKFLPFTIVRMKDIEGLPWMGDQVKGVIEEIIEEGESSRVKDVLNDERYKSFKLFTSVFGVGLKTSEKWYRMGFRTLEEVKADKSLKLTRMQKAGFLYYEDLISCVSKAEADAVSLIVKDTVWTFLPDALVTLTGGFRLGGKIGHDVDFLITSPGPREEEELLHKVIDLWKKQGLLLYYDLIESTFEKTKLPSRKVDALDHFQKCFAILKLHKQRVDNSSSDTSEESDEQEAKDWKAIRVDLVISPFEQYAYALLGWTGSRQFERDLRRYATHEKKMMLDNHALYDKTKRIFLKAESEEEIFAHLGLDYIEPWERNA

SEQ ID NO: 23 - protein, 389 aa, synthetic construct
MVQQDCSATSDTPESEASSFAAAKVSQYACQRRTTLNNCNKKFTDAFEILAENYEFRENEVPCLAFMRAASVLKFLPFTIVRMKDIEGLPCMGDQVRNVIEEIIEEGESSRAKDVLNDERYKSFKLFTSVFGVGLKTSEKWYRMGFRTLEEVKADKTLKLSRMQKAGFLYYEDLISCVSKAEADAVSLIVKDTVWTFLPDALVTITGGFRLGGKIGHDVDFLITSPGPREEEELLHKVIDLWKKQGLLLYYDLIESTFEKAKLPSRKVDALDHFQKCFAILKLHQQRVDNSSSDTSKESDKAEVKDWKAIRVDLVISPFEQYAYALLGWTGSRQFERDLRRYATHERKMMLDNHALYDKTKRIFLKAESEEEIFAHLGLDYIEPWERNA

SEQ ID NO: 24 - protein, 389 aa, synthetic construct
MVQQDCSATSDTPESEASSFAAAKVSQYACQRRTTLNNYNKKFTDAFEILAENYEFRENEVPCLEFLRAASVLKFLPFPIVRMKDIEGLPCMGDQVRNVIEEIIEEGESSRAKDVLNDERYKSFKLFTSVFGVGLKTSEKWYRMGFRTLEEVKADKTLKLSRMQKAGFLYYEDLVSCVSKAEADAVSLIVKDTVCTFLPDALVTITGGFRLGGKIGHDVDFLITNPGPREEEELLHKVVDLWKKQGLLLYCDIIESTFEKEKLPSRKVDALDHFQKCFAILKLHQQRVDNSSSNTSKESDKAEVKDWKAIRVDLVISPFEQYAYALLGWTGSRQFERDLRRYATHERKMMLDNHALYDKTKRIFLKAESEEEIFAHLGLDYIEPWERNA

SEQ ID NO: 25 - protein, 799 aa, synthetic construct
MGHHHHHHHHHHSSGHIDDDDKHMMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGSSGHMEQDQSPPLNTPESEVPSFIASKVSQYSCQRKTTLNNYNKKFTDAFEIMAENYEFKENEIFCLEFLRAASVLKFLPFPVTRMKDIQGLPCMGDRVRDVIEEIIEEGESSRAKEVLNDERYKSFKQFTSVFGVGVKTSEKWYRMGLRTLEEVKADKTLKLSKMQKAGFLYYEDLVSCVSKAEADAVSLIVKNTVCTFLPDALVTITGGFRRGKKIGHDIDFLITNPGPREDDELLHKVVDLWKKQGLLLYCDIIESTFVKEQLPSRKVDAMDHFQKCFAILKLYQPRVDNSSYNTSKKFDMAEVKDWKAIRVDLVITPFEQYAYALLGWTGSRQFGRDLRRYATHERKMILDNHALYDRRKRIFLKAGSEEEIFAHLGLDYVEPWERNA

SEQ ID NO: 26 - protein, 822 aa, synthetic construct
MGHHHHHHHHHHSSGHIDDDDKHMMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGRDHMVLHEYVNAAGITDGGSGGGSTSHMEQDQSPPLNTPESEVPSFIASKVSQYSCQRKTTLNNYNKKFTDAFEIMAENYEFKENEIFCLEFLRAASVLKFLPFPVTRMKDIQGLPCMGDRVRDVIEEIIEEGESSRAKEVLNDERYKSFKQFTSVFGVGVKTSEKWYRMGLRTLEEVKADKTLKLSKMQKAGFLYYEDLVSCVSKAEADAVSLIVKNTVCTFLPDALVTITGGFRLGGKIGHDIDFLITNPGPREDDELLHKVVDLWKKQGLLLYCDIIESTFVKEQLPSRKVDAMDHFQKCFAILKLYQPRVDNSSYNTSKKFDMAEVKDWKAIRVDLVITPFEQYAYALLGWTGSRQFGRDLRRYATHERKMILDNHALYDRRKRIFLKAGSEEEIFAHLGLDYVEPWERNA

SEQ ID NO: 27 - protein, 800 aa, synthetic construct
MGHHHHHHHHHHSSGHIDDDDKHMMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGSSGHMEQQDQSPTLNTPESEVPSFTASKVSQYSCQRKTTLNNYNKKFTDAFEIMAENYEFKENEIFCLEFLRAASVLKFLPFPVVRMKDIQGLPCMGDQVRDVIEEIIEEGESSRAKDVLNDERYKSFKQFTSVFGVGVKTSEKWYRMGLRTLEEVKADKTLKLSKMQKAGFLYYEDLVSCVSKAEADAVSLIVKNTVCTFLPDALVTITGGFRRGKKIGHDIDFLITNPGPREDDELLHKVVDLWKKQGLLLYCDIIESTFVKEQLPSRKVDAMDHFQKCFAILKLYQPRVDNSSYNTSKEFDMAEVKDWKAIRVDLVISPFEQYAYALLGWTGSRQFGRDLRRYATHERKMMLDNHALYDKRKRIFLKAGSEEEIFAHLGLDYVEPWERNA

SEQ ID NO: 28 - protein, 823 aa, synthetic construct
MGHHHHHHHHHHSSGHIDDDDKHMMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGRDHMVLHEYVNAAGITDGGSGGGSTSHMEQQDQSPTLNTPESEVPSFTASKVSQYSCQRKTTLNNYNKKFTDAFEIMAENYEFKENEIFCLEFLRAASVLKFLPFPVVRMKDIQGLPCMGDQVRDVIEEIIEEGESSRAKDVLNDERYKSFKQFTSVFGVGVKTSEKWYRMGLRTLEEVKADKTLKLSKMQKAGFLYYEDLVSCVSKAEADAVSLIVKNTVCTFLPDALVTITGGFRLGGKIGHDIDFLITNPGPREDDELLHKVVDLWKKQGLLLYCDIIESTFVKEQLPSRKVDAMDHFQKCFAILKLYQPRVDNSSYNTSKEFDMAEVKDWKAIRVDLVISPFEQYAYALLGWTGSRQFGRDLRRYATHERKMMLDNHALYDKRKRIFLKAGSEEEIFAHLGLDYVEPWERNA

SEQ ID NO: 29 - protein, 794 aa, synthetic construct
MGHHHHHHHHHHSSGHIDDDDKHMMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGSSGHMDTSSPSPERSAPTPASPVATVSQYACQRRTTLENHNKIFTDAFEILAENYEFNESEGPCLAFMRAASVLKSLPHAISSLKDLEGLPCLGDQTKAVIEDILECGQSSKVQDVLCDERYKAFKLFTSVFGVGLKTAEKWYRRGFRSLEEVKADNSIHFTKMQKAGFLYYDDISRAVSKAEAEAIGQIVEETVHLIVPDAIVTLTGGFRRGKEFGHDVDFLITTPETGKEEGLLHKVINRLKNQGILLYYDIVESTFDKTKLPSRRFEAMDHFQKCFAILKLNKELVEGDGQEQSDERASKDWKAIRVDLVVPPVDNFAFALLGWTGSRQFERDLRRYAHHEKKMLLDNHALYDKTKKVFLKAKTEEDIFAHLGLEYIEPWQRNA

SEQ ID NO: 30 - protein, 804 aa, synthetic construct
MGHHHHHHHHHHSSGHIDDDDKHMMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGSSGHMESRVASANPDPNAGMLKIQSPATKTISQYACQRRTTLNNHNQRFTDAFEILAKNYEFRENDGTCLTFMRATSVLKCLPFAIVSMKDTEGLPWIGDEVKGIMEEIIEDGESLEVQAVLNDERYQSFKLFTSVFGVGLKTAEKWYRMGFRTLSKIRSDKSLKLTKMQKAGFLYYEDLISCVSKAEADAVSLIVKEAVWTFLPDALVTITGGFRRGKEFGHDVDFLITSPGAEKEQEDQLLQKVTNLWEKQGLLLYYDLIESTFEDLKLPSRKVDALDHFQKCFLILKLYHQRGDKSKWEGPEGSNELEAKNWKAIRVDLVVCPYDRYAYALLGWSGSRQFERDLRRYATHEKKMMLDNHALYDKTKRIFLKAESEEEIFAHLGLEYIEPWERNA

SEQ ID NO: 31 - protein, 804 aa, synthetic construct
MGHHHHHHHHHHSSGHIDDDDKHMMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGSSGHMVRRDCSASPNPSTRMTKTPSLATKKISQYACQRRTTLNNCNQKFTDAFEILAENYEFRENEGTCLAFMRAASVLKCLPFTIVSMKDTEGLPWLGDQVKGIIEEIIEDGESSEVKAVLNDERYQSFKLFTSVFGVGLKTSEKWYRMGFRTLSKIRSDKSLKLTKMQKAGFLYYEDLISCVSKAEADAVSLIVKEAVWTFLPDALVTMTGGFRRGKKIGHDVDFLITSPGSGEEEEEQLLHKVINLWEKQGLLLYYDLIESTFEKLKLPSRKVDALDHFQKCFLILKLHHQRVDNSRYEGPEESNEQEAKNWKAIRVDLVMCPYERYAYALLGWTGSRQFERDLRRYATHEKKMMLDNHALYDKTKRIFLKAESEEEIFAHLGLDYIEPWERNA

SEQ ID NO: 32 - protein, 802 aa, synthetic construct
MGHHHHHHHHHHSSGHIDDDDKHMMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGSSGHMVQQDCSASSSSSTRPTETPSLAAAKVSQYACQRRTTLNNCNKKFTDAFEILAENYEFRENEGPCLAFMRAASVLKSLPFTIVRMKDIEGLPWLGDQVKGVIEEIIEDGESSKVKDVLNDERYKSFKLFTSVFGVGLKTSEKWYRMGFRTLEEIKADKSLKLTKMQKAGFLYYEDLISCVSKAEADAVSLIVKDTVWTFLPDALVTLTGGFRRGKKIGHDVDFLITSPGPGEEEELLHKVINLWKKQGLLLYYDLIESTFEKTKLPSRKVDALDHFQKCFAILKLHKQRVDNSSSDGPEESDEQEAKNWKAIRVDLVISPFEQYAYALLGWTGSRQFERDLRRYATHEKKMMLDNHALYDKTKRIFLKAESEEEIFAHLGLDYIEPWERNA

SEQ ID NO: 33 - protein, 804 aa, synthetic construct
MGHHHHHHHHHHSSGHIDDDDKHMMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGSSGHMVQQDCSASYQSSSSTRPTETSSLAAAKVSQYACQRRTTLNNCNKKFTDAFEILAENYEFRENEGPCLAFMRAASVLKFLPFTIVRMKDIEGLPWMGDQVKGVIEEIIEEGESSRVKDVLNDERYKSFKLFTSVFGVGLKTSEKWYRMGFRTLEEVKADKSLKLTRMQKAGFLYYEDLISCVSKAEADAVSLIVKDTVWTFLPDALVTLTGGFRRGKKIGHDVDFLITSPGPREEEELLHKVIDLWKKQGLLLYYDLIESTFEKTKLPSRKVDALDHFQKCFAILKLHKQRVDNSSSDTSEESDEQEAKDWKAIRVDLVISPFEQYAYALLGWTGSRQFERDLRRYATHEKKMMLDNHALYDKTKRIFLKAESEEEIFAHLGLDYIEPWERNA

SEQ ID NO: 34 - protein, 827 aa, synthetic construct
MGHHHHHHHHHHSSGHIDDDDKHMMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGRDHMVLHEYVNAAGITDGGSGGGSTSHMVQQDCSASYQSSSSTRPTETSSLAAAKVSQYACQRRTTLNNCNKKFTDAFEILAENYEFRENEGPCLAFMRAASVLKFLPFTIVRMKDIEGLPWMGDQVKGVIEEIIEEGESSRVKDVLNDERYKSFKLFTSVFGVGLKTSEKWYRMGFRTLEEVKADKSLKLTRMQKAGFLYYEDLISCVSKAEADAVSLIVKDTVWTFLPDALVTLTGGFRLGGKIGHDVDFLITSPGPREEEELLHKVIDLWKKQGLLLYYDLIESTFEKTKLPSRKVDALDHFQKCFAILKLHKQRVDNSSSDTSEESDEQEAKDWKAIRVDLVISPFEQYAYALLGWTGSRQFERDLRRYATHEKKMMLDNHALYDKTKRIFLKAESEEEIFAHLGLDYIEPWERNA

SEQ ID NO: 35 - protein, 823 aa, synthetic construct
MGHHHHHHHHHHSSGHIDDDDKHMMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGRDHMVLHEYVNAAGITDGGSGGGSTSHMVQQDCSATSDTPESEASSFAAAKVSQYACQRRTTLNNCNKKFTDAFEILAENYEFRENEVPCLAFMRAASVLKFLPFTIVRMKDIEGLPCMGDQVRNVIEEIIEEGESSRAKDVLNDERYKSFKLFTSVFGVGLKTSEKWYRMGFRTLEEVKADKTLKLSRMQKAGFLYYEDLISCVSKAEADAVSLIVKDTVWTFLPDALVTITGGFRLGGKIGHDVDFLITSPGPREEEELLHKVIDLWKKQGLLLYYDLIESTFEKAKLPSRKVDALDHFQKCFAILKLHQQRVDNSSSDTSKESDKAEVKDWKAIRVDLVISPFEQYAYALLGWTGSRQFERDLRRYATHERKMMLDNHALYDKTKRIFLKAESEEEIFAHLGLDYIEPWERNA

SEQ ID NO: 36 - protein, 823 aa, synthetic construct
MGHHHHHHHHHHSSGHIDDDDKHMMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGRDHMVLHEYVNAAGITDGGSGGGSTSHMVQQDCSATSDTPESEASSFAAAKVSQYACQRRTTLNNYNKKFTDAFEILAENYEFRENEVPCLEFLRAASVLKFLPFPIVRMKDIEGLPCMGDQVRNVIEEIIEEGESSRAKDVLNDERYKSFKLFTSVFGVGLKTSEKWYRMGFRTLEEVKADKTLKLSRMQKAGFLYYEDLVSCVSKAEADAVSLIVKDTVCTFLPDALVTITGGFRLGGKIGHDVDFLITNPGPREEEELLHKVVDLWKKQGLLLYCDIIESTFEKEKLPSRKVDALDHFQKCFAILKLHQQRVDNSSSNTSKESDKAEVKDWKAIRVDLVISPFEQYAYALLGWTGSRQFERDLRRYATHERKMMLDNHALYDKTKRIFLKAESEEEIFAHLGLDYIEPWERNA

SEQ ID NO: 37 - protein, 382 aa, synthetic construct
RTDYSATPNPGFQKTPPLAVKKISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 38 - protein, 504 aa, synthetic construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGELMRTDYSATPNPGFQKTPPLAVKKISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 39 - protein, 379 aa, synthetic construct
SPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 40 - protein, 807 aa, synthetic construct
MGHHHHHHHHHHSSGHIDDDDKHMMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGIEGRISHMSMGGRDIVDGSEFSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 41 - protein, 395 aa, synthetic construct
MGHHHHHHHHHHSSGHIDDDDKHMMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSS

SEQ ID NO: 42 - protein, 16 aa, synthetic construct
NNNNNNNNNNLGSSGH

SEQ ID NO: 43 - protein, 16 aa, synthetic construct
RDHMVLHEYVNAAGIT

SEQ ID NO: 44 - protein, 12 aa, synthetic construct
NNNNNNNNNNLG

SEQ ID NO: 45 - protein, 11 aa, synthetic construct
DGGSGGGSTSH

SEQ ID NO: 46 - protein, 119 aa, synthetic construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGG

SEQ ID NO: 47 - skipped

SEQ ID NO: 48 - protein, 495 aa, synthetic construct [sequence omitted]

SEQ ID NO: 49 - protein, 508 aa, synthetic construct
MNKEILAVVEAVSNEKALPREKIFEALESALATATKKKYEQEIDVRVQIDRKSGDFDTFRRWLVVDEVTQPTKEITLEAARYEDESLNLGDYVEDQIESVTFDRITTQTAKQVIVQKVREAERAMVVDQFREHEGEIITGVVKKVNRDNISLDLGNNAEAVILREDMLPRENFRPGDRVRGVLYSVRPEARGAQLFVTRSKPEMLIELFRIEVPEIGEEVIEIKAAARDPGSRAKIAVKTNDKRIDPVGACVGMRGARVQAVSTELGGERIDIVLWDDNPAQFVINAMAPADVASIVVDEDKHTMDIAVEAGNLAQAIGRNGQNVRLASQLSGWELNVMTVDDLQAKHQAEAHAAIDTFTKYLDIDEDFATVLVEEGFSTLEELAYVPMKELLEIEGLDEPTVEALRERAKNALATIAQAQEESLGDNKPADDLLNLEGVDRDLAFKLAARGVCTLEDLAEQGIDDLADIEGLTDEKAGALIMAARNICWFGDEATSGSGHHHHHHHH

SEQ ID NO: 50 - protein, 108 aa, synthetic construct
SDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 51 - protein, 120 aa, synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSHHHHHHHH

SEQ ID NO: 52 - protein, 218 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPK

SEQ ID NO: 53 - protein, 230 aa, synthetic construct
HHHHHHHHHHAGMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPK

SEQ ID NO: 54 - protein, 294 aa, synthetic construct
EIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEIS

SEQ ID NO: 55 - protein, 305 aa, synthetic construct
EIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGHHHHHHHHHH

SEQ ID NO: 56 - protein, 98 aa, synthetic construct
MSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGG

SEQ ID NO: 57 - protein, 395 aa, synthetic construct
MGHHHHHHHHHHSSGHIDDDDKHMMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTRITK

SEQ ID NO: 58 - protein, 25 aa, synthetic construct
MGHHHHHHHHHHSSGHIDDDDKHMM

SEQ ID NO: 59 - DNA, 16 bp, synthetic construct
  source 1..16: mol_type = other DNA
taatacgactcacttg

(19) United States
(12) Patent Application Publication
(10) Pub. No.: US 2026/0327581 A1
(43) Pub. Date: Oct. 22, 2026

(54) RECOMBINANT TERMINAL DEOXYNUCLEOTIDYL TRANSFERASE ENZYME

(71) Applicant: KURA BIOTECH, INC., Rancho Dominguez, CA (US)

(72) Inventors: Javier Ignacio CÁCERES MOLINA, Puerto Varas (CL); Andrea Magdalena CHANIQUE SALLUSTI, Puerto Varas (CL); Jennifer Hsin-wen HSIEH, Puerto Varas (CL); Cynthia Tamara SANHUEZA RIVERA,, Puerto Varas (CL); Daniela Julia CONTADOR ZANFORLÍN, Santiago (CL); Julián Esteban SÁEZ ARDURA, Temuco (CL); Tamara Lissette AKENTJEW FAURE, Puerto Varas (CL); Javier Andrés GIMPEL HENRÍQUEZ, Puerto Varas (CL); Marisol Andrea MORALES SOTO, Santiago (CL); Manuel ROZAS ANDREU, Puerto Varas (CL)

(73) Assignee: KURA BIOTECH, INC., Rancho Dominguez, CA (US)

(21) Appl. No.: 19/485,122

(22) PCT Filed: May 17, 2024
(86) PCT No.: PCT/US2024/030092

§ 371 Date: Nov. 17, 2025

(30) Foreign Application Priority Data
May 17, 2023 (CL) 202301425

(51) Int. Cl. C12N 9/12; C12N 15/11; C12P 19/34

(52) U.S. Cl. CPC C12N 9/1264; C12N 15/11; C12P 19/34; C12Y 207/07031

(57) ABSTRACT

Provided herein are, inter alia, are terminal deoxynucleotidyl transferase (TdT) peptides. These TdT peptides are capable of synthesizing oligonucleotides using nucleoside triphosphate or 3′-O-blocked nucleoside triphosphates with high fidelity and efficiency. Also provided herein are, inter alia, compositions, kits, and methods of using the TdT peptides provided herein including embodiments thereof. The compositions, kits, and methods provided herein are a novel and efficient alternative for the synthesis of oligonucleotides in various biotechnological applications.

### RELATED APPLICATION DATA

This application claims the benefit of priority under 35 U.S.C. § 119(e) of Chilean patent application No. 202301425, filed on May 17, 2023, which is hereby incorporated by reference in its entirety and for all purposes.

### SEQUENCE LISTING

The material in the accompanying Sequence Listing is hereby incorporated by reference in its entirety. The accompanying file, named “061524-508001WO_SL_ST26.xml” was created on May 17, 2024 and is 71,078 bytes in size.

### TECHNICAL FIELD

The present invention relates to the field of biotechnology and deoxyribonucleic acid (DNA)/ribonucleic acid (RNA) synthesis, specifically to new terminal deoxynucleotidyl transferase (TdT) peptides for its application in the biotechnological industry.

### BACKGROUND

DNA and RNA sequence synthesis is an essential process in biotechnology. Currently, oligonucleotide synthesis methods based on solid phase phosphoramidite chemistry are widely used. These methods are based on the successive addition of nucleotides using phosphoramidite derivatives as building blocks (Caruthers, M. (1985). Gene synthesis machines: DNA chemistry and its uses. Science, 230(4723), 281-285). The main advantage of these methods is the high purity and yield of the synthesized oligonucleotides, which allows for the acquisition of DNA sequences of moderate length for research purposes and biotechnological applications. However, these methods have noticeable limitations such as the use of multiple solvents and reagents, has limited efficacy for incorporating modified nucleotides, as well as a maximum length of synthetic oligonucleotides of about 150-200 bases.

These limitations have led to the search for alternative methods, such as enzymatic synthesis using template-independent DNA polymerases, which allow the controlled addition of reversibly terminated modified nucleotides to a growing nucleic acid chain. However, the native enzymes used in this process, such as terminal deoxynucleotidyl transferase (TdT), have limited efficacy in incorporating modified nucleotides, and particularly, reversibly terminated modified nucleotides.

Thus, several modified TdT have been described. For example, document US 2019/0390178 A1 describes TdT peptides from Mus musculus, Bos taurus, Homo sapiens, Gallus gallus, Monodelphis domestica, Elephantulus edwardii, Python bivittatus, Canis lupus, Condylura cristata, Ochotona princeps, Erinaceus europaeus, Tupaia chinensis, Ornithorhynchus anatinus, and Jaculus jaculus, having certain amino acid substitutions. The TdT peptides show better results on the extension percentage of DNA sequences than their respective wild-type enzymes. Document EP 1 350 842 A2 describes a truncated TdT derivative that is shorter than the wild type (from calf thymus) by up to 161 amino acids deleted from the N-terminus, and which has at least 20-fold increased enzymatic activity in a solution containing cobalt (II) ions compared to the wild-type TdT. Document US 2023/0357730 A1 describes a modified TdT from Lepisosteus oculatus having better ability to incorporate nucleotides with modifications.

Also, modified TdT with improved thermal stability has been described. For example, document CN 114921436 A describes a mutant TdT with improved thermal stability having six substitutions in the wild-type TdT sequence from Mus musculus. Specifically, the Tm value of the mutant TdT is 8° C. higher than the Tm value of the wild-type TdT.

Despite advances in the development of new TdT peptides, there are few technologies better than the chemical synthesis method due to the limitations of TdT that directly impact the turnaround time and cost of DNA or RNA synthesis products.

To date, there are no TdT peptides efficient and convenient enough to compete with chemical synthesis. Therefore, improved TdT are required to synthetize DNA and RNA sequences in an efficient and cost-effective manner. The compositions and methods provided herein, inter alia, address these and other problems in the art.

### BRIEF SUMMARY

In an aspect is provided a terminal deoxynucleotidyl transferase (TdT) peptide, wherein the peptide includes an amino acid sequence having at least 80% sequence identity to the amino acid sequence of SEQ ID NO:1.

In another aspect is provided a terminal deoxynucleotidyl transferase (TdT) peptide, wherein the peptide includes an amino acid sequence having at least 80% sequence identity to the amino acid sequence of SEQ ID NO:2.

In another aspect is provided a terminal deoxynucleotidyl transferase (TdT) peptide, wherein the peptide includes an amino acid sequence having at least 80% sequence identity to the amino acid sequence of SEQ ID NO:3.

In another aspect is provided a terminal deoxynucleotidyl transferase (TdT) peptide, wherein the peptide includes an amino acid sequence having at least 80% sequence identity to the amino acid sequence of SEQ ID NO:16.

In another aspect is provided a terminal deoxynucleotidyl transferase (TdT) peptide, wherein the peptide includes an amino acid sequence having at least 80% sequence identity to the amino acid sequence of SEQ ID NO:17.

In another aspect is provided a terminal deoxynucleotidyl transferase (TdT) peptide, wherein the peptide includes an amino acid sequence having at least 80% sequence identity to the amino acid sequence of SEQ ID NO:18.

In another aspect is provided a terminal deoxynucleotidyl transferase (TdT) peptide, wherein the peptide includes an amino acid sequence having at least 80% sequence identity to the amino acid sequence of SEQ ID NO:19.

In another aspect is provided a terminal deoxynucleotidyl transferase (TdT) peptide, wherein the peptide includes an amino acid sequence having at least 80% sequence identity to the amino acid sequence of SEQ ID NO:20.

In another aspect is provided a terminal deoxynucleotidyl transferase (TdT) peptide, wherein the peptide includes an amino acid sequence having at least 80% sequence identity to the amino acid sequence of SEQ ID NO:21.

In another aspect is provided a terminal deoxynucleotidyl transferase (TdT) peptide, wherein the peptide includes an amino acid sequence having at least 80% sequence identity to the amino acid sequence of SEQ ID NO:22.

In another aspect is provided a terminal deoxynucleotidyl transferase (TdT) peptide, wherein the peptide includes an amino acid sequence having at least 80% sequence identity to the amino acid sequence of SEQ ID NO:23.

In another aspect is provided a terminal deoxynucleotidyl transferase (TdT) peptide, wherein the peptide includes an amino acid sequence having at least 80% sequence identity to the amino acid sequence of SEQ ID NO:24.

In another aspect is provided a method for synthesizing an oligonucleotide, the method includes contacting the TdT peptide provided herein including embodiments thereof with (i) an oligonucleotide primer, wherein the oligonucleotide primer includes a free 3′-hydroxyl; and (ii) a nucleoside triphosphate or 3′-O-blocked nucleoside triphosphate under conditions that promote addition of the nucleotide triphosphate or 3′-O-blocked nucleoside triphosphate to the 3′ terminal end of the oligonucleotide primer, thereby forming an elongated oligonucleotide fragment.

In another aspect is provided a composition including the TdT peptide provided herein including embodiments thereof and an appropriate buffer.

In another aspect is provided a kit for synthesizing a target oligonucleotide, the kit including: a) the composition provided herein including embodiments thereof; b) a nucleotide triphosphate or a 3′-O-blocked nucleoside triphosphate; and c) an oligonucleotide primer including a free 3′-hydroxyl.

### BRIEF DESCRIPTION OF THE DRAWINGS

FIG. 1 Shows an agarose gel with 16 or 17 nt bands. The presence of 17 nt bands evidences the addition of the different modified nucleotides (blocked nucleotides, 3′-O—NH2) by the enzymes of the present invention compared to mouse TdT (mTdT) which shows 16 nt bands.

FIG. 2 Shows an agarose gel with the bands of ssDNA extensions with dNTPs produced by the TdT peptides of the present invention at a temperature of 37° C. for 30 minutes. 1 and 45 are control TdT peptides; 18 is SEQ ID NO:30; 20 is SEQ ID NO:32; 22 is SEQ ID NO: 25; 24 is SEQ ID NO:29; 25 is SEQ ID NO:27.

FIG. 3 Shows the ssDNA real time extension curves with a DNA intercalating dye. Reactions were incubated at 37° C. for 30 minutes. mTdT is mouse TdT; bTdT is bovine TdT; 24 is SEQ ID NO:29; and 22 is SEQ ID NO:25.

FIG. 4 Shows agarose gels with the bands of ssDNA extensions produced by the enzymes of the present invention at different temperatures for 30 minutes. mTdT is mouse TdT; TdT 24 is SEQ ID NO:29; and TdT 22 is SEQ ID NO:25.

FIG. 5 Shows the end-point quantification of the ssDNA extensions using an intercalating dye at different temperatures for enzymes of the present invention. mTdT is mouse TdT; TdT 24 is SEQ ID NO:29; and TdT 22 is SEQ ID NO:25.

FIG. 6 shows a graphic with the results of the activity of TdT peptides with natural deoxyribonucleotides. The x-axis shows the sequence number of TdT peptides, and the y-axis shows the number of turnovers for each deoxynucleotide tested. Bovine TdT (bTdT) was used as control.

FIG. 7 shows a graphic with the results of the activity of TdT peptides with natural ribonucleotides. The x-axis shows the sequence number of TdT peptides, and the y-axis shows the number of turnovers for each ribonucleotide tested. Bovine TdT (bTdT) was used as control.

FIG. 8 shows a graphic with the results of the activity of TdT peptides with blocked nucleotides. The x-axis shows the sequence number of TdT peptides, and the y-axis shows the number of turnovers for each blocked nucleotide tested. Bovine TdT (bTdT) was used as control.

FIG. 9 shows a graphic with the melting temperature (Tm) of each TdT peptide tested. The x-axis shows the sequence number of TdT peptides, and the y-axis shows the melting temperature (Tm) of each enzyme tested. Mouse TdT (mTdT) was used as control.

FIG. 10 shows data from a residual activity assay. TdT enzymes were incubated 15 min at different temperatures as displayed. 20 uM of enzymes were added to 2.5 uL reaction buffer and activity was measured at 37° C. 0.2 uM of starter oligo (random hexamer) was used, with 1 mM dNTPs and 4 uM of SYTO 13 as dye. Finally, after polymerization all enzymes were incubated 10 min at 70° C. as means to inactivate the enzyme.

### DETAILED DESCRIPTION

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine.; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

The term “modified nucleotides” is used herein according to its plain ordinary meaning and refers to nucleotides that have been chemically altered to include additional functional groups (e.g., fluorescent or biotinylated groups). In embodiments, the modified nucleotides are modified to enable their detection or purification. Typically, a nucleotide contains a single 5-carbon sugar moiety, a single nitrogenous base moiety and 1 to three phosphate moieties. In embodiments, a nucleotide includes a blocking moiety or a label moiety. In embodiments, a blocking moiety on a nucleotide prevents formation of a covalent bond between the 3′ hydroxyl moiety of the nucleotide and the 5′ phosphate of another nucleotide. In embodiments, a blocking moiety on a nucleotide can be reversible, whereby the blocking moiety can be removed or modified to allow the 3′ hydroxyl to form a covalent bond with the 5′ phosphate of another nucleotide. In embodiments, a blocking moiety can be effectively irreversible under particular conditions used in a method set forth herein. In embodiments, a label moiety of a nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both.

A “removable” group, e.g., a label or a blocking group or protecting group, refers to a chemical group that can be removed from a dNTP analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a dNTP of dNTP analogue.

“Reversible blocking groups” or “reversible terminators” include a blocking moiety located, for example, at the 3′ position of the nucleotide and may be a chemically cleavable moiety such as an allyl group, an azidomethyl group or a methoxymethyl group, or may be an enzymatically cleavable group such as a phosphate ester. Suitable nucleotide blocking moieties are described in applications WO 2004/018497, U.S. Pat. Nos. 7,057,026, 7,541,444, WO 96/07669, U.S. Pat. Nos. 5,763,594, 5,808,045, 5,872,244, and 6,232,465 the contents of which are incorporated herein by reference in their entirety. The nucleotides may be labelled or unlabelled. They may be modified with reversible terminators useful in methods provided herein and may be 3′-O-blocked reversible or 3′-unblocked reversible terminators. In nucleotides with 3′-O-blocked reversible terminators, the blocking group —OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH2 reversible terminator, a 3′-O-allyl reversible terminator, or a 3′-O-azidomethyl reversible terminator.

In embodiments, provided herein are polymerases capable of incorporating three differently sized reversible terminator probes linked to the 3′ oxygen: an A-Term, S-Term, and i-term. A-Term refers to azide-containing terminators (Guo J, et al. (2008) PNAS. Vol 105(27): 9145-9150); for example, having the formula:

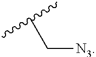 [SMILES: *CN=[N+]=[N-]]

S-Term refers to sulfide-containing terminators (WO 2017/058953); for example, having the formula

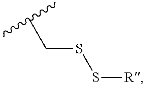

wherein R″ is unsubstituted C1-C4 alkyl. The i-term probe refers to an isomeric reversible terminator. For example, an i-term probe has the formula:

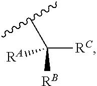

wherein R^{A} and R^{B} are hydrogen or alkyl, wherein at least one of R^{A} or R^{B} are hydrogen to yield a stereoisomeric probe, and R^{C} is the remainder of the reversible terminator.

In embodiments, the nucleotide is

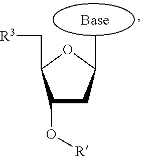

wherein Base is a Base as described herein, R³ is —OH, monophosphate, or polyphosphate or a nucleic acid, and R′ is a reversible terminator having the formula:

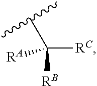

wherein R^{A} and R^{B} are hydrogen or alkyl and R^{C} is the remainder of the reversible terminator. In embodiments, the reversible terminator is

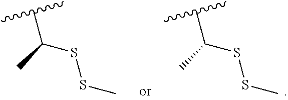

In embodiments, the reversible terminator is

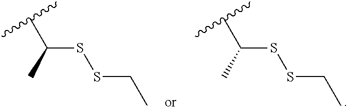

In embodiments, the reversible terminator is

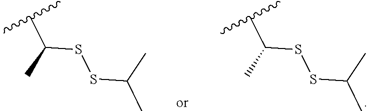

In embodiments, the reversible terminator is

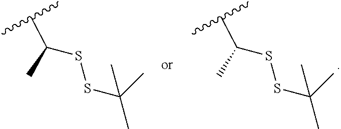

In embodiments, the reversible terminator is

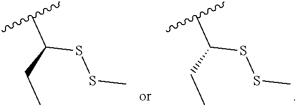

In embodiments, the reversible terminator is

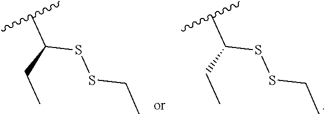

In embodiments, the reversible terminator is

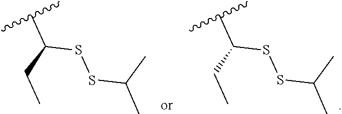

In embodiments, the reversible terminator is

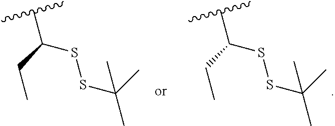

In embodiments, the reversible terminator is

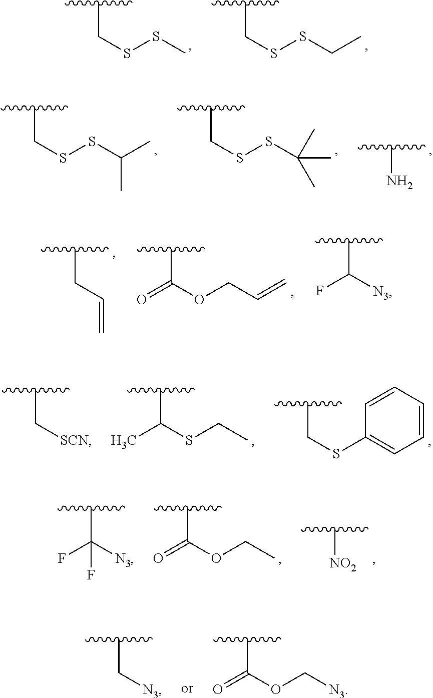

In nucleotides with 3′-unblocked reversible terminators, the termination group is linked to the base of the nucleotide as well as the label and functions not only as a reporter but as part of the reversible terminating group for termination of primer extension during sequencing. The 3′-unblocked reversible terminators are known in the art and include for example, the “virtual terminator” as described in U.S. Pat. No. 8,114,973 and the “Lightning terminator” as described in U.S. Pat. No. 10,041,115, the contents of which are incorporated herein by reference in their entirety.

The term “divalent cations” is used herein according to its plain ordinary meaning and refers to ions that carry a positive charge of +2 due to the loss of two electrons. In embodiments, divalent cations are formed when elements from the periodic table lose two valence electrons, resulting in a stable, positively charged ion. Common examples of divalent cations include calcium (Ca²⁺), magnesium (Mg²⁺), zinc (Zn²⁺), manganese (Mn²⁺), copper (Cu²⁺), nickel (Ni²⁺), iron (Fe²⁺), zinc (Zn²⁺), cobalt (Co²⁺), among others. Divalent cations play essential roles in various biological processes, such as enzyme function, cellular signaling, and the stabilization of macromolecular structures like nucleic acids and proteins.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “dNTP” is used herein according to its plain ordinary meaning and refers to deoxynucleoside triphosphate, the building block of DNA. In embodiments, dNTPs are comprised of a nitrogenous base, a deoxyribose sugar, and three phosphate groups. In embodiments, there are four types of nitrogenous bases in DNA: adenine (A), cytosine (C), guanine (G), and thymine (T). In embodiments, there are four types of dNTPs: dATP, dCTP, dGTP, and dTTP, each corresponding to their respective base.

The term “NTP” is used herein according to its plain ordinary meaning and refers to nucleoside triphosphate, the building block of RNA. In embodiments, NTPs are comprised of a nitrogenous base, a ribose sugar, and three phosphate groups. In embodiments, there are four types of nitrogenous bases in RNA: adenine (A), cytosine (C), guanine (G), and uracil (U). In embodiments, there are four types of NTPs: ATP, CTP, GTP, and UTP, each corresponding to their respective base.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety. In embodiments, the polypeptide is a TdT polypeptide. In embodiments, the peptide is a TdT peptide. In embodiments, the protein is a TdT protein. In embodiments, the TdT peptide is an enzyme. In embodiments, the TdT protein is an enzyme.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., Ras) in other proteins with different numbering systems. For example, by performing a simple sequence alignment with a protein (e.g., TdT) the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model corresponds to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

In embodiments, an amino acid in a TdT peptide may be conservatively substituted with an amino acid at the corresponding position in a reference TdT peptide. In embodiments this conservative substitution in the TdT peptide does not decrease the activity or function of the TdT peptide. In embodiments, the reference TdT peptide includes the amino acid sequence of SEQ ID NO:1, SEQ ID NO:2, SEQ ID NO:3, SEQ ID NO:16, SEQ ID NO: 17, SEQ ID NO:18, SEQ ID NO:19, SEQ ID NO:20, SEQ ID NO:21, SEQ ID NO:22, SEQ ID NO:23, or SEQ ID NO: 24. For example In embodiments, the reference TdT peptide includes the amino acid sequence of SEQ ID NO:1. In embodiments, the reference TdT peptide includes the amino acid sequence of SEQ ID NO:2. In embodiments, the reference TdT peptide includes the amino acid sequence of SEQ ID NO:3. In embodiments, the reference TdT peptide includes the amino acid sequence of SEQ ID NO:16. In embodiments, the reference TdT peptide includes the amino acid sequence of SEQ ID NO:17. In embodiments, the reference TdT peptide includes the amino acid sequence of SEQ ID NO:18. In embodiments, the reference TdT peptide includes the amino acid sequence of SEQ ID NO:19. In embodiments, the reference TdT peptide includes the amino acid sequence of SEQ ID NO:20. In embodiments, the reference TdT peptide includes the amino acid sequence of SEQ ID NO:21. In embodiments, the reference TdT peptide includes the amino acid sequence of SEQ ID NO:22. In embodiments, the reference TdT peptide includes the amino acid sequence of SEQ ID NO:23. In embodiments, the reference TdT peptide includes the amino acid sequence of SEQ ID NO:24. In embodiments, a TdT peptide including the amino acid sequence of any one of SEQ ID NOs: 1-3 or 16-24 can have an amino acid substituted for an amino acid at the corresponding position in any one of SEQ ID NOs: 1-3 or 16-24 as demonstrated in Table 1. For example, a TdT peptide including the amino acid sequence of SEQ ID NOs: 3, 16, 2, 17, or 18 can have the glutamic acid at position 2 substituted with a valine. Additionally, a TdT peptide including the amino acid sequence of SEQ ID NOs: 19, 20, 21, 22, 23, or 24 can have the valine at position 2 substituted with a glutamic acid. In embodiments, a TdT peptide including the amino acid sequence of SEQ ID NOs: 3, 16, 2, 17, 20, 21, 22, 23, or 24 can have the glutamine at position 3 substituted with a serine or an arginine. In embodiments, a TdT peptide including the amino acid sequence of SEQ ID NOs: 18 can have the serine at position 3 substituted with a glutamine. In embodiments, a TdT peptide including the amino acid sequence of SEQ ID NO:19 can have the arginine at position 3 substituted with a glutamine. In embodiments, each of the substitution of amino acid residues as set forth in Table 1 can be applied to a corresponding position in a TdT peptide including the amino acid sequence of any one of SEQ ID NOs: 1-3 or 16-24 as demonstrated in Table 1 and described herein.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)
(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST/or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. In embodiments, the percentage of sequence identity of a TdT peptide (e.g., a peptide comprising the amino acid sequence of any one of SEQ ID NOs: 1-3 or 16-24) provided herein only includes the percentage of sequence identity to the amino acids within SEQ ID NOs: 1-3 or 16-24. For example, a TdT peptide comprising at least 80% sequence identity with SEQ ID NO: 1 is said to have 80% sequence identity to SEQ ID NO:1 if it includes at least 80% of the amino acid sequence within SEQ ID NO:1. In embodiments, the percentage of sequence identity of the peptide does not include additional protein elements or protein domains in its calculation. For example, when a TdT peptide includes at least 80% sequence identity to SEQ ID NO:1, the 20% difference in the sequence identity to SEQ ID NO:1 cannot be due to the addition, deletion, insertion, or substitution of additional protein elements or protein domains. In embodiments, the percentage of sequence identity of a TdT peptide (e.g., a peptide comprising the amino acid sequence of any one of SEQ ID NOs:1-3 or 16-24) provided herein does not include a solubilizing amino acid sequence in its calculation. For example, when a peptide includes at least 80% sequence identity to SEQ ID NO:1, the 20% difference in sequence identity to SEQ ID NO: 1 cannot be due to the addition, deletion, insertion, or substitution of a solubilizing amino acid sequence. In embodiments, the percentage of sequence identity of a TdT peptide (e.g., a peptide comprising the amino acid sequence of any one of SEQ ID NOs: 1-3 or 16-24) provided herein does not include a reporter amino acid sequence in its calculation. In embodiments, the percentage of sequence identity of a TdT peptide (e.g., a peptide comprising the amino acid sequence of any one of SEQ ID NOs: 1-3 or 16-24) provided herein does not include a peptide linker in its calculation.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “deletion” refers to modification(s) to a polypeptide by removal of one or more amino acids from a reference polypeptide. In embodiments, deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining at least one of enzymatic activity or retaining the improved properties of a TdT peptide. In embodiments, deletions can be directed to the internal portions and/or terminal portions of the polypeptide. In embodiments, the deletion can comprise a continuous segment or can be discontinuous. In embodiments, the reference polypeptide is a naturally occurring TdT peptide or a recombinant TdT peptide. In embodiments, the reference polypeptide is a naturally occurring TdT peptide. In embodiments, the reference polypeptide is a recombinant TdT peptide. In embodiments, the reference polypeptide includes the amino acid sequence of SEQ ID NO:1, SEQ ID NO:2, SEQ ID NO:3, SEQ ID NO: 16, SEQ ID NO: 17, SEQ ID NO:18, SEQ ID NO: 19, SEQ ID NO:20, SEQ ID NO:21, SEQ ID NO:22, SEQ ID NO:23, or SEQ ID NO:24. In embodiments, the reference polypeptide includes the amino acid sequence of SEQ ID NO:1. In embodiments, the reference polypeptide includes the amino acid sequence of SEQ ID NO:2. In embodiments, the reference polypeptide includes the amino acid sequence of SEQ ID NO:3. In embodiments, the reference polypeptide includes the amino acid sequence of SEQ ID NO:16. In embodiments, the reference polypeptide includes the amino acid sequence of SEQ ID NO:17. In embodiments, the reference polypeptide includes the amino acid sequence of SEQ ID NO:18. In embodiments, the reference polypeptide includes the amino acid sequence of SEQ ID NO:19. In embodiments, the reference polypeptide includes the amino acid sequence of SEQ ID NO:20. In embodiments, the reference polypeptide includes the amino acid sequence of SEQ ID NO:21. In embodiments, the reference polypeptide includes the amino acid sequence of SEQ ID NO:22. In embodiments, the reference polypeptide includes the amino acid sequence of SEQ ID NO:23. In embodiments, the reference polypeptide includes the amino acid sequence of SEQ ID NO:24. In embodiments, a TdT peptide including the amino acid sequence of any one of SEQ ID NOs: 1-3 or 16-24 can have one or more amino acid residues deleted at a corresponding position in any one of SEQ ID NOs: 1-3 or 16-24 as demonstrated in Table 1. For example, a TdT peptide including the amino acid sequence of SEQ ID NO:2 or 17 can have the glutamine at position 4 deleted. Additionally, a TdT peptide including the amino acid sequence of SEQ ID NO:2 or 17 can have the glutamic acid at position 2, the glutamine at position 3, and the glutamine at position 4 deleted. In embodiments, each of the deletion of amino acid residues as set forth in Table 1 can be applied to a corresponding position in a TdT peptide including the amino acid sequence of any one of SEQ ID NOs: 1-3 or 16-24 as demonstrated in Table 1 and described herein.

The term “insertion” refers to modification(s) to a polypeptide by addition of one or more amino acids from a reference polypeptide. In embodiments, the novel TdT peptides comprise insertions of one or more amino acids to the naturally occurring polypeptide as well as insertions of one or more amino acids to other improved TdT polypeptides. In embodiments, insertions can occur in the internal portions of the polypeptide, or at the carboxy or amino terminus. In embodiments, insertions as used herein include fusion proteins as is known in the art. In embodiments, the insertion can be a contiguous segment of amino acids or separated by one or more of the amino acids in the naturally occurring polypeptide. In embodiments, the reference polypeptide is a naturally occurring TdT peptide or a recombinant TdT peptide. In embodiments, the reference polypeptide is a naturally occurring TdT peptide. In embodiments, the reference polypeptide is a recombinant TdT peptide. In embodiments, the reference polypeptide includes the amino acid sequence of SEQ ID NO:1, SEQ ID NO:2, SEQ ID NO:3, SEQ ID NO:16, SEQ ID NO:17, SEQ ID NO:18, SEQ ID NO:19, SEQ ID NO:20, SEQ ID NO: 21, SEQ ID NO:22, SEQ ID NO:23, or SEQ ID NO:24. In embodiments, the reference polypeptide includes the amino acid sequence of SEQ ID NO:1. In embodiments, the reference polypeptide includes the amino acid sequence of SEQ ID NO:2. In embodiments, the reference polypeptide includes the amino acid sequence of SEQ ID NO:3. In embodiments, the reference polypeptide includes the amino acid sequence of SEQ ID NO:16. In embodiments, the reference polypeptide includes the amino acid sequence of SEQ ID NO:17. In embodiments, the reference polypeptide includes the amino acid sequence of SEQ ID NO:18. In embodiments, the reference polypeptide includes the amino acid sequence of SEQ ID NO:19. In embodiments, the reference polypeptide includes the amino acid sequence of SEQ ID NO:20. In embodiments, the reference polypeptide includes the amino acid sequence of SEQ ID NO:21. In embodiments, the reference polypeptide includes the amino acid sequence of SEQ ID NO:22. In embodiments, the reference polypeptide includes the amino acid sequence of SEQ ID NO:23. In embodiments, the reference polypeptide includes the amino acid sequence of SEQ ID NO:24. In embodiments, a TdT peptide including the amino acid sequence of any one of SEQ ID NOs: 1-3 or 16-24 can have one or more amino acid residues inserted at a corresponding position in any one of SEQ ID NOs: 1-3 or 16-24 as demonstrated in Table 1. For example, a TdT peptide including the amino acid sequence of SEQ ID NO:1 can have a glutamine inserted at position 4. Additionally, a TdT peptide including the amino acid sequence of SEQ ID NO:1 can have a glutamic acid inserted at position 2, a glutamine inserted at position 3, and a glutamine inserted at position 4. In embodiments, each of the insertion of amino acid residues as set forth in Table 1 can be applied to a corresponding position in a TdT peptide including the amino acid sequence of any one of SEQ ID NOs:1-3 or 16-24 as demonstrated in Table 1 and described herein.

The term “solubilizing amino acid sequence” is used herein according to its plain ordinary meaning and refers to an amino acid sequence that increases solubility of a biomolecule. In embodiments the solubilizing amino acid sequence is attached to a peptide to increase its solubility. In embodiments, the solubilizing amino acid sequence does not alter the activity of the protein. In embodiments the solubilizing amino acid sequence is a maltose-binding protein (MBP) sequence, a small ubiquitin modified (SUMO) sequence, a thioredoxin (TrxA) sequence, a N-utilizing substance A (NusA) sequence, a Fasciola hepatica 8-kDa antigen (Fh8) sequence, a mutated dehalogenase (Halo) sequence, or a glutathione-S-transferase (GST) sequence. In embodiments, the solubilizing amino acid sequence includes the amino acid sequence of SEQ ID NO:41, SEQ ID NO:46, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO: 56, SEQ ID NO:57, or SEQ ID NO:58. In embodiments, the solubilizing amino acid sequence is attached to the N-terminus or C-terminus of a TdT amino acid sequence. IN embodiments, the solubilizing amino acid sequence is attached to the N-terminus or C-terminus of a reporter amino acid sequence. In embodiments, the solubilizing amino acid sequence is attached to a peptide through a peptide linker.

The term “peptide linker” is used herein according to its plain ordinary meaning and refers to an amino acid sequence between two peptides. In embodiments, the peptide linker may be between a solubilizing amino acid sequence and a TdT amino acid sequence. In embodiments, the peptide linker is between a solubilizing amino acid sequence and a reporter amino acid sequence. In embodiments, the peptide linker is between a reporter acid sequence and a TdT amino acid sequence. In embodiments, the peptide linker may be attached to the N-terminus or the C-terminus of a peptide. In embodiments, the peptide linker includes the amino acid sequence of SEQ ID NO:42, SEQ ID NO:44, SEQ ID NO:45, or SEQ ID NO:47.

The term “reporter amino acid sequence” is used herein according to its plain ordinary meaning and refers to an amino acid sequence which can be detected or measured experimentally. In embodiments, the reporter amino acid sequence is a label or detectable moiety. In embodiments, the reporter amino acid sequence is fluorescent protein. In embodiments, the reporter amino acid sequence is a green fluorescent protein. In embodiments, the reporter amino acid sequence is attached to the N-terminus or C-terminus of a peptide. In embodiments, the reporter amino acid sequence includes the amino acid sequence of SEQ ID NO: 43.

The term “terminal deoxynucleotidyl transferase (TdT) peptide” or “TdT amino acid sequence” is used herein according to its plain ordinary meaning and refers to a DNA polymerase, also known as nucleotidylexotransferase (DNTT) or terminal transferase, which maintains TdT activity (e.g., within at least 50%, 80%, 90%, 95%, 96%, 97%, 98%, 99% or 100% activity compared to the TdT protein). In embodiments, the TdT peptide has TdT enzymatic activity. In embodiments, the TdT peptide includes the amino acid sequence of SEQ ID NO: 1, SEQ ID NO:2, SEQ ID NO:3, SEQ ID NO:16, SEQ ID NO:17, SEQ ID NO:18, SEQ ID NO: 19, SEQ ID NO:20, SEQ ID NO:21, SEQ ID NO:22, SEQ ID NO:23, or SEQ ID NO:24.

The term “oligonucleotide primer” is used herein according to its plain ordinary meaning and refers to an oligonucleotide that acts as an initiator for oligonucleotide synthesis. In embodiments, the oligonucleotide primer includes a free 3′-hydroxyl. In embodiments, a TdT peptide catalyzes the addition of a nucleotide triphosphate or 3′-O-blocked nucleoside triphosphate to the 3′ terminal end of the oligonucleotide primer.

The term “elongated oligonucleotide fragment” is used herein according to its plain ordinary meaning and refers to an oligonucleotide synthesized by addition of nucleotide triphosphate or 3′-O-blocked nucleoside triphosphate to the 3′ terminal end of the oligonucleotide primer.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide specifically reactive with a target peptide. Any appropriate method known in the art for conjugating a peptide to the label may be employed, e.g., using methods described in Hermanson, Bioconjugate Techniques 1996, Academic Press, Inc., San Diego.

When the label or detectable moiety is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups. The chelate is normally linked to the PSMA antibody or functional antibody fragment by a group, which enables the formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium and copper, respectively. Other ring-type chelates such as macrocyclic polyethers, which are of interest for stably binding nuclides, such as ²²³Ra for RAIT may be used. In certain embodiments, chelating moieties may be used to attach a PET imaging agent, such as an A1-¹⁸F complex, to a targeting molecule for use in PET analysis.

“Contacting” is used in accordance with its plain ordinary meaning and refers to the process of allowing at least two distinct species (e.g. antibodies and antigens) to become sufficiently proximal to react, interact, or physically touch. It should be appreciated, however, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a pharmaceutical composition as provided herein and a cell. In embodiments contacting includes, for example, allowing a pharmaceutical composition as described herein to interact with a cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

The term “solid support” is used herein according to its plain ordinary meaning and refers to a material that allows the binding of molecules of interest, such as oligonucleotides or proteins. Examples of solid supports include, but are not limited to, agarose gels, polyacrylamide gels, magnetic beads, silica-based resins, glass slides, nitrocellulose membranes, and polystyrene or polypropylene multi-well plates. In embodiments, solid supports may be functionalized with a variety of chemical groups, such as amino, carboxyl, hydroxyl, or thiol groups, to facilitate the covalent or non-covalent attachment of the molecules of interest.

The term “reducing agent”, “reductant”, or “reducer” is used herein according to its plain ordinary meaning and refers to a chemical substance that donates electrons to another substance during a redox (reduction-oxidation) reaction. In embodiments, the reducing agent causes the reduction of a substance. In embodiments, the reducing agent itself undergoes oxidation during the process of donating electrons, which involves the loss of electrons. Reducing agents play a significant role in numerous chemical and biochemical reactions, including electron transfer processes, energy metabolism, and the synthesis or degradation of various biomolecules. Examples of reducing agents include, but are not limited to, hydrogen, metals, and organic compounds containing hydride or thiol groups. In embodiments, the reducing agent includes dithiothreitol, beta-mercaptoethanol, or tris(2-carboxyethyl)phosphine.

The term “detergent” is used herein according to its plain ordinary meaning and refers to a class of amphiphilic compounds that have both hydrophilic (water-soluble) and lipophilic (fat-soluble) properties. In embodiments, detergents are commonly used for solubilizing, emulsifying, or dispersing biological molecules, such as proteins, lipids, and nucleic acids. Detergents play a crucial role in various laboratory applications, including cell lysis, protein extraction, membrane protein solubilization, and the stabilization of protein-protein interactions. In embodiments, the chemical structure of detergents includes a polar head group and a hydrophobic tail. In embodiments, detergents are capable of interacting with both aqueous and lipid environments. In embodiments, the detergent is an anionic detergent, a cationic detergent, a nonionic detergent, or a zwitterionic detergent. Examples of suitable detergents include, but are not limited to, triton X-100, tween20, tween 80, sarkosyl, igepal CA-630, and NP-40.

The term “cryopreservative” or “cryoprotectant” is used herein according to its plain ordinary meaning and refers to a substance used to protect biological samples. In embodiments, the cryopreservative protects a biological sample from damage caused by the formation of ice crystals during the freezing process. Cryopreservation is a widely employed technique for the long-term storage of biological materials at extremely low temperatures, typically below the freezing point of water. In embodiments, cryopreservatives stabilizes molecules. In embodiments, cryopreservatives minimize ice crystal formation. In embodiments, cryopreservatives preserve the integrity and/or viability of a biological sample during freezing, storage, and subsequent thawing. In embodiments, the cryopreservative is glycerol, dimethyl sulfoxide (DMSO), ethylene glycol, or propylene glycol. In embodiments, the cryopreservative and its concentration may vary depending on the specific biological material being preserved and the desired preservation conditions. In embodiments the cryopreservative includes glycerol, trehalose, or mannitol.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Recombinant TdT Peptide Compositions

Provided herein are, inter alia, recombinant terminal deoxynucleotidyl transferase (TdT) peptides. The recombinant peptides provided herein include, for example, novel TdT enzymes and are, inter alia, useful for synthesizing oligonucleotides. The TdT peptides provided herein are capable of synthesizing oligonucleotides with high fidelity and efficiency. When compared with a commercial bovine TdT (bTdT), the TdT peptides provided herein had a surprisingly better activity using either natural nucleotides (dNTPs or NTPs) or blocked nucleotides (NH₂-dNTPs). Even more, several of the TdT peptides provided herein have an increased thermostability compared to the mouse TdT (mTdT). Thus, in an aspect is provided a terminal deoxynucleotidyl transferase (TdT) peptide, wherein the peptide includes an amino acid sequence having at least 80% sequence identity to the amino acid sequence of SEQ ID NO:1.

In embodiments, the peptide includes an amino acid sequence having at least 81% sequence identity to the amino acid sequence of SEQ ID NO:1. In embodiments, the peptide includes an amino acid sequence having at least 82% sequence identity to the amino acid sequence of SEQ ID NO:1. In embodiments, the peptide includes an amino acid sequence having at least 83% sequence identity to the amino acid sequence of SEQ ID NO:1. In embodiments, the peptide includes an amino acid sequence having at least 84% sequence identity to the amino acid sequence of SEQ ID NO:1. In embodiments, the peptide includes an amino acid sequence having at least 85% sequence identity to the amino acid sequence of SEQ ID NO:1. In embodiments, the peptide includes an amino acid sequence having at least 86% sequence identity to the amino acid sequence of SEQ ID NO:1. In embodiments, the peptide includes an amino acid sequence having at least 87% sequence identity to the amino acid sequence of SEQ ID NO: 1. In embodiments, the peptide includes an amino acid sequence having at least 88% sequence identity to the amino acid sequence of SEQ ID NO:1. In embodiments, the peptide includes an amino acid sequence having at least 89% sequence identity to the amino acid sequence of SEQ ID NO:1. In embodiments, the peptide includes an amino acid sequence having at least 90% sequence identity to the amino acid sequence of SEQ ID NO:1. In embodiments, the peptide includes an amino acid sequence having at least 91% sequence identity to the amino acid sequence of SEQ ID NO: 1. In embodiments, the peptide includes an amino acid sequence having at least 92% sequence identity to the amino acid sequence of SEQ ID NO:1. In embodiments, the peptide includes an amino acid sequence having at least 93% sequence identity to the amino acid sequence of SEQ ID NO:1. In embodiments, the peptide includes an amino acid sequence having at least 94% sequence identity to the amino acid sequence of SEQ ID NO:1. In embodiments, the peptide includes an amino acid sequence having at least 95% sequence identity to the amino acid sequence of SEQ ID NO: 1. In embodiments, the peptide includes an amino acid sequence having at least 96% sequence identity to the amino acid sequence of SEQ ID NO:1. In embodiments, the peptide includes an amino acid sequence having at least 97% sequence identity to the amino acid sequence of SEQ ID NO:1. In embodiments, the peptide includes an amino acid sequence having at least 98% sequence identity to the amino acid sequence of SEQ ID NO:1. In embodiments, the peptide includes an amino acid sequence having at least 99% sequence identity to the amino acid sequence of SEQ ID NO:1. In embodiments, the peptide includes an amino acid sequence having at least 100% sequence identity to the amino acid sequence of SEQ ID NO:1.

In embodiments, the peptide is an amino acid sequence having at least 80% sequence identity to the amino acid sequence of SEQ ID NO:1. In embodiments, the peptide is an amino acid sequence having at least 81% sequence identity to the amino acid sequence of SEQ ID NO: 1. In embodiments, the peptide is an amino acid sequence having at least 82% sequence identity to the amino acid sequence of SEQ ID NO:1. In embodiments, the peptide is an amino acid sequence having at least 83% sequence identity to the amino acid sequence of SEQ ID NO: 1. In embodiments, the peptide is an amino acid sequence having at least 84% sequence identity to the amino acid sequence of SEQ ID NO:1. In embodiments, the peptide is an amino acid sequence having at least 85% sequence identity to the amino acid sequence of SEQ ID NO: 1. In embodiments, the peptide is an amino acid sequence having at least 86% sequence identity to the amino acid sequence of SEQ ID NO:1. In embodiments, the peptide is an amino acid sequence having at least 87% sequence identity to the amino acid sequence of SEQ ID NO: 1. In embodiments, the peptide is an amino acid sequence having at least 88% sequence identity to the amino acid sequence of SEQ ID NO:1. In embodiments, the peptide is an amino acid sequence having at least 89% sequence identity to the amino acid sequence of SEQ ID NO: 1. In embodiments, the peptide is an amino acid sequence having at least 90% sequence identity to the amino acid sequence of SEQ ID NO:1. In embodiments, the peptide is an amino acid sequence having at least 91% sequence identity to the amino acid sequence of SEQ ID NO: 1. In embodiments, the peptide is an amino acid sequence having at least 92% sequence identity to the amino acid sequence of SEQ ID NO:1. In embodiments, the peptide is an amino acid sequence having at least 93% sequence identity to the amino acid sequence of SEQ ID NO: 1. In embodiments, the peptide is an amino acid sequence having at least 94% sequence identity to the amino acid sequence of SEQ ID NO:1. In embodiments, the peptide is an amino acid sequence having at least 95% sequence identity to the amino acid sequence of SEQ ID NO: 1. In embodiments, the peptide is an amino acid sequence having at least 96% sequence identity to the amino acid sequence of SEQ ID NO:1. In embodiments, the peptide is an amino acid sequence having at least 97% sequence identity to the amino acid sequence of SEQ ID NO: 1. In embodiments, the peptide is an amino acid sequence having at least 98% sequence identity to the amino acid sequence of SEQ ID NO:1. In embodiments, the peptide is an amino acid sequence having at least 99% sequence identity to the amino acid sequence of SEQ ID NO: 1. In embodiments, the peptide is an amino acid sequence having at least 100% sequence identity to the amino acid sequence of SEQ ID NO:1.

In embodiments, the peptide includes an amino acid sequence having 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to 250, 300, 350, 375, 380, 381, or 382 contiguous amino acids of the sequence of SEQ ID NO:1. In embodiments, the peptide includes a sequence having 80% sequence identity to 250, 300, 350, 375, 380, 381, or 382 contiguous amino acids of the sequence of SEQ ID NO:1. In embodiments, the peptide includes a sequence having 81% sequence identity to 250, 300, 350, 375, 380, 381, or 382 contiguous amino acids of the sequence of SEQ ID NO: 1. In embodiments, the peptide includes a sequence having 82% sequence identity to 250, 300, 350, 375, 380, 381, or 382 contiguous amino acids of the sequence of SEQ ID NO:1. In embodiments, the peptide includes a sequence having 83% sequence identity to 250, 300, 350, 375, 380, 381, or 382 contiguous amino acids of the sequence of SEQ ID NO:1. In embodiments, the peptide includes a sequence having 84% sequence identity to 250, 300, 350, 375, 380, 381, or 382 contiguous amino acids of the sequence of SEQ ID NO:1. In embodiments, the peptide includes a sequence having 85% sequence identity to 250, 300, 350, 375, 380, 381, or 382 contiguous amino acids of the sequence of SEQ ID NO:1. In embodiments, the peptide includes a sequence having 86% sequence identity to 250, 300, 350, 375, 380, 381, or 382 contiguous amino acids of the sequence of SEQ ID NO:1. In embodiments, the peptide includes a sequence having 87% sequence identity to 250, 300, 350, 375, 380, 381, or 382 contiguous amino acids of the sequence of SEQ ID NO:1. In embodiments, the peptide includes a sequence having 88% sequence identity to 250, 300, 350, 375, 380, 381, or 382 contiguous amino acids of the sequence of SEQ ID NO:1. In embodiments, the peptide includes a sequence having 89% sequence identity to 250, 300, 350, 375, 380, 381, or 382 contiguous amino acids of the sequence of SEQ ID NO: 1. In embodiments, the peptide includes a sequence having 90% sequence identity to 250, 300, 350, 375, 380, 381, or 382 contiguous amino acids of the sequence of SEQ ID NO:1. In embodiments, the peptide includes a sequence having 91% sequence identity to 250, 300, 350, 375, 380, 381, or 382 contiguous amino acids of the sequence of SEQ ID NO:1. In embodiments, the peptide includes a sequence having 92% sequence identity to 250, 300, 350, 375, 380, 381, or 382 contiguous amino acids of the sequence of SEQ ID NO: 1. In embodiments, the peptide includes a sequence having 93% sequence identity to 250, 300, 350, 375, 380, 381, or 382 contiguous amino acids of the sequence of SEQ ID NO:1. In embodiments, the peptide includes a sequence having 94% sequence identity to 250, 300, 350, 375, 380, 381, or 382 contiguous amino acids of the sequence of SEQ ID NO:1. In embodiments, the peptide includes a sequence having 95% sequence identity to 250, 300, 350, 375, 380, 381, or 382 contiguous amino acids of the sequence of SEQ ID NO:1. In embodiments, the peptide includes a sequence having 96% sequence identity to 250, 300, 350, 375, 380, 381, or 382 contiguous amino acids of the sequence of SEQ ID NO:1. In embodiments, the peptide includes a sequence having 97% sequence identity to 250, 300, 350, 375, 380, 381, or 382 contiguous amino acids of the sequence of SEQ ID NO: 1. In embodiments, the peptide includes a sequence having 98% sequence identity to 250, 300, 350, 375, 380, 381, or 382 contiguous amino acids of the sequence of SEQ ID NO:1. In embodiments, the peptide includes a sequence having 99% sequence identity to 250, 300, 350, 375, 380, 381, or 382 contiguous amino acids of the sequence of SEQ ID NO:1. In embodiments, the peptide includes a sequence having 100% sequence identity to 250, 300, 350, 375, 380, 381, or 382 contiguous amino acids of the sequence of SEQ ID NO:1.

In embodiments, the peptide includes the amino acid sequence of SEQ ID NO:1. In embodiments, the peptide has the amino acid sequence of SEQ ID NO:1. In one further embodiment, the peptide is referred to as N10.

In embodiments, the peptide may further include additional protein elements or protein domains. In embodiments, the peptide further includes additional protein elements. In embodiments, the peptide further includes additional protein domains. In embodiments, the additional protein elements or protein domains may include solubilizing amino acid sequences, reporter amino acid sequences, peptide linkers, labels, label moieties, or detectable moieities. In embodiments, the additional protein domain includes a solubilizing amino acid sequence, a reporter amino acid sequence, a peptide linker, a label, a label moiety, or a detectable moiety. In embodiments, the additional protein domain includes a solubilizing amino acid sequence. In embodiments, the additional protein domain includes a reporter amino acid sequence. In embodiments, the additional protein domain includes a peptide linker. In embodiments, the additional protein domain includes a label. In embodiments, the additional protein domain includes a label moiety. In embodiments, the additional protein domain includes a detectable moiety.

In embodiments, the peptide further includes a solubilizing amino acid sequence, wherein the solubilizing amino acid sequence is a maltose-binding protein (MBP) sequence, a small ubiquitin modified (SUMO) sequence, a thioredoxin (TrxA) sequence, a N-utilizing substance A (NusA) sequence, a Fasciola hepatica 8-kDa antigen (Fh8) sequence, a mutated dehalogenase (Halo) sequence, or a glutathione-S-transferase (GST) sequence.

In embodiments, the peptide further includes a solubilizing amino acid sequence, wherein the solubilizing amino acid sequence is a maltose-binding protein (MBP) sequence. In embodiments, the peptide further includes a solubilizing amino acid sequence, wherein the solubilizing amino acid sequence is a small ubiquitin modified (SUMO) sequence. In embodiments, the peptide further includes a solubilizing amino acid sequence, wherein the solubilizing amino acid sequence is a thioredoxin (TrxA) sequence. In embodiments, the peptide further includes a solubilizing amino acid sequence, wherein the solubilizing amino acid sequence is a N-utilizing substance A (NusA) sequence. In embodiments, the peptide further includes a solubilizing amino acid sequence, wherein the solubilizing amino acid sequence is a Fasciola hepatica 8-kDa antigen (Fh8) sequence. In embodiments, the peptide further includes a solubilizing amino acid sequence, wherein the solubilizing amino acid sequence is a mutated dehalogenase (Halo) sequence. In embodiments, the peptide further includes a solubilizing amino acid sequence, wherein the solubilizing amino acid sequence is a glutathione-S-transferase (GST) sequence.

In embodiments, the peptide further includes a peptide linker. In embodiments, the peptide linker includes the amino acid sequence of SEQ ID NO:42, SEQ ID NO:44, SEQ ID NO: 45, or SEQ ID NO:47. In embodiments, the peptide linker includes the amino acid sequence of SEQ ID NO:42. In embodiments, the peptide linker includes the amino acid sequence of SEQ ID NO: 44. In embodiments, the peptide linker includes the amino acid sequence of SEQ ID NO: 45. In embodiments, the peptide linker includes the amino acid sequence of SEQ ID NO:47. In embodiments, the peptide linker is the amino acid sequence of SEQ ID NO:42, SEQ ID NO: 44, SEQ ID NO:45, or SEQ ID NO:47. In embodiments, the peptide linker is the amino acid sequence of SEQ ID NO:42. In embodiments, the peptide linker is the amino acid sequence of SEQ ID NO:44. In embodiments, the peptide linker is the amino acid sequence of SEQ ID NO: 45. In embodiments, the peptide linker is the amino acid sequence of SEQ ID NO:47.

In embodiments, the peptide includes a solubilizing amino acid sequence attached to the amino acid sequence of SEQ ID NO:1 through a peptide linker. In embodiments, the solubilizing amino acid sequence includes the amino acid sequence of SEQ ID NO:41 and the peptide linker includes the amino acid sequence of SEQ ID NO:42. In embodiments, the peptide includes the amino acid sequence of SEQ ID NO:25. In embodiments, the peptide is the amino acid sequence of SEQ ID NO:25. In one further embodiment, the peptide is referred to as MBP_N62. In one embodiment, the peptide is referred to as TdT 22 or clone 22.

In another aspect is provided a terminal deoxynucleotidyl transferase (TdT) peptide, wherein the peptide includes an amino acid sequence having at least 80% sequence identity to the amino acid sequence of SEQ ID NO:2. In embodiments, the peptide includes an amino acid sequence having at least 81% sequence identity to the amino acid sequence of SEQ ID NO:2. In embodiments, the peptide includes an amino acid sequence having at least 82% sequence identity to the amino acid sequence of SEQ ID NO:2. In embodiments, the peptide includes an amino acid sequence having at least 83% sequence identity to the amino acid sequence of SEQ ID NO: 2. In embodiments, the peptide includes an amino acid sequence having at least 84% sequence identity to the amino acid sequence of SEQ ID NO:2. In embodiments, the peptide includes an amino acid sequence having at least 85% sequence identity to the amino acid sequence of SEQ ID NO:2. In embodiments, the peptide includes an amino acid sequence having at least 86% sequence identity to the amino acid sequence of SEQ ID NO:2. In embodiments, the peptide includes an amino acid sequence having at least 87% sequence identity to the amino acid sequence of SEQ ID NO:2. In embodiments, the peptide includes an amino acid sequence having at least 88% sequence identity to the amino acid sequence of SEQ ID NO:2. In embodiments, the peptide includes an amino acid sequence having at least 89% sequence identity to the amino acid sequence of SEQ ID NO:2. In embodiments, the peptide includes an amino acid sequence having at least 90% sequence identity to the amino acid sequence of SEQ ID NO:2. In embodiments, the peptide includes an amino acid sequence having at least 91% sequence identity to the amino acid sequence of SEQ ID NO:2. In embodiments, the peptide includes an amino acid sequence having at least 92% sequence identity to the amino acid sequence of SEQ ID NO:2. In embodiments, the peptide includes an amino acid sequence having at least 93% sequence identity to the amino acid sequence of SEQ ID NO:2. In embodiments, the peptide includes an amino acid sequence having at least 94% sequence identity to the amino acid sequence of SEQ ID NO:2. In embodiments, the peptide includes an amino acid sequence having at least 95% sequence identity to the amino acid sequence of SEQ ID NO:2. In embodiments, the peptide includes an amino acid sequence having at least 96% sequence identity to the amino acid sequence of SEQ ID NO:2. In embodiments, the peptide includes an amino acid sequence having at least 97% sequence identity to the amino acid sequence of SEQ ID NO:2. In embodiments, the peptide includes an amino acid sequence having at least 98% sequence identity to the amino acid sequence of SEQ ID NO:2. In embodiments, the peptide includes an amino acid sequence having at least 99% sequence identity to the amino acid sequence of SEQ ID NO:2. In embodiments, the peptide includes an amino acid sequence having at least 100% sequence identity to the amino acid sequence of SEQ ID NO:2.

In embodiments, the peptide is an amino acid sequence having at least 80% sequence identity to the amino acid sequence of SEQ ID NO:2. In embodiments, the peptide is an amino acid sequence having at least 81% sequence identity to the amino acid sequence of SEQ ID NO: 2. In embodiments, the peptide is an amino acid sequence having at least 82% sequence identity to the amino acid sequence of SEQ ID NO:2. In embodiments, the peptide is an amino acid sequence having at least 83% sequence identity to the amino acid sequence of SEQ ID NO: 2. In embodiments, the peptide is an amino acid sequence having at least 84% sequence identity to the amino acid sequence of SEQ ID NO:2. In embodiments, the peptide is an amino acid sequence having at least 85% sequence identity to the amino acid sequence of SEQ ID NO: 2. In embodiments, the peptide is an amino acid sequence having at least 86% sequence identity to the amino acid sequence of SEQ ID NO:2. In embodiments, the peptide is an amino acid sequence having at least 87% sequence identity to the amino acid sequence of SEQ ID NO: 2. In embodiments, the peptide is an amino acid sequence having at least 88% sequence identity to the amino acid sequence of SEQ ID NO:2. In embodiments, the peptide is an amino acid sequence having at least 89% sequence identity to the amino acid sequence of SEQ ID NO: 2. In embodiments, the peptide is an amino acid sequence having at least 90% sequence identity to the amino acid sequence of SEQ ID NO:2. In embodiments, the peptide is an amino acid sequence having at least 91% sequence identity to the amino acid sequence of SEQ ID NO: 2. In embodiments, the peptide is an amino acid sequence having at least 92% sequence identity to the amino acid sequence of SEQ ID NO:2. In embodiments, the peptide is an amino acid sequence having at least 93% sequence identity to the amino acid sequence of SEQ ID NO: 2. In embodiments, the peptide is an amino acid sequence having at least 94% sequence identity to the amino acid sequence of SEQ ID NO:2. In embodiments, the peptide is an amino acid sequence having at least 95% sequence identity to the amino acid sequence of SEQ ID NO: 2. In embodiments, the peptide is an amino acid sequence having at least 96% sequence identity to the amino acid sequence of SEQ ID NO:2. In embodiments, the peptide is an amino acid sequence having at least 97% sequence identity to the amino acid sequence of SEQ ID NO: 2. In embodiments, the peptide is an amino acid sequence having at least 98% sequence identity to the amino acid sequence of SEQ ID NO:2. In embodiments, the peptide is an amino acid sequence having at least 99% sequence identity to the amino acid sequence of SEQ ID NO: 2. In embodiments, the peptide is an amino acid sequence having at least 100% sequence identity to the amino acid sequence of SEQ ID NO:2.

In embodiments, the peptide includes an amino acid sequence having 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to 250, 300, 350, 375, 380, 381, 382, 383, 384, 385, 386, 387, 388 contiguous amino acids of the sequence of SEQ ID NO:2. In embodiments, the peptide includes a sequence having 80% sequence identity to 250, 300, 350, 375, 380, 381, 382, 383, 384, 385, 386, 387, 388 contiguous amino acids of the sequence of SEQ ID NO:2. In embodiments, the peptide includes a sequence having 81% sequence identity to 250, 300, 350, 375, 380, 381, 382, 383, 384, 385, 386, 387, 388 contiguous amino acids of the sequence of SEQ ID NO: 2. In embodiments, the peptide includes a sequence having 82% sequence identity to 250, 300, 350, 375, 380, 381, 382, 383, 384, 385, 386, 387, 388 contiguous amino acids of the sequence of SEQ ID NO:2. In embodiments, the peptide includes a sequence having 83% sequence identity to 250, 300, 350, 375, 380, 381, 382, 383, 384, 385, 386, 387, 388 contiguous amino acids of the sequence of SEQ ID NO:2. In embodiments, the peptide includes a sequence having 84% sequence identity to 250, 300, 350, 375, 380, 381, 382, 383, 384, 385, 386, 387, 388 contiguous amino acids of the sequence of SEQ ID NO:2. In embodiments, the peptide includes a sequence having 85% sequence identity to 250, 300, 350, 375, 380, 381, 382, 383, 384, 385, 386, 387, 388 contiguous amino acids of the sequence of SEQ ID NO:2. In embodiments, the peptide includes a sequence having 86% sequence identity to 250, 300, 350, 375, 380, 381, 382, 383, 384, 385, 386, 387, 388 contiguous amino acids of the sequence of SEQ ID NO:2. In embodiments, the peptide includes a sequence having 87% sequence identity to 250, 300, 350, 375, 380, 381, 382, 383, 384, 385, 386, 387, 388 contiguous amino acids of the sequence of SEQ ID NO: 2. In embodiments, the peptide includes a sequence having 88% sequence identity to 250, 300, 350, 375, 380, 381, 382, 383, 384, 385, 386, 387, 388 contiguous amino acids of the sequence of SEQ ID NO:2. In embodiments, the peptide includes a sequence having 89% sequence identity 250, 300, 350, 375, 380, 381, 382, 383, 384, 385, 386, 387, 388 contiguous amino acids of the sequence of SEQ ID NO:2. In embodiments, the peptide includes a sequence having 90% sequence identity to 250, 300, 350, 375, 380, 381, 382, 383, 384, 385, 386, 387, 388 contiguous amino acids of the sequence of SEQ ID NO:2. In embodiments, the peptide includes a sequence having 91% sequence identity to 250, 300, 350, 375, 380, 381, 382, 383, 384, 385, 386, 387, 388 contiguous amino acids of the sequence of SEQ ID NO:2. In embodiments, the peptide includes a sequence having 92% sequence identity to 250, 300, 350, 375, 380, 381, 382, 383, 384, 385, 386, 387, 388 contiguous amino acids of the sequence of SEQ ID NO:2. In embodiments, the peptide includes a sequence having 93% sequence identity to 250, 300, 350, 375, 380, 381, 382, 383, 384, 385, 386, 387, 388 contiguous amino acids of the sequence of SEQ ID NO: 2. In embodiments, the peptide includes a sequence having 94% sequence identity to 250, 300, 350, 375, 380, 381, 382, 383, 384, 385, 386, 387, 388 contiguous amino acids of the sequence of SEQ ID NO:2. In embodiments, the peptide includes a sequence having 95% sequence identity to 250, 300, 350, 375, 380, 381, 382, 383, 384, 385, 386, 387, 388 contiguous amino acids of the sequence of SEQ ID NO:2. In embodiments, the peptide includes a sequence having 96% sequence identity to 250, 300, 350, 375, 380, 381, 382, 383, 384, 385, 386, 387, 388 contiguous amino acids of the sequence of SEQ ID NO:2. In embodiments, the peptide includes a sequence having 97% sequence identity to 250, 300, 350, 375, 380, 381, 382, 383, 384, 385, 386, 387, 388 contiguous amino acids of the sequence of SEQ ID NO:2. In embodiments, the peptide includes a sequence having 98% sequence identity to 250, 300, 350, 375, 380, 381, 382, 383, 384, 385, 386, 387, 388 contiguous amino acids of the sequence of SEQ ID NO:2. In embodiments, the peptide includes a sequence having 99% sequence identity to 250, 300, 350, 375, 380, 381, 382, 383, 384, 385, 386, 387, 388 contiguous amino acids of the sequence of SEQ ID NO: 2. In embodiments, the peptide includes a sequence having 100% sequence identity to 250, 300, 350, 375, 380, 381, 382, 383, 384, 385, 386, 387, 388 contiguous amino acids of the sequence of SEQ ID NO:2.

In embodiments, the peptide includes the amino acid sequence of SEQ ID NO:2. In embodiments, the peptide has the amino acid sequence of SEQ ID NO:2. In one further embodiment, the peptide is referred to as N54.

In embodiments, the peptide may further include additional protein elements or protein domains. In embodiments, the peptide further includes additional protein elements. In embodiments, the peptide further includes additional protein domains. In embodiments, the additional protein elements or protein domains may include solubilizing amino acid sequences, reporter amino acid sequences, labels, label moieties, or detectable moieities. In embodiments, the additional protein domains include a solubilizing amino acid sequence, a reporter amino acid sequence, a label, a label moiety, or a detectable moiety. In embodiments, the additional protein domain includes a solubilizing amino acid sequence. In embodiments, the additional protein domain includes a reporter amino acid sequence. In embodiments, the additional protein domain includes a label. In embodiments, the additional protein domain includes a label moiety. In embodiments, the additional protein domain includes a detectable moiety.

In embodiments, the peptide further includes a solubilizing amino acid sequence, wherein the solubilizing amino acid sequence is a maltose-binding protein (MBP) sequence, a small ubiquitin modified (SUMO) sequence, a thioredoxin (TrxA) sequence, a N-utilizing substance A (NusA) sequence, a Fasciola hepatica 8-kDa antigen (Fh8) sequence, a mutated dehalogenase (Halo) sequence, or a glutathione-S-transferase (GST) sequence.

In embodiments, the peptide further includes a solubilizing amino acid sequence, wherein the solubilizing amino acid sequence is a maltose-binding protein (MBP) sequence. In embodiments, the peptide further includes a solubilizing amino acid sequence, wherein the solubilizing amino acid sequence is a small ubiquitin modified (SUMO) sequence. In embodiments, the peptide further includes a solubilizing amino acid sequence, wherein the solubilizing amino acid sequence is a thioredoxin (TrxA) sequence. In embodiments, the peptide further includes a solubilizing amino acid sequence, wherein the solubilizing amino acid sequence is a N-utilizing substance A (NusA) sequence. In embodiments, the peptide further includes a solubilizing amino acid sequence, wherein the solubilizing amino acid sequence is a Fasciola hepatica 8-kDa antigen (Fh8) sequence. In embodiments, the peptide further includes a solubilizing amino acid sequence, wherein the solubilizing amino acid sequence is a mutated dehalogenase (Halo) sequence. In embodiments, the peptide further includes a solubilizing amino acid sequence, wherein the solubilizing amino acid sequence is a glutathione-S-transferase (GST) sequence.

In embodiments, the peptide further includes a peptide linker. In embodiments, the peptide linker includes the amino acid sequence of SEQ ID NO:42, SEQ ID NO:44, SEQ ID NO: 45, or SEQ ID NO:47. In embodiments, the peptide linker includes the amino acid sequence of SEQ ID NO:42. In embodiments, the peptide linker includes the amino acid sequence of SEQ ID NO: 44. In embodiments, the peptide linker includes the amino acid sequence of SEQ ID NO: 45. In embodiments, the peptide linker includes the amino acid sequence of SEQ ID NO:47. In embodiments, the peptide linker is the amino acid sequence of SEQ ID NO:42, SEQ ID NO: 44, SEQ ID NO:45, or SEQ ID NO:47. In embodiments, the peptide linker is the amino acid sequence of SEQ ID NO:42. In embodiments, the peptide linker is the amino acid sequence of SEQ ID NO:44. In embodiments, the peptide linker is the amino acid sequence of SEQ ID NO: 45. In embodiments, the peptide linker is the amino acid sequence of SEQ ID NO:47.

In embodiments, the peptide includes a solubilizing amino acid sequence attached to the amino acid sequence of SEQ ID NO:2 through a peptide linker. In embodiments, the solubilizing amino acid sequence includes the amino acid sequence of SEQ ID NO:41 and the peptide linker includes the amino acid sequence of SEQ ID NO:42. In embodiments, the peptide includes the amino acid sequence of SEQ ID NO:29. In embodiments, the peptide is the amino acid sequence of SEQ ID NO:29. In one further embodiment, the peptide is referred to as MBP_N10. In one embodiment, the peptide is referred to as TdT 24 or clone 24.

In another aspect is provided a terminal deoxynucleotidyl transferase (TdT) peptide, wherein the peptide includes an amino acid sequence having at least 80% sequence identity to the amino acid sequence of SEQ ID NO:3. In embodiments, the peptide includes an amino acid sequence having at least 81% sequence identity to the amino acid sequence of SEQ ID NO:3. In embodiments, the peptide includes an amino acid sequence having at least 82% sequence identity to the amino acid sequence of SEQ ID NO:3. In embodiments, the peptide includes an amino acid sequence having at least 83% sequence identity to the amino acid sequence of SEQ ID NO: 3. In embodiments, the peptide includes an amino acid sequence having at least 84% sequence identity to the amino acid sequence of SEQ ID NO:3. In embodiments, the peptide includes an amino acid sequence having at least 85% sequence identity to the amino acid sequence of SEQ ID NO:3. In embodiments, the peptide includes an amino acid sequence having at least 86% sequence identity to the amino acid sequence of SEQ ID NO:3. In embodiments, the peptide includes an amino acid sequence having at least 87% sequence identity to the amino acid sequence of SEQ ID NO:3. In embodiments, the peptide includes an amino acid sequence having at least 88% sequence identity to the amino acid sequence of SEQ ID NO:3. In embodiments, the peptide includes an amino acid sequence having at least 89% sequence identity to the amino acid sequence of SEQ ID NO:3. In embodiments, the peptide includes an amino acid sequence having at least 90% sequence identity to the amino acid sequence of SEQ ID NO:3. In embodiments, the peptide includes an amino acid sequence having at least 91% sequence identity to the amino acid sequence of SEQ ID NO:3. In embodiments, the peptide includes an amino acid sequence having at least 92% sequence identity to the amino acid sequence of SEQ ID NO:3. In embodiments, the peptide includes an amino acid sequence having at least 93% sequence identity to the amino acid sequence of SEQ ID NO:3. In embodiments, the peptide includes an amino acid sequence having at least 94% sequence identity to the amino acid sequence of SEQ ID NO:3. In embodiments, the peptide includes an amino acid sequence having at least 95% sequence identity to the amino acid sequence of SEQ ID NO:3. In embodiments, the peptide includes an amino acid sequence having at least 96% sequence identity to the amino acid sequence of SEQ ID NO:3. In embodiments, the peptide includes an amino acid sequence having at least 97% sequence identity to the amino acid sequence of SEQ ID NO:3. In embodiments, the peptide includes an amino acid sequence having at least 98% sequence identity to the amino acid sequence of SEQ ID NO:3. In embodiments, the peptide includes an amino acid sequence having at least 99% sequence identity to the amino acid sequence of SEQ ID NO:3. In embodiments, the peptide includes an amino acid sequence having at least 100% sequence identity to the amino acid sequence of SEQ ID NO:3.

In embodiments, the peptide is an amino acid sequence having at least 80% sequence identity to the amino acid sequence of SEQ ID NO:3. In embodiments, the peptide is an amino acid sequence having at least 81% sequence identity to the amino acid sequence of SEQ ID NO: 3. In embodiments, the peptide is an amino acid sequence having at least 82% sequence identity to the amino acid sequence of SEQ ID NO:3. In embodiments, the peptide is an amino acid sequence having at least 83% sequence identity to the amino acid sequence of SEQ ID NO: 3. In embodiments, the peptide is an amino acid sequence having at least 84% sequence identity to the amino acid sequence of SEQ ID NO:3. In embodiments, the peptide is an amino acid sequence having at least 85% sequence identity to the amino acid sequence of SEQ ID NO: 3. In embodiments, the peptide is an amino acid sequence having at least 86% sequence identity to the amino acid sequence of SEQ ID NO:3. In embodiments, the peptide is an amino acid sequence having at least 87% sequence identity to the amino acid sequence of SEQ ID NO: 3. In embodiments, the peptide is an amino acid sequence having at least 88% sequence identity to the amino acid sequence of SEQ ID NO:3. In embodiments, the peptide is an amino acid sequence having at least 89% sequence identity to the amino acid sequence of SEQ ID NO: 3. In embodiments, the peptide is an amino acid sequence having at least 90% sequence identity to the amino acid sequence of SEQ ID NO:3. In embodiments, the peptide is an amino acid sequence having at least 91% sequence identity to the amino acid sequence of SEQ ID NO: 3. In embodiments, the peptide is an amino acid sequence having at least 92% sequence identity to the amino acid sequence of SEQ ID NO:3. In embodiments, the peptide is an amino acid sequence having at least 93% sequence identity to the amino acid sequence of SEQ ID NO: 3. In embodiments, the peptide is an amino acid sequence having at least 94% sequence identity to the amino acid sequence of SEQ ID NO:3. In embodiments, the peptide is an amino acid sequence having at least 95% sequence identity to the amino acid sequence of SEQ ID NO: 3. In embodiments, the peptide is an amino acid sequence having at least 96% sequence identity to the amino acid sequence of SEQ ID NO:3. In embodiments, the peptide is an amino acid sequence having at least 97% sequence identity to the amino acid sequence of SEQ ID NO: 3. In embodiments, the peptide is an amino acid sequence having at least 98% sequence identity to the amino acid sequence of SEQ ID NO:3. In embodiments, the peptide is an amino acid sequence having at least 99% sequence identity to the amino acid sequence of SEQ ID NO: 3. In embodiments, the peptide is an amino acid sequence having at least 100% sequence identity to the amino acid sequence of SEQ ID NO:3.

In embodiments, the peptide includes an amino acid sequence having 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to 250, 300, 350, 375, 380, 381, 382, 383, 384, 385, 386, or 387 contiguous amino acids of the sequence of SEQ ID NO:3. In embodiments, the peptide includes a sequence having 80% sequence identity to 250, 300, 350, 375, 380, 381, 382, 383, 384, 385, 386, or 387 contiguous amino acids of the sequence of SEQ ID NO:3. In embodiments, the peptide includes a sequence having 81% sequence identity to 250, 300, 350, 375, 380, 381, 382, 383, 384, 385, 386, or 387 contiguous amino acids of the sequence of SEQ ID NO:3. In embodiments, the peptide includes a sequence having 82% sequence identity to 250, 300, 350, 375, 380, 381, 382, 383, 384, 385, 386, or 387 contiguous amino acids of the sequence of SEQ ID NO: 3. In embodiments, the peptide includes a sequence having 83% sequence identity to 250, 300, 350, 375, 380, 381, 382, 383, 384, 385, 386, or 387 contiguous amino acids of the sequence of SEQ ID NO:3. In embodiments, the peptide includes a sequence having 84% sequence identity to 250, 300, 350, 375, 380, 381, 382, 383, 384, 385, 386, or 387 contiguous amino acids of the sequence of SEQ ID NO:3. In embodiments, the peptide includes a sequence having 85% sequence identity to 250, 300, 350, 375, 380, 381, 382, 383, 384, 385, 386, or 387 contiguous amino acids of the sequence of SEQ ID NO:3. In embodiments, the peptide includes a sequence having 86% sequence identity to 250, 300, 350, 375, 380, 381, 382, 383, 384, 385, 386, or 387 contiguous amino acids of the sequence of SEQ ID NO:3. In embodiments, the peptide includes a sequence having 87% sequence identity to 250, 300, 350, 375, 380, 381, 382, 383, 384, 385, 386, or 387 contiguous amino acids of the sequence of SEQ ID NO:3. In embodiments, the peptide includes a sequence having 88% sequence identity to 250, 300, 350, 375, 380, 381, 382, 383, 384, 385, 386, or 387 contiguous amino acids of the sequence of SEQ ID NO:3. In embodiments, the peptide includes a sequence having 89% sequence identity to 250, 300, 350, 375, 380, 381, 382, 383, 384, 385, 386, or 387 contiguous amino acids of the sequence of SEQ ID NO: 3. In embodiments, the peptide includes a sequence having 90% sequence identity to 250, 300, 350, 375, 380, 381, 382, 383, 384, 385, 386, or 387 contiguous amino acids of the sequence of SEQ ID NO:3. In embodiments, the peptide includes a sequence having 91% sequence identity to 250, 300, 350, 375, 380, 381, 382, 383, 384, 385, 386, or 387 contiguous amino acids of the sequence of SEQ ID NO:3. In embodiments, the peptide includes a sequence having 92% sequence identity to 250, 300, 350, 375, 380, 381, 382, 383, 384, 385, 386, or 387 contiguous amino acids of the sequence of SEQ ID NO:3. In embodiments, the peptide includes a sequence having 93% sequence identity to 250, 300, 350, 375, 380, 381, 382, 383, 384, 385, 386, or 387 contiguous amino acids of the sequence of SEQ ID NO:3. In embodiments, the peptide includes a sequence having 94% sequence identity to 250, 300, 350, 375, 380, 381, 382, 383, 384, 385, 386, or 387 contiguous amino acids of the sequence of SEQ ID NO:3. In embodiments, the peptide includes a sequence having 95% sequence identity to 250, 300, 350, 375, 380, 381, 382, 383, 384, 385, 386, or 387 contiguous amino acids of the sequence of SEQ ID NO:3. In embodiments, the peptide includes a sequence having 96% sequence identity to 250, 300, 350, 375, 380, 381, 382, 383, 384, 385, 386, or 387 contiguous amino acids of the sequence of SEQ ID NO: 3. In embodiments, the peptide includes a sequence having 97% sequence identity to 250, 300, 350, 375, 380, 381, 382, 383, 384, 385, 386, or 387 contiguous amino acids of the sequence of SEQ ID NO:3. In embodiments, the peptide includes a sequence having 98% sequence identity to 250, 300, 350, 375, 380, 381, 382, 383, 384, 385, 386, or 387 contiguous amino acids of the sequence of SEQ ID NO:3. In embodiments, the peptide includes a sequence having 99% sequence identity to 250, 300, 350, 375, 380, 381, 382, 383, 384, 385, 386, or 387 contiguous amino acids of the sequence of SEQ ID NO:3. In embodiments, the peptide includes a sequence having 100% sequence identity to 250, 300, 350, 375, 380, 381, 382, 383, 384, 385, 386, or 387 contiguous amino acids of the sequence of SEQ ID NO:3.

In embodiments, the peptide includes the amino acid sequence of SEQ ID NO:3. In embodiments, the peptide has the amino acid sequence of SEQ ID NO:3. In one further embodiment, the peptide is referred to as N62.

In embodiments, the peptide may further include additional protein elements or protein domains. In embodiments, the peptide further includes additional protein elements. In embodiments, the peptide further includes additional protein domains. In embodiments, the additional protein elements or protein domains may include solubilizing amino acid sequences, reporter amino acid sequences, peptide linkers, labels, label moieties, or detectable moieities. In embodiments, the additional protein domain includes a solubilizing amino acid sequence, a reporter amino acid sequence, a peptide linker, a label, a label moiety, or a detectable moiety. In embodiments, the additional protein domain includes a solubilizing amino acid sequence. In embodiments, the additional protein domain includes a reporter amino acid sequence. In embodiments, the additional protein domain includes a peptide linker. In embodiments, the additional protein domain includes a label. In embodiments, the additional protein domain includes a label moiety. In embodiments, the additional protein domain includes a detectable moiety.

In embodiments, the peptide further includes a solubilizing amino acid sequence, wherein the solubilizing amino acid sequence is a maltose-binding protein (MBP) sequence, a small ubiquitin modified (SUMO) sequence, a thioredoxin (TrxA) sequence, a N-utilizing substance A (NusA) sequence, a Fasciola hepatica 8-kDa antigen (Fh8) sequence, a mutated dehalogenase (Halo) sequence, or a glutathione-S-transferase (GST) sequence.

In embodiments, the peptide further includes a solubilizing amino acid sequence, wherein the solubilizing amino acid sequence is a maltose-binding protein (MBP) sequence. In embodiments, the peptide further includes a solubilizing amino acid sequence, wherein the solubilizing amino acid sequence is a small ubiquitin modified (SUMO) sequence. In embodiments, the peptide further includes a solubilizing amino acid sequence, wherein the solubilizing amino acid sequence is a thioredoxin (TrxA) sequence. In embodiments, the peptide further includes a solubilizing amino acid sequence, wherein the solubilizing amino acid sequence is a N-utilizing substance A (NusA) sequence. In embodiments, the peptide further includes a solubilizing amino acid sequence, wherein the solubilizing amino acid sequence is a Fasciola hepatica 8-kDa antigen (Fh8) sequence. In embodiments, the peptide further includes a solubilizing amino acid sequence, wherein the solubilizing amino acid sequence is a mutated dehalogenase (Halo) sequence. In embodiments, the peptide further includes a solubilizing amino acid sequence, wherein the solubilizing amino acid sequence is a glutathione-S-transferase (GST) sequence.

In embodiments, the peptide further includes a solubilizing amino acid sequence, wherein the solubilizing amino acid sequence is a maltose-binding protein (MBP) sequence, a small ubiquitin modified (SUMO) sequence, a thioredoxin (TrxA) sequence, a N-utilizing substance A (NusA) sequence, a Fasciola hepatica 8-kDa antigen (Fh8) sequence, a mutated dehalogenase (Halo) sequence, or a glutathione-S-transferase (GST) sequence.

In embodiments, the peptide further includes a solubilizing amino acid sequence, wherein the solubilizing amino acid sequence is a maltose-binding protein (MBP) sequence. In embodiments, the peptide further includes a solubilizing amino acid sequence, wherein the solubilizing amino acid sequence is a small ubiquitin modified (SUMO) sequence. In embodiments, the peptide further includes a solubilizing amino acid sequence, wherein the solubilizing amino acid sequence is a thioredoxin (TrxA) sequence. In embodiments, the peptide further includes a solubilizing amino acid sequence, wherein the solubilizing amino acid sequence is a N-utilizing substance A (NusA) sequence. In embodiments, the peptide further includes a solubilizing amino acid sequence, wherein the solubilizing amino acid sequence is a Fasciola hepatica 8-kDa antigen (Fh8) sequence. In embodiments, the peptide further includes a solubilizing amino acid sequence, wherein the solubilizing amino acid sequence is a mutated dehalogenase (Halo) sequence. In embodiments, the peptide further includes a solubilizing amino acid sequence, wherein the solubilizing amino acid sequence is a glutathione-S-transferase (GST) sequence.

In embodiments, the peptide further includes a peptide linker. In embodiments, the peptide linker includes the amino acid sequence of SEQ ID NO:42, SEQ ID NO:44, SEQ ID NO: 45, or SEQ ID NO:47. In embodiments, the peptide linker includes the amino acid sequence of SEQ ID NO:42. In embodiments, the peptide linker includes the amino acid sequence of SEQ ID NO: 44. In embodiments, the peptide linker includes the amino acid sequence of SEQ ID NO: 45. In embodiments, the peptide linker includes the amino acid sequence of SEQ ID NO:47. In embodiments, the peptide linker is the amino acid sequence of SEQ ID NO:42, SEQ ID NO: 44, SEQ ID NO:45, or SEQ ID NO:47. In embodiments, the peptide linker is the amino acid sequence of SEQ ID NO:42. In embodiments, the peptide linker is the amino acid sequence of SEQ ID NO:44. In embodiments, the peptide linker is the amino acid sequence of SEQ ID NO: 45. In embodiments, the peptide linker is the amino acid sequence of SEQ ID NO:47.

In embodiments, the peptide includes a solubilizing amino acid sequence attached to the amino acid sequence of SEQ ID NO:3 through a peptide linker. In embodiments, the solubilizing amino acid sequence includes the amino acid sequence of SEQ ID NO:41 and the peptide linker includes the amino acid sequence of SEQ ID NO:42. In embodiments, the peptide includes the amino acid sequence of SEQ ID NO:27. In embodiments, the peptide is the amino acid sequence of SEQ ID NO:27. In one further embodiment, the peptide is referred to as MBP_N54. In one embodiment, the peptide is referred to as TdT 25 or clone 25.

In another aspect is provided a terminal deoxynucleotidyl transferase (TdT) peptide, wherein the peptide includes an amino acid sequence having at least 80% sequence identity to the amino acid sequence of SEQ ID NO:16. In embodiments, the peptide includes an amino acid sequence having at least 81% sequence identity to the amino acid sequence of SEQ ID NO:16. In embodiments, the peptide includes an amino acid sequence having at least 82% sequence identity to the amino acid sequence of SEQ ID NO:16. In embodiments, the peptide includes an amino acid sequence having at least 83% sequence identity to the amino acid sequence of SEQ ID NO: 16. In embodiments, the peptide includes an amino acid sequence having at least 84% sequence identity to the amino acid sequence of SEQ ID NO: 16. In embodiments, the peptide includes an amino acid sequence having at least 85% sequence identity to the amino acid sequence of SEQ ID NO:16. In embodiments, the peptide includes an amino acid sequence having at least 86% sequence identity to the amino acid sequence of SEQ ID NO:16. In embodiments, the peptide includes an amino acid sequence having at least 87% sequence identity to the amino acid sequence of SEQ ID NO: 16. In embodiments, the peptide includes an amino acid sequence having at least 88% sequence identity to the amino acid sequence of SEQ ID NO: 16. In embodiments, the peptide includes an amino acid sequence having at least 89% sequence identity to the amino acid sequence of SEQ ID NO:16. In embodiments, the peptide includes an amino acid sequence having at least 90% sequence identity to the amino acid sequence of SEQ ID NO:16. In embodiments, the peptide includes an amino acid sequence having at least 91% sequence identity to the amino acid sequence of SEQ ID NO:16. In embodiments, the peptide includes an amino acid sequence having at least 92% sequence identity to the amino acid sequence of SEQ ID NO:16. In embodiments, the peptide includes an amino acid sequence having at least 93% sequence identity to the amino acid sequence of SEQ ID NO: 16. In embodiments, the peptide includes an amino acid sequence having at least 94% sequence identity to the amino acid sequence of SEQ ID NO:16. In embodiments, the peptide includes an amino acid sequence having at least 95% sequence identity to the amino acid sequence of SEQ ID NO:16. In embodiments, the peptide includes an amino acid sequence having at least 96% sequence identity to the amino acid sequence of SEQ ID NO:16. In embodiments, the peptide includes an amino acid sequence having at least 97% sequence identity to the amino acid sequence of SEQ ID NO: 16. In embodiments, the peptide includes an amino acid sequence having at least 98% sequence identity to the amino acid sequence of SEQ ID NO: 16. In embodiments, the peptide includes an amino acid sequence having at least 99% sequence identity to the amino acid sequence of SEQ ID NO: 16. In embodiments, the peptide includes an amino acid sequence having at least 100% sequence identity to the amino acid sequence of SEQ ID NO: 16.

In embodiments, the peptide is an amino acid sequence having at least 80% sequence identity to the amino acid sequence of SEQ ID NO:16. In embodiments, the peptide is an amino acid sequence having at least 81% sequence identity to the amino acid sequence of SEQ ID NO: 16. In embodiments, the peptide is an amino acid sequence having at least 82% sequence identity to the amino acid sequence of SEQ ID NO:16. In embodiments, the peptide is an amino acid sequence having at least 83% sequence identity to the amino acid sequence of SEQ ID NO: 16. In embodiments, the peptide is an amino acid sequence having at least 84% sequence identity to the amino acid sequence of SEQ ID NO:16. In embodiments, the peptide is an amino acid sequence having at least 85% sequence identity to the amino acid sequence of SEQ ID NO: 16. In embodiments, the peptide is an amino acid sequence having at least 86% sequence identity to the amino acid sequence of SEQ ID NO:16. In embodiments, the peptide is an amino acid sequence having at least 87% sequence identity to the amino acid sequence of SEQ ID NO: 16. In embodiments, the peptide is an amino acid sequence having at least 88% sequence identity to the amino acid sequence of SEQ ID NO:16. In embodiments, the peptide is an amino acid sequence having at least 89% sequence identity to the amino acid sequence of SEQ ID NO: 16. In embodiments, the peptide is an amino acid sequence having at least 90% sequence identity to the amino acid sequence of SEQ ID NO:16. In embodiments, the peptide is an amino acid sequence having at least 91% sequence identity to the amino acid sequence of SEQ ID NO: 16. In embodiments, the peptide is an amino acid sequence having at least 92% sequence identity to the amino acid sequence of SEQ ID NO:16. In embodiments, the peptide is an amino acid sequence having at least 93% sequence identity to the amino acid sequence of SEQ ID NO: 16. In embodiments, the peptide is an amino acid sequence having at least 94% sequence identity to the amino acid sequence of SEQ ID NO:16. In embodiments, the peptide is an amino acid sequence having at least 95% sequence identity to the amino acid sequence of SEQ ID NO: 16. In embodiments, the peptide is an amino acid sequence having at least 96% sequence identity to the amino acid sequence of SEQ ID NO:16. In embodiments, the peptide is an amino acid sequence having at least 97% sequence identity to the amino acid sequence of SEQ ID NO: 16. In embodiments, the peptide is an amino acid sequence having at least 98% sequence identity to the amino acid sequence of SEQ ID NO:16. In embodiments, the peptide is an amino acid sequence having at least 99% sequence identity to the amino acid sequence of SEQ ID NO: 16. In embodiments, the peptide is an amino acid sequence having at least 100% sequence identity to the amino acid sequence of SEQ ID NO:16.

In embodiments, the peptide includes an amino acid sequence having 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to 250, 300, 350, 375, 380, 381, 382, 383, 384, 385, 386, or 387 contiguous amino acids of the sequence of SEQ ID NO:16. In embodiments, the peptide includes a sequence having 80% sequence identity to 250, 300, 350, 375, 380, 381, 382, 383, 384, 385, 386, or 387 contiguous amino acids of the sequence of SEQ ID NO:16. In embodiments, the peptide includes a sequence having 81% sequence identity to 250, 300, 350, 375, 380, 381, 382, 383, 384, 385, 386, or 387 contiguous amino acids of the sequence of SEQ ID NO:16. In embodiments, the peptide includes a sequence having 82% sequence identity to 250, 300, 350, 375, 380, 381, 382, 383, 384, 385, 386, or 387 contiguous amino acids of the sequence of SEQ ID NO: 16. In embodiments, the peptide includes a sequence having 83% sequence identity to 250, 300, 350, 375, 380, 381, 382, 383, 384, 385, 386, or 387 contiguous amino acids of the sequence of SEQ ID NO:16. In embodiments, the peptide includes a sequence having 84% sequence identity to 250, 300, 350, 375, 380, 381, 382, 383, 384, 385, 386, or 387 contiguous amino acids of the sequence of SEQ ID NO:16. In embodiments, the peptide includes a sequence having 85% sequence identity to 250, 300, 350, 375, 380, 381, 382, 383, 384, 385, 386, or 387 contiguous amino acids of the sequence of SEQ ID NO:16. In embodiments, the peptide includes a sequence having 86% sequence identity to 250, 300, 350, 375, 380, 381, 382, 383, 384, 385, 386, or 387 contiguous amino acids of the sequence of SEQ ID NO:16. In embodiments, the peptide includes a sequence having 87% sequence identity to 250, 300, 350, 375, 380, 381, 382, 383, 384, 385, 386, or 387 contiguous amino acids of the sequence of SEQ ID NO:16. In embodiments, the peptide includes a sequence having 88% sequence identity to 250, 300, 350, 375, 380, 381, 382, 383, 384, 385, 386, or 387 contiguous amino acids of the sequence of SEQ ID NO: 16. In embodiments, the peptide includes a sequence having 89% sequence identity to 250, 300, 350, 375, 380, 381, 382, 383, 384, 385, 386, or 387 contiguous amino acids of the sequence of SEQ ID NO:16. In embodiments, the peptide includes a sequence having 90% sequence identity to 250, 300, 350, 375, 380, 381, 382, 383, 384, 385, 386, or 387 contiguous amino acids of the sequence of SEQ ID NO:16. In embodiments, the peptide includes a sequence having 91% sequence identity to 250, 300, 350, 375, 380, 381, 382, 383, 384, 385, 386, or 387 contiguous amino acids of the sequence of SEQ ID NO:16. In embodiments, the peptide includes a sequence having 92% sequence identity to 250, 300, 350, 375, 380, 381, 382, 383, 384, 385, 386, or 387 contiguous amino acids of the sequence of SEQ ID NO:16. In embodiments, the peptide includes a sequence having 93% sequence identity to 250, 300, 350, 375, 380, 381, 382, 383, 384, 385, 386, or 387 contiguous amino acids of the sequence of SEQ ID NO:16. In embodiments, the peptide includes a sequence having 94% sequence identity to 250, 300, 350, 375, 380, 381, 382, 383, 384, 385, 386, or 387 contiguous amino acids of the sequence of SEQ ID NO: 16. In embodiments, the peptide includes a sequence having 95% sequence identity to 250, 300, 350, 375, 380, 381, 382, 383, 384, 385, 386, or 387 contiguous amino acids of the sequence of SEQ ID NO:16. In embodiments, the peptide includes a sequence having 96% sequence identity to 250, 300, 350, 375, 380, 381, 382, 383, 384, 385, 386, or 387 contiguous amino acids of the sequence of SEQ ID NO:16. In embodiments, the peptide includes a sequence having 97% sequence identity to 250, 300, 350, 375, 380, 381, 382, 383, 384, 385, 386, or 387 contiguous amino acids of the sequence of SEQ ID NO:16. In embodiments, the peptide includes a sequence having 98% sequence identity to 250, 300, 350, 375, 380, 381, 382, 383, 384, 385, 386, or 387 contiguous amino acids of the sequence of SEQ ID NO:16. In embodiments, the peptide includes a sequence having 99% sequence identity to 250, 300, 350, 375, 380, 381, 382, 383, 384, 385, 386, or 387 contiguous amino acids of the sequence of SEQ ID NO:16. In embodiments, the peptide includes a sequence having 100% sequence identity to 250, 300, 350, 375, 380, 381, 382, 383, 384, 385, 386, or 387 contiguous amino acids of the sequence of SEQ ID NO: 16.

In embodiments, the peptide includes the amino acid sequence of SEQ ID NO:16. In embodiments, the peptide has the amino acid sequence of SEQ ID NO:16. In one further embodiment, the peptide is referred to as N62dm.

In embodiments, the peptide may further include additional protein elements or protein domains. In embodiments, the peptide further includes additional protein elements. In embodiments, the peptide further includes additional protein domains. In embodiments, the additional protein elements or protein domains may include solubilizing amino acid sequences, reporter amino acid sequences, peptide linkers, labels, label moieties, or detectable moieities. In embodiments, the additional protein domain includes a solubilizing amino acid sequence, a reporter amino acid sequence, a peptide linker, a label, a label moiety, or a detectable moiety. In embodiments, the additional protein domain includes a solubilizing amino acid sequence. In embodiments, the additional protein domain includes a reporter amino acid sequence. In embodiments, the additional protein domain includes a peptide linker. In embodiments, the additional protein domain includes a label. In embodiments, the additional protein domain includes a label moiety. In embodiments, the additional protein domain includes a detectable moiety.

In embodiments, the peptide further includes a solubilizing amino acid sequence, wherein the solubilizing amino acid sequence is a maltose-binding protein (MBP) sequence, a small ubiquitin modified (SUMO) sequence, a thioredoxin (TrxA) sequence, a N-utilizing substance A (NusA) sequence, a Fasciola hepatica 8-kDa antigen (Fh8) sequence, a mutated dehalogenase (Halo) sequence, or a glutathione-S-transferase (GST) sequence.

In embodiments, the peptide further includes a solubilizing amino acid sequence, wherein the solubilizing amino acid sequence is a maltose-binding protein (MBP) sequence. In embodiments, the peptide further includes a solubilizing amino acid sequence, wherein the solubilizing amino acid sequence is a small ubiquitin modified (SUMO) sequence. In embodiments, the peptide further includes a solubilizing amino acid sequence, wherein the solubilizing amino acid sequence is a thioredoxin (TrxA) sequence. In embodiments, the peptide further includes a solubilizing amino acid sequence, wherein the solubilizing amino acid sequence is a N-utilizing substance A (NusA) sequence. In embodiments, the peptide further includes a solubilizing amino acid sequence, wherein the solubilizing amino acid sequence is a Fasciola hepatica 8-kDa antigen (Fh8) sequence. In embodiments, the peptide further includes a solubilizing amino acid sequence, wherein the solubilizing amino acid sequence is a mutated dehalogenase (Halo) sequence. In embodiments, the peptide further includes a solubilizing amino acid sequence, wherein the solubilizing amino acid sequence is a glutathione-S-transferase (GST) sequence.

In embodiments, the peptide further includes a first peptide linker and a second peptide linker. In embodiments, the first peptide linker includes the amino acid sequence of SEQ ID NO: 42, SEQ ID NO:44, SEQ ID NO:45, or SEQ ID NO:47. In embodiments, the first peptide linker includes the amino acid sequence of SEQ ID NO:42. In embodiments, the first peptide linker includes the amino acid sequence of SEQ ID NO:44. In embodiments, the first peptide linker includes the amino acid sequence of SEQ ID NO:45. In embodiments, the first peptide linker includes the amino acid sequence of SEQ ID NO:47. In embodiments, the first peptide linker is the amino acid sequence of SEQ ID NO:42, SEQ ID NO:44, SEQ ID NO:45, or SEQ ID NO: 47. In embodiments, the first peptide linker is the amino acid sequence of SEQ ID NO:42. In embodiments, the first peptide linker is the amino acid sequence of SEQ ID NO:44. In embodiments, the first peptide linker is the amino acid sequence of SEQ ID NO:45. In embodiments, the first peptide linker is the amino acid sequence of SEQ ID NO:47.

In embodiments, the second peptide linker includes the amino acid sequence of SEQ ID NO: 42, SEQ ID NO:44, SEQ ID NO:45, or SEQ ID NO:47. In embodiments, the second peptide linker includes the amino acid sequence of SEQ ID NO:42. In embodiments, the second peptide linker includes the amino acid sequence of SEQ ID NO:44. In embodiments, the second peptide linker includes the amino acid sequence of SEQ ID NO:45. In embodiments, the second peptide linker includes the amino acid sequence of SEQ ID NO:47. In embodiments, the second peptide linker is the amino acid sequence of SEQ ID NO:42, SEQ ID NO:44, SEQ ID NO:45, or SEQ ID NO: 47. In embodiments, the second peptide linker is the amino acid sequence of SEQ ID NO:42. In embodiments, the second peptide linker is the amino acid sequence of SEQ ID NO:44. In embodiments, the second peptide linker is the amino acid sequence of SEQ ID NO:45. In embodiments, the second peptide linker is the amino acid sequence of SEQ ID NO:47.

In embodiments, the peptide further includes a reporter amino acid sequence. In embodiments the reporter amino acid sequence is a fluorescent protein or fragment thereof. In embodiments, the reporter amino acid sequence is a green fluorescent protein. In embodiments, the reporter amino acid sequence includes the amino acid sequence of SEQ ID NO:43. In embodiments, the reporter amino acid sequence is the amino acid sequence of SEQ ID NO:43.

In embodiments, the peptide includes a solubilizing amino acid sequence attached to the N-terminus of a reporter amino acid sequence via a first peptide linker, wherein the reporter amino acid sequence is bound to the N-terminus of the amino acid sequence of SEQ ID NO:16 via a second peptide linker. In embodiments, the solubilizing amino acid includes the amino acid sequence of SEQ ID NO:41; the first peptide linker includes the amino acid sequence of SEQ ID NO: 44; the reporter amino acid sequence includes the amino acid sequence of SEQ ID NO:43; and the second peptide linker includes the amino acid sequence of SEQ ID NO:45. In embodiments, the peptide includes the amino acid sequence of SEQ ID NO:26. In embodiments, the peptide is the amino acid sequence of SEQ ID NO:26. In one further embodiment, the peptide is referred to as MBP_N62dm.

In another aspect is provided a terminal deoxynucleotidyl transferase (TdT) peptide, wherein the peptide includes an amino acid sequence having at least 80% sequence identity to the amino acid sequence of SEQ ID NO:17. In embodiments, the peptide includes an amino acid sequence having at least 81% sequence identity to the amino acid sequence of SEQ ID NO:17. In embodiments, the peptide includes an amino acid sequence having at least 82% sequence identity to the amino acid sequence of SEQ ID NO: 17. In embodiments, the peptide includes an amino acid sequence having at least 83% sequence identity to the amino acid sequence of SEQ ID NO: 17. In embodiments, the peptide includes an amino acid sequence having at least 84% sequence identity to the amino acid sequence of SEQ ID NO:17. In embodiments, the peptide includes an amino acid sequence having at least 85% sequence identity to the amino acid sequence of SEQ ID NO:17. In embodiments, the peptide includes an amino acid sequence having at least 86% sequence identity to the amino acid sequence of SEQ ID NO: 17. In embodiments, the peptide includes an amino acid sequence having at least 87% sequence identity to the amino acid sequence of SEQ ID NO: 17. In embodiments, the peptide includes an amino acid sequence having at least 88% sequence identity to the amino acid sequence of SEQ ID NO: 17. In embodiments, the peptide includes an amino acid sequence having at least 89% sequence identity to the amino acid sequence of SEQ ID NO:17. In embodiments, the peptide includes an amino acid sequence having at least 90% sequence identity to the amino acid sequence of SEQ ID NO:17. In embodiments, the peptide includes an amino acid sequence having at least 91% sequence identity to the amino acid sequence of SEQ ID NO:17. In embodiments, the peptide includes an amino acid sequence having at least 92% sequence identity to the amino acid sequence of SEQ ID NO:17. In embodiments, the peptide includes an amino acid sequence having at least 93% sequence identity to the amino acid sequence of SEQ ID NO: 17. In embodiments, the peptide includes an amino acid sequence having at least 94% sequence identity to the amino acid sequence of SEQ ID NO:17. In embodiments, the peptide includes an amino acid sequence having at least 95% sequence identity to the amino acid sequence of SEQ ID NO:17. In embodiments, the peptide includes an amino acid sequence having at least 96% sequence identity to the amino acid sequence of SEQ ID NO:17. In embodiments, the peptide includes an amino acid sequence having at least 97% sequence identity to the amino acid sequence of SEQ ID NO: 17. In embodiments, the peptide includes an amino acid sequence having at least 98% sequence identity to the amino acid sequence of SEQ ID NO: 17. In embodiments, the peptide includes an amino acid sequence having at least 99% sequence identity to the amino acid sequence of SEQ ID NO:17. In embodiments, the peptide includes an amino acid sequence having at least 100% sequence identity to the amino acid sequence of SEQ ID NO:17.

In embodiments, the peptide is an amino acid sequence having at least 80% sequence identity to the amino acid sequence of SEQ ID NO:17. In embodiments, the peptide is an amino acid sequence having at least 81% sequence identity to the amino acid sequence of SEQ ID NO: 17. In embodiments, the peptide is an amino acid sequence having at least 82% sequence identity to the amino acid sequence of SEQ ID NO:17. In embodiments, the peptide is an amino acid sequence having at least 83% sequence identity to the amino acid sequence of SEQ ID NO: 17. In embodiments, the peptide is an amino acid sequence having at least 84% sequence identity to the amino acid sequence of SEQ ID NO:17. In embodiments, the peptide is an amino acid sequence having at least 85% sequence identity to the amino acid sequence of SEQ ID NO: 17. In embodiments, the peptide is an amino acid sequence having at least 86% sequence identity to the amino acid sequence of SEQ ID NO:17. In embodiments, the peptide is an amino acid sequence having at least 87% sequence identity to the amino acid sequence of SEQ ID NO: 17. In embodiments, the peptide is an amino acid sequence having at least 88% sequence identity to the amino acid sequence of SEQ ID NO:17. In embodiments, the peptide is an amino acid sequence having at least 89% sequence identity to the amino acid sequence of SEQ ID NO: 17. In embodiments, the peptide is an amino acid sequence having at least 90% sequence identity to the amino acid sequence of SEQ ID NO:17. In embodiments, the peptide is an amino acid sequence having at least 91% sequence identity to the amino acid sequence of SEQ ID NO: 17. In embodiments, the peptide is an amino acid sequence having at least 92% sequence identity to the amino acid sequence of SEQ ID NO:17. In embodiments, the peptide is an amino acid sequence having at least 93% sequence identity to the amino acid sequence of SEQ ID NO: 17. In embodiments, the peptide is an amino acid sequence having at least 94% sequence identity to the amino acid sequence of SEQ ID NO:17. In embodiments, the peptide is an amino acid sequence having at least 95% sequence identity to the amino acid sequence of SEQ ID NO: 17. In embodiments, the peptide is an amino acid sequence having at least 96% sequence identity to the amino acid sequence of SEQ ID NO:17. In embodiments, the peptide is an amino acid sequence having at least 97% sequence identity to the amino acid sequence of SEQ ID NO: 17. In embodiments, the peptide is an amino acid sequence having at least 98% sequence identity to the amino acid sequence of SEQ ID NO:17. In embodiments, the peptide is an amino acid sequence having at least 99% sequence identity to the amino acid sequence of SEQ ID NO: 17. In embodiments, the peptide is an amino acid sequence having at least 100% sequence identity to the amino acid sequence of SEQ ID NO:17.

In embodiments, the peptide includes an amino acid sequence having 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to 250, 300, 350, 375, 380, 381, 382, 383, 384, 385, 386, 387, 388 contiguous amino acids of the sequence of SEQ ID NO:17. In embodiments, the peptide includes a sequence having 80% sequence identity to 250, 300, 350, 375, 380, 381, 382, 383, 384, 385, 386, 387, 388 contiguous amino acids of the sequence of SEQ ID NO:17. In embodiments, the peptide includes a sequence having 81% sequence identity to 250, 300, 350, 375, 380, 381, 382, 383, 384, 385, 386, 387, 388 contiguous amino acids of the sequence of SEQ ID NO: 17. In embodiments, the peptide includes a sequence having 82% sequence identity to 250, 300, 350, 375, 380, 381, 382, 383, 384, 385, 386, 387, 388 contiguous amino acids of the sequence of SEQ ID NO:17. In embodiments, the peptide includes a sequence having 83% sequence identity to 250, 300, 350, 375, 380, 381, 382, 383, 384, 385, 386, 387, 388 contiguous amino acids of the sequence of SEQ ID NO:17. In embodiments, the peptide includes a sequence having 84% sequence identity to 250, 300, 350, 375, 380, 381, 382, 383, 384, 385, 386, 387, 388 contiguous amino acids of the sequence of SEQ ID NO:17. In embodiments, the peptide includes a sequence having 85% sequence identity to 250, 300, 350, 375, 380, 381, 382, 383, 384, 385, 386, 387, 388 contiguous amino acids of the sequence of SEQ ID NO:17. In embodiments, the peptide includes a sequence having 86% sequence identity to 250, 300, 350, 375, 380, 381, 382, 383, 384, 385, 386, 387, 388 contiguous amino acids of the sequence of SEQ ID NO:17. In embodiments, the peptide includes a sequence having 87% sequence identity to 250, 300, 350, 375, 380, 381, 382, 383, 384, 385, 386, 387, 388 contiguous amino acids of the sequence of SEQ ID NO: 17. In embodiments, the peptide includes a sequence having 88% sequence identity to 250, 300, 350, 375, 380, 381, 382, 383, 384, 385, 386, 387, 388 contiguous amino acids of the sequence of SEQ ID NO:17. In embodiments, the peptide includes a sequence having 89% sequence identity 250, 300, 350, 375, 380, 381, 382, 383, 384, 385, 386, 387, 388 contiguous amino acids of the sequence of SEQ ID NO:17. In embodiments, the peptide includes a sequence having 90% sequence identity to 250, 300, 350, 375, 380, 381, 382, 383, 384, 385, 386, 387, 388 contiguous amino acids of the sequence of SEQ ID NO:17. In embodiments, the peptide includes a sequence having 91% sequence identity to 250, 300, 350, 375, 380, 381, 382, 383, 384, 385, 386, 387, 388 contiguous amino acids of the sequence of SEQ ID NO:17. In embodiments, the peptide includes a sequence having 92% sequence identity to 250, 300, 350, 375, 380, 381, 382, 383, 384, 385, 386, 387, 388 contiguous amino acids of the sequence of SEQ ID NO:17. In embodiments, the peptide includes a sequence having 93% sequence identity to 250, 300, 350, 375, 380, 381, 382, 383, 384, 385, 386, 387, 388 contiguous amino acids of the sequence of SEQ ID NO: 17. In embodiments, the peptide includes a sequence having 94% sequence identity to 250, 300, 350, 375, 380, 381, 382, 383, 384, 385, 386, 387, 388 contiguous amino acids of the sequence of SEQ ID NO:17. In embodiments, the peptide includes a sequence having 95% sequence identity to 250, 300, 350, 375, 380, 381, 382, 383, 384, 385, 386, 387, 388 contiguous amino acids of the sequence of SEQ ID NO:17. In embodiments, the peptide includes a sequence having 96% sequence identity to 250, 300, 350, 375, 380, 381, 382, 383, 384, 385, 386, 387, 388 contiguous amino acids of the sequence of SEQ ID NO:17. In embodiments, the peptide includes a sequence having 97% sequence identity to 250, 300, 350, 375, 380, 381, 382, 383, 384, 385, 386, 387, 388 contiguous amino acids of the sequence of SEQ ID NO:17. In embodiments, the peptide includes a sequence having 98% sequence identity to 250, 300, 350, 375, 380, 381, 382, 383, 384, 385, 386, 387, 388 contiguous amino acids of the sequence of SEQ ID NO:17. In embodiments, the peptide includes a sequence having 99% sequence identity to 250, 300, 350, 375, 380, 381, 382, 383, 384, 385, 386, 387, 388 contiguous amino acids of the sequence of SEQ ID NO: 17. In embodiments, the peptide includes a sequence having 100% sequence identity to 250, 300, 350, 375, 380, 381, 382, 383, 384, 385, 386, 387, 388 contiguous amino acids of the sequence of SEQ ID NO:17.

In embodiments, the peptide includes the amino acid sequence of SEQ ID NO:17. In embodiments, the peptide has the amino acid sequence of SEQ ID NO:17. In one further embodiment, the peptide is referred to as N54dm.

In embodiments, the peptide may further include additional protein elements or protein domains. In embodiments, the peptide further includes additional protein elements. In embodiments, the peptide further includes additional protein domains. In embodiments, the additional protein elements or protein domains may include solubilizing amino acid sequences, reporter amino acid sequences, peptide linkers, labels, label moieties, or detectable moieities. In embodiments, the additional protein domain includes a solubilizing amino acid sequence, a reporter amino acid sequence, a peptide linker, a label, a label moiety, or a detectable moiety. In embodiments, the additional protein domain includes a solubilizing amino acid sequence. In embodiments, the additional protein domain includes a reporter amino acid sequence. In embodiments, the additional protein domain includes a peptide linker. In embodiments, the additional protein domain includes a label. In embodiments, the additional protein domain includes a label moiety. In embodiments, the additional protein domain includes a detectable moiety.

In embodiments, the peptide further includes a solubilizing amino acid sequence, wherein the solubilizing amino acid sequence is a maltose-binding protein (MBP) sequence, a small ubiquitin modified (SUMO) sequence, a thioredoxin (TrxA) sequence, a N-utilizing substance A (NusA) sequence, a Fasciola hepatica 8-kDa antigen (Fh8) sequence, a mutated dehalogenase (Halo) sequence, or a glutathione-S-transferase (GST) sequence.

In embodiments, the peptide further includes a solubilizing amino acid sequence, wherein the solubilizing amino acid sequence is a maltose-binding protein (MBP) sequence. In embodiments, the peptide further includes a solubilizing amino acid sequence, wherein the solubilizing amino acid sequence is a small ubiquitin modified (SUMO) sequence. In embodiments, the peptide further includes a solubilizing amino acid sequence, wherein the solubilizing amino acid sequence is a thioredoxin (TrxA) sequence. In embodiments, the peptide further includes a solubilizing amino acid sequence, wherein the solubilizing amino acid sequence is a N-utilizing substance A (NusA) sequence. In embodiments, the peptide further includes a solubilizing amino acid sequence, wherein the solubilizing amino acid sequence is a Fasciola hepatica 8-kDa antigen (Fh8) sequence. In embodiments, the peptide further includes a solubilizing amino acid sequence, wherein the solubilizing amino acid sequence is a mutated dehalogenase (Halo) sequence. In embodiments, the peptide further includes a solubilizing amino acid sequence, wherein the solubilizing amino acid sequence is a glutathione-S-transferase (GST) sequence.

In embodiments, the peptide further includes a first peptide linker and a second peptide linker. In embodiments, the first peptide linker includes the amino acid sequence of SEQ ID NO: 42, SEQ ID NO:44, SEQ ID NO:45, or SEQ ID NO:47. In embodiments, the first peptide linker includes the amino acid sequence of SEQ ID NO:42. In embodiments, the first peptide linker includes the amino acid sequence of SEQ ID NO:44. In embodiments, the first peptide linker includes the amino acid sequence of SEQ ID NO:45. In embodiments, the first peptide linker includes the amino acid sequence of SEQ ID NO:47. In embodiments, the first peptide linker is the amino acid sequence of SEQ ID NO:42, SEQ ID NO:44, SEQ ID NO:45, or SEQ ID NO: 47. In embodiments, the first peptide linker is the amino acid sequence of SEQ ID NO:42. In embodiments, the first peptide linker is the amino acid sequence of SEQ ID NO:44. In embodiments, the first peptide linker is the amino acid sequence of SEQ ID NO:45. In embodiments, the first peptide linker is the amino acid sequence of SEQ ID NO:47.

In embodiments, the second peptide linker includes the amino acid sequence of SEQ ID NO: 42, SEQ ID NO:44, SEQ ID NO:45, or SEQ ID NO:47. In embodiments, the second peptide linker includes the amino acid sequence of SEQ ID NO:42. In embodiments, the second peptide linker includes the amino acid sequence of SEQ ID NO:44. In embodiments, the second peptide linker includes the amino acid sequence of SEQ ID NO:45. In embodiments, the second peptide linker includes the amino acid sequence of SEQ ID NO:47. In embodiments, the second peptide linker is the amino acid sequence of SEQ ID NO:42, SEQ ID NO:44, SEQ ID NO:45, or SEQ ID NO: 47. In embodiments, the second peptide linker is the amino acid sequence of SEQ ID NO:42. In embodiments, the second peptide linker is the amino acid sequence of SEQ ID NO:44. In embodiments, the second peptide linker is the amino acid sequence of SEQ ID NO:45. In embodiments, the second peptide linker is the amino acid sequence of SEQ ID NO:47.

In embodiments, the peptide further includes a reporter amino acid sequence. In embodiments the reporter amino acid sequence is a fluorescent protein or fragment thereof. In embodiments, the reporter amino acid sequence is a green fluorescent protein. In embodiments, the reporter amino acid sequence includes the amino acid sequence of SEQ ID NO:43. In embodiments, the reporter amino acid sequence is the amino acid sequence of SEQ ID NO:43.

In embodiments, the peptide includes a solubilizing amino acid sequence attached to the N-terminus of a reporter amino acid sequence via a first peptide linker, wherein the reporter amino acid sequence is bound to the N-terminus of the amino acid sequence of SEQ ID NO:17 via a second peptide linker. In embodiments, the solubilizing amino acid includes the amino acid sequence of SEQ ID NO:41; the first peptide linker includes the amino acid sequence of SEQ ID NO: 44; the reporter amino acid sequence includes the amino acid sequence of SEQ ID NO:43; and the second peptide linker includes the amino acid sequence of SEQ ID NO:45. In embodiments, the peptide includes the amino acid sequence of SEQ ID NO:28. In embodiments, the peptide is the amino acid sequence of SEQ ID NO:28. In one further embodiment, the peptide is referred to as MBP_N54dm.

In another aspect is provided a terminal deoxynucleotidyl transferase (TdT) peptide, wherein the peptide includes an amino acid sequence having at least 80% sequence identity to the amino acid sequence of SEQ ID NO:18. In embodiments, the peptide includes an amino acid sequence having at least 81% sequence identity to the amino acid sequence of SEQ ID NO:18. In embodiments, the peptide includes an amino acid sequence having at least 82% sequence identity to the amino acid sequence of SEQ ID NO:18. In embodiments, the peptide includes an amino acid sequence having at least 83% sequence identity to the amino acid sequence of SEQ ID NO: 18. In embodiments, the peptide includes an amino acid sequence having at least 84% sequence identity to the amino acid sequence of SEQ ID NO:18. In embodiments, the peptide includes an amino acid sequence having at least 85% sequence identity to the amino acid sequence of SEQ ID NO:18. In embodiments, the peptide includes an amino acid sequence having at least 86% sequence identity to the amino acid sequence of SEQ ID NO:18. In embodiments, the peptide includes an amino acid sequence having at least 87% sequence identity to the amino acid sequence of SEQ ID NO: 18. In embodiments, the peptide includes an amino acid sequence having at least 88% sequence identity to the amino acid sequence of SEQ ID NO: 18. In embodiments, the peptide includes an amino acid sequence having at least 89% sequence identity to the amino acid sequence of SEQ ID NO:18. In embodiments, the peptide includes an amino acid sequence having at least 90% sequence identity to the amino acid sequence of SEQ ID NO:18. In embodiments, the peptide includes an amino acid sequence having at least 91% sequence identity to the amino acid sequence of SEQ ID NO:18. In embodiments, the peptide includes an amino acid sequence having at least 92% sequence identity to the amino acid sequence of SEQ ID NO:18. In embodiments, the peptide includes an amino acid sequence having at least 93% sequence identity to the amino acid sequence of SEQ ID NO: 18. In embodiments, the peptide includes an amino acid sequence having at least 94% sequence identity to the amino acid sequence of SEQ ID NO:18. In embodiments, the peptide includes an amino acid sequence having at least 95% sequence identity to the amino acid sequence of SEQ ID NO:18. In embodiments, the peptide includes an amino acid sequence having at least 96% sequence identity to the amino acid sequence of SEQ ID NO:18. In embodiments, the peptide includes an amino acid sequence having at least 97% sequence identity to the amino acid sequence of SEQ ID NO:18. In embodiments, the peptide includes an amino acid sequence having at least 98% sequence identity to the amino acid sequence of SEQ ID NO: 18. In embodiments, the peptide includes an amino acid sequence having at least 99% sequence identity to the amino acid sequence of SEQ ID NO: 18. In embodiments, the peptide includes an amino acid sequence having at least 100% sequence identity to the amino acid sequence of SEQ ID NO:18.

In embodiments, the peptide is an amino acid sequence having at least 80% sequence identity to the amino acid sequence of SEQ ID NO:18. In embodiments, the peptide is an amino acid sequence having at least 81% sequence identity to the amino acid sequence of SEQ ID NO: 18. In embodiments, the peptide is an amino acid sequence having at least 82% sequence identity to the amino acid sequence of SEQ ID NO:18. In embodiments, the peptide is an amino acid sequence having at least 83% sequence identity to the amino acid sequence of SEQ ID NO: 18. In embodiments, the peptide is an amino acid sequence having at least 84% sequence identity to the amino acid sequence of SEQ ID NO:18. In embodiments, the peptide is an amino acid sequence having at least 85% sequence identity to the amino acid sequence of SEQ ID NO: 18. In embodiments, the peptide is an amino acid sequence having at least 86% sequence identity to the amino acid sequence of SEQ ID NO:18. In embodiments, the peptide is an amino acid sequence having at least 87% sequence identity to the amino acid sequence of SEQ ID NO: 18. In embodiments, the peptide is an amino acid sequence having at least 88% sequence identity to the amino acid sequence of SEQ ID NO:18. In embodiments, the peptide is an amino acid sequence having at least 89% sequence identity to the amino acid sequence of SEQ ID NO: 18. In embodiments, the peptide is an amino acid sequence having at least 90% sequence identity to the amino acid sequence of SEQ ID NO:18. In embodiments, the peptide is an amino acid sequence having at least 91% sequence identity to the amino acid sequence of SEQ ID NO: 18. In embodiments, the peptide is an amino acid sequence having at least 92% sequence identity to the amino acid sequence of SEQ ID NO:18. In embodiments, the peptide is an amino acid sequence having at least 93% sequence identity to the amino acid sequence of SEQ ID NO: 18. In embodiments, the peptide is an amino acid sequence having at least 94% sequence identity to the amino acid sequence of SEQ ID NO:18. In embodiments, the peptide is an amino acid sequence having at least 95% sequence identity to the amino acid sequence of SEQ ID NO: 18. In embodiments, the peptide is an amino acid sequence having at least 96% sequence identity to the amino acid sequence of SEQ ID NO:18. In embodiments, the peptide is an amino acid sequence having at least 97% sequence identity to the amino acid sequence of SEQ ID NO: 18. In embodiments, the peptide is an amino acid sequence having at least 98% sequence identity to the amino acid sequence of SEQ ID NO:18. In embodiments, the peptide is an amino acid sequence having at least 99% sequence identity to the amino acid sequence of SEQ ID NO: 18. In embodiments, the peptide is an amino acid sequence having at least 100% sequence identity to the amino acid sequence of SEQ ID NO:18.

In embodiments, the peptide includes an amino acid sequence having 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to 250, 300, 350, 375, 380, 385, 386, 387, 388, 389, 390, 391, or 392 contiguous amino acids of the sequence of SEQ ID NO:18. In embodiments, the peptide includes a sequence having 80% sequence identity to 250, 300, 350, 375, 380, 385, 386, 387, 388, 389, 390, 391, or 392 contiguous amino acids of the sequence of SEQ ID NO:18. In embodiments, the peptide includes a sequence having 81% sequence identity to 250, 300, 350, 375, 380, 385, 386, 387, 388, 389, 390, 391, or 392 contiguous amino acids of the sequence of SEQ ID NO:18. In embodiments, the peptide includes a sequence having 82% sequence identity to 250, 300, 350, 375, 380, 385, 386, 387, 388, 389, 390, 391, or 392 contiguous amino acids of the sequence of SEQ ID NO:18. In embodiments, the peptide includes a sequence having 83% sequence identity to 250, 300, 350, 375, 380, 385, 386, 387, 388, 389, 390, 391, or 392 contiguous amino acids of the sequence of SEQ ID NO:18. In embodiments, the peptide includes a sequence having 84% sequence identity to 250, 300, 350, 375, 380, 385, 386, 387, 388, 389, 390, 391, or 392 contiguous amino acids of the sequence of SEQ ID NO:18. In embodiments, the peptide includes a sequence having 85% sequence identity to 250, 300, 350, 375, 380, 385, 386, 387, 388, 389, 390, 391, or 392 contiguous amino acids of the sequence of SEQ ID NO:18. In embodiments, the peptide includes a sequence having 86% sequence identity to 250, 300, 350, 375, 380, 385, 386, 387, 388, 389, 390, 391, or 392 contiguous amino acids of the sequence of SEQ ID NO:18. In embodiments, the peptide includes a sequence having 87% sequence identity to 250, 300, 350, 375, 380, 385, 386, 387, 388, 389, 390, 391, or 392 contiguous amino acids of the sequence of SEQ ID NO:18. In embodiments, the peptide includes a sequence having 88% sequence identity to 250, 300, 350, 375, 380, 385, 386, 387, 388, 389, 390, 391, or 392 contiguous amino acids of the sequence of SEQ ID NO:18. In embodiments, the peptide includes a sequence having 89% sequence identity to 250, 300, 350, 375, 380, 385, 386, 387, 388, 389, 390, 391, or 392 contiguous amino acids of the sequence of SEQ ID NO:18. In embodiments, the peptide includes a sequence having 90% sequence identity to 250, 300, 350, 375, 380, 385, 386, 387, 388, 389, 390, 391, or 392 contiguous amino acids of the sequence of SEQ ID NO:18. In embodiments, the peptide includes a sequence having 91% sequence identity to 250, 300, 350, 375, 380, 385, 386, 387, 388, 389, 390, 391, or 392 contiguous amino acids of the sequence of SEQ ID NO:18. In embodiments, the peptide includes a sequence having 92% sequence identity to 250, 300, 350, 375, 380, 385, 386, 387, 388, 389, 390, 391, or 392 contiguous amino acids of the sequence of SEQ ID NO:18. In embodiments, the peptide includes a sequence having 93% sequence identity to 250, 300, 350, 375, 380, 385, 386, 387, 388, 389, 390, 391, or 392 contiguous amino acids of the sequence of SEQ ID NO:18. In embodiments, the peptide includes a sequence having 94% sequence identity to 250, 300, 350, 375, 380, 385, 386, 387, 388, 389, 390, 391, or 392 contiguous amino acids of the sequence of SEQ ID NO:18. In embodiments, the peptide includes a sequence having 95% sequence identity to 250, 300, 350, 375, 380, 385, 386, 387, 388, 389, 390, 391, or 392 contiguous amino acids of the sequence of SEQ ID NO:18. In embodiments, the peptide includes a sequence having 96% sequence identity to 250, 300, 350, 375, 380, 385, 386, 387, 388, 389, 390, 391, or 392 contiguous amino acids of the sequence of SEQ ID NO:18. In embodiments, the peptide includes a sequence having 97% sequence identity to 250, 300, 350, 375, 380, 385, 386, 387, 388, 389, 390, 391, or 392 contiguous amino acids of the sequence of SEQ ID NO:18. In embodiments, the peptide includes a sequence having 98% sequence identity to 250, 300, 350, 375, 380, 385, 386, 387, 388, 389, 390, 391, or 392 contiguous amino acids of the sequence of SEQ ID NO:18. In embodiments, the peptide includes a sequence having 99% sequence identity to 250, 300, 350, 375, 380, 385, 386, 387, 388, 389, 390, 391, or 392 contiguous amino acids of the sequence of SEQ ID NO:18. In embodiments, the peptide includes a sequence having 100% sequence identity to 250, 300, 350, 375, 380, 385, 386, 387, 388, 389, 390, 391, or 392 contiguous amino acids of the sequence of SEQ ID NO:18.

In embodiments, the peptide includes the amino acid sequence of SEQ ID NO:18. In embodiments, the peptide has the amino acid sequence of SEQ ID NO:18. In one further embodiment, the peptide is referred to as NO.

In embodiments, the peptide may further include additional protein elements or protein domains. In embodiments, the peptide further includes additional protein elements. In embodiments, the peptide further includes additional protein domains. In embodiments, the additional protein elements or protein domains may include solubilizing amino acid sequences, reporter amino acid sequences, peptide linkers, labels, label moieties, or detectable moieities. In embodiments, the additional protein domain includes a solubilizing amino acid sequence, a reporter amino acid sequence, a peptide linker, a label, a label moiety, or a detectable moiety. In embodiments, the additional protein domain includes a solubilizing amino acid sequence. In embodiments, the additional protein domain includes a reporter amino acid sequence. In embodiments, the additional protein domain includes a peptide linker. In embodiments, the additional protein domain includes a label. In embodiments, the additional protein domain includes a label moiety. In embodiments, the additional protein domain includes a detectable moiety.

In embodiments, the peptide further includes a solubilizing amino acid sequence, wherein the solubilizing amino acid sequence is a maltose-binding protein (MBP) sequence, a small ubiquitin modified (SUMO) sequence, a thioredoxin (TrxA) sequence, a N-utilizing substance A (NusA) sequence, a Fasciola hepatica 8-kDa antigen (Fh8) sequence, a mutated dehalogenase (Halo) sequence, or a glutathione-S-transferase (GST) sequence.

In embodiments, the peptide further includes a solubilizing amino acid sequence, wherein the solubilizing amino acid sequence is a maltose-binding protein (MBP) sequence. In embodiments, the peptide further includes a solubilizing amino acid sequence, wherein the solubilizing amino acid sequence is a small ubiquitin modified (SUMO) sequence. In embodiments, the peptide further includes a solubilizing amino acid sequence, wherein the solubilizing amino acid sequence is a thioredoxin (TrxA) sequence. In embodiments, the peptide further includes a solubilizing amino acid sequence, wherein the solubilizing amino acid sequence is a N-utilizing substance A (NusA) sequence. In embodiments, the peptide further includes a solubilizing amino acid sequence, wherein the solubilizing amino acid sequence is a Fasciola hepatica 8-kDa antigen (Fh8) sequence. In embodiments, the peptide further includes a solubilizing amino acid sequence, wherein the solubilizing amino acid sequence is a mutated dehalogenase (Halo) sequence. In embodiments, the peptide further includes a solubilizing amino acid sequence, wherein the solubilizing amino acid sequence is a glutathione-S-transferase (GST) sequence.

In embodiments, the peptide further includes a peptide linker. In embodiments, the peptide linker includes the amino acid sequence of SEQ ID NO:42, SEQ ID NO:44, SEQ ID NO: 45, or SEQ ID NO:47. In embodiments, the peptide linker includes the amino acid sequence of SEQ ID NO:42. In embodiments, the peptide linker includes the amino acid sequence of SEQ ID NO: 44. In embodiments, the peptide linker includes the amino acid sequence of SEQ ID NO: 45. In embodiments, the peptide linker includes the amino acid sequence of SEQ ID NO:47. In embodiments, the peptide linker is the amino acid sequence of SEQ ID NO:42, SEQ ID NO: 44, SEQ ID NO:45, or SEQ ID NO:47. In embodiments, the peptide linker is the amino acid sequence of SEQ ID NO:42. In embodiments, the peptide linker is the amino acid sequence of SEQ ID NO:44. In embodiments, the peptide linker is the amino acid sequence of SEQ ID NO: 45. In embodiments, the peptide linker is the amino acid sequence of SEQ ID NO:47.

In embodiments, the peptide includes a solubilizing amino acid sequence attached to the amino acid sequence of SEQ ID NO:18 through a peptide linker. In embodiments, the solubilizing amino acid sequence includes the amino acid sequence of SEQ ID NO:41 and the peptide linker includes the amino acid sequence of SEQ ID NO:42. In embodiments, the peptide includes the amino acid sequence of SEQ ID NO:30. In embodiments, the peptide is the amino acid sequence of SEQ ID NO:30. In one further embodiment, the peptide is referred to as MBP_N0. In one embodiment, the peptide is referred to as TdT 18 or clone 18.

In another aspect is provided a terminal deoxynucleotidyl transferase (TdT) peptide, wherein the peptide includes an amino acid sequence having at least 80% sequence identity to the amino acid sequence of SEQ ID NO:19. In embodiments, the peptide includes an amino acid sequence having at least 81% sequence identity to the amino acid sequence of SEQ ID NO:19. In embodiments, the peptide includes an amino acid sequence having at least 82% sequence identity to the amino acid sequence of SEQ ID NO:19. In embodiments, the peptide includes an amino acid sequence having at least 83% sequence identity to the amino acid sequence of SEQ ID NO: 19. In embodiments, the peptide includes an amino acid sequence having at least 84% sequence identity to the amino acid sequence of SEQ ID NO: 19. In embodiments, the peptide includes an amino acid sequence having at least 85% sequence identity to the amino acid sequence of SEQ ID NO:19. In embodiments, the peptide includes an amino acid sequence having at least 86% sequence identity to the amino acid sequence of SEQ ID NO:19. In embodiments, the peptide includes an amino acid sequence having at least 87% sequence identity to the amino acid sequence of SEQ ID NO:19. In embodiments, the peptide includes an amino acid sequence having at least 88% sequence identity to the amino acid sequence of SEQ ID NO: 19. In embodiments, the peptide includes an amino acid sequence having at least 89% sequence identity to the amino acid sequence of SEQ ID NO:19. In embodiments, the peptide includes an amino acid sequence having at least 90% sequence identity to the amino acid sequence of SEQ ID NO:19. In embodiments, the peptide includes an amino acid sequence having at least 91% sequence identity to the amino acid sequence of SEQ ID NO:19. In embodiments, the peptide includes an amino acid sequence having at least 92% sequence identity to the amino acid sequence of SEQ ID NO:19. In embodiments, the peptide includes an amino acid sequence having at least 93% sequence identity to the amino acid sequence of SEQ ID NO: 19. In embodiments, the peptide includes an amino acid sequence having at least 94% sequence identity to the amino acid sequence of SEQ ID NO: 19. In embodiments, the peptide includes an amino acid sequence having at least 95% sequence identity to the amino acid sequence of SEQ ID NO:19. In embodiments, the peptide includes an amino acid sequence having at least 96% sequence identity to the amino acid sequence of SEQ ID NO:19. In embodiments, the peptide includes an amino acid sequence having at least 97% sequence identity to the amino acid sequence of SEQ ID NO:19. In embodiments, the peptide includes an amino acid sequence having at least 98% sequence identity to the amino acid sequence of SEQ ID NO: 19. In embodiments, the peptide includes an amino acid sequence having at least 99% sequence identity to the amino acid sequence of SEQ ID NO:19. In embodiments, the peptide includes an amino acid sequence having at least 100% sequence identity to the amino acid sequence of SEQ ID NO:19.

In embodiments, the peptide is an amino acid sequence having at least 80% sequence identity to the amino acid sequence of SEQ ID NO:19. In embodiments, the peptide is an amino acid sequence having at least 81% sequence identity to the amino acid sequence of SEQ ID NO: 19. In embodiments, the peptide is an amino acid sequence having at least 82% sequence identity to the amino acid sequence of SEQ ID NO:19. In embodiments, the peptide is an amino acid sequence having at least 83% sequence identity to the amino acid sequence of SEQ ID NO: 19. In embodiments, the peptide is an amino acid sequence having at least 84% sequence identity to the amino acid sequence of SEQ ID NO:19. In embodiments, the peptide is an amino acid sequence having at least 85% sequence identity to the amino acid sequence of SEQ ID NO: 19. In embodiments, the peptide is an amino acid sequence having at least 86% sequence identity to the amino acid sequence of SEQ ID NO:19. In embodiments, the peptide is an amino acid sequence having at least 87% sequence identity to the amino acid sequence of SEQ ID NO: 19. In embodiments, the peptide is an amino acid sequence having at least 88% sequence identity to the amino acid sequence of SEQ ID NO:19. In embodiments, the peptide is an amino acid sequence having at least 89% sequence identity to the amino acid sequence of SEQ ID NO: 19. In embodiments, the peptide is an amino acid sequence having at least 90% sequence identity to the amino acid sequence of SEQ ID NO:19. In embodiments, the peptide is an amino acid sequence having at least 91% sequence identity to the amino acid sequence of SEQ ID NO: 19. In embodiments, the peptide is an amino acid sequence having at least 92% sequence identity to the amino acid sequence of SEQ ID NO:19. In embodiments, the peptide is an amino acid sequence having at least 93% sequence identity to the amino acid sequence of SEQ ID NO: 19. In embodiments, the peptide is an amino acid sequence having at least 94% sequence identity to the amino acid sequence of SEQ ID NO:19. In embodiments, the peptide is an amino acid sequence having at least 95% sequence identity to the amino acid sequence of SEQ ID NO: 19. In embodiments, the peptide is an amino acid sequence having at least 96% sequence identity to the amino acid sequence of SEQ ID NO:19. In embodiments, the peptide is an amino acid sequence having at least 97% sequence identity to the amino acid sequence of SEQ ID NO: 19. In embodiments, the peptide is an amino acid sequence having at least 98% sequence identity to the amino acid sequence of SEQ ID NO:19. In embodiments, the peptide is an amino acid sequence having at least 99% sequence identity to the amino acid sequence of SEQ ID NO: 19. In embodiments, the peptide is an amino acid sequence having at least 100% sequence identity to the amino acid sequence of SEQ ID NO:19.

In embodiments, the peptide includes an amino acid sequence having 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to 250, 300, 350, 375, 380, 385, 386, 387, 388, 389, 390, 391, or 392 contiguous amino acids of the sequence of SEQ ID NO:19. In embodiments, the peptide includes a sequence having 80% sequence identity to 250, 300, 350, 375, 380, 385, 386, 387, 388, 389, 390, 391, or 392 contiguous amino acids of the sequence of SEQ ID NO:19. In embodiments, the peptide includes a sequence having 81% sequence identity to 250, 300, 350, 375, 380, 385, 386, 387, 388, 389, 390, 391, or 392 contiguous amino acids of the sequence of SEQ ID NO:19. In embodiments, the peptide includes a sequence having 82% sequence identity to 250, 300, 350, 375, 380, 385, 386, 387, 388, 389, 390, 391, or 392 contiguous amino acids of the sequence of SEQ ID NO:19. In embodiments, the peptide includes a sequence having 83% sequence identity to 250, 300, 350, 375, 380, 385, 386, 387, 388, 389, 390, 391, or 392 contiguous amino acids of the sequence of SEQ ID NO:19. In embodiments, the peptide includes a sequence having 84% sequence identity to 250, 300, 350, 375, 380, 385, 386, 387, 388, 389, 390, 391, or 392 contiguous amino acids of the sequence of SEQ ID NO:19. In embodiments, the peptide includes a sequence having 85% sequence identity to 250, 300, 350, 375, 380, 385, 386, 387, 388, 389, 390, 391, or 392 contiguous amino acids of the sequence of SEQ ID NO:19. In embodiments, the peptide includes a sequence having 86% sequence identity to 250, 300, 350, 375, 380, 385, 386, 387, 388, 389, 390, 391, or 392 contiguous amino acids of the sequence of SEQ ID NO:19. In embodiments, the peptide includes a sequence having 87% sequence identity to 250, 300, 350, 375, 380, 385, 386, 387, 388, 389, 390, 391, or 392 contiguous amino acids of the sequence of SEQ ID NO:19. In embodiments, the peptide includes a sequence having 88% sequence identity to 250, 300, 350, 375, 380, 385, 386, 387, 388, 389, 390, 391, or 392 contiguous amino acids of the sequence of SEQ ID NO:19. In embodiments, the peptide includes a sequence having 89% sequence identity to 250, 300, 350, 375, 380, 385, 386, 387, 388, 389, 390, 391, or 392 contiguous amino acids of the sequence of SEQ ID NO:19. In embodiments, the peptide includes a sequence having 90% sequence identity to 250, 300, 350, 375, 380, 385, 386, 387, 388, 389, 390, 391, or 392 contiguous amino acids of the sequence of SEQ ID NO:19. In embodiments, the peptide includes a sequence having 91% sequence identity to 250, 300, 350, 375, 380, 385, 386, 387, 388, 389, 390, 391, or 392 contiguous amino acids of the sequence of SEQ ID NO:19. In embodiments, the peptide includes a sequence having 92% sequence identity to 250, 300, 350, 375, 380, 385, 386, 387, 388, 389, 390, 391, or 392 contiguous amino acids of the sequence of SEQ ID NO:19. In embodiments, the peptide includes a sequence having 93% sequence identity to 250, 300, 350, 375, 380, 385, 386, 387, 388, 389, 390, 391, or 392 contiguous amino acids of the sequence of SEQ ID NO:19. In embodiments, the peptide includes a sequence having 94% sequence identity to 250, 300, 350, 375, 380, 385, 386, 387, 388, 389, 390, 391, or 392 contiguous amino acids of the sequence of SEQ ID NO:19. In embodiments, the peptide includes a sequence having 95% sequence identity to 250, 300, 350, 375, 380, 385, 386, 387, 388, 389, 390, 391, or 392 contiguous amino acids of the sequence of SEQ ID NO:19. In embodiments, the peptide includes a sequence having 96% sequence identity to 250, 300, 350, 375, 380, 385, 386, 387, 388, 389, 390, 391, or 392 contiguous amino acids of the sequence of SEQ ID NO:19. In embodiments, the peptide includes a sequence having 97% sequence identity to 250, 300, 350, 375, 380, 385, 386, 387, 388, 389, 390, 391, or 392 contiguous amino acids of the sequence of SEQ ID NO:19. In embodiments, the peptide includes a sequence having 98% sequence identity to 250, 300, 350, 375, 380, 385, 386, 387, 388, 389, 390, 391, or 392 contiguous amino acids of the sequence of SEQ ID NO:19. In embodiments, the peptide includes a sequence having 99% sequence identity to 250, 300, 350, 375, 380, 385, 386, 387, 388, 389, 390, 391, or 392 contiguous amino acids of the sequence of SEQ ID NO:19. In embodiments, the peptide includes a sequence having 100% sequence identity to 250, 300, 350, 375, 380, 385, 386, 387, 388, 389, 390, 391, or 392 contiguous amino acids of the sequence of SEQ ID NO:19.

In embodiments, the peptide includes the amino acid sequence of SEQ ID NO:19. In embodiments, the peptide has the amino acid sequence of SEQ ID NO:19. In one further embodiment, the peptide is referred to as N1.

In embodiments, the peptide may further include additional protein elements or protein domains. In embodiments, the peptide further includes additional protein elements. In embodiments, the peptide further includes additional protein domains. In embodiments, the additional protein elements or protein domains may include solubilizing amino acid sequences, reporter amino acid sequences, peptide linkers, labels, label moieties, or detectable moieities. In embodiments, the additional protein domain includes a solubilizing amino acid sequence, a reporter amino acid sequence, a peptide linker, a label, a label moiety, or a detectable moiety. In embodiments, the additional protein domain includes a solubilizing amino acid sequence. In embodiments, the additional protein domain includes a reporter amino acid sequence. In embodiments, the additional protein domain includes a peptide linker. In embodiments, the additional protein domain includes a label. In embodiments, the additional protein domain includes a label moiety. In embodiments, the additional protein domain includes a detectable moiety.

In embodiments, the peptide further includes a solubilizing amino acid sequence, wherein the solubilizing amino acid sequence is a maltose-binding protein (MBP) sequence, a small ubiquitin modified (SUMO) sequence, a thioredoxin (TrxA) sequence, a N-utilizing substance A (NusA) sequence, a Fasciola hepatica 8-kDa antigen (Fh8) sequence, a mutated dehalogenase (Halo) sequence, or a glutathione-S-transferase (GST) sequence.

In embodiments, the peptide further includes a solubilizing amino acid sequence, wherein the solubilizing amino acid sequence is a maltose-binding protein (MBP) sequence. In embodiments, the peptide further includes a solubilizing amino acid sequence, wherein the solubilizing amino acid sequence is a small ubiquitin modified (SUMO) sequence. In embodiments, the peptide further includes a solubilizing amino acid sequence, wherein the solubilizing amino acid sequence is a thioredoxin (TrxA) sequence. In embodiments, the peptide further includes a solubilizing amino acid sequence, wherein the solubilizing amino acid sequence is a N-utilizing substance A (NusA) sequence. In embodiments, the peptide further includes a solubilizing amino acid sequence, wherein the solubilizing amino acid sequence is a Fasciola hepatica 8-kDa antigen (Fh8) sequence. In embodiments, the peptide further includes a solubilizing amino acid sequence, wherein the solubilizing amino acid sequence is a mutated dehalogenase (Halo) sequence. In embodiments, the peptide further includes a solubilizing amino acid sequence, wherein the solubilizing amino acid sequence is a glutathione-S-transferase (GST) sequence.

In embodiments, the peptide further includes a peptide linker. In embodiments, the peptide linker includes the amino acid sequence of SEQ ID NO:42, SEQ ID NO:44, SEQ ID NO: 45, or SEQ ID NO:47. In embodiments, the peptide linker includes the amino acid sequence of SEQ ID NO:42. In embodiments, the peptide linker includes the amino acid sequence of SEQ ID NO: 44. In embodiments, the peptide linker includes the amino acid sequence of SEQ ID NO: 45. In embodiments, the peptide linker includes the amino acid sequence of SEQ ID NO:47. In embodiments, the peptide linker is the amino acid sequence of SEQ ID NO:42, SEQ ID NO: 44, SEQ ID NO:45, or SEQ ID NO:47. In embodiments, the peptide linker is the amino acid sequence of SEQ ID NO:42. In embodiments, the peptide linker is the amino acid sequence of SEQ ID NO:44. In embodiments, the peptide linker is the amino acid sequence of SEQ ID NO: 45. In embodiments, the peptide linker is the amino acid sequence of SEQ ID NO:47.

In embodiments, the peptide includes a solubilizing amino acid sequence attached to the amino acid sequence of SEQ ID NO:19 through a peptide linker. In embodiments, the solubilizing amino acid sequence includes the amino acid sequence of SEQ ID NO:41 and the peptide linker includes the amino acid sequence of SEQ ID NO:42. In embodiments, the peptide includes the amino acid sequence of SEQ ID NO:31. In embodiments, the peptide is the amino acid sequence of SEQ ID NO:31. In one further embodiment, the peptide is referred to as MBP_N1.

In another aspect is provided a terminal deoxynucleotidyl transferase (TdT) peptide, wherein the peptide includes an amino acid sequence having at least 80% sequence identity to the amino acid sequence of SEQ ID NO:20. In embodiments, the peptide includes an amino acid sequence having at least 81% sequence identity to the amino acid sequence of SEQ ID NO:20. In embodiments, the peptide includes an amino acid sequence having at least 82% sequence identity to the amino acid sequence of SEQ ID NO:20. In embodiments, the peptide includes an amino acid sequence having at least 83% sequence identity to the amino acid sequence of SEQ ID NO: 20. In embodiments, the peptide includes an amino acid sequence having at least 84% sequence identity to the amino acid sequence of SEQ ID NO:20. In embodiments, the peptide includes an amino acid sequence having at least 85% sequence identity to the amino acid sequence of SEQ ID NO:20. In embodiments, the peptide includes an amino acid sequence having at least 86% sequence identity to the amino acid sequence of SEQ ID NO:20. In embodiments, the peptide includes an amino acid sequence having at least 87% sequence identity to the amino acid sequence of SEQ ID NO:20. In embodiments, the peptide includes an amino acid sequence having at least 88% sequence identity to the amino acid sequence of SEQ ID NO: 20. In embodiments, the peptide includes an amino acid sequence having at least 89% sequence identity to the amino acid sequence of SEQ ID NO:20. In embodiments, the peptide includes an amino acid sequence having at least 90% sequence identity to the amino acid sequence of SEQ ID NO:20. In embodiments, the peptide includes an amino acid sequence having at least 91% sequence identity to the amino acid sequence of SEQ ID NO:20. In embodiments, the peptide includes an amino acid sequence having at least 92% sequence identity to the amino acid sequence of SEQ ID NO:20. In embodiments, the peptide includes an amino acid sequence having at least 93% sequence identity to the amino acid sequence of SEQ ID NO: 20. In embodiments, the peptide includes an amino acid sequence having at least 94% sequence identity to the amino acid sequence of SEQ ID NO:20. In embodiments, the peptide includes an amino acid sequence having at least 95% sequence identity to the amino acid sequence of SEQ ID NO:20. In embodiments, the peptide includes an amino acid sequence having at least 96% sequence identity to the amino acid sequence of SEQ ID NO:20. In embodiments, the peptide includes an amino acid sequence having at least 97% sequence identity to the amino acid sequence of SEQ ID NO:20. In embodiments, the peptide includes an amino acid sequence having at least 98% sequence identity to the amino acid sequence of SEQ ID NO: 20. In embodiments, the peptide includes an amino acid sequence having at least 99% sequence identity to the amino acid sequence of SEQ ID NO:20. In embodiments, the peptide includes an amino acid sequence having at least 100% sequence identity to the amino acid sequence of SEQ ID NO:20.

In embodiments, the peptide is an amino acid sequence having at least 80% sequence identity to the amino acid sequence of SEQ ID NO:20. In embodiments, the peptide is an amino acid sequence having at least 81% sequence identity to the amino acid sequence of SEQ ID NO: 20. In embodiments, the peptide is an amino acid sequence having at least 82% sequence identity to the amino acid sequence of SEQ ID NO:20. In embodiments, the peptide is an amino acid sequence having at least 83% sequence identity to the amino acid sequence of SEQ ID NO: 20. In embodiments, the peptide is an amino acid sequence having at least 84% sequence identity to the amino acid sequence of SEQ ID NO:20. In embodiments, the peptide is an amino acid sequence having at least 85% sequence identity to the amino acid sequence of SEQ ID NO: 20. In embodiments, the peptide is an amino acid sequence having at least 86% sequence identity to the amino acid sequence of SEQ ID NO:20. In embodiments, the peptide is an amino acid sequence having at least 87% sequence identity to the amino acid sequence of SEQ ID NO: 20. In embodiments, the peptide is an amino acid sequence having at least 88% sequence identity to the amino acid sequence of SEQ ID NO:20. In embodiments, the peptide is an amino acid sequence having at least 89% sequence identity to the amino acid sequence of SEQ ID NO: 20. In embodiments, the peptide is an amino acid sequence having at least 90% sequence identity to the amino acid sequence of SEQ ID NO:20. In embodiments, the peptide is an amino acid sequence having at least 91% sequence identity to the amino acid sequence of SEQ ID NO: 20. In embodiments, the peptide is an amino acid sequence having at least 92% sequence identity to the amino acid sequence of SEQ ID NO:20. In embodiments, the peptide is an amino acid sequence having at least 93% sequence identity to the amino acid sequence of SEQ ID NO: 20. In embodiments, the peptide is an amino acid sequence having at least 94% sequence identity to the amino acid sequence of SEQ ID NO:20. In embodiments, the peptide is an amino acid sequence having at least 95% sequence identity to the amino acid sequence of SEQ ID NO: 20. In embodiments, the peptide is an amino acid sequence having at least 96% sequence identity to the amino acid sequence of SEQ ID NO:20. In embodiments, the peptide is an amino acid sequence having at least 97% sequence identity to the amino acid sequence of SEQ ID NO: 20. In embodiments, the peptide is an amino acid sequence having at least 98% sequence identity to the amino acid sequence of SEQ ID NO:20. In embodiments, the peptide is an amino acid sequence having at least 99% sequence identity to the amino acid sequence of SEQ ID NO: 20. In embodiments, the peptide is an amino acid sequence having at least 100% sequence identity to the amino acid sequence of SEQ ID NO:20.

In embodiments, the peptide includes an amino acid sequence having 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to 250, 300, 350, 375, 380, 385, 386, 387, 388, 389, or 390 contiguous amino acids of the sequence of SEQ ID NO:20. In embodiments, the peptide includes a sequence having 80% sequence identity to 250, 300, 350, 375, 380, 385, 386, 387, 388, 389, or 390 contiguous amino acids of the sequence of SEQ ID NO:20. In embodiments, the peptide includes a sequence having 81% sequence identity to 250, 300, 350, 375, 380, 385, 386, 387, 388, 389, or 390 contiguous amino acids of the sequence of SEQ ID NO:20. In embodiments, the peptide includes a sequence having 82% sequence identity to 250, 300, 350, 375, 380, 385, 386, 387, 388, 389, or 390 contiguous amino acids of the sequence of SEQ ID NO:20. In embodiments, the peptide includes a sequence having 83% sequence identity to 250, 300, 350, 375, 380, 385, 386, 387, 388, 389, or 390 contiguous amino acids of the sequence of SEQ ID NO: 20. In embodiments, the peptide includes a sequence having 84% sequence identity to 250, 300, 350, 375, 380, 385, 386, 387, 388, 389, or 390 contiguous amino acids of the sequence of SEQ ID NO:20. In embodiments, the peptide includes a sequence having 85% sequence identity to 250, 300, 350, 375, 380, 385, 386, 387, 388, 389, or 390 contiguous amino acids of the sequence of SEQ ID NO:20. In embodiments, the peptide includes a sequence having 86% sequence identity to 250, 300, 350, 375, 380, 385, 386, 387, 388, 389, or 390 contiguous amino acids of the sequence of SEQ ID NO:20. In embodiments, the peptide includes a sequence having 87% sequence identity to 250, 300, 350, 375, 380, 385, 386, 387, 388, 389, or 390 contiguous amino acids of the sequence of SEQ ID NO:20. In embodiments, the peptide includes a sequence having 88% sequence identity to 250, 300, 350, 375, 380, 385, 386, 387, 388, 389, or 390 contiguous amino acids of the sequence of SEQ ID NO:20. In embodiments, the peptide includes a sequence having 89% sequence identity to 250, 300, 350, 375, 380, 385, 386, 387, 388, 389, or 390 contiguous amino acids of the sequence of SEQ ID NO:20. In embodiments, the peptide includes a sequence having 90% sequence identity to 250, 300, 350, 375, 380, 385, 386, 387, 388, 389, or 390 contiguous amino acids of the sequence of SEQ ID NO:20. In embodiments, the peptide includes a sequence having 91% sequence identity to 250, 300, 350, 375, 380, 385, 386, 387, 388, 389, or 390 contiguous amino acids of the sequence of SEQ ID NO: 20. In embodiments, the peptide includes a sequence having 92% sequence identity to 250, 300, 350, 375, 380, 385, 386, 387, 388, 389, or 390 contiguous amino acids of the sequence of SEQ ID NO:20. In embodiments, the peptide includes a sequence having 93% sequence identity to 250, 300, 350, 375, 380, 385, 386, 387, 388, 389, or 390 contiguous amino acids of the sequence of SEQ ID NO:20. In embodiments, the peptide includes a sequence having 94% sequence identity to 250, 300, 350, 375, 380, 385, 386, 387, 388, 389, or 390 contiguous amino acids of the sequence of SEQ ID NO:20. In embodiments, the peptide includes a sequence having 95% sequence identity to 250, 300, 350, 375, 380, 385, 386, 387, 388, 389, or 390 contiguous amino acids of the sequence of SEQ ID NO:20. In embodiments, the peptide includes a sequence having 96% sequence identity to 250, 300, 350, 375, 380, 385, 386, 387, 388, 389, or 390 contiguous amino acids of the sequence of SEQ ID NO:20. In embodiments, the peptide includes a sequence having 97% sequence identity to 250, 300, 350, 375, 380, 385, 386, 387, 388, 389, or 390 contiguous amino acids of the sequence of SEQ ID NO:20. In embodiments, the peptide includes a sequence having 98% sequence identity to 250, 300, 350, 375, 380, 385, 386, 387, 388, 389, or 390 contiguous amino acids of the sequence of SEQ ID NO:20. In embodiments, the peptide includes a sequence having 99% sequence identity to 250, 300, 350, 375, 380, 385, 386, 387, 388, 389, or 390 contiguous amino acids of the sequence of SEQ ID NO: 20. In embodiments, the peptide includes a sequence having 100% sequence identity to 250, 300, 350, 375, 380, 385, 386, 387, 388, 389, or 390 contiguous amino acids of the sequence of SEQ ID NO:20.

In embodiments, the peptide includes the amino acid sequence of SEQ ID NO:20. In embodiments, the peptide has the amino acid sequence of SEQ ID NO:20. In one further embodiment, the peptide is referred to as N3.

In embodiments, the peptide may further include additional protein elements or protein domains. In embodiments, the peptide further includes additional protein elements. In embodiments, the peptide further includes additional protein domains. In embodiments, the additional protein elements or protein domains may include solubilizing amino acid sequences, reporter amino acid sequences, peptide linkers, labels, label moieties, or detectable moieities. In embodiments, the additional protein domain includes a solubilizing amino acid sequence, a reporter amino acid sequence, a peptide linker, a label, a label moiety, or a detectable moiety. In embodiments, the additional protein domain includes a solubilizing amino acid sequence. In embodiments, the additional protein domain includes a reporter amino acid sequence. In embodiments, the additional protein domain includes a peptide linker. In embodiments, the additional protein domain includes a label. In embodiments, the additional protein domain includes a label moiety. In embodiments, the additional protein domain includes a detectable moiety.

In embodiments, the peptide further includes a solubilizing amino acid sequence, wherein the solubilizing amino acid sequence is a maltose-binding protein (MBP) sequence, a small ubiquitin modified (SUMO) sequence, a thioredoxin (TrxA) sequence, a N-utilizing substance A (NusA) sequence, a Fasciola hepatica 8-kDa antigen (Fh8) sequence, a mutated dehalogenase (Halo) sequence, or a glutathione-S-transferase (GST) sequence.

In embodiments, the peptide further includes a solubilizing amino acid sequence, wherein the solubilizing amino acid sequence is a maltose-binding protein (MBP) sequence. In embodiments, the peptide further includes a solubilizing amino acid sequence, wherein the solubilizing amino acid sequence is a small ubiquitin modified (SUMO) sequence. In embodiments, the peptide further includes a solubilizing amino acid sequence, wherein the solubilizing amino acid sequence is a thioredoxin (TrxA) sequence. In embodiments, the peptide further includes a solubilizing amino acid sequence, wherein the solubilizing amino acid sequence is a N-utilizing substance A (NusA) sequence. In embodiments, the peptide further includes a solubilizing amino acid sequence, wherein the solubilizing amino acid sequence is a Fasciola hepatica 8-kDa antigen (Fh8) sequence. In embodiments, the peptide further includes a solubilizing amino acid sequence, wherein the solubilizing amino acid sequence is a mutated dehalogenase (Halo) sequence. In embodiments, the peptide further includes a solubilizing amino acid sequence, wherein the solubilizing amino acid sequence is a glutathione-S-transferase (GST) sequence.

In embodiments, the peptide further includes a peptide linker. In embodiments, the peptide linker includes the amino acid sequence of SEQ ID NO:42, SEQ ID NO:44, SEQ ID NO: 45, or SEQ ID NO:47. In embodiments, the peptide linker includes the amino acid sequence of SEQ ID NO:42. In embodiments, the peptide linker includes the amino acid sequence of SEQ ID NO: 44. In embodiments, the peptide linker includes the amino acid sequence of SEQ ID NO: 45. In embodiments, the peptide linker includes the amino acid sequence of SEQ ID NO:47. In embodiments, the peptide linker is the amino acid sequence of SEQ ID NO:42, SEQ ID NO: 44, SEQ ID NO:45, or SEQ ID NO:47. In embodiments, the peptide linker is the amino acid sequence of SEQ ID NO:42. In embodiments, the peptide linker is the amino acid sequence of SEQ ID NO:44. In embodiments, the peptide linker is the amino acid sequence of SEQ ID NO: 45. In embodiments, the peptide linker is the amino acid sequence of SEQ ID NO:47.

In embodiments, the peptide includes a solubilizing amino acid sequence attached to the amino acid sequence of SEQ ID NO:20 through a peptide linker. In embodiments, the solubilizing amino acid sequence includes the amino acid sequence of SEQ ID NO:41 and the peptide linker includes the amino acid sequence of SEQ ID NO:42. In embodiments, the peptide includes the amino acid sequence of SEQ ID NO:32. In embodiments, the peptide is the amino acid sequence of SEQ ID NO:32. In one further embodiment, the peptide is referred to as MBP_N3. In one embodiment, the peptide is referred to as TdT 20 or clone 20.

In another aspect is provided a terminal deoxynucleotidyl transferase (TdT) peptide, wherein the peptide includes an amino acid sequence having at least 80% sequence identity to the amino acid sequence of SEQ ID NO:21. In embodiments, the peptide includes an amino acid sequence having at least 81% sequence identity to the amino acid sequence of SEQ ID NO:21. In embodiments, the peptide includes an amino acid sequence having at least 82% sequence identity to the amino acid sequence of SEQ ID NO:21. In embodiments, the peptide includes an amino acid sequence having at least 83% sequence identity to the amino acid sequence of SEQ ID NO: 21. In embodiments, the peptide includes an amino acid sequence having at least 84% sequence identity to the amino acid sequence of SEQ ID NO:21. In embodiments, the peptide includes an amino acid sequence having at least 85% sequence identity to the amino acid sequence of SEQ ID NO:21. In embodiments, the peptide includes an amino acid sequence having at least 86% sequence identity to the amino acid sequence of SEQ ID NO:21. In embodiments, the peptide includes an amino acid sequence having at least 87% sequence identity to the amino acid sequence of SEQ ID NO:21. In embodiments, the peptide includes an amino acid sequence having at least 88% sequence identity to the amino acid sequence of SEQ ID NO: 21. In embodiments, the peptide includes an amino acid sequence having at least 89% sequence identity to the amino acid sequence of SEQ ID NO:21. In embodiments, the peptide includes an amino acid sequence having at least 90% sequence identity to the amino acid sequence of SEQ ID NO:21. In embodiments, the peptide includes an amino acid sequence having at least 91% sequence identity to the amino acid sequence of SEQ ID NO:21. In embodiments, the peptide includes an amino acid sequence having at least 92% sequence identity to the amino acid sequence of SEQ ID NO:21. In embodiments, the peptide includes an amino acid sequence having at least 93% sequence identity to the amino acid sequence of SEQ ID NO: 21. In embodiments, the peptide includes an amino acid sequence having at least 94% sequence identity to the amino acid sequence of SEQ ID NO:21. In embodiments, the peptide includes an amino acid sequence having at least 95% sequence identity to the amino acid sequence of SEQ ID NO:21. In embodiments, the peptide includes an amino acid sequence having at least 96% sequence identity to the amino acid sequence of SEQ ID NO:21. In embodiments, the peptide includes an amino acid sequence having at least 97% sequence identity to the amino acid sequence of SEQ ID NO:21. In embodiments, the peptide includes an amino acid sequence having at least 98% sequence identity to the amino acid sequence of SEQ ID NO: 21. In embodiments, the peptide includes an amino acid sequence having at least 99% sequence identity to the amino acid sequence of SEQ ID NO:21. In embodiments, the peptide includes an amino acid sequence having at least 100% sequence identity to the amino acid sequence of SEQ ID NO:21.

In embodiments, the peptide is an amino acid sequence having at least 80% sequence identity to the amino acid sequence of SEQ ID NO:21. In embodiments, the peptide is an amino acid sequence having at least 81% sequence identity to the amino acid sequence of SEQ ID NO: 21. In embodiments, the peptide is an amino acid sequence having at least 82% sequence identity to the amino acid sequence of SEQ ID NO:21. In embodiments, the peptide is an amino acid sequence having at least 83% sequence identity to the amino acid sequence of SEQ ID NO: 21. In embodiments, the peptide is an amino acid sequence having at least 84% sequence identity to the amino acid sequence of SEQ ID NO:21. In embodiments, the peptide is an amino acid sequence having at least 85% sequence identity to the amino acid sequence of SEQ ID NO: 21. In embodiments, the peptide is an amino acid sequence having at least 86% sequence identity to the amino acid sequence of SEQ ID NO:21. In embodiments, the peptide is an amino acid sequence having at least 87% sequence identity to the amino acid sequence of SEQ ID NO: 21. In embodiments, the peptide is an amino acid sequence having at least 88% sequence identity to the amino acid sequence of SEQ ID NO:21. In embodiments, the peptide is an amino acid sequence having at least 89% sequence identity to the amino acid sequence of SEQ ID NO: 21. In embodiments, the peptide is an amino acid sequence having at least 90% sequence identity to the amino acid sequence of SEQ ID NO:21. In embodiments, the peptide is an amino acid sequence having at least 91% sequence identity to the amino acid sequence of SEQ ID NO: 21. In embodiments, the peptide is an amino acid sequence having at least 92% sequence identity to the amino acid sequence of SEQ ID NO:21. In embodiments, the peptide is an amino acid sequence having at least 93% sequence identity to the amino acid sequence of SEQ ID NO: 21. In embodiments, the peptide is an amino acid sequence having at least 94% sequence identity to the amino acid sequence of SEQ ID NO:21. In embodiments, the peptide is an amino acid sequence having at least 95% sequence identity to the amino acid sequence of SEQ ID NO: 21. In embodiments, the peptide is an amino acid sequence having at least 96% sequence identity to the amino acid sequence of SEQ ID NO:21. In embodiments, the peptide is an amino acid sequence having at least 97% sequence identity to the amino acid sequence of SEQ ID NO: 21. In embodiments, the peptide is an amino acid sequence having at least 98% sequence identity to the amino acid sequence of SEQ ID NO:21. In embodiments, the peptide is an amino acid sequence having at least 99% sequence identity to the amino acid sequence of SEQ ID NO: 21. In embodiments, the peptide is an amino acid sequence having at least 100% sequence identity to the amino acid sequence of SEQ ID NO:21.

In embodiments, the peptide includes an amino acid sequence having 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to 250, 300, 350, 375, 380, 385, 386, 387, 388, 389, 390, 391, or 392 contiguous amino acids of the sequence of SEQ ID NO:21. In embodiments, the peptide includes a sequence having 80% sequence identity to 250, 300, 350, 375, 380, 385, 386, 387, 388, 389, 390, 391, or 392 contiguous amino acids of the sequence of SEQ ID NO:21. In embodiments, the peptide includes a sequence having 81% sequence identity to 250, 300, 350, 375, 380, 385, 386, 387, 388, 389, 390, 391, or 392 contiguous amino acids of the sequence of SEQ ID NO:21. In embodiments, the peptide includes a sequence having 82% sequence identity to 250, 300, 350, 375, 380, 385, 386, 387, 388, 389, 390, 391, or 392 contiguous amino acids of the sequence of SEQ ID NO:21. In embodiments, the peptide includes a sequence having 83% sequence identity to 250, 300, 350, 375, 380, 385, 386, 387, 388, 389, 390, 391, or 392 contiguous amino acids of the sequence of SEQ ID NO:21. In embodiments, the peptide includes a sequence having 84% sequence identity to 250, 300, 350, 375, 380, 385, 386, 387, 388, 389, 390, 391, or 392 contiguous amino acids of the sequence of SEQ ID NO:21. In embodiments, the peptide includes a sequence having 85% sequence identity to 250, 300, 350, 375, 380, 385, 386, 387, 388, 389, 390, 391, or 392 contiguous amino acids of the sequence of SEQ ID NO:21. In embodiments, the peptide includes a sequence having 86% sequence identity to 250, 300, 350, 375, 380, 385, 386, 387, 388, 389, 390, 391, or 392 contiguous amino acids of the sequence of SEQ ID NO:21. In embodiments, the peptide includes a sequence having 87% sequence identity to 250, 300, 350, 375, 380, 385, 386, 387, 388, 389, 390, 391, or 392 contiguous amino acids of the sequence of SEQ ID NO:21. In embodiments, the peptide includes a sequence having 88% sequence identity to 250, 300, 350, 375, 380, 385, 386, 387, 388, 389, 390, 391, or 392 contiguous amino acids of the sequence of SEQ ID NO:21. In embodiments, the peptide includes a sequence having 89% sequence identity to 250, 300, 350, 375, 380, 385, 386, 387, 388, 389, 390, 391, or 392 contiguous amino acids of the sequence of SEQ ID NO:21. In embodiments, the peptide includes a sequence having 90% sequence identity to 250, 300, 350, 375, 380, 385, 386, 387, 388, 389, 390, 391, or 392 contiguous amino acids of the sequence of SEQ ID NO:21. In embodiments, the peptide includes a sequence having 91% sequence identity to 250, 300, 350, 375, 380, 385, 386, 387, 388, 389, 390, 391, or 392 contiguous amino acids of the sequence of SEQ ID NO:21. In embodiments, the peptide includes a sequence having 92% sequence identity to 250, 300, 350, 375, 380, 385, 386, 387, 388, 389, 390, 391, or 392 contiguous amino acids of the sequence of SEQ ID NO:21. In embodiments, the peptide includes a sequence having 93% sequence identity to 250, 300, 350, 375, 380, 385, 386, 387, 388, 389, 390, 391, or 392 contiguous amino acids of the sequence of SEQ ID NO:21. In embodiments, the peptide includes a sequence having 94% sequence identity to 250, 300, 350, 375, 380, 385, 386, 387, 388, 389, 390, 391, or 392 contiguous amino acids of the sequence of SEQ ID NO:21. In embodiments, the peptide includes a sequence having 95% sequence identity to 250, 300, 350, 375, 380, 385, 386, 387, 388, 389, 390, 391, or 392 contiguous amino acids of the sequence of SEQ ID NO:21. In embodiments, the peptide includes a sequence having 96% sequence identity to 250, 300, 350, 375, 380, 385, 386, 387, 388, 389, 390, 391, or 392 contiguous amino acids of the sequence of SEQ ID NO:21. In embodiments, the peptide includes a sequence having 97% sequence identity to 250, 300, 350, 375, 380, 385, 386, 387, 388, 389, 390, 391, or 392 contiguous amino acids of the sequence of SEQ ID NO:21. In embodiments, the peptide includes a sequence having 98% sequence identity to 250, 300, 350, 375, 380, 385, 386, 387, 388, 389, 390, 391, or 392 contiguous amino acids of the sequence of SEQ ID NO:21. In embodiments, the peptide includes a sequence having 99% sequence identity to 250, 300, 350, 375, 380, 385, 386, 387, 388, 389, 390, 391, or 392 contiguous amino acids of the sequence of SEQ ID NO:21. In embodiments, the peptide includes a sequence having 100% sequence identity to 250, 300, 350, 375, 380, 385, 386, 387, 388, 389, 390, 391, or 392 contiguous amino acids of the sequence of SEQ ID NO:21.

In embodiments, the peptide includes the amino acid sequence of SEQ ID NO:21. In embodiments, the peptide has the amino acid sequence of SEQ ID NO:21. In one further embodiment, the peptide is referred to as N44.

In embodiments, the peptide may further include additional protein elements or protein domains. In embodiments, the peptide further includes additional protein elements. In embodiments, the peptide further includes additional protein domains. In embodiments, the additional protein elements or protein domains may include solubilizing amino acid sequences, reporter amino acid sequences, peptide linkers, labels, label moieties, or detectable moieities. In embodiments, the additional protein domain includes a solubilizing amino acid sequence, a reporter amino acid sequence, a peptide linker, a label, a label moiety, or a detectable moiety. In embodiments, the additional protein domain includes a solubilizing amino acid sequence. In embodiments, the additional protein domain includes a reporter amino acid sequence. In embodiments, the additional protein domain includes a peptide linker. In embodiments, the additional protein domain includes a label. In embodiments, the additional protein domain includes a label moiety. In embodiments, the additional protein domain includes a detectable moiety.

In embodiments, the peptide further includes a solubilizing amino acid sequence, wherein the solubilizing amino acid sequence is a maltose-binding protein (MBP) sequence, a small ubiquitin modified (SUMO) sequence, a thioredoxin (TrxA) sequence, a N-utilizing substance A (NusA) sequence, a Fasciola hepatica 8-kDa antigen (Fh8) sequence, a mutated dehalogenase (Halo) sequence, or a glutathione-S-transferase (GST) sequence.

In embodiments, the peptide further includes a solubilizing amino acid sequence, wherein the solubilizing amino acid sequence is a maltose-binding protein (MBP) sequence. In embodiments, the peptide further includes a solubilizing amino acid sequence, wherein the solubilizing amino acid sequence is a small ubiquitin modified (SUMO) sequence. In embodiments, the peptide further includes a solubilizing amino acid sequence, wherein the solubilizing amino acid sequence is a thioredoxin (TrxA) sequence. In embodiments, the peptide further includes a solubilizing amino acid sequence, wherein the solubilizing amino acid sequence is a N-utilizing substance A (NusA) sequence. In embodiments, the peptide further includes a solubilizing amino acid sequence, wherein the solubilizing amino acid sequence is a Fasciola hepatica 8-kDa antigen (Fh8) sequence. In embodiments, the peptide further includes a solubilizing amino acid sequence, wherein the solubilizing amino acid sequence is a mutated dehalogenase (Halo) sequence. In embodiments, the peptide further includes a solubilizing amino acid sequence, wherein the solubilizing amino acid sequence is a glutathione-S-transferase (GST) sequence.

In embodiments, the peptide further includes a peptide linker. In embodiments, the peptide linker includes the amino acid sequence of SEQ ID NO:42, SEQ ID NO:44, SEQ ID NO: 45, or SEQ ID NO:47. In embodiments, the peptide linker includes the amino acid sequence of SEQ ID NO:42. In embodiments, the peptide linker includes the amino acid sequence of SEQ ID NO: 44. In embodiments, the peptide linker includes the amino acid sequence of SEQ ID NO: 45. In embodiments, the peptide linker includes the amino acid sequence of SEQ ID NO:47. In embodiments, the peptide linker is the amino acid sequence of SEQ ID NO:42, SEQ ID NO: 44, SEQ ID NO:45, or SEQ ID NO:47. In embodiments, the peptide linker is the amino acid sequence of SEQ ID NO:42. In embodiments, the peptide linker is the amino acid sequence of SEQ ID NO:44. In embodiments, the peptide linker is the amino acid sequence of SEQ ID NO: 45. In embodiments, the peptide linker is the amino acid sequence of SEQ ID NO:47.

In embodiments, the peptide includes a solubilizing amino acid sequence attached to the amino acid sequence of SEQ ID NO:21 through a peptide linker. In embodiments, the solubilizing amino acid sequence includes the amino acid sequence of SEQ ID NO:41 and the peptide linker includes the amino acid sequence of SEQ ID NO:42. In embodiments, the peptide includes the amino acid sequence of SEQ ID NO:33. In embodiments, the peptide is the amino acid sequence of SEQ ID NO:33. In one further embodiment, the peptide is referred to as MBP_N44.

In another aspect is provided a terminal deoxynucleotidyl transferase (TdT) peptide, wherein the peptide includes an amino acid sequence having at least 80% sequence identity to the amino acid sequence of SEQ ID NO:22. In embodiments, the peptide includes an amino acid sequence having at least 81% sequence identity to the amino acid sequence of SEQ ID NO:22. In embodiments, the peptide includes an amino acid sequence having at least 82% sequence identity to the amino acid sequence of SEQ ID NO:22. In embodiments, the peptide includes an amino acid sequence having at least 83% sequence identity to the amino acid sequence of SEQ ID NO: 22. In embodiments, the peptide includes an amino acid sequence having at least 84% sequence identity to the amino acid sequence of SEQ ID NO:22. In embodiments, the peptide includes an amino acid sequence having at least 85% sequence identity to the amino acid sequence of SEQ ID NO:22. In embodiments, the peptide includes an amino acid sequence having at least 86% sequence identity to the amino acid sequence of SEQ ID NO:22. In embodiments, the peptide includes an amino acid sequence having at least 87% sequence identity to the amino acid sequence of SEQ ID NO:22. In embodiments, the peptide includes an amino acid sequence having at least 88% sequence identity to the amino acid sequence of SEQ ID NO: 22. In embodiments, the peptide includes an amino acid sequence having at least 89% sequence identity to the amino acid sequence of SEQ ID NO:22. In embodiments, the peptide includes an amino acid sequence having at least 90% sequence identity to the amino acid sequence of SEQ ID NO:22. In embodiments, the peptide includes an amino acid sequence having at least 91% sequence identity to the amino acid sequence of SEQ ID NO:22. In embodiments, the peptide includes an amino acid sequence having at least 92% sequence identity to the amino acid sequence of SEQ ID NO:22. In embodiments, the peptide includes an amino acid sequence having at least 93% sequence identity to the amino acid sequence of SEQ ID NO: 22. In embodiments, the peptide includes an amino acid sequence having at least 94% sequence identity to the amino acid sequence of SEQ ID NO:22. In embodiments, the peptide includes an amino acid sequence having at least 95% sequence identity to the amino acid sequence of SEQ ID NO:22. In embodiments, the peptide includes an amino acid sequence having at least 96% sequence identity to the amino acid sequence of SEQ ID NO:22. In embodiments, the peptide includes an amino acid sequence having at least 97% sequence identity to the amino acid sequence of SEQ ID NO:22. In embodiments, the peptide includes an amino acid sequence having at least 98% sequence identity to the amino acid sequence of SEQ ID NO: 22. In embodiments, the peptide includes an amino acid sequence having at least 99% sequence identity to the amino acid sequence of SEQ ID NO:22. In embodiments, the peptide includes an amino acid sequence having at least 100% sequence identity to the amino acid sequence of SEQ ID NO:22.

In embodiments, the peptide is an amino acid sequence having at least 80% sequence identity to the amino acid sequence of SEQ ID NO:22. In embodiments, the peptide is an amino acid sequence having at least 81% sequence identity to the amino acid sequence of SEQ ID NO: 22. In embodiments, the peptide is an amino acid sequence having at least 82% sequence identity to the amino acid sequence of SEQ ID NO:22. In embodiments, the peptide is an amino acid sequence having at least 83% sequence identity to the amino acid sequence of SEQ ID NO: 22. In embodiments, the peptide is an amino acid sequence having at least 84% sequence identity to the amino acid sequence of SEQ ID NO:22. In embodiments, the peptide is an amino acid sequence having at least 85% sequence identity to the amino acid sequence of SEQ ID NO: 22. In embodiments, the peptide is an amino acid sequence having at least 86% sequence identity to the amino acid sequence of SEQ ID NO:22. In embodiments, the peptide is an amino acid sequence having at least 87% sequence identity to the amino acid sequence of SEQ ID NO: 22. In embodiments, the peptide is an amino acid sequence having at least 88% sequence identity to the amino acid sequence of SEQ ID NO:22. In embodiments, the peptide is an amino acid sequence having at least 89% sequence identity to the amino acid sequence of SEQ ID NO: 22. In embodiments, the peptide is an amino acid sequence having at least 90% sequence identity to the amino acid sequence of SEQ ID NO:22. In embodiments, the peptide is an amino acid sequence having at least 91% sequence identity to the amino acid sequence of SEQ ID NO: 22. In embodiments, the peptide is an amino acid sequence having at least 92% sequence identity to the amino acid sequence of SEQ ID NO:22. In embodiments, the peptide is an amino acid sequence having at least 93% sequence identity to the amino acid sequence of SEQ ID NO: 22. In embodiments, the peptide is an amino acid sequence having at least 94% sequence identity to the amino acid sequence of SEQ ID NO:22. In embodiments, the peptide is an amino acid sequence having at least 95% sequence identity to the amino acid sequence of SEQ ID NO: 22. In embodiments, the peptide is an amino acid sequence having at least 96% sequence identity to the amino acid sequence of SEQ ID NO:22. In embodiments, the peptide is an amino acid sequence having at least 97% sequence identity to the amino acid sequence of SEQ ID NO: 22. In embodiments, the peptide is an amino acid sequence having at least 98% sequence identity to the amino acid sequence of SEQ ID NO:22. In embodiments, the peptide is an amino acid sequence having at least 99% sequence identity to the amino acid sequence of SEQ ID NO: 22. In embodiments, the peptide is an amino acid sequence having at least 100% sequence identity to the amino acid sequence of SEQ ID NO:22.

In embodiments, the peptide includes an amino acid sequence having 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to 250, 300, 350, 375, 380, 385, 386, 387, 388, 389, 390, 391, or 392 contiguous amino acids of the sequence of SEQ ID NO:22. In embodiments, the peptide includes a sequence having 80% sequence identity to 250, 300, 350, 375, 380, 385, 386, 387, 388, 389, 390, 391, or 392 contiguous amino acids of the sequence of SEQ ID NO:22. In embodiments, the peptide includes a sequence having 81% sequence identity to 250, 300, 350, 375, 380, 385, 386, 387, 388, 389, 390, 391, or 392 contiguous amino acids of the sequence of SEQ ID NO:22. In embodiments, the peptide includes a sequence having 82% sequence identity to 250, 300, 350, 375, 380, 385, 386, 387, 388, 389, 390, 391, or 392 contiguous amino acids of the sequence of SEQ ID NO:22. In embodiments, the peptide includes a sequence having 83% sequence identity to 250, 300, 350, 375, 380, 385, 386, 387, 388, 389, 390, 391, or 392 contiguous amino acids of the sequence of SEQ ID NO:22. In embodiments, the peptide includes a sequence having 84% sequence identity to 250, 300, 350, 375, 380, 385, 386, 387, 388, 389, 390, 391, or 392 contiguous amino acids of the sequence of SEQ ID NO:22. In embodiments, the peptide includes a sequence having 85% sequence identity to 250, 300, 350, 375, 380, 385, 386, 387, 388, 389, 390, 391, or 392 contiguous amino acids of the sequence of SEQ ID NO:22. In embodiments, the peptide includes a sequence having 86% sequence identity to 250, 300, 350, 375, 380, 385, 386, 387, 388, 389, 390, 391, or 392 contiguous amino acids of the sequence of SEQ ID NO:22. In embodiments, the peptide includes a sequence having 87% sequence identity to 250, 300, 350, 375, 380, 385, 386, 387, 388, 389, 390, 391, or 392 contiguous amino acids of the sequence of SEQ ID NO:22. In embodiments, the peptide includes a sequence having 88% sequence identity to 250, 300, 350, 375, 380, 385, 386, 387, 388, 389, 390, 391, or 392 contiguous amino acids of the sequence of SEQ ID NO:22. In embodiments, the peptide includes a sequence having 89% sequence identity to 250, 300, 350, 375, 380, 385, 386, 387, 388, 389, 390, 391, or 392 contiguous amino acids of the sequence of SEQ ID NO:22. In embodiments, the peptide includes a sequence having 90% sequence identity to 250, 300, 350, 375, 380, 385, 386, 387, 388, 389, 390, 391, or 392 contiguous amino acids of the sequence of SEQ ID NO:22. In embodiments, the peptide includes a sequence having 91% sequence identity to 250, 300, 350, 375, 380, 385, 386, 387, 388, 389, 390, 391, or 392 contiguous amino acids of the sequence of SEQ ID NO:22. In embodiments, the peptide includes a sequence having 92% sequence identity to 250, 300, 350, 375, 380, 385, 386, 387, 388, 389, 390, 391, or 392 contiguous amino acids of the sequence of SEQ ID NO:22. In embodiments, the peptide includes a sequence having 93% sequence identity to 250, 300, 350, 375, 380, 385, 386, 387, 388, 389, 390, 391, or 392 contiguous amino acids of the sequence of SEQ ID NO:22. In embodiments, the peptide includes a sequence having 94% sequence identity to 250, 300, 350, 375, 380, 385, 386, 387, 388, 389, 390, 391, or 392 contiguous amino acids of the sequence of SEQ ID NO:22. In embodiments, the peptide includes a sequence having 95% sequence identity to 250, 300, 350, 375, 380, 385, 386, 387, 388, 389, 390, 391, or 392 contiguous amino acids of the sequence of SEQ ID NO:22. In embodiments, the peptide includes a sequence having 96% sequence identity to 250, 300, 350, 375, 380, 385, 386, 387, 388, 389, 390, 391, or 392 contiguous amino acids of the sequence of SEQ ID NO:22. In embodiments, the peptide includes a sequence having 97% sequence identity to 250, 300, 350, 375, 380, 385, 386, 387, 388, 389, 390, 391, or 392 contiguous amino acids of the sequence of SEQ ID NO:22. In embodiments, the peptide includes a sequence having 98% sequence identity to 250, 300, 350, 375, 380, 385, 386, 387, 388, 389, 390, 391, or 392 contiguous amino acids of the sequence of SEQ ID NO:22. In embodiments, the peptide includes a sequence having 99% sequence identity to 250, 300, 350, 375, 380, 385, 386, 387, 388, 389, 390, 391, or 392 contiguous amino acids of the sequence of SEQ ID NO:22. In embodiments, the peptide includes a sequence having 100% sequence identity to 250, 300, 350, 375, 380, 385, 386, 387, 388, 389, 390, 391, or 392 contiguous amino acids of the sequence of SEQ ID NO:22.

In embodiments, the peptide includes the amino acid sequence of SEQ ID NO:22. In embodiments, the peptide has the amino acid sequence of SEQ ID NO:22. In one further embodiment, the peptide is referred to as N44dm.

In embodiments, the peptide may further include additional protein elements or protein domains. In embodiments, the peptide further includes additional protein elements. In embodiments, the peptide further includes additional protein domains. In embodiments, the additional protein elements or protein domains may include solubilizing amino acid sequences, reporter amino acid sequences, peptide linkers, labels, label moieties, or detectable moieities. In embodiments, the additional protein domain includes a solubilizing amino acid sequence, a reporter amino acid sequence, a peptide linker, a label, a label moiety, or a detectable moiety. In embodiments, the additional protein domain includes a solubilizing amino acid sequence. In embodiments, the additional protein domain includes a reporter amino acid sequence. In embodiments, the additional protein domain includes a peptide linker. In embodiments, the additional protein domain includes a label. In embodiments, the additional protein domain includes a label moiety. In embodiments, the additional protein domain includes a detectable moiety.

In embodiments, the peptide further includes a solubilizing amino acid sequence, wherein the solubilizing amino acid sequence is a maltose-binding protein (MBP) sequence, a small ubiquitin modified (SUMO) sequence, a thioredoxin (TrxA) sequence, a N-utilizing substance A (NusA) sequence, a Fasciola hepatica 8-kDa antigen (Fh8) sequence, a mutated dehalogenase (Halo) sequence, or a glutathione-S-transferase (GST) sequence.

In embodiments, the peptide further includes a solubilizing amino acid sequence, wherein the solubilizing amino acid sequence is a maltose-binding protein (MBP) sequence. In embodiments, the peptide further includes a solubilizing amino acid sequence, wherein the solubilizing amino acid sequence is a small ubiquitin modified (SUMO) sequence. In embodiments, the peptide further includes a solubilizing amino acid sequence, wherein the solubilizing amino acid sequence is a thioredoxin (TrxA) sequence. In embodiments, the peptide further includes a solubilizing amino acid sequence, wherein the solubilizing amino acid sequence is a N-utilizing substance A (NusA) sequence. In embodiments, the peptide further includes a solubilizing amino acid sequence, wherein the solubilizing amino acid sequence is a Fasciola hepatica 8-kDa antigen (Fh8) sequence. In embodiments, the peptide further includes a solubilizing amino acid sequence, wherein the solubilizing amino acid sequence is a mutated dehalogenase (Halo) sequence. In embodiments, the peptide further includes a solubilizing amino acid sequence, wherein the solubilizing amino acid sequence is a glutathione-S-transferase (GST) sequence.

In embodiments, the peptide further includes a first peptide linker and a second peptide linker. In embodiments, the first peptide linker includes the amino acid sequence of SEQ ID NO: 42, SEQ ID NO:44, SEQ ID NO:45, or SEQ ID NO:47. In embodiments, the first peptide linker includes the amino acid sequence of SEQ ID NO:42. In embodiments, the first peptide linker includes the amino acid sequence of SEQ ID NO:44. In embodiments, the first peptide linker includes the amino acid sequence of SEQ ID NO:45. In embodiments, the first peptide linker includes the amino acid sequence of SEQ ID NO:47. In embodiments, the first peptide linker is the amino acid sequence of SEQ ID NO:42, SEQ ID NO:44, SEQ ID NO:45, or SEQ ID NO: 47. In embodiments, the first peptide linker is the amino acid sequence of SEQ ID NO:42. In embodiments, the first peptide linker is the amino acid sequence of SEQ ID NO:44. In embodiments, the first peptide linker is the amino acid sequence of SEQ ID NO:45. In embodiments, the first peptide linker is the amino acid sequence of SEQ ID NO:47.

In embodiments, the second peptide linker includes the amino acid sequence of SEQ ID NO: 42, SEQ ID NO:44, SEQ ID NO:45, or SEQ ID NO:47. In embodiments, the second peptide linker includes the amino acid sequence of SEQ ID NO:42. In embodiments, the second peptide linker includes the amino acid sequence of SEQ ID NO:44. In embodiments, the second peptide linker includes the amino acid sequence of SEQ ID NO:45. In embodiments, the second peptide linker includes the amino acid sequence of SEQ ID NO:47. In embodiments, the second peptide linker is the amino acid sequence of SEQ ID NO:42, SEQ ID NO:44, SEQ ID NO:45, or SEQ ID NO: 47. In embodiments, the second peptide linker is the amino acid sequence of SEQ ID NO:42. In embodiments, the second peptide linker is the amino acid sequence of SEQ ID NO:44. In embodiments, the second peptide linker is the amino acid sequence of SEQ ID NO:45. In embodiments, the second peptide linker is the amino acid sequence of SEQ ID NO:47.

In embodiments, the peptide further includes a reporter amino acid sequence. In embodiments the reporter amino acid sequence is a fluorescent protein or fragment thereof. In embodiments, the reporter amino acid sequence is a green fluorescent protein. In embodiments, the reporter amino acid sequence includes the amino acid sequence of SEQ ID NO:43. In embodiments, the reporter amino acid sequence is the amino acid sequence of SEQ ID NO:43.

In embodiments, the peptide includes a solubilizing amino acid sequence attached to the N-terminus of a reporter amino acid sequence via a first peptide linker, wherein the reporter amino acid sequence is bound to the N-terminus of the amino acid sequence of SEQ ID NO:22 via a second peptide linker. In embodiments, the solubilizing amino acid includes the amino acid sequence of SEQ ID NO:41; the first peptide linker includes the amino acid sequence of SEQ ID NO: 44; the reporter amino acid sequence includes the amino acid sequence of SEQ ID NO:43; and the second peptide linker includes the amino acid sequence of SEQ ID NO:45. In embodiments, the peptide includes the amino acid sequence of SEQ ID NO:34. In embodiments, the peptide is the amino acid sequence of SEQ ID NO:34. In one further embodiment, the peptide is referred to as MBP_N44dm.

In another aspect is provided a terminal deoxynucleotidyl transferase (TdT) peptide, wherein the peptide includes an amino acid sequence having at least 80% sequence identity to the amino acid sequence of SEQ ID NO:23. In embodiments, the peptide includes an amino acid sequence having at least 81% sequence identity to the amino acid sequence of SEQ ID NO:23. In embodiments, the peptide includes an amino acid sequence having at least 82% sequence identity to the amino acid sequence of SEQ ID NO:23. In embodiments, the peptide includes an amino acid sequence having at least 83% sequence identity to the amino acid sequence of SEQ ID NO: 23. In embodiments, the peptide includes an amino acid sequence having at least 84% sequence identity to the amino acid sequence of SEQ ID NO:23. In embodiments, the peptide includes an amino acid sequence having at least 85% sequence identity to the amino acid sequence of SEQ ID NO:23. In embodiments, the peptide includes an amino acid sequence having at least 86% sequence identity to the amino acid sequence of SEQ ID NO:23. In embodiments, the peptide includes an amino acid sequence having at least 87% sequence identity to the amino acid sequence of SEQ ID NO:23. In embodiments, the peptide includes an amino acid sequence having at least 88% sequence identity to the amino acid sequence of SEQ ID NO: 23. In embodiments, the peptide includes an amino acid sequence having at least 89% sequence identity to the amino acid sequence of SEQ ID NO:23. In embodiments, the peptide includes an amino acid sequence having at least 90% sequence identity to the amino acid sequence of SEQ ID NO:23. In embodiments, the peptide includes an amino acid sequence having at least 91% sequence identity to the amino acid sequence of SEQ ID NO:23. In embodiments, the peptide includes an amino acid sequence having at least 92% sequence identity to the amino acid sequence of SEQ ID NO:23. In embodiments, the peptide includes an amino acid sequence having at least 93% sequence identity to the amino acid sequence of SEQ ID NO: 23. In embodiments, the peptide includes an amino acid sequence having at least 94% sequence identity to the amino acid sequence of SEQ ID NO:23. In embodiments, the peptide includes an amino acid sequence having at least 95% sequence identity to the amino acid sequence of SEQ ID NO:23. In embodiments, the peptide includes an amino acid sequence having at least 96% sequence identity to the amino acid sequence of SEQ ID NO:23. In embodiments, the peptide includes an amino acid sequence having at least 97% sequence identity to the amino acid sequence of SEQ ID NO:23. In embodiments, the peptide includes an amino acid sequence having at least 98% sequence identity to the amino acid sequence of SEQ ID NO: 23. In embodiments, the peptide includes an amino acid sequence having at least 99% sequence identity to the amino acid sequence of SEQ ID NO:23. In embodiments, the peptide includes an amino acid sequence having at least 100% sequence identity to the amino acid sequence of SEQ ID NO:23.

In embodiments, the peptide is an amino acid sequence having at least 80% sequence identity to the amino acid sequence of SEQ ID NO:23. In embodiments, the peptide is an amino acid sequence having at least 81% sequence identity to the amino acid sequence of SEQ ID NO: 23. In embodiments, the peptide is an amino acid sequence having at least 82% sequence identity to the amino acid sequence of SEQ ID NO:23. In embodiments, the peptide is an amino acid sequence having at least 83% sequence identity to the amino acid sequence of SEQ ID NO: 23. In embodiments, the peptide is an amino acid sequence having at least 84% sequence identity to the amino acid sequence of SEQ ID NO:23. In embodiments, the peptide is an amino acid sequence having at least 85% sequence identity to the amino acid sequence of SEQ ID NO: 23. In embodiments, the peptide is an amino acid sequence having at least 86% sequence identity to the amino acid sequence of SEQ ID NO:23. In embodiments, the peptide is an amino acid sequence having at least 87% sequence identity to the amino acid sequence of SEQ ID NO: 23. In embodiments, the peptide is an amino acid sequence having at least 88% sequence identity to the amino acid sequence of SEQ ID NO:23. In embodiments, the peptide is an amino acid sequence having at least 89% sequence identity to the amino acid sequence of SEQ ID NO: 23. In embodiments, the peptide is an amino acid sequence having at least 90% sequence identity to the amino acid sequence of SEQ ID NO:23. In embodiments, the peptide is an amino acid sequence having at least 91% sequence identity to the amino acid sequence of SEQ ID NO: 23. In embodiments, the peptide is an amino acid sequence having at least 92% sequence identity to the amino acid sequence of SEQ ID NO:23. In embodiments, the peptide is an amino acid sequence having at least 93% sequence identity to the amino acid sequence of SEQ ID NO: 23. In embodiments, the peptide is an amino acid sequence having at least 94% sequence identity to the amino acid sequence of SEQ ID NO:23. In embodiments, the peptide is an amino acid sequence having at least 95% sequence identity to the amino acid sequence of SEQ ID NO: 23. In embodiments, the peptide is an amino acid sequence having at least 96% sequence identity to the amino acid sequence of SEQ ID NO:23. In embodiments, the peptide is an amino acid sequence having at least 97% sequence identity to the amino acid sequence of SEQ ID NO: 23. In embodiments, the peptide is an amino acid sequence having at least 98% sequence identity to the amino acid sequence of SEQ ID NO:23. In embodiments, the peptide is an amino acid sequence having at least 99% sequence identity to the amino acid sequence of SEQ ID NO: 23. In embodiments, the peptide is an amino acid sequence having at least 100% sequence identity to the amino acid sequence of SEQ ID NO:23.

In embodiments, the peptide includes an amino acid sequence having 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to 250, 300, 350, 375, 380, 381, 382, 383, 384, 385, 386, 387, 388 contiguous amino acids of the sequence of SEQ ID NO:23. In embodiments, the peptide includes a sequence having 80% sequence identity to 250, 300, 350, 375, 380, 381, 382, 383, 384, 385, 386, 387, 388 contiguous amino acids of the sequence of SEQ ID NO:23. In embodiments, the peptide includes a sequence having 81% sequence identity to 250, 300, 350, 375, 380, 381, 382, 383, 384, 385, 386, 387, 388 contiguous amino acids of the sequence of SEQ ID NO: 23. In embodiments, the peptide includes a sequence having 82% sequence identity to 250, 300, 350, 375, 380, 381, 382, 383, 384, 385, 386, 387, 388 contiguous amino acids of the sequence of SEQ ID NO:23. In embodiments, the peptide includes a sequence having 83% sequence identity to 250, 300, 350, 375, 380, 381, 382, 383, 384, 385, 386, 387, 388 contiguous amino acids of the sequence of SEQ ID NO:23. In embodiments, the peptide includes a sequence having 84% sequence identity to 250, 300, 350, 375, 380, 381, 382, 383, 384, 385, 386, 387, 388 contiguous amino acids of the sequence of SEQ ID NO:23. In embodiments, the peptide includes a sequence having 85% sequence identity to 250, 300, 350, 375, 380, 381, 382, 383, 384, 385, 386, 387, 388 contiguous amino acids of the sequence of SEQ ID NO:23. In embodiments, the peptide includes a sequence having 86% sequence identity to 250, 300, 350, 375, 380, 381, 382, 383, 384, 385, 386, 387, 388 contiguous amino acids of the sequence of SEQ ID NO:23. In embodiments, the peptide includes a sequence having 87% sequence identity to 250, 300, 350, 375, 380, 381, 382, 383, 384, 385, 386, 387, 388 contiguous amino acids of the sequence of SEQ ID NO: 23. In embodiments, the peptide includes a sequence having 88% sequence identity to 250, 300, 350, 375, 380, 381, 382, 383, 384, 385, 386, 387, 388 contiguous amino acids of the sequence of SEQ ID NO:23. In embodiments, the peptide includes a sequence having 89% sequence identity 250, 300, 350, 375, 380, 381, 382, 383, 384, 385, 386, 387, 388 contiguous amino acids of the sequence of SEQ ID NO:23. In embodiments, the peptide includes a sequence having 90% sequence identity to 250, 300, 350, 375, 380, 381, 382, 383, 384, 385, 386, 387, 388 contiguous amino acids of the sequence of SEQ ID NO:23. In embodiments, the peptide includes a sequence having 91% sequence identity to 250, 300, 350, 375, 380, 381, 382, 383, 384, 385, 386, 387, 388 contiguous amino acids of the sequence of SEQ ID NO:23. In embodiments, the peptide includes a sequence having 92% sequence identity to 250, 300, 350, 375, 380, 381, 382, 383, 384, 385, 386, 387, 388 contiguous amino acids of the sequence of SEQ ID NO:23. In embodiments, the peptide includes a sequence having 93% sequence identity to 250, 300, 350, 375, 380, 381, 382, 383, 384, 385, 386, 387, 388 contiguous amino acids of the sequence of SEQ ID NO: 23. In embodiments, the peptide includes a sequence having 94% sequence identity to 250, 300, 350, 375, 380, 381, 382, 383, 384, 385, 386, 387, 388 contiguous amino acids of the sequence of SEQ ID NO:23. In embodiments, the peptide includes a sequence having 95% sequence identity to 250, 300, 350, 375, 380, 381, 382, 383, 384, 385, 386, 387, 388 contiguous amino acids of the sequence of SEQ ID NO:23. In embodiments, the peptide includes a sequence having 96% sequence identity to 250, 300, 350, 375, 380, 381, 382, 383, 384, 385, 386, 387, 388 contiguous amino acids of the sequence of SEQ ID NO:23. In embodiments, the peptide includes a sequence having 97% sequence identity to 250, 300, 350, 375, 380, 381, 382, 383, 384, 385, 386, 387, 388 contiguous amino acids of the sequence of SEQ ID NO:23. In embodiments, the peptide includes a sequence having 98% sequence identity to 250, 300, 350, 375, 380, 381, 382, 383, 384, 385, 386, 387, 388 contiguous amino acids of the sequence of SEQ ID NO:23. In embodiments, the peptide includes a sequence having 99% sequence identity to 250, 300, 350, 375, 380, 381, 382, 383, 384, 385, 386, 387, 388 contiguous amino acids of the sequence of SEQ ID NO: 23. In embodiments, the peptide includes a sequence having 100% sequence identity to 250, 300, 350, 375, 380, 381, 382, 383, 384, 385, 386, 387, 388 contiguous amino acids of the sequence of SEQ ID NO:23.

In embodiments, the peptide includes the amino acid sequence of SEQ ID NO:23. In embodiments, the peptide has the amino acid sequence of SEQ ID NO:23. In one further embodiment, the peptide is referred to as N49dm.

In embodiments, the peptide may further include additional protein elements or protein domains. In embodiments, the peptide further includes additional protein elements. In embodiments, the peptide further includes additional protein domains. In embodiments, the additional protein elements or protein domains may include solubilizing amino acid sequences, reporter amino acid sequences, peptide linkers, labels, label moieties, or detectable moieities. In embodiments, the additional protein domain includes a solubilizing amino acid sequence, a reporter amino acid sequence, a peptide linker, a label, a label moiety, or a detectable moiety. In embodiments, the additional protein domain includes a solubilizing amino acid sequence. In embodiments, the additional protein domain includes a reporter amino acid sequence. In embodiments, the additional protein domain includes a peptide linker. In embodiments, the additional protein domain includes a label. In embodiments, the additional protein domain includes a label moiety. In embodiments, the additional protein domain includes a detectable moiety.

In embodiments, the peptide further includes a solubilizing amino acid sequence, wherein the solubilizing amino acid sequence is a maltose-binding protein (MBP) sequence, a small ubiquitin modified (SUMO) sequence, a thioredoxin (TrxA) sequence, a N-utilizing substance A (NusA) sequence, a Fasciola hepatica 8-kDa antigen (Fh8) sequence, a mutated dehalogenase (Halo) sequence, or a glutathione-S-transferase (GST) sequence.

In embodiments, the peptide further includes a solubilizing amino acid sequence, wherein the solubilizing amino acid sequence is a maltose-binding protein (MBP) sequence. In embodiments, the peptide further includes a solubilizing amino acid sequence, wherein the solubilizing amino acid sequence is a small ubiquitin modified (SUMO) sequence. In embodiments, the peptide further includes a solubilizing amino acid sequence, wherein the solubilizing amino acid sequence is a thioredoxin (TrxA) sequence. In embodiments, the peptide further includes a solubilizing amino acid sequence, wherein the solubilizing amino acid sequence is a N-utilizing substance A (NusA) sequence. In embodiments, the peptide further includes a solubilizing amino acid sequence, wherein the solubilizing amino acid sequence is a Fasciola hepatica 8-kDa antigen (Fh8) sequence. In embodiments, the peptide further includes a solubilizing amino acid sequence, wherein the solubilizing amino acid sequence is a mutated dehalogenase (Halo) sequence. In embodiments, the peptide further includes a solubilizing amino acid sequence, wherein the solubilizing amino acid sequence is a glutathione-S-transferase (GST) sequence.

In embodiments, the peptide further includes a first peptide linker and a second peptide linker. In embodiments, the first peptide linker includes the amino acid sequence of SEQ ID NO: 42, SEQ ID NO:44, SEQ ID NO:45, or SEQ ID NO:47. In embodiments, the first peptide linker includes the amino acid sequence of SEQ ID NO:42. In embodiments, the first peptide linker includes the amino acid sequence of SEQ ID NO:44. In embodiments, the first peptide linker includes the amino acid sequence of SEQ ID NO:45. In embodiments, the first peptide linker includes the amino acid sequence of SEQ ID NO:47. In embodiments, the first peptide linker is the amino acid sequence of SEQ ID NO:42, SEQ ID NO:44, SEQ ID NO:45, or SEQ ID NO: 47. In embodiments, the first peptide linker is the amino acid sequence of SEQ ID NO:42. In embodiments, the first peptide linker is the amino acid sequence of SEQ ID NO:44. In embodiments, the first peptide linker is the amino acid sequence of SEQ ID NO:45. In embodiments, the first peptide linker is the amino acid sequence of SEQ ID NO:47.

In embodiments, the second peptide linker includes the amino acid sequence of SEQ ID NO: 42, SEQ ID NO:44, SEQ ID NO:45, or SEQ ID NO:47. In embodiments, the second peptide linker includes the amino acid sequence of SEQ ID NO:42. In embodiments, the second peptide linker includes the amino acid sequence of SEQ ID NO:44. In embodiments, the second peptide linker includes the amino acid sequence of SEQ ID NO:45. In embodiments, the second peptide linker includes the amino acid sequence of SEQ ID NO:47. In embodiments, the second peptide linker is the amino acid sequence of SEQ ID NO:42, SEQ ID NO:44, SEQ ID NO:45, or SEQ ID NO: 47. In embodiments, the second peptide linker is the amino acid sequence of SEQ ID NO:42. In embodiments, the second peptide linker is the amino acid sequence of SEQ ID NO:44. In embodiments, the second peptide linker is the amino acid sequence of SEQ ID NO:45. In embodiments, the second peptide linker is the amino acid sequence of SEQ ID NO:47.

In embodiments, the peptide further includes a reporter amino acid sequence. In embodiments the reporter amino acid sequence is a fluorescent protein or fragment thereof. In embodiments, the reporter amino acid sequence is a green fluorescent protein. In embodiments, the reporter amino acid sequence includes the amino acid sequence of SEQ ID NO:43. In embodiments, the reporter amino acid sequence is the amino acid sequence of SEQ ID NO:43.

In embodiments, the peptide includes a solubilizing amino acid sequence attached to the N-terminus of a reporter amino acid sequence via a first peptide linker, wherein the reporter amino acid sequence is bound to the N-terminus of the amino acid sequence of SEQ ID NO:23 via a second peptide linker. In embodiments, the solubilizing amino acid includes the amino acid sequence of SEQ ID NO:41; the first peptide linker includes the amino acid sequence of SEQ ID NO: 44; the reporter amino acid sequence includes the amino acid sequence of SEQ ID NO:43; and the second peptide linker includes the amino acid sequence of SEQ ID NO:45. In embodiments, the peptide includes the amino acid sequence of SEQ ID NO:35. In embodiments, the peptide is the amino acid sequence of SEQ ID NO:35. In one further embodiment, the peptide is referred to as MBP_N49dm.

In another aspect is provided a terminal deoxynucleotidyl transferase (TdT) peptide, wherein the peptide includes an amino acid sequence having at least 80% sequence identity to the amino acid sequence of SEQ ID NO:24. In embodiments, the peptide includes an amino acid sequence having at least 81% sequence identity to the amino acid sequence of SEQ ID NO:24. In embodiments, the peptide includes an amino acid sequence having at least 82% sequence identity to the amino acid sequence of SEQ ID NO:24. In embodiments, the peptide includes an amino acid sequence having at least 83% sequence identity to the amino acid sequence of SEQ ID NO: 24. In embodiments, the peptide includes an amino acid sequence having at least 84% sequence identity to the amino acid sequence of SEQ ID NO:24. In embodiments, the peptide includes an amino acid sequence having at least 85% sequence identity to the amino acid sequence of SEQ ID NO:24. In embodiments, the peptide includes an amino acid sequence having at least 86% sequence identity to the amino acid sequence of SEQ ID NO:24. In embodiments, the peptide includes an amino acid sequence having at least 87% sequence identity to the amino acid sequence of SEQ ID NO:24. In embodiments, the peptide includes an amino acid sequence having at least 88% sequence identity to the amino acid sequence of SEQ ID NO: 24. In embodiments, the peptide includes an amino acid sequence having at least 89% sequence identity to the amino acid sequence of SEQ ID NO:24. In embodiments, the peptide includes an amino acid sequence having at least 90% sequence identity to the amino acid sequence of SEQ ID NO:24. In embodiments, the peptide includes an amino acid sequence having at least 91% sequence identity to the amino acid sequence of SEQ ID NO:24. In embodiments, the peptide includes an amino acid sequence having at least 92% sequence identity to the amino acid sequence of SEQ ID NO:24. In embodiments, the peptide includes an amino acid sequence having at least 93% sequence identity to the amino acid sequence of SEQ ID NO: 24. In embodiments, the peptide includes an amino acid sequence having at least 94% sequence identity to the amino acid sequence of SEQ ID NO:24. In embodiments, the peptide includes an amino acid sequence having at least 95% sequence identity to the amino acid sequence of SEQ ID NO:24. In embodiments, the peptide includes an amino acid sequence having at least 96% sequence identity to the amino acid sequence of SEQ ID NO:24. In embodiments, the peptide includes an amino acid sequence having at least 97% sequence identity to the amino acid sequence of SEQ ID NO:24. In embodiments, the peptide includes an amino acid sequence having at least 98% sequence identity to the amino acid sequence of SEQ ID NO: 24. In embodiments, the peptide includes an amino acid sequence having at least 99% sequence identity to the amino acid sequence of SEQ ID NO:24. In embodiments, the peptide includes an amino acid sequence having at least 100% sequence identity to the amino acid sequence of SEQ ID NO:24.

In embodiments, the peptide is an amino acid sequence having at least 80% sequence identity to the amino acid sequence of SEQ ID NO:24. In embodiments, the peptide is an amino acid sequence having at least 81% sequence identity to the amino acid sequence of SEQ ID NO: 24. In embodiments, the peptide is an amino acid sequence having at least 82% sequence identity to the amino acid sequence of SEQ ID NO:24. In embodiments, the peptide is an amino acid sequence having at least 83% sequence identity to the amino acid sequence of SEQ ID NO: 24. In embodiments, the peptide is an amino acid sequence having at least 84% sequence identity to the amino acid sequence of SEQ ID NO:24. In embodiments, the peptide is an amino acid sequence having at least 85% sequence identity to the amino acid sequence of SEQ ID NO: 24. In embodiments, the peptide is an amino acid sequence having at least 86% sequence identity to the amino acid sequence of SEQ ID NO:24. In embodiments, the peptide is an amino acid sequence having at least 87% sequence identity to the amino acid sequence of SEQ ID NO: 24. In embodiments, the peptide is an amino acid sequence having at least 88% sequence identity to the amino acid sequence of SEQ ID NO:24. In embodiments, the peptide is an amino acid sequence having at least 89% sequence identity to the amino acid sequence of SEQ ID NO: 24. In embodiments, the peptide is an amino acid sequence having at least 90% sequence identity to the amino acid sequence of SEQ ID NO:24. In embodiments, the peptide is an amino acid sequence having at least 91% sequence identity to the amino acid sequence of SEQ ID NO: 24. In embodiments, the peptide is an amino acid sequence having at least 92% sequence identity to the amino acid sequence of SEQ ID NO:24. In embodiments, the peptide is an amino acid sequence having at least 93% sequence identity to the amino acid sequence of SEQ ID NO: 24. In embodiments, the peptide is an amino acid sequence having at least 94% sequence identity to the amino acid sequence of SEQ ID NO:24. In embodiments, the peptide is an amino acid sequence having at least 95% sequence identity to the amino acid sequence of SEQ ID NO: 24. In embodiments, the peptide is an amino acid sequence having at least 96% sequence identity to the amino acid sequence of SEQ ID NO:24. In embodiments, the peptide is an amino acid sequence having at least 97% sequence identity to the amino acid sequence of SEQ ID NO: 24. In embodiments, the peptide is an amino acid sequence having at least 98% sequence identity to the amino acid sequence of SEQ ID NO:24. In embodiments, the peptide is an amino acid sequence having at least 99% sequence identity to the amino acid sequence of SEQ ID NO: 24. In embodiments, the peptide is an amino acid sequence having at least 100% sequence identity to the amino acid sequence of SEQ ID NO:24.

In embodiments, the peptide includes an amino acid sequence having 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to 250, 300, 350, 375, 380, 381, 382, 383, 384, 385, 386, 387, 388 contiguous amino acids of the sequence of SEQ ID NO:24. In embodiments, the peptide includes a sequence having 80% sequence identity to 250, 300, 350, 375, 380, 381, 382, 383, 384, 385, 386, 387, 388 contiguous amino acids of the sequence of SEQ ID NO:24. In embodiments, the peptide includes a sequence having 81% sequence identity to 250, 300, 350, 375, 380, 381, 382, 383, 384, 385, 386, 387, 388 contiguous amino acids of the sequence of SEQ ID NO: 24. In embodiments, the peptide includes a sequence having 82% sequence identity to 250, 300, 350, 375, 380, 381, 382, 383, 384, 385, 386, 387, 388 contiguous amino acids of the sequence of SEQ ID NO:24. In embodiments, the peptide includes a sequence having 83% sequence identity to 250, 300, 350, 375, 380, 381, 382, 383, 384, 385, 386, 387, 388 contiguous amino acids of the sequence of SEQ ID NO:24. In embodiments, the peptide includes a sequence having 84% sequence identity to 250, 300, 350, 375, 380, 381, 382, 383, 384, 385, 386, 387, 388 contiguous amino acids of the sequence of SEQ ID NO:24. In embodiments, the peptide includes a sequence having 85% sequence identity to 250, 300, 350, 375, 380, 381, 382, 383, 384, 385, 386, 387, 388 contiguous amino acids of the sequence of SEQ ID NO:24. In embodiments, the peptide includes a sequence having 86% sequence identity to 250, 300, 350, 375, 380, 381, 382, 383, 384, 385, 386, 387, 388 contiguous amino acids of the sequence of SEQ ID NO:24. In embodiments, the peptide includes a sequence having 87% sequence identity to 250, 300, 350, 375, 380, 381, 382, 383, 384, 385, 386, 387, 388 contiguous amino acids of the sequence of SEQ ID NO: 24. In embodiments, the peptide includes a sequence having 88% sequence identity to 250, 300, 350, 375, 380, 381, 382, 383, 384, 385, 386, 387, 388 contiguous amino acids of the sequence of SEQ ID NO:24. In embodiments, the peptide includes a sequence having 89% sequence identity 250, 300, 350, 375, 380, 381, 382, 383, 384, 385, 386, 387, 388 contiguous amino acids of the sequence of SEQ ID NO:24. In embodiments, the peptide includes a sequence having 90% sequence identity to 250, 300, 350, 375, 380, 381, 382, 383, 384, 385, 386, 387, 388 contiguous amino acids of the sequence of SEQ ID NO:24. In embodiments, the peptide includes a sequence having 91% sequence identity to 250, 300, 350, 375, 380, 381, 382, 383, 384, 385, 386, 387, 388 contiguous amino acids of the sequence of SEQ ID NO:24. In embodiments, the peptide includes a sequence having 92% sequence identity to 250, 300, 350, 375, 380, 381, 382, 383, 384, 385, 386, 387, 388 contiguous amino acids of the sequence of SEQ ID NO:24. In embodiments, the peptide includes a sequence having 93% sequence identity to 250, 300, 350, 375, 380, 381, 382, 383, 384, 385, 386, 387, 388 contiguous amino acids of the sequence of SEQ ID NO: 24. In embodiments, the peptide includes a sequence having 94% sequence identity to 250, 300, 350, 375, 380, 381, 382, 383, 384, 385, 386, 387, 388 contiguous amino acids of the sequence of SEQ ID NO:24. In embodiments, the peptide includes a sequence having 95% sequence identity to 250, 300, 350, 375, 380, 381, 382, 383, 384, 385, 386, 387, 388 contiguous amino acids of the sequence of SEQ ID NO:24. In embodiments, the peptide includes a sequence having 96% sequence identity to 250, 300, 350, 375, 380, 381, 382, 383, 384, 385, 386, 387, 388 contiguous amino acids of the sequence of SEQ ID NO:24. In embodiments, the peptide includes a sequence having 97% sequence identity to 250, 300, 350, 375, 380, 381, 382, 383, 384, 385, 386, 387, 388 contiguous amino acids of the sequence of SEQ ID NO:24. In embodiments, the peptide includes a sequence having 98% sequence identity to 250, 300, 350, 375, 380, 381, 382, 383, 384, 385, 386, 387, 388 contiguous amino acids of the sequence of SEQ ID NO:24. In embodiments, the peptide includes a sequence having 99% sequence identity to 250, 300, 350, 375, 380, 381, 382, 383, 384, 385, 386, 387, 388 contiguous amino acids of the sequence of SEQ ID NO: 24. In embodiments, the peptide includes a sequence having 100% sequence identity to 250, 300, 350, 375, 380, 381, 382, 383, 384, 385, 386, 387, 388 contiguous amino acids of the sequence of SEQ ID NO:24.

In embodiments, the peptide includes the amino acid sequence of SEQ ID NO:24. In embodiments, the peptide has the amino acid sequence of SEQ ID NO:24. In one further embodiment, the peptide is referred to as N51dm.

In embodiments, the peptide may further include additional protein elements or protein domains. In embodiments, the peptide further includes additional protein elements. In embodiments, the peptide further includes additional protein domains. In embodiments, the additional protein elements or protein domains may include solubilizing amino acid sequences, reporter amino acid sequences, peptide linkers, labels, label moieties, or detectable moieities. In embodiments, the additional protein domain includes a solubilizing amino acid sequence, a reporter amino acid sequence, a peptide linker, a label, a label moiety, or a detectable moiety. In embodiments, the additional protein domain includes a solubilizing amino acid sequence. In embodiments, the additional protein domain includes a reporter amino acid sequence. In embodiments, the additional protein domain includes a peptide linker. In embodiments, the additional protein domain includes a label. In embodiments, the additional protein domain includes a label moiety. In embodiments, the additional protein domain includes a detectable moiety.

In embodiments, the peptide further includes a solubilizing amino acid sequence, wherein the solubilizing amino acid sequence is a maltose-binding protein (MBP) sequence, a small ubiquitin modified (SUMO) sequence, a thioredoxin (TrxA) sequence, a N-utilizing substance A (NusA) sequence, a Fasciola hepatica 8-kDa antigen (Fh8) sequence, a mutated dehalogenase (Halo) sequence, or a glutathione-S-transferase (GST) sequence.

In embodiments, the peptide further includes a solubilizing amino acid sequence, wherein the solubilizing amino acid sequence is a maltose-binding protein (MBP) sequence. In embodiments, the peptide further includes a solubilizing amino acid sequence, wherein the solubilizing amino acid sequence is a small ubiquitin modified (SUMO) sequence. In embodiments, the peptide further includes a solubilizing amino acid sequence, wherein the solubilizing amino acid sequence is a thioredoxin (TrxA) sequence. In embodiments, the peptide further includes a solubilizing amino acid sequence, wherein the solubilizing amino acid sequence is a N-utilizing substance A (NusA) sequence. In embodiments, the peptide further includes a solubilizing amino acid sequence, wherein the solubilizing amino acid sequence is a Fasciola hepatica 8-kDa antigen (Fh8) sequence. In embodiments, the peptide further includes a solubilizing amino acid sequence, wherein the solubilizing amino acid sequence is a mutated dehalogenase (Halo) sequence. In embodiments, the peptide further includes a solubilizing amino acid sequence, wherein the solubilizing amino acid sequence is a glutathione-S-transferase (GST) sequence.

In embodiments, the peptide further includes a solubilizing amino acid sequence. In embodiments, the solubilizing amino acid sequence is a maltose-binding protein (MBP) sequence, a small ubiquitin modified (SUMO) sequence, a thioredoxin (TrxA) sequence, a N-utilizing substance A (NusA) sequence, a Fasciola hepatica 8-kDa antigen (Fh8) sequence, a mutated dehalogenase (Halo) sequence, or a glutathione-S-transferase (GST) sequence. In embodiments, the solubilizing amino acid sequence is a maltose-binding protein (MBP) sequence. In embodiments, the solubilizing amino acid sequence is a small ubiquitin modified (SUMO) sequence. In embodiments, the solubilizing amino acid sequence is a thioredoxin (TrxA) sequence. In embodiments, the solubilizing amino acid sequence is a N-utilizing substance A (NusA) sequence. In embodiments, the solubilizing amino acid sequence is a Fasciola hepatica 8-kDa antigen (Fh8) sequence. In embodiments, the solubilizing amino acid sequence is a mutated dehalogenase (Halo) sequence. In embodiments, the solubilizing amino acid sequence is a glutathione-S-transferase (GST) sequence.

In embodiments, the solubilizing amino acid sequence includes the amino acid sequence of SEQ ID NO:41, SEQ ID NO:46, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO: 56, SEQ ID NO:57, or SEQ ID NO:58. In embodiments, the solubilizing amino acid sequence includes the amino acid sequence of SEQ ID NO:41. In embodiments, the solubilizing amino acid sequence includes the amino acid sequence of SEQ ID NO:46. In embodiments, the solubilizing amino acid sequence includes the amino acid sequence of SEQ ID NO:48. In embodiments, the solubilizing amino acid sequence includes the amino acid sequence of SEQ ID NO: 49. In embodiments, the solubilizing amino acid sequence includes the amino acid sequence of SEQ ID NO:50. In embodiments, the solubilizing amino acid sequence includes the amino acid sequence of SEQ ID NO:51. In embodiments, the solubilizing amino acid sequence includes the amino acid sequence of SEQ ID NO:52. In embodiments, the solubilizing amino acid sequence includes the amino acid sequence of SEQ ID NO:53. In embodiments, the solubilizing amino acid sequence includes the amino acid sequence of SEQ ID NO:54. In embodiments, the solubilizing amino acid sequence includes the amino acid sequence of SEQ ID NO:55. In embodiments, the solubilizing amino acid sequence includes the amino acid sequence of SEQ ID NO: 56. In embodiments, the solubilizing amino acid sequence includes the amino acid sequence of SEQ ID NO:57. In embodiments, the solubilizing amino acid sequence includes the amino acid sequence of SEQ ID NO:58.

In embodiments, the solubilizing amino acid sequence is the amino acid sequence of SEQ ID NO:41, SEQ ID NO:46, SEQ ID NO:48, SEQ ID NO:49, SEQ ID NO:50, SEQ ID NO: 51, SEQ ID NO:52, SEQ ID NO:53, SEQ ID NO:54, SEQ ID NO:55, SEQ ID NO:56, SEQ ID NO: 57, or SEQ ID NO:58. In embodiments, the solubilizing amino acid sequence is the amino acid sequence of SEQ ID NO:41. In embodiments, the solubilizing amino acid sequence is the amino acid sequence of SEQ ID NO:46. In embodiments, the solubilizing amino acid sequence is the amino acid sequence of SEQ ID NO:48. In embodiments, the solubilizing amino acid sequence is the amino acid sequence of SEQ ID NO:49. In embodiments, the solubilizing amino acid sequence is the amino acid sequence of SEQ ID NO:50. In embodiments, the solubilizing amino acid sequence is the amino acid sequence of SEQ ID NO:51. In embodiments, the solubilizing amino acid sequence is the amino acid sequence of SEQ ID NO:52. In embodiments, the solubilizing amino acid sequence is the amino acid sequence of SEQ ID NO: 53. In embodiments, the solubilizing amino acid sequence is the amino acid sequence of SEQ ID NO:54. In embodiments, the solubilizing amino acid sequence is the amino acid sequence of SEQ ID NO:55. In embodiments, the solubilizing amino acid sequence is the amino acid sequence of SEQ ID NO:56. In embodiments, the solubilizing amino acid sequence is the amino acid sequence of SEQ ID NO:57. In embodiments, the solubilizing amino acid sequence is the amino acid sequence of SEQ ID NO:58.

In embodiments, the peptide further includes a first peptide linker and a second peptide linker. In embodiments, the first peptide linker includes the amino acid sequence of SEQ ID NO: 42, SEQ ID NO:44, SEQ ID NO:45, or SEQ ID NO:47. In embodiments, the first peptide linker includes the amino acid sequence of SEQ ID NO:42. In embodiments, the first peptide linker includes the amino acid sequence of SEQ ID NO:44. In embodiments, the first peptide linker includes the amino acid sequence of SEQ ID NO:45. In embodiments, the first peptide linker includes the amino acid sequence of SEQ ID NO:47. In embodiments, the first peptide linker is the amino acid sequence of SEQ ID NO:42, SEQ ID NO:44, SEQ ID NO:45, or SEQ ID NO: 47. In embodiments, the first peptide linker is the amino acid sequence of SEQ ID NO:42. In embodiments, the first peptide linker is the amino acid sequence of SEQ ID NO:44. In embodiments, the first peptide linker is the amino acid sequence of SEQ ID NO:45. In embodiments, the first peptide linker is the amino acid sequence of SEQ ID NO:47.

In embodiments, the second peptide linker includes the amino acid sequence of SEQ ID NO: 42, SEQ ID NO:44, SEQ ID NO:45, or SEQ ID NO:47. In embodiments, the second peptide linker includes the amino acid sequence of SEQ ID NO:42. In embodiments, the second peptide linker includes the amino acid sequence of SEQ ID NO:44. In embodiments, the second peptide linker includes the amino acid sequence of SEQ ID NO:45. In embodiments, the second peptide linker includes the amino acid sequence of SEQ ID NO:47. In embodiments, the second peptide linker is the amino acid sequence of SEQ ID NO:42, SEQ ID NO:44, SEQ ID NO:45, or SEQ ID NO: 47. In embodiments, the second peptide linker is the amino acid sequence of SEQ ID NO:42. In embodiments, the second peptide linker is the amino acid sequence of SEQ ID NO:44. In embodiments, the second peptide linker is the amino acid sequence of SEQ ID NO:45. In embodiments, the second peptide linker is the amino acid sequence of SEQ ID NO:47.

In embodiments, the peptide further includes a reporter amino acid sequence. In embodiments the reporter amino acid sequence is a fluorescent protein or fragment thereof. In embodiments, the reporter amino acid sequence is a green fluorescent protein. In embodiments, the reporter amino acid sequence includes the amino acid sequence of SEQ ID NO:43. In embodiments, the reporter amino acid sequence is the amino acid sequence of SEQ ID NO:43.

In embodiments, the peptide includes a solubilizing amino acid sequence attached to the N-terminus of a reporter amino acid sequence via a first peptide linker, wherein the reporter amino acid sequence is bound to the N-terminus of the amino acid sequence of SEQ ID NO:24 via a second peptide linker. In embodiments, the solubilizing amino acid includes the amino acid sequence of SEQ ID NO:41; the first peptide linker includes the amino acid sequence of SEQ ID NO: 44; the reporter amino acid sequence includes the amino acid sequence of SEQ ID NO:43; and the second peptide linker includes the amino acid sequence of SEQ ID NO:45. In embodiments, the peptide includes the amino acid sequence of SEQ ID NO:36. In embodiments, the peptide is the amino acid sequence of SEQ ID NO:36. In one further embodiment, the peptide is referred to as MBP_N51dm.

In embodiments, the peptide has increased activity for naturally occurring deoxyribonucleotides relative to a naturally occurring TdT peptide or a recombinant TdT peptide. In embodiments, the peptide has increased activity for naturally occurring deoxyribonucleotides relative to a naturally occurring TdT peptide. In embodiments, the peptide has increased activity for naturally occurring deoxyribonucleotides relative to a recombinant TdT peptide.

In embodiments, the peptide has increased activity for blocked deoxyribonucleotides relative to a naturally occurring TdT peptide or a recombinant TdT peptide. In embodiments, the peptide has increased activity for blocked deoxyribonucleotides relative to a naturally occurring TdT peptide. In embodiments, the peptide has increased activity for blocked deoxyribonucleotides relative to a recombinant TdT peptide.

In embodiments, the peptide has the ability to accommodate the incorporation of a nucleotide triphosphate. In embodiments, the peptide has the ability to accommodate the incorporation of a nucleotide triphosphate including a deoxyribose modification or a ribose 3′ modifications. In embodiments, the peptide has the ability to accommodate the incorporation of a nucleotide triphosphate including a deoxyribose modification. In embodiments, the peptide has the ability to accommodate the incorporation of a nucleotide triphosphate including a ribose 3′ modification. In embodiments, the peptide has the ability to accommodate the incorporation of a nucleotide triphosphate including a deoxyribose modification and a ribose 3′ modification. In embodiments, the peptide has the ability to accommodate the incorporation of a nucleotide triphosphate including a deoxyribose modification, a ribose 3′ modification, an additional sugar modification, a phosphate modification, or a nitrogenous base modification. In embodiments, the peptide has the ability to accommodate the incorporation of a nucleotide triphosphate including a deoxyribose modification, a ribose 3′ modification, an additional sugar modification, a phosphate modification, and a nitrogenous base modification. In embodiments, the peptide has the ability to accommodate the incorporation of a nucleotide triphosphate including a deoxyribose modification. In embodiments, the peptide has the ability to accommodate the incorporation of a nucleotide triphosphate including a ribose 3′ modification. In embodiments, the peptide has the ability to accommodate the incorporation of a nucleotide triphosphate including an additional sugar modification. In embodiments, the peptide has the ability to accommodate the incorporation of a nucleotide triphosphate including a sugar modification. In embodiments, the peptide has the ability to accommodate the incorporation of a nucleotide triphosphate including a phosphate modification. In embodiments, the peptide has the ability to accommodate the incorporation of a nucleotide triphosphate including a nitrogenous base modification.

In embodiments, the peptide is capable of synthesizing an oligonucleotide that is at least 5 nucleotides in length. In embodiments, the peptide is capable of synthesizing an oligonucleotide that is at least 10 nucleotides in length. In embodiments, the peptide is capable of synthesizing an oligonucleotide that is at least 25 nucleotides in length. In embodiments, the peptide is capable of synthesizing an oligonucleotide that is at least 50 nucleotides in length. In embodiments, the peptide is capable of synthesizing an oligonucleotide that is at least 75 nucleotides in length. In embodiments, the peptide is capable of synthesizing an oligonucleotide that is at least 100 nucleotides in length. In embodiments, the peptide is capable of synthesizing an oligonucleotide that is at least 200 nucleotides in length. In embodiments, the peptide is capable of synthesizing an oligonucleotide that is at least 300 in length. In embodiments, the peptide is capable of synthesizing an oligonucleotide that is at least 400 nucleotides in length. In embodiments, the peptide is capable of synthesizing an oligonucleotide that is at least 500 nucleotides in length. In embodiments, the peptide is capable of synthesizing an oligonucleotide that is at least 600 nucleotides in length. In embodiments, the peptide is capable of synthesizing an oligonucleotide that is at least 700 nucleotides in length. In embodiments, the peptide is capable of synthesizing an oligonucleotide that is at least 800 nucleotides in length. In embodiments, the peptide is capable of synthesizing an oligonucleotide that is at least 900 nucleotides in length. In embodiments, the peptide is capable of synthesizing an oligonucleotide that is at least 1000 nucleotides in length. In embodiments, the peptide is capable of synthesizing an oligonucleotide that is at least 1250 nucleotides in length. In embodiments, the peptide is capable of synthesizing an oligonucleotide that is at least 1500 nucleotides in length. In embodiments, the peptide is capable of synthesizing an oligonucleotide that is at least 1750 nucleotides in length. In embodiments, the peptide is capable of synthesizing an oligonucleotide that is at least 2000 nucleotides in length.

In embodiments, the starting oligonucleotide is at least 3 nucleotides in length. In embodiments, the starting oligonucleotide is at least 5 nucleotides in length. In embodiments, the starting oligonucleotide is at least 10 nucleotides in length. In embodiments, the starting oligonucleotide is at least 25 nucleotides in length. In embodiments, the starting oligonucleotide is at least 50 nucleotides in length. In embodiments, the starting oligonucleotide is at least 75 nucleotides in length. In embodiments, the starting oligonucleotide is at least 100 nucleotides in length. In embodiments, the starting oligonucleotide is at least 200 nucleotides in length. In embodiments, the starting oligonucleotide is at least 300 in length. In embodiments, the starting oligonucleotide is at least 400 nucleotides in length. In embodiments, the starting oligonucleotide is at least 500 nucleotides in length. In embodiments, the starting oligonucleotide is at least 600 nucleotides in length. In embodiments, the starting oligonucleotide is at least 700 nucleotides in length. In embodiments, the starting oligonucleotide is at least 800 nucleotides in length. In embodiments, the starting oligonucleotide is at least 900 nucleotides in length. In embodiments, the starting oligonucleotide is at least 1000 nucleotides in length. In embodiments, the starting oligonucleotide is at least 1250 nucleotides in length. In embodiments, the starting oligonucleotide is at least 1500 nucleotides in length. In embodiments, the starting oligonucleotide is at least 1750 nucleotides in length. In embodiments, the starting oligonucleotide is at least 2000 nucleotides in length.

In embodiments, the peptide has minimal variation in its binding affinity or catalytic rate for a nitrogenous base. In embodiments, the peptide has minimal variation in its binding affinity or catalytic rate for each nitrogenous base. In embodiments, the peptide has minimal variation in its binding affinity and catalytic rate for a nitrogenous base. In embodiments, the peptide has minimal variation in its binding affinity and catalytic rate for each nitrogenous base. In embodiments, the peptide has minimal variation in its binding affinity for a nitrogenous base. In embodiments, the peptide has minimal variation in its binding affinity for each nitrogenous base. In embodiments, the peptide has minimal variation in its catalytic rate for a nitrogenous base. In embodiments, the peptide has minimal variation in its catalytic rate for each nitrogenous base.

In embodiments, the affinity of the peptide for a nitrogenous base is less than 25% different relative to the affinity of the peptide for another nitrogenous base. In embodiments, the affinity of the peptide for a nitrogenous base is less than 20% different relative to the affinity of the peptide for another nitrogenous base. In embodiments, the affinity of the peptide for a nitrogenous base is less than 15% different relative to the affinity of the peptide for another nitrogenous base. In embodiments, the affinity of the peptide for a nitrogenous base is less than 10% different relative to the affinity of the peptide for another nitrogenous base. In embodiments, the affinity of the peptide for a nitrogenous base is less than 9% different relative to the affinity of the peptide for another nitrogenous base. In embodiments, the affinity of the peptide for a nitrogenous base is less than 8% different relative to the affinity of the peptide for another nitrogenous base. In embodiments, the affinity of the peptide for a nitrogenous base is less than 7% different relative to the affinity of the peptide for another nitrogenous base. In embodiments, the affinity of the peptide for a nitrogenous base is less than 6% different relative to the affinity of the peptide for another nitrogenous base. In embodiments, the affinity of the peptide for a nitrogenous base is less than 5% different relative to the affinity of the peptide for another nitrogenous base. In embodiments, the affinity of the peptide for a nitrogenous base is less than 4% different relative to the affinity of the peptide for another nitrogenous base. In embodiments, the affinity of the peptide for a nitrogenous base is less than 3% different relative to the affinity of the peptide for another nitrogenous base. In embodiments, the affinity of the peptide for a nitrogenous base is less than 2% different relative to the affinity of the peptide for another nitrogenous base. In embodiments, the affinity of the peptide for a nitrogenous base is less than 1% different relative to the affinity of the peptide for another nitrogenous base.

In embodiments, the catalytic rate of the peptide for a nitrogenous base is less than 25% different relative to the catalytic rate of the peptide for another nitrogenous base. In embodiments, the catalytic rate of the peptide for a nitrogenous base is less than 20% different relative to the catalytic rate of the peptide for another nitrogenous base. In embodiments, the catalytic rate of the peptide for a nitrogenous base is less than 15% different relative to the catalytic rate of the peptide for another nitrogenous base. In embodiments, the catalytic rate of the peptide for a nitrogenous base is less than 10% different relative to the catalytic rate of the peptide for another nitrogenous base. In embodiments, the catalytic rate of the peptide for a nitrogenous base is less than 9% different relative to the catalytic rate of the peptide for another nitrogenous base. In embodiments, the catalytic rate of the peptide for a nitrogenous base is less than 8% different relative to the catalytic rate of the peptide for another nitrogenous base. In embodiments, the catalytic rate of the peptide for a nitrogenous base is less than 7% different relative to the catalytic rate of the peptide for another nitrogenous base. In embodiments, the catalytic rate of the peptide for a nitrogenous base is less than 6% different relative to the catalytic rate of the peptide for another nitrogenous base. In embodiments, the catalytic rate of the peptide for a nitrogenous base is less than 5% different relative to the catalytic rate of the peptide for another nitrogenous base. In embodiments, the catalytic rate of the peptide for a nitrogenous base is less than 4% different relative to the catalytic rate of the peptide for another nitrogenous base. In embodiments, the catalytic rate of the peptide for a nitrogenous base is less than 3% different relative to the catalytic rate of the peptide for another nitrogenous base. In embodiments, the catalytic rate of the peptide for a nitrogenous base is less than 2% different relative to the catalytic rate of the peptide for another nitrogenous base. In embodiments, the catalytic rate of the peptide for a nitrogenous base is less than 1% different relative to the catalytic rate of the peptide for another nitrogenous base.

In embodiments, the peptide has increased thermostability relative to a naturally occurring TdT peptide or a recombinant TdT peptide. In embodiments, the peptide has increased thermostability relative to a naturally occurring TdT peptide. In embodiments, the peptide has increased thermostability relative to a recombinant TdT peptide. In embodiments, the increased thermostability of the peptide reduces enzymatic degradation. In embodiments, the increased thermostability of the peptide reduces the loss of peptide activity due to a temperature change. In embodiments, the increased thermostability of the peptide improves the peptide's stability in storage.

In embodiments, the peptide exhibits improved stability in an appropriate buffer at an appropriate temperature. In embodiments, the peptide exhibits improved stability in an appropriate buffer. In embodiments, the appropriate buffer is Tris, acetate, 4-(2-hydroxyethyl)-1-piperazineethanesulfonic acid (HEPES), 3-(N-morpholino) propanesulfonic acid (MOPS), cacodylate, or a phosphate buffer. In embodiments, the appropriate buffer is Tris, acetate. In embodiments, the appropriate buffer is 4-(2-hydroxyethyl)-1-piperazineethanesulfonic acid (HEPES). In embodiments, the appropriate buffer is 3-(N-morpholino) propanesulfonic acid (MOPS). In embodiments, the appropriate buffer is cacodylate. In embodiments, the appropriate buffer is a phosphate buffer.

In embodiments the appropriate buffer has a pH value between about pH 7.0 and about pH 8.0. In embodiments the appropriate buffer has a pH value between about pH 7.1 and about pH 8.0. In embodiments the appropriate buffer has a pH value between about pH 7.2 and about pH 8.0. In embodiments the appropriate buffer has a pH value between about pH 7.3 and about pH 8.0. In embodiments the appropriate buffer has a pH value between about pH 7.4 and about pH 8.0. In embodiments the appropriate buffer has a pH value between about pH 7.5 and about pH 8.0. In embodiments the appropriate buffer has a pH value between about pH 7.6 and about pH 8.0. In embodiments the appropriate buffer has a pH value between about pH 7.7 and about pH 8.0. In embodiments the appropriate buffer has a pH value between about pH 7.8 and about pH 8.0. In embodiments the appropriate buffer has a pH value between about pH 7.9 and about pH 8.0.

In embodiments the appropriate buffer has a pH value between about pH 7.0 and about pH 7.9. In embodiments the appropriate buffer has a pH value between about pH 7.0 and about pH 7.8. In embodiments the appropriate buffer has a pH value between about pH 7.0 and about pH 7.7. In embodiments the appropriate buffer has a pH value between about pH 7.0 and about pH 7.6. In embodiments the appropriate buffer has a pH value between about pH 7.0 and about pH 7.5. In embodiments the appropriate buffer has a pH value between about pH 7.0 and about pH 7.4. In embodiments the appropriate buffer has a pH value between about pH 7.0 and about pH 7.3. In embodiments the appropriate buffer has a pH value between about pH 7.0 and about pH 7.2. In embodiments the appropriate buffer has a pH value between about pH 7.0 and about pH 7.1.

In embodiments the appropriate buffer has a pH value between pH 7.0 and pH 8.0. In embodiments the appropriate buffer has a pH value between pH 7.1 and pH 8.0. In embodiments the appropriate buffer has a pH value between pH 7.2 and pH 8.0. In embodiments the appropriate buffer has a pH value between pH 7.3 and pH 8.0. In embodiments the appropriate buffer has a pH value between pH 7.4 and pH 8.0. In embodiments the appropriate buffer has a pH value between pH 7.5 and pH 8.0. In embodiments the appropriate buffer has a pH value between pH 7.6 and pH 8.0. In embodiments the appropriate buffer has a pH value between pH 7.7 and pH 8.0. In embodiments the appropriate buffer has a pH value between pH 7.8 and pH 8.0. In embodiments the appropriate buffer has a pH value between pH 7.9 and pH 8.0.

In embodiments the appropriate buffer has a pH value between pH 7.0 and pH 7.9. In embodiments the appropriate buffer has a pH value between pH 7.0 and pH 7.8. In embodiments the appropriate buffer has a pH value between pH 7.0 and pH 7.7. In embodiments the appropriate buffer has a pH value between pH 7.0 and pH 7.6. In embodiments the appropriate buffer has a pH value between pH 7.0 and pH 7.5. In embodiments the appropriate buffer has a pH value between pH 7.0 and pH 7.4. In embodiments the appropriate buffer has a pH value between pH 7.0 and pH 7.3. In embodiments the appropriate buffer has a pH value between pH 7.0 and pH 7.2. In embodiments the appropriate buffer has a pH value between pH 7.0 and pH 7.1.

In embodiments, the peptide exhibits improved stability at an appropriate temperature. In embodiments, the appropriate temperature is 4° C. In embodiments, the peptide is stable when stored at 4° C. In embodiments, the peptide is stable for at least 4 days, 8 days, 15 days, or 30 days. In embodiments, the peptide is stable for at least 4 days. In embodiments, the peptide is stable for at least 8 days. In embodiments, the peptide is stable for at least 15 days. In embodiments, the peptide is stable for at least 30 days.

In embodiments, the peptide is thermostable up to temperatures of 37° C. In embodiments, the peptide is thermostable up to temperatures of 38° C. In embodiments, the peptide is thermostable up to temperatures of 39° C. In embodiments, the peptide is thermostable up to temperatures of 40° C. In embodiments, the peptide is thermostable up to temperatures of 41° C. In embodiments, the peptide is thermostable up to temperatures of 42° C. In embodiments, the peptide is thermostable up to temperatures of 43° C. In embodiments, the peptide is thermostable up to temperatures of 44° C. In embodiments, the peptide is thermostable up to temperatures of 45° C. In embodiments, the peptide is thermostable up to temperatures of 46° C. In embodiments, the peptide is thermostable up to temperatures of 47° C. In embodiments, the peptide is thermostable up to temperatures of 48° C. In embodiments, the peptide is thermostable up to temperatures of 49° C. In embodiments, the peptide is thermostable up to temperatures of 50° C. In embodiments, the peptide is thermostable up to temperatures of 51° C. In embodiments, the peptide is thermostable up to temperatures of 52° C. In embodiments, the peptide is thermostable up to temperatures of 53° C. In embodiments, the peptide is thermostable up to temperatures of 54° C. In embodiments, the peptide is thermostable up to temperatures of 55° C. In embodiments, the peptide is thermostable up to temperatures of 56° C. In embodiments, the peptide is thermostable up to temperatures of 57° C.

In embodiments, the peptide has an increased melting temperature (T_{M}) relative to a naturally occurring TdT peptide or a recombinant TdT peptide. In embodiments, the peptide has an increased melting temperature (T_{M}) relative to a naturally occurring TdT peptide. In embodiments, the peptide has an increased melting temperature (T_{M}) relative to a recombinant TdT peptide.

In embodiments, the peptide has a T_{M} between about 35° C. to about 60° C. In embodiments, the peptide has a T_{M} between about 36° C. to about 60° C. In embodiments, the peptide has a T_{M} between about 37° C. to about 60° C. In embodiments, the peptide has a T_{M} between about 38° C. to about 60° C. In embodiments, the peptide has a T_{M} between about 39° C. to about 60° C. In embodiments, the peptide has a T_{M} between about 40° C. to about 60° C. In embodiments, the peptide has a T_{M} between about 41° C. to about 60° C. In embodiments, the peptide has a T_{M} between about 42° C. to about 60° C. In embodiments, the peptide has a T_{M} between about 43° C. to about 60° C. In embodiments, the peptide has a T_{M} between about 44° C. to about 60° C. In embodiments, the peptide has a T_{M} between about 45° C. to about 60° C. In embodiments, the peptide has a T_{M} between about 46° C. to about 60° C. In embodiments, the peptide has a T_{M} between about 47° C. to about 60° C. In embodiments, the peptide has a T_{M} between about 48° C. to about 60° C. In embodiments, the peptide has a T_{M} between about 49° C. to about 60° C. In embodiments, the peptide has a T_{M} between about 50° C. to about 60° C. In embodiments, the peptide has a T_{M} between about 51° C. to about 60° C. In embodiments, the peptide has a T_{M} between about 52° C. to about 60° C. In embodiments, the peptide has a T_{M} between about 53° C. to about 60° C. In embodiments, the peptide has a T_{M} between about 54° C. to about 60° C. In embodiments, the peptide has a T_{M} between about 55° C. to about 60° C. In embodiments, the peptide has a T_{M} between about 56° C. to about 60° C. In embodiments, the peptide has a T_{M} between about 57° C. to about 60° C. In embodiments, the peptide has a T_{M} between about 58° C. to about 60° C. In embodiments, the peptide has a T_{M} between about 59° C. to about 60° C.

In embodiments, the peptide has a T_{M} between about 35° C. to about 59° C. In embodiments, the peptide has a T_{M} between about 35° C. to about 58° C. In embodiments, the peptide has a T_{M} between about 35° C. to about 57° C. In embodiments, the peptide has a T_{M} between about 35° C. to about 56° C. In embodiments, the peptide has a T_{M} between about 35° C. to about 55° C. In embodiments, the peptide has a T_{M} between about 35° C. to about 54° C. In embodiments, the peptide has a T_{M} between about 35° C. to about 53° C. In embodiments, the peptide has a T_{M} between about 35° C. to about 52° C. In embodiments, the peptide has a T_{M} between about 35° C. to about 51° C. In embodiments, the peptide has a T_{M} between about 35° C. to about 50° C. In embodiments, the peptide has a T_{M} between about 35° C. to about 49° C. In embodiments, the peptide has a T_{M} between about 35° C. to about 48° C. In embodiments, the peptide has a T_{M} between about 35° C. to about 47° C. In embodiments, the peptide has a T_{M} between about 35° C. to about 46° C. In embodiments, the peptide has a T_{M} between about 35° C. to about 45° C. In embodiments, the peptide has a T_{M} between about 35° C. to about 44° C. In embodiments, the peptide has a T_{M} between about 35° C. to about 43° C. In embodiments, the peptide has a T_{M} between about 35° C. to about 42° C. In embodiments, the peptide has a T_{M} between about 35° C. to about 41° C. In embodiments, the peptide has a T_{M} between about 35° C. to about 40° C. In embodiments, the peptide has a T_{M} between about 35° C. to about 39° C. In embodiments, the peptide has a T_{M} between about 35° C. to about 38° C. In embodiments, the peptide has a T_{M} between about 35° C. to about 37° C. In embodiments, the peptide has a T_{M} between about 35° C. to about 36° C.

In embodiments, the peptide has a T_{M} between 35° C. to 60° C. In embodiments, the peptide has a T_{M} between 36° C. to 60° C. In embodiments, the peptide has a T_{M} between 37° C. to 60° C. In embodiments, the peptide has a T_{M} between 38° C. to 60° C. In embodiments, the peptide has a T_{M} between 39° C. to 60° C. In embodiments, the peptide has a T_{M} between 40° C. to 60° C. In embodiments, the peptide has a T_{M} between 41° C. to 60° C. In embodiments, the peptide has a T_{M} between 42° C. to 60° C. In embodiments, the peptide has a T_{M} between 43° C. to 60° C. In embodiments, the peptide has a T_{M} between 44° C. to 60° C. In embodiments, the peptide has a T_{M} between 45° C. to 60° C. In embodiments, the peptide has a T_{M} between 46° C. to 60° C. In embodiments, the peptide has a T_{M} between 47° C. to 60° C. In embodiments, the peptide has a T_{M} between 48° C. to 60° C. In embodiments, the peptide has a T_{M} between 49° C. to 60° C. In embodiments, the peptide has a T_{M} between 50° C. to 60° C. In embodiments, the peptide has a T_{M} between 51° C. to 60° C. In embodiments, the peptide has a T_{M} between 52° C. to 60° C. In embodiments, the peptide has a T_{M} between 53° C. to 60° C. In embodiments, the peptide has a T_{M} between 54° C. to 60° C. In embodiments, the peptide has a T_{M} between 55° C. to 60° C. In embodiments, the peptide has a T_{M} between 56° C. to 60° C. In embodiments, the peptide has a T_{M} between 57° C. to 60° C. In embodiments, the peptide has a T_{M} between 58° C. to 60° C. In embodiments, the peptide has a T_{M} between 59° C. to 60° C. In embodiments, the peptide has a T_{M} between 35° C. to 59° C. In embodiments, the peptide has a T_{M} between 35° C. to 58° C. In embodiments, the peptide has a T_{M} between 35° C. to 57° C. In embodiments, the peptide has a T_{M} between 35° C. to 56° C. In embodiments, the peptide has a T_{M} between 35° C. to 55° C. In embodiments, the peptide has a T_{M} between 35° C. to 54° C. In embodiments, the peptide has a T_{M} between 35° C. to 53° C. In embodiments, the peptide has a T_{M} between 35° C. to 52° C. In embodiments, the peptide has a T_{M} between 35° C. to 51° C. In embodiments, the peptide has a T_{M} between 35° C. to 50° C. In embodiments, the peptide has a T_{M} between 35° C. to 49° C. In embodiments, the peptide has a T_{M} between 35° C. to 48° C. In embodiments, the peptide has a T_{M} between 35° C. to 47° C. In embodiments, the peptide has a T_{M} between 35° C. to 46° C. In embodiments, the peptide has a T_{M} between 35° C. to 45° C. In embodiments, the peptide has a T_{M} between 35° C. to 44° C. In embodiments, the peptide has a T_{M} between 35° C. to 43° C. In embodiments, the peptide has a T_{M} between 35° C. to 42° C. In embodiments, the peptide has a T_{M} between 35° C. to 41° C. In embodiments, the peptide has a T_{M} between 35° C. to 40° C. In embodiments, the peptide has a Tm between 35° C. to 39° C. In embodiments, the peptide has a T_{M} between 35° C. to 38° C. In embodiments, the peptide has a T_{M} between 35° C. to 37° C. In embodiments, the peptide has a T_{M} between 35° C. to 36° C.

In embodiments, the peptide has a T_{M} of 40° C. In embodiments, the peptide has a T_{M} of 48° C. In embodiments, the peptide has a T_{M} of 41° C. In embodiments, the peptide has a T_{M} of 48° C. In embodiments, the peptide has a T_{M} of 48.5° C. In embodiments, the peptide has a T_{M} of 50° C. In embodiments, the peptide has a T_{M} of 51° C. In embodiments, the peptide has a T_{M} of 53.5° C. In embodiments, the peptide has a T_{M} of 56° C.

In embodiments, the peptide is capable of completing the addition of a blocked nucleotide in less than 5 minutes. In embodiments, the peptide is capable of completing the addition of a blocked nucleotide in less than 4 minutes. In embodiments, the peptide is capable of completing the addition of a blocked nucleotide in less than 3 minutes. In embodiments, the peptide is capable of completing the addition of a blocked nucleotide in less than 2 minutes. In embodiments, the peptide is capable of completing the addition of a blocked nucleotide in less than 1 minute. In embodiments, the peptide is capable of completing the addition of a blocked nucleotide in less than 50 seconds. In embodiments, the peptide is capable of completing the addition of a blocked nucleotide in less than 45 seconds. In embodiments, the peptide is capable of completing the addition of a blocked nucleotide in less than 30 seconds.

### Methods of Use

The recombinant peptides (e.g., TdT peptides) provided herein including embodiments thereof may be used for synthesizing an oligonucleotide. Any of the TdT peptides provided herein including embodiments thereof may be used for methods of synthesizing an oligonucleotide. Thus, in an aspect is provided a method for synthesizing an oligonucleotide, the method includes contacting the TdT peptide provided herein including embodiments thereof with (i) an oligonucleotide primer, wherein the oligonucleotide primer includes a free 3′-hydroxyl; and (ii) a nucleoside triphosphate or 3′-O-blocked nucleoside triphosphate under conditions that promote addition of the nucleotide triphosphate or 3′-O-blocked nucleoside triphosphate to the 3′ terminal end of the oligonucleotide primer, thereby forming an elongated oligonucleotide fragment.

In embodiments, the method is repeated to add additional nucleotides, and wherein the additional nucleotide triphosphates are added to the 3′ terminal end of the elongated oligonucleotide fragment.

In embodiments, the method further includes deblocking the elongated oligonucleotide fragment and allowing the addition of additional 3′-O-blocked nucleoside triphosphates to the 3′ terminal end of the elongated oligonucleotide fragment.

In embodiments, the oligonucleotide primer is immobilized on a solid support. In embodiments, the elongated oligonucleotide fragment is isolated from the solid support. In embodiments, the solid support is an agarose gel, a polyacrylamide gel, a magnetic bead, a silica-based resin, a glass slide, a nitrocellulose membrane, a polystyrene multi-well plate, or a polypropylene multi-well plate. In embodiments, the solid support is an agarose gel. In embodiments, the solid support is a polyacrylamide gel. In embodiments, the solid support is a magnetic bead. In embodiments, the solid support is a silica-based resin. In embodiments, the solid support is a glass slide. In embodiments, the solid support is a nitrocellulose membrane. In embodiments, the solid support is a polystyrene multi-well plate. In embodiments, the solid support is a polypropylene multi-well plate.

In embodiments, the elongated oligonucleotide fragment is at least 5 nucleotides in length. In embodiments, the elongated oligonucleotide fragment is at least 10 nucleotides in length. In embodiments, the elongated oligonucleotide fragment is at least 25 nucleotides in length. In embodiments, the elongated oligonucleotide fragment is at least 50 nucleotides in length. In embodiments, the elongated oligonucleotide fragment is at least 75 nucleotides in length. In embodiments, the elongated oligonucleotide fragment is at least 100 nucleotides in length. In embodiments, the elongated oligonucleotide fragment is at least 200 nucleotides in length. In embodiments, the elongated oligonucleotide fragment is at least 300 in length. In embodiments, the elongated oligonucleotide fragment is at least 400 nucleotides in length. In embodiments, the elongated oligonucleotide fragment is at least 500 nucleotides in length. In embodiments, the elongated oligonucleotide fragment is at least 600 nucleotides in length. In embodiments, the elongated oligonucleotide fragment is at least 700 nucleotides in length. In embodiments, the elongated oligonucleotide fragment is at least 800 nucleotides in length. In embodiments, the elongated oligonucleotide fragment is at least 900 nucleotides in length. In embodiments, the elongated oligonucleotide fragment is at least 1000 nucleotides in length. In embodiments, the elongated oligonucleotide fragment is at least 1250 nucleotides in length. In embodiments, the elongated oligonucleotide fragment is at least 1500 nucleotides in length. In embodiments, the elongated oligonucleotide fragment is at least 1750 nucleotides in length. In embodiments, the elongated oligonucleotide fragment is at least 2000 nucleotides in length.

In embodiments, the oligonucleotide primer is at least 3 nucleotides in length. In embodiments, the oligonucleotide primer is at least 5 nucleotides in length. In embodiments, the oligonucleotide primer is at least 10 nucleotides in length. In embodiments, the oligonucleotide primer is at least 25 nucleotides in length. In embodiments, the oligonucleotide primer is at least 50 nucleotides in length. In embodiments, the oligonucleotide primer is at least 75 nucleotides in length. In embodiments, the oligonucleotide primer is at least 100 nucleotides in length. In embodiments, the oligonucleotide primer is at least 200 nucleotides in length. In embodiments, the oligonucleotide primer is at least 300 in length. In embodiments, the oligonucleotide primer is at least 400 nucleotides in length. In embodiments, the oligonucleotide primer is at least 500 nucleotides in length. In embodiments, the oligonucleotide primer is at least 600 nucleotides in length. In embodiments, the oligonucleotide primer is at least 700 nucleotides in length. In embodiments, the oligonucleotide primer is at least 800 nucleotides in length. In embodiments, the oligonucleotide primer is at least 900 nucleotides in length. In embodiments, the oligonucleotide primer is at least 1000 nucleotides in length. In embodiments, the oligonucleotide primer is at least 1250 nucleotides in length. In embodiments, the oligonucleotide primer is at least 1500 nucleotides in length. In embodiments, the oligonucleotide primer is at least 1750 nucleotides in length. In embodiments, the oligonucleotide primer is at least 2000 nucleotides in length.

In embodiments, the TdT peptide has minimal variation in its binding affinity or catalytic rate for a nitrogenous base. In embodiments, the TdT peptide has minimal variation in its binding affinity or catalytic rate for each nitrogenous base. In embodiments, the TdT peptide has minimal variation in its binding affinity and catalytic rate for a nitrogenous base. In embodiments, the TdT peptide has minimal variation in its binding affinity and catalytic rate for each nitrogenous base. In embodiments, the TdT peptide has minimal variation in its binding affinity for a nitrogenous base. In embodiments, the TdT peptide has minimal variation in its binding affinity for each nitrogenous base. In embodiments, the TdT peptide has minimal variation in its catalytic rate for a nitrogenous base. In embodiments, the TdT peptide has minimal variation in its catalytic rate for each nitrogenous base.

In embodiments, the affinity of the TdT peptide for a nitrogenous base is less than 25% different relative to the affinity of the TdT peptide for another nitrogenous base. In embodiments, the affinity of the TdT peptide for a nitrogenous base is less than 20% different relative to the affinity of the TdT peptide for another nitrogenous base. In embodiments, the affinity of the TdT peptide for a nitrogenous base is less than 15% different relative to the affinity of the TdT peptide for another nitrogenous base. In embodiments, the affinity of the TdT peptide for a nitrogenous base is less than 10% different relative to the affinity of the TdT peptide for another nitrogenous base. In embodiments, the affinity of the TdT peptide for a nitrogenous base is less than 9% different relative to the affinity of the TdT peptide for another nitrogenous base. In embodiments, the affinity of the TdT peptide for a nitrogenous base is less than 8% different relative to the affinity of the TdT peptide for another nitrogenous base. In embodiments, the affinity of the TdT peptide for a nitrogenous base is less than 7% different relative to the affinity of the TdT peptide for another nitrogenous base. In embodiments, the affinity of the TdT peptide for a nitrogenous base is less than 6% different relative to the affinity of the TdT peptide for another nitrogenous base. In embodiments, the affinity of the TdT peptide for a nitrogenous base is less than 5% different relative to the affinity of the TdT peptide for another nitrogenous base. In embodiments, the affinity of the TdT peptide for a nitrogenous base is less than 4% different relative to the affinity of the TdT peptide for another nitrogenous base. In embodiments, the affinity of the TdT peptide for a nitrogenous base is less than 3% different relative to the affinity of the TdT peptide for another nitrogenous base. In embodiments, the affinity of the TdT peptide for a nitrogenous base is less than 2% different relative to the affinity of the TdT peptide for another nitrogenous base. In embodiments, the affinity of the TdT peptide for a nitrogenous base is less than 1% different relative to the affinity of the TdT peptide for another nitrogenous base.

In embodiments, the catalytic rate of the TdT peptide for a nitrogenous base is less than 25% different relative to the catalytic rate of the TdT peptide for another nitrogenous base. In embodiments, the catalytic rate of the TdT peptide for a nitrogenous base is less than 20% different relative to the catalytic rate of the TdT peptide for another nitrogenous base. In embodiments, the catalytic rate of the TdT peptide for a nitrogenous base is less than 15% different relative to the catalytic rate of the TdT peptide for another nitrogenous base. In embodiments, the catalytic rate of the TdT peptide for a nitrogenous base is less than 10% different relative to the catalytic rate of the TdT peptide for another nitrogenous base. In embodiments, the catalytic rate of the TdT peptide for a nitrogenous base is less than 9% different relative to the catalytic rate of the TdT peptide for another nitrogenous base. In embodiments, the catalytic rate of the TdT peptide for a nitrogenous base is less than 8% different relative to the catalytic rate of the TdT peptide for another nitrogenous base. In embodiments, the catalytic rate of the TdT peptide for a nitrogenous base is less than 7% different relative to the catalytic rate of the TdT peptide for another nitrogenous base. In embodiments, the catalytic rate of the TdT peptide for a nitrogenous base is less than 6% different relative to the catalytic rate of the TdT peptide for another nitrogenous base. In embodiments, the catalytic rate of the TdT peptide for a nitrogenous base is less than 5% different relative to the catalytic rate of the TdT peptide for another nitrogenous base. In embodiments, the catalytic rate of the TdT peptide for a nitrogenous base is less than 4% different relative to the catalytic rate of the TdT peptide for another nitrogenous base. In embodiments, the catalytic rate of the TdT peptide for a nitrogenous base is less than 3% different relative to the catalytic rate of the TdT peptide for another nitrogenous base. In embodiments, the catalytic rate of the TdT peptide for a nitrogenous base is less than 2% different relative to the catalytic rate of the TdT peptide for another nitrogenous base. In embodiments, the catalytic rate of the TdT peptide for a nitrogenous base is less than 1% different relative to the catalytic rate of the TdT peptide for another nitrogenous base.

In embodiments, the TdT peptide is capable of completing the addition of a blocked nucleotide in less than 5 minutes. In embodiments, the TdT peptide is capable of completing the addition of a blocked nucleotide in less than 4 minutes. In embodiments, the TdT peptide is capable of completing the addition of a blocked nucleotide in less than 3 minutes. In embodiments, the TdT peptide is capable of completing the addition of a blocked nucleotide in less than 2 minutes. In embodiments, the TdT peptide is capable of completing the addition of a blocked nucleotide in less than 1 minute. In embodiments, the TdT peptide is capable of completing the addition of a blocked nucleotide in less than 50 seconds. In embodiments, the TdT peptide is capable of completing the addition of a blocked nucleotide in less than 45 seconds. In embodiments, the TdT peptide is capable of completing the addition of a blocked nucleotide in less than 30 seconds.

In embodiments, the peptide exhibits improved stability in an appropriate buffer at an appropriate temperature. In embodiments, the peptide exhibits improved stability in an appropriate buffer. In embodiments, the appropriate buffer is Tris, acetate, 4-(2-hydroxyethyl)-1-piperazineethanesulfonic acid (HEPES), 3-(N-morpholino) propanesulfonic acid (MOPS), cacodylate, or a phosphate buffer. In embodiments, the appropriate buffer is Tris, acetate. In embodiments, the appropriate buffer is 4-(2-hydroxyethyl)-1-piperazineethanesulfonic acid (HEPES). In embodiments, the appropriate buffer is 3-(N-morpholino) propanesulfonic acid (MOPS). In embodiments, the appropriate buffer is cacodylate. In embodiments, the appropriate buffer is a phosphate buffer.

In embodiments the appropriate buffer has a pH value between about pH 7.0 and about pH 8.0. In embodiments the appropriate buffer has a pH value between about pH 7.1 and about pH 8.0. In embodiments the appropriate buffer has a pH value between about pH 7.2 and about pH 8.0. In embodiments the appropriate buffer has a pH value between about pH 7.3 and about pH 8.0. In embodiments the appropriate buffer has a pH value between about pH 7.4 and about pH 8.0. In embodiments the appropriate buffer has a pH value between about pH 7.5 and about pH 8.0. In embodiments the appropriate buffer has a pH value between about pH 7.6 and about pH 8.0. In embodiments the appropriate buffer has a pH value between about pH 7.7 and about pH 8.0. In embodiments the appropriate buffer has a pH value between about pH 7.8 and about pH 8.0. In embodiments the appropriate buffer has a pH value between about pH 7.9 and about pH 8.0.

In embodiments the appropriate buffer has a pH value between about pH 7.0 and about pH 7.9. In embodiments the appropriate buffer has a pH value between about pH 7.0 and about pH 7.8. In embodiments the appropriate buffer has a pH value between about pH 7.0 and about pH 7.7. In embodiments the appropriate buffer has a pH value between about pH 7.0 and about pH 7.6. In embodiments the appropriate buffer has a pH value between about pH 7.0 and about pH 7.5. In embodiments the appropriate buffer has a pH value between about pH 7.0 and about pH 7.4. In embodiments the appropriate buffer has a pH value between about pH 7.0 and about pH 7.3. In embodiments the appropriate buffer has a pH value between about pH 7.0 and about pH 7.2. In embodiments the appropriate buffer has a pH value between about pH 7.0 and about pH 7.1.

In embodiments the appropriate buffer has a pH value between pH 7.0 and pH 8.0. In embodiments the appropriate buffer has a pH value between pH 7.1 and pH 8.0. In embodiments the appropriate buffer has a pH value between pH 7.2 and pH 8.0. In embodiments the appropriate buffer has a pH value between pH 7.3 and pH 8.0. In embodiments the appropriate buffer has a pH value between pH 7.4 and pH 8.0. In embodiments the appropriate buffer has a pH value between pH 7.5 and pH 8.0. In embodiments the appropriate buffer has a pH value between pH 7.6 and pH 8.0. In embodiments the appropriate buffer has a pH value between pH 7.7 and pH 8.0. In embodiments the appropriate buffer has a pH value between pH 7.8 and pH 8.0. In embodiments the appropriate buffer has a pH value between pH 7.9 and pH 8.0.

In embodiments the appropriate buffer has a pH value between pH 7.0 and pH 7.9. In embodiments the appropriate buffer has a pH value between pH 7.0 and pH 7.8. In embodiments the appropriate buffer has a pH value between pH 7.0 and pH 7.7. In embodiments the appropriate buffer has a pH value between pH 7.0 and pH 7.6. In embodiments the appropriate buffer has a pH value between pH 7.0 and pH 7.5. In embodiments the appropriate buffer has a pH value between pH 7.0 and pH 7.4. In embodiments the appropriate buffer has a pH value between pH 7.0 and pH 7.3. In embodiments the appropriate buffer has a pH value between pH 7.0 and pH 7.2. In embodiments the appropriate buffer has a pH value between pH 7.0 and pH 7.1.

In embodiments, the TdT peptide includes the amino acid sequence of SEQ ID NO:1, SEQ ID NO:2, SEQ ID NO:3, SEQ ID NO: 16, SEQ ID NO:17, SEQ ID NO:18, SEQ ID NO:19, SEQ ID NO:20, SEQ ID NO:21, SEQ ID NO:22, SEQ ID NO:23, or SEQ ID NO:24. In embodiments, the TdT peptide includes the amino acid sequence of SEQ ID NO:1. In embodiments, the TdT peptide includes the amino acid sequence of SEQ ID NO:2. In embodiments, the TdT peptide includes the amino acid sequence of SEQ ID NO:3. In embodiments, the TdT peptide includes the amino acid sequence of SEQ ID NO:16. In embodiments, the TdT peptide includes the amino acid sequence of SEQ ID NO:17. In embodiments, the TdT peptide includes the amino acid sequence of SEQ ID NO:18. In embodiments, the TdT peptide includes the amino acid sequence of SEQ ID NO:19. In embodiments, the TdT peptide includes the amino acid sequence of SEQ ID NO:20. In embodiments, the TdT peptide includes the amino acid sequence of SEQ ID NO:21. In embodiments, the TdT peptide includes the amino acid sequence of SEQ ID NO:22. In embodiments, the TdT peptide includes the amino acid sequence of SEQ ID NO:23. In embodiments, the TdT peptide includes the amino acid sequence of SEQ ID NO:24.

In embodiments, the TdT peptide is the amino acid sequence of SEQ ID NO:1, SEQ ID NO: 2, SEQ ID NO:3, SEQ ID NO: 16, SEQ ID NO: 17, SEQ ID NO: 18, SEQ ID NO: 19, SEQ ID NO: 20, SEQ ID NO:21, SEQ ID NO:22, SEQ ID NO:23, or SEQ ID NO:24. In embodiments, the TdT peptide is the amino acid sequence of SEQ ID NO:1. In embodiments, the TdT peptide is the amino acid sequence of SEQ ID NO:2. In embodiments, the TdT peptide is the amino acid sequence of SEQ ID NO:3. In embodiments, the TdT peptide is the amino acid sequence of SEQ ID NO: 16. In embodiments, the TdT peptide is the amino acid sequence of SEQ ID NO:17. In embodiments, the TdT peptide is the amino acid sequence of SEQ ID NO:18. In embodiments, the TdT peptide is the amino acid sequence of SEQ ID NO: 19. In embodiments, the TdT peptide is the amino acid sequence of SEQ ID NO:20. In embodiments, the TdT peptide is the amino acid sequence of SEQ ID NO:21. In embodiments, the TdT peptide is the amino acid sequence of SEQ ID NO:22. In embodiments, the TdT peptide is the amino acid sequence of SEQ ID NO:23. In embodiments, the TdT peptide is the amino acid sequence of SEQ ID NO:24.

### Compositions and Kits

The recombinant peptides (e.g., TdT peptides) provided herein including embodiments thereof may be used in compositions and kits for synthesizing oligonucleotides. Thus, in an aspect is provided a composition including the TdT peptide provided herein including embodiments thereof and an appropriate buffer.

In embodiments, the appropriate buffer is Tris, acetate, 4-(2-hydroxyethyl)-1-piperazineethanesulfonic acid (HEPES), 3-(N-morpholino) propanesulfonic acid (MOPS), cacodylate, or a phosphate buffer. In embodiments, the appropriate buffer is Tris, acetate. In embodiments, the appropriate buffer is 4-(2-hydroxyethyl)-1-piperazineethanesulfonic acid (HEPES). In embodiments, the appropriate buffer is 3-(N-morpholino) propanesulfonic acid (MOPS). In embodiments, the appropriate buffer is cacodylate. In embodiments, the appropriate buffer is a phosphate buffer.

In embodiments the appropriate buffer has a pH value between about pH 7.0 and about pH 8.0. In embodiments the appropriate buffer has a pH value between about pH 7.1 and about pH 8.0. In embodiments the appropriate buffer has a pH value between about pH 7.2 and about pH 8.0. In embodiments the appropriate buffer has a pH value between about pH 7.3 and about pH 8.0. In embodiments the appropriate buffer has a pH value between about pH 7.4 and about pH 8.0. In embodiments the appropriate buffer has a pH value between about pH 7.5 and about pH 8.0. In embodiments the appropriate buffer has a pH value between about pH 7.6 and about pH 8.0. In embodiments the appropriate buffer has a pH value between about pH 7.7 and about pH 8.0. In embodiments the appropriate buffer has a pH value between about pH 7.8 and about pH 8.0. In embodiments the appropriate buffer has a pH value between about pH 7.9 and about pH 8.0.

In embodiments the appropriate buffer has a pH value between about pH 7.0 and about pH 7.9. In embodiments the appropriate buffer has a pH value between about pH 7.0 and about pH 7.8. In embodiments the appropriate buffer has a pH value between about pH 7.0 and about pH 7.7. In embodiments the appropriate buffer has a pH value between about pH 7.0 and about pH 7.6. In embodiments the appropriate buffer has a pH value between about pH 7.0 and about pH 7.5. In embodiments the appropriate buffer has a pH value between about pH 7.0 and about pH 7.4. In embodiments the appropriate buffer has a pH value between about pH 7.0 and about pH 7.3. In embodiments the appropriate buffer has a pH value between about pH 7.0 and about pH 7.2. In embodiments the appropriate buffer has a pH value between about pH 7.0 and about pH 7.1.

In embodiments the appropriate buffer has a pH value between pH 7.0 and pH 8.0. In embodiments the appropriate buffer has a pH value between pH 7.1 and pH 8.0. In embodiments the appropriate buffer has a pH value between pH 7.2 and pH 8.0. In embodiments the appropriate buffer has a pH value between pH 7.3 and pH 8.0. In embodiments the appropriate buffer has a pH value between pH 7.4 and pH 8.0. In embodiments the appropriate buffer has a pH value between pH 7.5 and pH 8.0. In embodiments the appropriate buffer has a pH value between pH 7.6 and pH 8.0. In embodiments the appropriate buffer has a pH value between pH 7.7 and pH 8.0. In embodiments the appropriate buffer has a pH value between pH 7.8 and pH 8.0. In embodiments the appropriate buffer has a pH value between pH 7.9 and pH 8.0.

In embodiments the appropriate buffer has a pH value between pH 7.0 and pH 7.9. In embodiments the appropriate buffer has a pH value between pH 7.0 and pH 7.8. In embodiments the appropriate buffer has a pH value between pH 7.0 and pH 7.7. In embodiments the appropriate buffer has a pH value between pH 7.0 and pH 7.6. In embodiments the appropriate buffer has a pH value between pH 7.0 and pH 7.5. In embodiments the appropriate buffer has a pH value between pH 7.0 and pH 7.4. In embodiments the appropriate buffer has a pH value between pH 7.0 and pH 7.3. In embodiments the appropriate buffer has a pH value between pH 7.0 and pH 7.2. In embodiments the appropriate buffer has a pH value between pH 7.0 and pH 7.1

In another aspect is provided a kit for synthesizing a target oligonucleotide, the kit including: a) the composition provided herein including embodiments thereof; b) a nucleotide triphosphate or a 3′-O-blocked nucleoside triphosphate; and c) an oligonucleotide primer including a free 3′-hydroxyl.

In embodiments, the oligonucleotide primer is immobilized on a solid support. In embodiments, the solid support is an agarose gel, a polyacrylamide gel, a magnetic bead, a silica-based resin, a glass slide, a nitrocellulose membrane, a polystyrene multi-well plate, or a polypropylene multi-well plate. In embodiments, the solid support is an agarose gel. In embodiments, the solid support is a polyacrylamide gel. In embodiments, the solid support is a magnetic bead. In embodiments, the solid support is a silica-based resin. In embodiments, the solid support is a glass slide. In embodiments, the solid support is a nitrocellulose membrane. In embodiments, the solid support is a polystyrene multi-well plate. In embodiments, the solid support is a polypropylene multi-well plate.

In embodiments, the kit further includes a reducing agent, a detergent, and a cryopreservative. In embodiments, the kit further includes a reducing agent. In embodiments, the kit further includes a detergent. In embodiments, the kit further includes a cryopreservative.

In embodiments, the reducing agent includes dithiothreitol, beta-mercaptoethanol, or tris(2-carboxyethyl)phosphine. In embodiments, the reducing agent includes dithiothreitol. In embodiments, the reducing agent includes beta-mercaptoethanol. In embodiments, the reducing agent includes tris(2-carboxyethyl)phosphine. In embodiments, the reducing agent is dithiothreitol or beta-mercaptoethanol, tris(2-carboxyethyl)phosphine. In embodiments, the reducing agent is dithiothreitol. In embodiments, the reducing agent is beta-mercaptoethanol. In embodiments, the reducing agent is tris(2-carboxyethyl)phosphine.

In embodiments, the detergent includes triton X-100, tween20, tween 80, sarkosyl, igepal CA-630, or NP-40. In embodiments, the detergent includes triton X-100. In embodiments, the detergent includes tween20. In embodiments, the detergent includes tween 80. In embodiments, the detergent includes sarkosyl. In embodiments, the detergent includes igepal CA-630. In embodiments, the detergent includes NP-40 In embodiments, the detergent is triton X-100, tween20, tween 80, sarkosyl, igepal CA-630, or NP-40. In embodiments, the detergent is triton X-100. In embodiments, the detergent includes tween20. In embodiments, the detergent is tween 80. In embodiments, the detergent is sarkosyl. In embodiments, the detergent is igepal CA-630. In embodiments, the detergent is NP-40.

In embodiments the cryopreservative includes glycerol, trehalose, or mannitol. In embodiments the cryopreservative includes glycerol. In embodiments the cryopreservative includes trehalose. In embodiments the cryopreservative includes mannitol. In embodiments the cryopreservative is glycerol, trehalose, or mannitol. In embodiments the cryopreservative is glycerol. In embodiments the cryopreservative is trehalose. In embodiments the cryopreservative is mannitol.

In embodiments, the TdT peptide includes the amino acid sequence of SEQ ID NO:1, SEQ ID NO:2, SEQ ID NO:3, SEQ ID NO: 16, SEQ ID NO: 17, SEQ ID NO:18, SEQ ID NO: 19, SEQ ID NO:20, SEQ ID NO:21, SEQ ID NO:22, SEQ ID NO:23, or SEQ ID NO:24. In embodiments, the TdT peptide includes the amino acid sequence of SEQ ID NO:1. In embodiments, the TdT peptide includes the amino acid sequence of SEQ ID NO:2. In embodiments, the TdT peptide includes the amino acid sequence of SEQ ID NO:3. In embodiments, the TdT peptide includes the amino acid sequence of SEQ ID NO:16. In embodiments, the TdT peptide includes the amino acid sequence of SEQ ID NO:17. In embodiments, the TdT peptide includes the amino acid sequence of SEQ ID NO:18. In embodiments, the TdT peptide includes the amino acid sequence of SEQ ID NO:19. In embodiments, the TdT peptide includes the amino acid sequence of SEQ ID NO:20. In embodiments, the TdT peptide includes the amino acid sequence of SEQ ID NO:21. In embodiments, the TdT peptide includes the amino acid sequence of SEQ ID NO:22. In embodiments, the TdT peptide includes the amino acid sequence of SEQ ID NO:23. In embodiments, the TdT peptide includes the amino acid sequence of SEQ ID NO:24.

In embodiments, the TdT peptide is the amino acid sequence of SEQ ID NO:1, SEQ ID NO: 2, SEQ ID NO:3, SEQ ID NO:16, SEQ ID NO:17, SEQ ID NO: 18, SEQ ID NO: 19, SEQ ID NO: 20, SEQ ID NO:21, SEQ ID NO:22, SEQ ID NO:23, or SEQ ID NO:24. In embodiments, the TdT peptide is the amino acid sequence of SEQ ID NO: 1. In embodiments, the TdT peptide is the amino acid sequence of SEQ ID NO:2. In embodiments, the TdT peptide is the amino acid sequence of SEQ ID NO:3. In embodiments, the TdT peptide is the amino acid sequence of SEQ ID NO: 16. In embodiments, the TdT peptide is the amino acid sequence of SEQ ID NO:17. In embodiments, the TdT peptide is the amino acid sequence of SEQ ID NO:18. In embodiments, the TdT peptide is the amino acid sequence of SEQ ID NO:19. In embodiments, the TdT peptide is the amino acid sequence of SEQ ID NO:20. In embodiments, the TdT peptide is the amino acid sequence of SEQ ID NO:21. In embodiments, the TdT peptide is the amino acid sequence of SEQ ID NO:22. In embodiments, the TdT peptide is the amino acid sequence of SEQ ID NO:23. In embodiments, the TdT peptide is the amino acid sequence of SEQ ID NO:24.

In embodiments, the TdT peptide includes the amino acid sequence of SEQ ID NO:25, SEQ ID NO:26, SEQ ID NO:27, SEQ ID NO:28, SEQ ID NO:29, SEQ ID NO:30, SEQ ID NO: 31, SEQ ID NO:32, SEQ ID NO:33, SEQ ID NO:34, SEQ ID NO:35, or SEQ ID NO:36. In embodiments, the TdT peptide includes the amino acid sequence of SEQ ID NO:25. In embodiments, the TdT peptide includes the amino acid sequence of SEQ ID NO:26. In embodiments, the TdT peptide includes the amino acid sequence of SEQ ID NO:27. In embodiments, the TdT peptide includes the amino acid sequence of SEQ ID NO:28. In embodiments, the TdT peptide includes the amino acid sequence of SEQ ID NO:29. In embodiments, the TdT peptide includes the amino acid sequence of SEQ ID NO:30. In embodiments, the TdT peptide includes the amino acid sequence of SEQ ID NO:31. In embodiments, the TdT peptide includes the amino acid sequence of SEQ ID NO:32. In embodiments, the TdT peptide includes the amino acid sequence of SEQ ID NO:33. In embodiments, the TdT peptide includes the amino acid sequence of SEQ ID NO:34. In embodiments, the TdT peptide includes the amino acid sequence of SEQ ID NO:35. In embodiments, the TdT peptide includes the amino acid sequence of SEQ ID NO:36.

In embodiments, the TdT peptide is the amino acid sequence of SEQ ID NO:25, SEQ ID NO: 26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, or SEQ ID NO:36. In embodiments, the TdT peptide is the amino acid sequence of SEQ ID NO:25. In embodiments, the TdT peptide is the amino acid sequence of SEQ ID NO:26. In embodiments, the TdT peptide is the amino acid sequence of SEQ ID NO:27. In embodiments, the TdT peptide is the amino acid sequence of SEQ ID NO:28. In embodiments, the TdT peptide is the amino acid sequence of SEQ ID NO:29. In embodiments, the TdT peptide is the amino acid sequence of SEQ ID NO:30. In embodiments, the TdT peptide is the amino acid sequence of SEQ ID NO:31. In embodiments, the TdT peptide is the amino acid sequence of SEQ ID NO:32. In embodiments, the TdT peptide is the amino acid sequence of SEQ ID NO:33. In embodiments, the TdT peptide is the amino acid sequence of SEQ ID NO:34. In embodiments, the TdT peptide is the amino acid sequence of SEQ ID NO:35. In embodiments, the TdT peptide is the amino acid sequence of SEQ ID NO:36.

### EXAMPLES

The following examples are intended to illustrate the invention and its preferred embodiments, but in no circumstance should they be considered to restrict the scope of the invention, which will be defined by the tenor of the claims attached herein.

### Example 1: Modified Nucleotides Addition

In accordance with the present invention, the additions of modified nucleotides by the enzymes of the present invention is exemplified by the following experiment.

A Terminal deoxynucleotidyl transferase (TdT) reaction was carried out for SEQ ID NO:1 (N62), SEQ ID NO:2 (N54), SEQ ID NO:3 (N62), and mouse TdT (mTdT), involving a 10 μl mixture containing a FAM-labeled oligonucleotide of 16 nucleotides in length, in combination with the four reversibly blocked nucleotides (ONH2) at a final concentration of 40 μM of ONH2. The reaction was incubated for 5 minutes at 37° C.

As a control, a parallel reaction was conducted in the absence of dNTPs for all enzymes tested. To denature the reaction products and dissociate potential enzyme-substrate complexes, 2.5 μl of 5×Urea buffer was added to each sample, followed by incubation for 5 minutes at 95° C.

Subsequently, 2 μl aliquots of the denatured reaction products were subjected to electrophoretic separation on a 20% SDS-PAGE Urea gel to assess the extent of the TdT reaction. To evaluate the successful addition of a single nucleotide, a positive control containing a mixture of 16 and 17 nucleotide-long oligonucleotides was loaded alongside the experimental samples on the gel. The electrophoretic migration of the reaction products was compared to the positive control to determine the efficiency and specificity of the engineered enzymes in the TdT reaction.

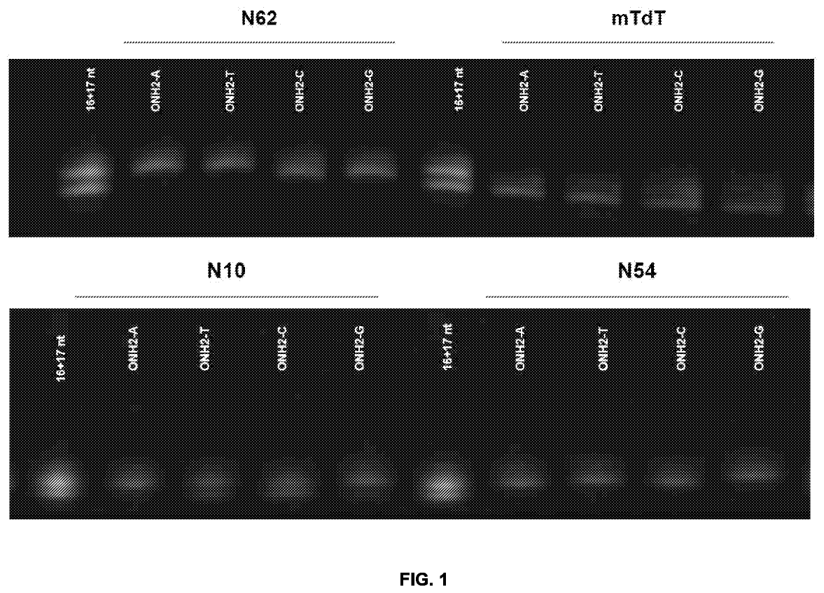

As seen in FIG. 1, SEQ ID NO:1 (N10), SEQ ID NO:2 (N54), SEQ ID NO:3 (N62), showed noticeable incorporation of the modified nucleotides as the 17 nt band is visible in all samples, while in the mouse TdT (mTdT) case, there are noticeable 16 nt bands instead.

### Example 2: Oligonucleotide Extension at Different Temperatures

First, extension reaction mixtures were prepared for enzymes of sequence SEQ ID NO: 1, SEQ ID NO:2, and SEQ ID NO:3, mTdT (mouse), bTdT (bovine), and the negative control without enzyme (NEC) in separate test tubes. These mixtures contained random hexamer oligonucleotides (0.01 μM), dNTPs (1 mM), and the appropriate reaction buffer. All mixtures were brought to an enzyme concentration of 0.08 μg/μL.

Next, the reaction mixtures were incubated at 37, 41, 45, 49, 53, and 57° C. for polymerization for 30 minutes in a thermal block.

A 1% denaturing agarose gel was prepared, and the samples from the reaction mixtures were loaded into the agarose gel wells along with the molecular weight marker (MW).

Then, the electrophoresis equipment was connected, and the run was performed at a voltage of 100 V for a sufficient time to obtain a good separation of the bands. After the run, the gel was stained and DNA bands were visualized under ultraviolet light using a UV transilluminator.

Subsequently, the polymerization activity of the candidate enzymes was analyzed by comparing the intensity and position of the bands with the positive controls (mTdT and bTdT) and the negative control (NEC).

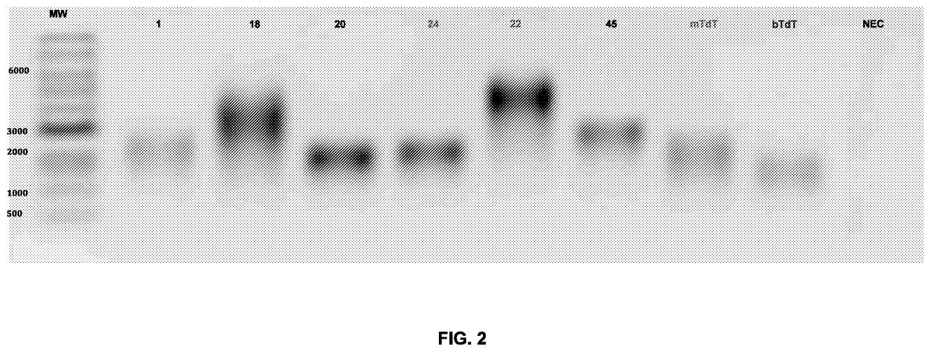

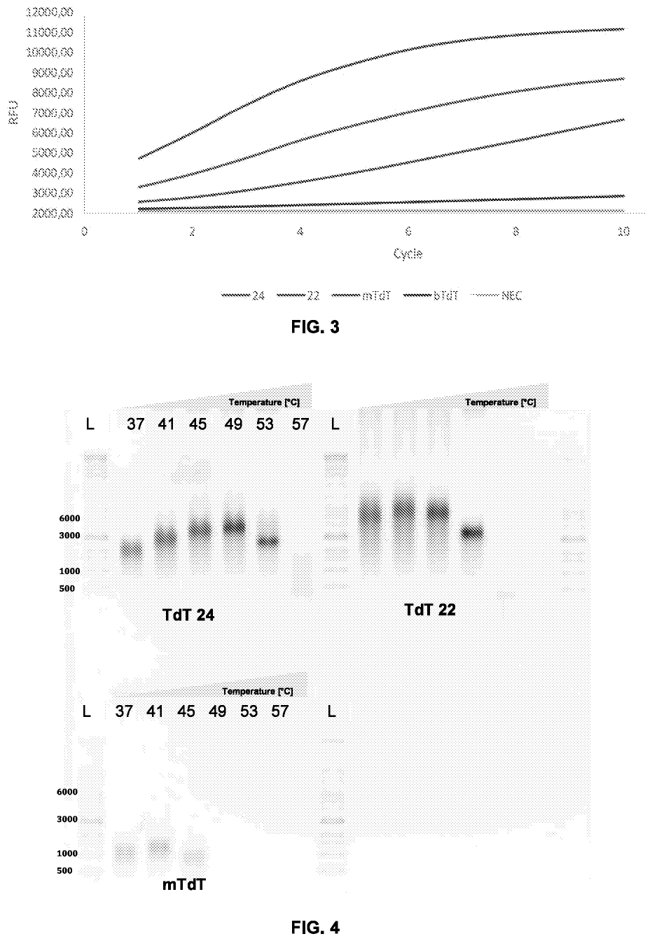

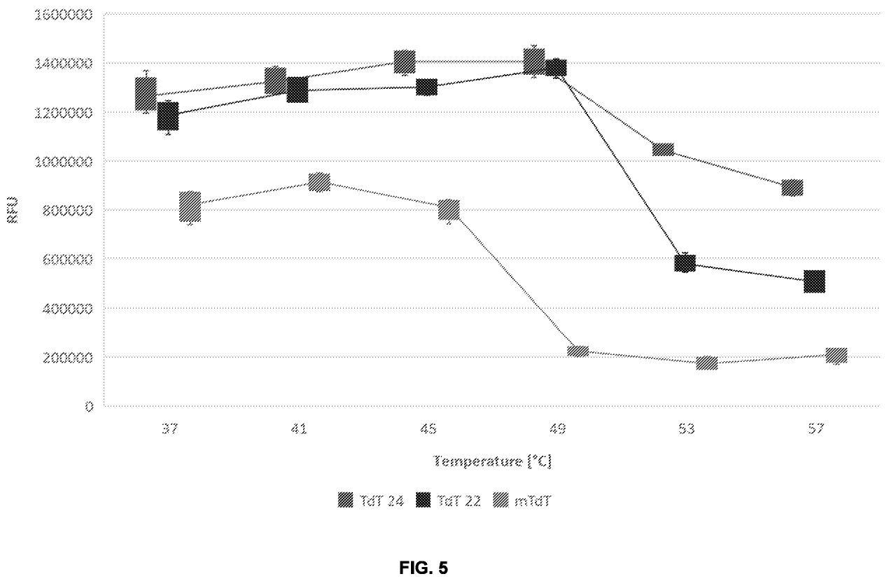

This allowed for the identification of enzymes with higher polymerization activity when compared to the controls, as can be seen in FIGS. 2 and 3, and the determination of enzyme thermostability, as can be seen in FIGS. 4 and 5.

### Example 3: Generation, Expression, and Purification of TdT Peptides

TABLE 1
Alignment of the amino acid sequences TdT peptides provided herein.

| Position | | | | | | | | | | | | |
|---|---|---|---|---|---|---|---|---|---|---|---|---|
| with | | | | | | | | | | | | |
| respect | SEQ | SEQ | SEQ | SEQ | SEQ | SEQ | SEQ | SEQ | SEQ | SEQ | SEQ | SEQ |
| to SEQ | ID | ID | ID | ID | ID | ID | ID | ID | ID | ID | ID | ID |
| ID NO: | NO: | NO: | NO: | NO: | NO: | NO: | NO: | NO: | NO: | NO: | NO: | NO: |
| 3 | 3 | 16 | 2 | 17 | 1 | 18 | 19 | 20 | 21 | 22 | 23 | 24 |
| 1 | M | M | M | M | M | M | M | M | M | M | M | M |
| 2 | E | E | E | E | — | E | V | V | V | V | V | V |
| 3 | Q | Q | Q | Q | — | S | R | Q | Q | Q | Q | Q |
| | — | — | Q | Q | — | R | R | Q | Q | Q | Q | Q |
| 4 | D | D | D | D | D | V | D | D | D | D | D | D |
| | — | — | — | — | — | A | C | C | C | C | C | C |
| | — | — | — | — | — | S | S | S | S | S | S | S |
| | — | — | — | — | — | A | A | A | A | A | A | A |
| | — | — | — | — | — | N | S | S | S | S | T | T |
| 5 | Q | Q | Q | Q | T | P | P | — | Y | Y | S | S |
| 6 | S | S | S | S | S | D | N | — | Q | Q | D | D |
| 7 | P | P | P | P | S | P | P | S | S | S | — | — |
| 8 | P | P | T | T | P | — | — | S | S | S | — | — |
| 9 | L | L | L | L | S | — | — | S | S | S | — | — |
| 10 | N | N | N | N | P | N | S | S | S | S | — | — |
| 11 | T | T | T | T | E | A | T | T | T | T | T | T |
| 12 | P | P | P | P | R | G | R | R | R | R | P | P |
| 13 | E | E | E | E | S | M | M | P | P | P | E | E |
| 14 | S | S | S | S | A | L | T | T | T | T | S | S |
| 15 | E | E | E | E | P | K | K | E | E | E | E | E |
| 16 | V | V | V | V | T | I | T | T | T | T | A | A |
| 17 | P | P | P | P | P | Q | P | P | S | S | S | S |
| 18 | S | S | S | S | A | S | S | S | S | S | S | S |
| 19 | F | F | F | F | S | P | L | L | L | L | F | F |
| 20 | I | I | T | T | P | A | A | A | A | A | A | A |
| 21 | A | A | A | A | V | T | T | A | A | A | A | A |
| 22 | S | S | S | S | A | K | K | A | A | A | A | A |
| 23 | K | K | K | K | T | T | K | K | K | K | K | K |
| 24 | V | V | V | V | V | I | I | V | V | V | V | V |
| 25 | S | S | S | S | S | S | S | S | S | S | S | S |
| 26 | Q | Q | Q | Q | Q | Q | Q | Q | Q | Q | Q | Q |
| 27 | Y | Y | Y | Y | Y | Y | Y | Y | Y | Y | Y | Y |
| 28 | S | S | S | S | A | A | A | A | A | A | A | A |
| 29 | C | C | C | C | C | C | C | C | C | C | C | C |
| 30 | Q | Q | Q | Q | Q | Q | Q | Q | Q | Q | Q | Q |
| 31 | R | R | R | R | R | R | R | R | R | R | R | R |
| 32 | K | K | K | K | R | R | R | R | R | R | R | R |
| 33 | T | T | T | T | T | T | T | T | T | T | T | T |
| 34 | T | T | T | T | T | T | T | T | T | T | T | T |
| 35 | L | L | L | L | L | L | L | L | L | L | L | L |
| 36 | N | N | N | N | E | N | N | N | N | N | N | N |
| 37 | N | N | N | N | N | N | N | N | N | N | N | N |
| 38 | Y | Y | Y | Y | H | H | C | C | C | C | C | Y |
| 39 | N | N | N | N | N | N | N | N | N | N | N | N |
| 40 | K | K | K | K | K | Q | Q | K | K | K | K | K |
| 41 | K | K | K | K | I | R | K | K | K | K | K | K |
| 42 | F | F | F | F | F | F | F | F | F | F | F | F |
| 43 | T | T | T | T | T | T | T | T | T | T | T | T |
| 44 | D | D | D | D | D | D | D | D | D | D | D | D |
| 45 | A | A | A | A | A | A | A | A | A | A | A | A |
| 46 | F | F | F | F | F | F | F | F | F | F | F | F |
| 47 | E | E | E | E | E | E | E | E | E | E | E | E |
| 48 | I | I | I | I | I | I | I | I | I | I | I | I |
| 49 | M | M | M | M | L | L | L | L | L | L | L | L |
| 50 | A | A | A | A | A | A | A | A | A | A | A | A |
| 51 | E | E | E | E | E | K | E | E | E | E | E | E |
| 52 | N | N | N | N | N | N | N | N | N | N | N | N |
| 53 | Y | Y | Y | Y | Y | Y | Y | Y | Y | Y | Y | Y |
| 54 | E | E | E | E | E | E | E | E | E | E | E | E |
| 55 | F | F | F | F | F | F | F | F | F | F | F | F |
| 56 | K | K | K | K | N | R | R | R | R | R | R | R |
| 57 | E | E | E | E | E | F | E | F | E | E | F | E |
| 58 | N | N | N | N | S | N | N | N | N | N | N | N |
| 59 | E | E | E | E | E | D | E | E | E | E | E | E |
| 60 | I | I | I | I | G | G | G | G | G | G | V | V |
| 61 | F | F | F | F | P | T | T | P | P | P | P | P |
| 62 | C | C | C | C | C | C | C | C | C | C | C | C |
| 63 | L | L | L | L | L | L | L | L | L | L | L | L |
| 64 | E | E | E | E | A | T | A | A | A | A | A | E |
| 65 | F | F | F | F | F | F | F | F | F | F | F | F |
| 66 | L | L | L | L | M | M | M | M | M | M | M | L |
| 67 | R | R | R | R | R | R | R | R | R | R | R | R |
| 68 | A | A | A | A | A | A | A | A | A | A | A | A |
| 69 | A | A | A | A | A | T | A | A | A | A | A | A |
| 70 | S | S | S | S | S | S | S | S | S | S | S | S |
| 71 | V | V | V | V | V | V | V | V | V | V | V | V |
| 72 | L | L | L | L | L | L | L | L | L | L | L | L |
| 73 | K | K | K | K | K | K | K | K | K | K | K | K |
| 74 | F | F | F | F | S | C | C | S | F | F | F | F |
| 75 | L | L | L | L | L | L | L | L | L | L | L | L |
| 76 | P | P | P | P | P | P | P | P | P | P | P | P |
| 77 | F | F | F | F | H | F | F | F | F | F | F | F |
| 78 | P | P | P | P | A | A | T | T | T | T | T | P |
| 79 | V | V | V | V | I | I | I | I | I | I | I | I |
| 80 | T | T | V | V | S | V | V | V | V | V | V | V |
| 81 | R | R | R | R | S | S | S | R | R | R | R | R |
| 82 | M | M | M | M | L | M | M | M | M | M | M | M |
| 83 | K | K | K | K | K | K | K | K | K | K | K | K |
| 84 | D | D | D | D | D | D | D | D | D | D | D | D |
| 85 | I | I | I | I | L | T | T | I | I | I | I | I |
| 86 | Q | Q | Q | Q | E | E | E | E | E | E | E | E |
| 87 | G | G | G | G | G | G | G | G | G | G | G | G |
| 88 | L | L | L | L | L | L | L | L | L | L | L | L |
| 89 | P | P | P | P | P | P | P | P | P | P | P | P |
| 90 | C | C | C | C | C | W | W | W | W | W | C | C |
| 91 | M | M | M | M | L | I | L | L | M | M | M | M |
| 92 | G | G | G | G | G | G | G | G | G | G | G | G |
| 93 | D | D | D | D | D | D | D | D | D | D | D | D |
| 94 | R | R | Q | Q | Q | E | Q | Q | Q | Q | Q | Q |
| 95 | V | V | V | V | T | V | V | V | V | V | V | V |
| 96 | R | R | R | R | K | K | K | K | K | K | R | R |
| 97 | D | D | D | D | A | G | G | G | G | G | N | N |
| 98 | V | V | V | V | V | I | I | V | V | V | V | V |
| 99 | I | I | I | I | I | M | I | I | I | I | I | I |
| 100 | E | E | E | E | E | E | E | E | E | E | E | E |
| 101 | E | E | E | E | D | E | E | E | E | E | E | E |
| 102 | I | I | I | I | I | I | I | I | I | I | I | I |
| 103 | I | I | I | I | L | I | I | I | I | I | I | I |
| 104 | E | E | E | E | E | E | E | E | E | E | E | E |
| 105 | E | E | E | E | C | D | D | D | E | E | E | E |
| 106 | G | G | G | G | G | G | G | G | G | G | G | G |
| 107 | E | E | E | E | Q | E | E | E | E | F | E | E |
| 108 | S | S | S | S | S | S | S | S | S | S | S | S |
| 109 | S | S | S | S | S | L | S | S | S | S | S | S |
| 110 | R | R | R | R | K | E | E | K | R | R | R | R |
| 111 | A | A | A | A | V | V | V | V | V | V | A | A |
| 112 | K | K | K | K | Q | Q | K | K | K | K | K | K |
| 113 | E | E | D | D | D | A | A | D | D | D | D | D |
| 114 | V | V | V | V | V | V | V | V | V | V | V | V |
| 115 | L | L | L | L | L | L | L | L | L | L | L | L |
| 116 | N | N | N | N | C | N | N | N | N | N | N | N |
| 117 | D | D | D | D | D | D | D | D | D | D | D | D |
| 118 | E | E | F | E | E | E | E | F | E | F | E | F |
| 119 | R | R | R | R | R | R | R | R | R | R | R | R |
| 120 | Y | Y | Y | Y | Y | Y | Y | Y | Y | Y | Y | Y |
| 121 | K | K | K | K | K | Q | Q | K | K | K | K | K |
| 122 | S | S | S | S | A | S | S | S | S | S | S | S |
| 123 | F | F | F | F | F | F | F | F | F | F | F | F |
| 124 | K | K | K | K | K | K | K | K | K | K | K | K |
| 125 | Q | Q | Q | Q | L | L | L | L | L | L | L | L |
| 126 | F | F | F | F | F | F | F | F | F | F | F | F |
| 127 | T | T | T | T | T | T | T | T | T | T | T | T |
| 128 | S | S | S | S | S | S | S | S | S | S | S | S |
| 129 | V | V | V | V | V | V | V | V | V | V | V | V |
| 130 | F | F | F | F | F | F | F | F | F | F | F | F |
| 131 | G | G | G | G | G | G | G | G | G | G | G | G |
| 132 | V | V | V | V | V | V | V | V | V | V | V | V |
| 133 | G | G | G | G | G | G | G | G | G | G | G | G |
| 134 | V | V | V | V | L | L | L | L | L | L | L | L |
| 135 | K | K | K | K | K | K | K | K | K | K | K | K |
| 136 | T | T | T | T | T | T | T | T | T | T | T | T |
| 137 | S | S | S | S | A | A | S | S | S | S | S | S |
| 138 | E | E | E | E | E | E | E | E | E | E | E | E |
| 139 | K | K | K | K | K | K | K | K | K | K | K | K |
| 140 | W | W | W | W | W | W | W | W | W | W | W | W |
| 141 | Y | Y | Y | Y | Y | Y | Y | Y | Y | Y | Y | Y |
| 142 | R | R | R | R | R | R | R | R | R | R | R | R |
| 143 | M | M | M | M | R | M | M | M | M | M | M | M |
| 144 | G | G | G | G | G | G | G | G | G | G | G | G |
| 145 | L | L | L | L | F | F | F | F | F | F | F | F |
| 146 | R | R | R | R | R | R | R | R | R | R | R | R |
| 147 | T | T | T | T | S | T | T | T | T | T | T | T |
| 148 | L | L | L | L | L | L | L | L | L | L | L | L |
| 149 | E | E | E | E | E | S | S | E | E | E | E | E |
| 150 | E | E | E | E | E | K | K | E | E | E | E | E |
| 151 | V | V | V | V | V | I | I | I | V | V | V | V |
| 152 | K | K | K | K | K | R | R | K | K | K | K | K |
| 153 | A | A | A | A | A | S | S | A | A | A | A | A |
| 154 | D | D | D | D | D | D | D | D | D | D | D | D |
| 155 | K | K | K | K | N | K | K | K | K | K | K | K |
| 156 | T | T | T | T | S | S | S | S | S | S | T | T |
| 157 | L | L | L | L | I | L | L | L | L | L | L | L |
| 158 | K | K | K | K | H | K | K | K | K | K | K | K |
| 159 | L | L | L | L | F | L | L | L | L | L | L | L |
| 160 | S | S | S | S | T | T | T | T | T | T | S | S |
| 161 | K | K | K | K | K | K | K | K | R | R | R | R |
| 162 | M | M | M | M | M | M | M | M | M | M | M | M |
| 163 | Q | Q | Q | Q | Q | Q | Q | Q | Q | Q | Q | Q |
| 164 | K | K | K | K | K | K | K | K | K | K | K | K |
| 165 | A | A | A | A | A | A | A | A | A | A | A | A |
| 166 | G | G | G | G | G | G | G | G | G | G | G | G |
| 167 | F | F | F | F | F | F | F | F | F | F | F | F |
| 168 | L | L | L | L | L | L | L | L | L | L | L | L |
| 169 | Y | Y | Y | Y | Y | Y | Y | Y | Y | Y | Y | Y |
| 170 | Y | Y | Y | Y | Y | Y | Y | Y | Y | Y | Y | Y |
| 171 | E | E | E | E | D | E | E | E | E | E | E | E |
| 172 | D | D | D | D | D | D | D | D | D | D | D | D |
| 173 | L | L | L | L | I | L | L | L | L | L | L | L |
| 174 | V | V | V | V | S | I | I | I | I | I | I | V |
| 175 | S | S | S | S | R | S | S | S | S | S | S | S |
| 176 | C | C | C | C | A | C | C | C | C | C | C | C |
| 177 | V | V | V | V | V | V | V | V | V | V | V | V |
| 178 | S | S | S | S | S | S | S | S | S | S | S | S |
| 179 | K | K | K | K | K | K | K | K | K | K | K | K |
| 180 | A | A | A | A | A | A | A | A | A | A | A | A |
| 181 | E | E | E | E | E | E | E | E | E | E | E | E |
| 182 | A | A | A | A | A | A | A | A | A | A | A | A |
| 183 | D | D | D | D | E | D | D | D | D | D | D | D |
| 184 | A | A | A | A | A | A | A | A | A | A | A | A |
| 185 | V | V | V | V | I | V | V | V | V | V | V | V |
| 186 | S | S | S | S | G | S | S | S | S | S | S | S |
| 187 | L | L | L | L | Q | L | L | L | L | L | L | L |
| 188 | I | I | I | I | I | I | I | I | I | I | I | I |
| 189 | V | V | V | V | V | V | V | V | V | V | V | V |
| 190 | K | K | K | K | E | K | K | K | K | K | K | K |
| 191 | N | N | N | N | E | E | E | D | D | D | D | D |
| 192 | T | T | T | T | T | A | A | T | T | T | T | T |
| 193 | V | V | V | V | V | V | V | V | V | V | V | V |
| 194 | C | C | C | C | H | W | W | W | W | W | W | C |
| 195 | T | T | T | T | L | T | T | T | T | T | T | T |
| 196 | F | F | F | F | I | F | F | F | F | F | F | F |
| 197 | L | L | L | L | V | L | L | L | L | L | L | L |
| 198 | P | P | P | P | P | P | P | P | P | P | P | P |
| 199 | D | D | D | D | D | D | D | D | D | D | D | D |
| 200 | A | A | A | A | A | A | A | A | A | A | A | A |
| 201 | L | L | L | L | I | L | L | L | L | L | L | L |
| 202 | V | V | V | V | V | V | V | V | V | V | V | V |
| 203 | T | T | T | T | T | T | T | T | T | T | T | T |
| 204 | I | I | I | I | L | I | M | L | L | L | I | I |
| 205 | T | T | T | T | T | T | T | T | T | T | T | T |
| 206 | G | G | G | G | G | G | G | G | G | G | G | G |
| 207 | G | G | G | G | G | G | G | G | G | G | ? | G |
| 208 | F | F | F | F | F | F | F | F | F | F | F | F |
| 209 | R | R | R | R | R | R | R | R | R | R | R | R |
| 210 | R | L | R | L | R | R | R | R | R | L | L | L |
| 211 | G | G | G | G | G | G | G | G | G | G | G | G |
| 212 | K | G | K | G | K | K | K | K | K | G | G | G |
| 213 | K | K | K | K | E | E | K | K | K | K | K | K |
| 214 | I | I | I | I | F | F | I | I | I | I | I | I |
| 215 | G | G | G | G | G | G | G | G | G | G | G | G |
| 216 | H | H | H | H | H | H | H | H | H | H | H | H |
| 217 | D | D | D | D | D | D | D | D | D | D | D | D |
| 218 | I | I | I | I | V | V | V | V | V | V | V | V |
| 219 | D | D | D | D | D | D | D | D | D | D | D | D |
| 220 | F | F | F | F | F | F | F | F | F | F | F | F |
| 221 | L | L | L | L | L | L | L | L | L | L | L | L |
| 222 | I | I | I | I | I | I | I | I | I | I | I | I |
| 223 | T | T | T | T | T | T | T | T | T | T | T | T |
| 224 | N | N | N | N | T | S | S | S | S | S | S | N |
| 225 | P | P | P | P | P | P | P | P | P | P | P | P |
| 226 | G | G | G | G | E | G | G | G | G | G | G | G |
| 227 | P | P | P | P | T | A | S | P | P | P | P | P |
| 228 | R | R | R | R | G | E | G | G | R | R | R | R |
| 229 | E | E | E | E | K | K | E | E | E | E | E | E |
| | — | — | — | — | — | E | E | — | — | — | — | — |
| | — | — | — | — | — | Q | E | — | — | — | — | — |
| 230 | D | D | D | D | E | E | E | E | E | E | E | E |
| 231 | D | D | D | D | E | D | E | F | F | E | E | E |
| 232 | E | E | E | E | G | Q | Q | E | E | E | E | E |
| 233 | L | L | L | L | L | L | L | L | L | L | L | L |
| 234 | L | L | L | L | L | L | L | L | L | L | L | L |
| 235 | H | H | H | H | H | Q | H | H | H | H | H | H |
| 236 | K | K | K | K | K | K | K | K | K | K | K | K |
| 237 | V | V | V | V | V | V | V | V | V | V | V | V |
| 238 | V | V | V | V | I | T | I | I | I | I | I | V |
| 239 | D | D | D | D | N | N | N | N | D | D | D | D |
| 240 | L | L | L | L | R | L | L | L | L | L | L | L |
| 241 | W | W | W | W | L | W | W | W | W | W | W | W |
| 242 | K | K | K | K | K | E | E | K | K | K | K | K |
| 243 | K | K | K | K | N | K | K | K | K | K | K | K |
| 244 | Q | Q | Q | Q | Q | Q | Q | Q | Q | Q | Q | Q |
| 245 | G | G | G | G | G | G | G | G | G | G | G | G |
| 246 | L | L | L | L | I | L | L | L | L | L | L | L |
| 247 | L | L | L | L | L | L | L | L | L | L | L | L |
| 248 | L | L | L | L | L | L | L | L | L | L | L | L |
| 249 | Y | Y | Y | Y | Y | Y | Y | Y | Y | Y | Y | Y |
| 250 | C | C | C | C | Y | Y | Y | Y | Y | Y | Y | C |
| 251 | D | D | D | D | D | D | D | D | D | D | D | D |
| 252 | I | I | I | I | I | L | L | L | L | L | L | I |
| 253 | I | I | I | I | V | I | I | I | I | I | I | I |
| 254 | E | E | E | E | E | E | E | E | E | E | E | E |
| 255 | S | S | S | S | S | S | S | S | S | S | S | S |
| 256 | T | T | T | T | T | T | T | T | T | T | T | T |
| 257 | F | F | F | F | F | F | F | F | F | F | F | F |
| 258 | V | V | V | V | D | E | E | E | E | E | E | E |
| 259 | K | K | K | K | K | D | K | K | K | K | K | K |
| 260 | E | E | E | E | T | L | L | T | T | T | A | E |
| 261 | Q | 0 | Q | Q | K | K | K | K | K | K | K | K |
| 262 | L | L | L | L | L | L | L | L | L | L | L | L |
| 263 | P | P | P | P | P | P | P | P | P | P | P | P |
| 264 | S | S | S | S | S | S | S | S | S | S | S | S |
| 265 | R | R | R | R | R | R | R | R | R | R | R | R |
| 266 | K | K | K | K | R | K | K | K | K | K | K | K |
| 267 | V | V | V | V | F | V | V | V | V | V | V | V |
| 268 | D | D | D | D | E | D | D | D | D | D | D | D |
| 269 | A | A | A | A | A | A | A | A | A | A | A | A |
| 270 | M | M | M | M | M | L | L | L | L | L | L | L |
| 271 | D | D | D | D | D | D | D | D | D | D | D | D |
| 272 | H | H | H | H | H | H | H | H | H | H | H | H |
| 273 | F | F | F | F | F | F | F | F | F | F | F | F |
| 274 | Q | Q | Q | Q | Q | Q | Q | Q | Q | Q | Q | Q |
| 275 | K | K | K | K | K | K | K | K | K | K | K | K |
| 276 | C | C | C | C | C | C | C | C | C | C | C | C |
| 277 | F | F | F | F | F | F | F | F | F | F | F | F |
| 278 | A | A | A | A | A | L | L | A | A | A | A | A |
| 279 | I | I | I | I | I | I | I | I | I | I | I | I |
| 280 | L | L | L | L | L | L | L | L | L | L | L | L |
| 281 | K | K | K | K | K | K | K | K | K | K | K | K |
| 282 | L | L | L | L | L | L | L | L | L | L | L | L |
| 283 | Y | Y | Y | Y | N | Y | H | H | H | H | H | H |
| 284 | Q | Q | Q | Q | K | H | H | K | K | K | Q | Q |
| 285 | P | P | P | P | E | Q | Q | Q | Q | Q | Q | Q |
| 286 | R | R | R | R | L | R | R | R | R | R | R | R |
| 287 | V | V | V | V | V | G | V | V | V | V | V | V |
| 288 | D | D | D | D | E | D | D | D | D | D | D | D |
| 289 | N | N | N | N | G | K | N | N | N | N | N | N |
| 290 | S | S | S | S | — | S | S | S | S | S | S | S |
| 291 | S | S | S | S | — | K | R | S | S | S | S | S |
| 292 | Y | Y | Y | Y | — | W | Y | S | S | S | S | S |
| 293 | N | N | N | N | D | E | E | D | D | D | D | N |
| 294 | T | T | T | T | G | G | G | G | T | T | T | T |
| 295 | S | S | S | S | Q | P | P | P | S | S | S | S |
| 296 | K | K | K | K | E | E | E | E | E | E | K | K |
| 297 | K | K | E | E | Q | G | E | E | E | E | E | E |
| 298 | F | F | F | F | S | S | S | S | S | S | S | S |
| 299 | D | D | D | D | D | N | N | D | D | D | D | D |
| 300 | M | M | M | M | E | E | E | E | E | E | K | K |
| 301 | A | A | A | A | R | L | Q | Q | Q | Q | A | A |
| 302 | E | E | E | E | A | E | E | E | E | E | E | E |
| 303 | V | V | V | V | S | A | A | A | A | A | V | V |
| 304 | K | K | K | K | K | K | K | K | K | K | K | K |
| 305 | D | D | D | D | D | N | N | N | D | D | D | D |
| 306 | W | W | W | W | W | W | W | W | W | W | W | W |
| 307 | K | K | K | K | K | K | K | K | K | K | K | K |
| 308 | A | A | A | A | A | A | A | A | A | A | A | A |
| 309 | I | I | I | I | I | I | I | I | I | I | I | I |
| 310 | R | R | R | R | R | R | R | R | R | R | R | R |
| 311 | V | V | V | V | V | V | V | V | V | V | V | V |
| 312 | D | D | D | D | D | D | D | D | D | D | D | D |
| 313 | L | L | L | L | L | L | L | L | L | L | L | L |
| 314 | V | V | V | V | V | V | V | V | V | V | V | V |
| 315 | I | I | I | I | V | V | M | I | I | I | I | I |
| 316 | T | T | S | S | P | C | C | S | S | S | S | S |
| 317 | P | P | P | P | P | P | P | P | P | P | P | P |
| 318 | F | F | F | F | V | Y | Y | F | F | F | F | F |
| 319 | E | E | E | E | D | D | E | E | E | E | E | E |
| 320 | Q | Q | Q | Q | N | R | R | Q | Q | Q | Q | Q |
| 321 | Y | Y | Y | Y | F | Y | Y | Y | Y | Y | Y | Y |
| 322 | A | A | A | A | A | A | A | A | A | A | A | A |
| 323 | Y | Y | Y | Y | F | Y | Y | Y | Y | Y | Y | Y |
| 324 | A | A | A | A | A | A | A | A | A | A | A | A |
| 325 | L | L | L | L | L | L | L | L | L | L | L | L |
| 326 | L | L | L | L | L | L | L | L | L | L | L | L |
| 327 | G | G | G | G | G | G | G | G | G | G | G | G |
| 328 | W | W | W | W | W | W | W | W | W | W | W | W |
| 329 | T | T | T | T | T | S | T | T | T | T | T | T |
| 330 | G | G | G | G | G | G | G | G | G | G | G | G |
| 331 | S | S | S | S | S | S | S | S | S | S | S | S |
| 332 | R | R | R | R | R | R | R | R | R | R | R | R |
| 333 | Q | Q | Q | Q | Q | Q | Q | Q | Q | Q | Q | Q |
| 334 | F | F | F | F | F | F | F | F | F | F | F | F |
| 335 | G | G | G | G | E | E | E | E | E | E | E | E |
| 336 | R | R | R | R | R | R | R | R | R | R | R | R |
| 337 | D | D | D | D | D | D | D | D | D | D | D | D |
| 338 | L | L | L | L | L | L | L | L | L | L | L | L |
| 339 | R | R | R | R | R | R | R | R | R | R | R | R |
| 340 | R | R | R | R | R | R | R | R | R | R | R | R |
| 341 | Y | Y | Y | Y | Y | Y | Y | Y | Y | Y | Y | Y |
| 342 | A | A | A | A | A | A | A | A | A | A | A | A |
| 343 | T | T | T | T | H | T | T | T | T | T | T | T |
| 344 | H | H | H | H | H | H | H | H | H | H | H | H |
| 345 | E | E | F | E | E | F | E | E | E | E | E | E |
| 346 | R | R | R | R | K | K | K | K | K | K | R | R |
| 347 | K | K | K | K | K | K | K | K | K | K | K | K |
| 348 | M | M | M | M | M | M | M | M | M | M | M | M |
| 349 | I | I | M | M | L | M | M | M | M | M | M | M |
| 350 | L | L | L | L | L | L | L | L | L | L | L | L |
| 351 | D | D | D | D | D | D | D | D | D | D | D | D |
| 352 | N | N | N | N | N | N | N | N | N | N | N | N |
| 353 | H | H | H | H | H | H | H | H | H | H | H | H |
| 354 | A | A | A | A | A | A | A | A | A | A | A | A |
| 355 | L | L | I | L | L | L | L | L | L | L | L | L |
| 356 | Y | Y | Y | Y | Y | Y | Y | Y | Y | Y | Y | Y |
| 357 | D | D | D | D | D | D | D | D | D | D | D | D |
| 358 | R | R | K | K | K | K | K | K | K | K | K | K |
| 359 | R | R | R | R | T | T | T | T | T | T | T | T |
| 360 | K | K | K | K | K | K | K | K | K | K | K | K |
| 361 | R | R | R | R | K | R | R | R | R | R | R | R |
| 362 | I | I | I | I | V | I | I | I | I | I | I | I |
| 363 | F | F | F | F | F | F | F | F | F | F | F | F |
| 364 | L | L | L | L | L | L | L | L | L | L | L | L |
| 365 | K | K | K | K | K | K | K | K | K | K | K | K |
| 366 | A | A | A | A | A | A | A | A | A | A | A | A |
| 367 | G | G | G | G | K | E | E | E | E | E | E | E |
| 368 | S | S | S | S | T | S | S | S | S | S | S | S |
| 369 | E | E | E | E | E | E | E | E | E | E | E | E |
| 370 | E | E | E | F | E | E | E | E | E | E | E | E |
| 371 | E | E | E | E | D | E | E | E | E | E | E | F |
| 372 | I | I | I | I | I | I | I | I | I | I | I | I |
| 373 | F | F | F | F | F | F | F | F | F | F | F | F |
| 374 | A | A | A | A | A | A | A | A | A | A | A | A |
| 375 | H | H | H | H | H | H | H | H | H | H | H | H |
| 376 | L | L | L | L | L | L | L | L | L | L | L | L |
| 377 | G | G | G | G | G | G | G | G | G | G | G | G |
| 378 | L | L | L | L | L | L | L | L | L | L | L | L |
| 379 | D | D | D | D | E | E | D | D | D | D | D | D |
| 380 | Y | Y | Y | Y | Y | Y | Y | Y | Y | Y | Y | Y |
| 381 | V | V | V | V | I | I | I | I | I | I | I | I |
| 382 | E | F | E | E | E | E | E | E | E | E | E | E |
| 383 | P | P | P | P | P | P | P | P | P | P | P | P |
| 384 | W | W | W | W | W | W | W | W | W | W | W | W |
| 385 | E | E | E | E | Q | E | E | E | E | E | E | E |
| 386 | R | R | R | R | R | R | R | R | R | R | R | R |
| 387 | N | N | N | N | N | N | N | N | N | N | N | N |
| 388 | A | A | A | A | A | A | A | A | A | A | A | A |

An MBP fusion tag was added to each TdT peptide designed for experimental purposes. The amino acid sequence of each TdT peptide of SEQ ID NO: 1 to 3 and 16 to 24 is shown with the MPB tag in SEQ ID NO: 25 to 36, respectively. Therefore, the equivalence of sequences is as following:
SEQ ID NO: 25 is MBP+SEQ ID NO: 3;
SEQ ID NO: 29 is MBP+SEQ ID NO: 1;
SEQ ID NO: 27 is MBP+SEQ ID NO: 2;
SEQ ID NO: 26 is MBP+SEQ ID NO: 16;
SEQ ID NO: 28 is MBP+SEQ ID NO: 17;
SEQ ID NO: 30 is MBP+SEQ ID NO: 18;
SEQ ID NO: 31 is MBP+SEQ ID NO: 19;
SEQ ID NO: 32 is MBP+SEQ ID NO: 20; SEQ ID NO: 33 is MBP+SEQ ID NO: 21;
SEQ ID NO: 34 is MBP+SEQ ID NO: 22; SEQ ID NO: 35 is MBP+SEQ ID NO: 23;
SEQ ID NO: 36 is MBP+SEQ ID NO: 24.

It is important to note that MBP sequence does not affect the activity of the enzymes. Thus, the results of the following experiments carried out with the enzymes having the sequences as shown in SEQ ID NO: 25 to 36 are equivalent to the enzymes having the sequences as shown in SEQ ID NO: 1 to 3 and 16 to 24.

### Expression Strain Generation

The nucleotide sequences of the TdT peptides having an MBP fusion tag (amino acid sequence of SEQ ID No: 25 to 36) were ordered at Twist Bioscience cloned in pET28a(+) plasmid. The plasmids were used to transform E. coli BL21 Gold (DE3) (Novagen). Growing colonies on plates with kanamycin are isolated and named Ec-CTdT.

### Expression

The Ec-CTdT strains have been used for inoculating 50 mL falcon tubes with 6 mL of LB media supplemented with appropriate amounts of kanamycin. After overnight growth at 37° C., appropriate volumes of these pre-cultures have been used to inoculate 1 L Erlenmeyer flasks with 250 L TB (Terrific Broth) media with kanamycin. The flasks were put at 37° C. under strong agitation and the optical density (OD) of the different cultures were regularly checked. After reaching an OD comprised between 0.4 and 0.6, the temperature was decreased to 15° C. After 30 minutes of incubation at 15° C. each flask was supplemented by the addition of IPTG (Isopropyl β-D-1-thiogalactopyranoside, Sigma-Aldrich) to a final concentration of 0.2 mM. The flasks were put back to agitation under a controlled temperature of 15° C. After overnight expression, the cells were harvested in several pellets. Pellets expressing the same peptides were pooled and stored at −20° C., eventually for several months.

### Extraction

Pellets were resuspended in lysis buffer composed of 100 mM tris-HCL (Sigma-Aldrich) pH 8, 50 mM NaCl (Sigma-Aldrich), 5 mM imidazole (Sigma-Aldrich), MgCl₂ to 2.5 mM final concentration and CaCl₂) to 0.1 mM final concentration. Pellets were resuspended by vigorous mechanical agitation and cells were lysed by sonication of samples in short bursts of 30 s 40% amplitude, followed by intervals of 30 s for cooling for 5 minutes in total. Samples were then incubated at 37° C. during 30 min and then debris were removed by centrifugation at 4° C. for 30 min at 10000 rpm. The centrifugate is passed through a 0.2 μm filter to remove any debris before column purification.

### Purification

A one-step affinity procedure was used to purify the produced and extracted TdT peptides enzymes. A Ni-NTA affinity column (GE Healthcare) is used to bind the enzymes. Initially, the column was washed and equilibrated with 15 column volumes of 100 mM tris-HCL (Sigma-Aldrich) pH 8, 50 mM NaCl (Sigma-Aldrich), 5 mM imidazole (Sigma-Aldrich). The enzymes were bound to the column after equilibration. Then a washing buffer, composed of 100 mM tris-HCL (Sigma-Aldrich) pH 8, 50 mM NaCl (Sigma-Aldrich), 5 mM imidazole (Sigma-Aldrich), was applied to the column for 15 column volumes. After wash, the enzymes were eluted with 100 mM tris-HCL (Sigma-Aldrich) pH 8, 50 mM NaCl (Sigma-Aldrich), 500 mM imidazole (Sigma-Aldrich). Fractions corresponding to the highest concentration of TdT peptides were collected and pooled in a single sample. The pooled fractions were loaded concentration filters (Amicon Ultra-30, Merck Millipore) and the buffer was exchanged by sequential rounds of centrifugation and addition of storage buffer 2×(Tris HCl 100 mM pH 7.5, NaCl 100 mM, β-mercaptoethanol 2 mM). Concentrated enzymes were distributed in small aliquots, 50% glycerol final was added, and those aliquots were then frozen at −20 C and stored for a long term. 5 μL of various fraction of the purified enzymes were analyzed in SDS-PAGE gels.

### Example 4: Evaluation of the Activity of TdT Peptides with Natural Deoxyribonucleotides (dNTPs)

### Activity Test

Activity of enzymes generated, expressed, and purified according to example 1 (SEQ ID NO: 25 to 36) was evaluated through the following assay (Table 2). The activity of the TdT peptides was compared with a commercial bovine TdT (bTdT).

TABLE 2
Activity test of TdT peptides with natural
deoxyribonucleotides (dNTPs)

| | Stock | | Final | | To add | |
|---|---|---|---|---|---|---|
| Reagent | Concentration | | Concentration | | 1 reaction | |
| dH₂O above MM | | | | | 16.02 | μL |
| Buffer | 10x | | 1x | | 2 | μL |
| CoCl₂ (mM) | 100 | mM | 2 | mM | 0.4 | μL |
| Fluorescent primer DNA | 100 | μM | 2 | μM | 0.4 | μL |
| dNTPs | 100 | μM | 4 | μM | 0.8 | μL |
| Pyrophosphatase (U/mL) | 100 | (U/mL) | 0.5 | (U/mL) | 0.1 | μL |
| TdT (μM) | 10 | μM | 0.05 | μM | 1 | μL |

The 10× buffer comprises Tris-Acetate 250 mM, K-acetate 2 M, pH 7.6. The dNTPs used were dATP, dGTP, dCTP, and dTTP. Reactions were incubated at 37° C. for 1 min and quenched by heating at 95° C. for 5 minutes. The fluorescent (FAM or Fluorescein amidites) primer DNA was 5′-TAATACGACTCACTTG-3′ (SEQ ID NO:59).

### Analysis

The analysis involved polyacrylamide gel. Samples from activity tests were analyzed through a 7 M urea, polyacrylamide 20% gel. Gels were made just before the analysis by pouring polyacrylamide inside glass plates and letting it polymerize. The gel inside the glass plates was mounted on an adapted tank filled with TBE buffer for the electrophoresis step. The samples analyzed were loaded on the top of the gel. A tension of 300 V was applied between the top and bottom of the gel for 1 h at room temperature. Once migrated according to the sample target size, the system was dismounted, and gel fluorescence was scanned through with a gel documentation imaging system (Clinx Science Instruments). After image acquisition, GelAnalyzer software was used to analyze the number of turnovers of the deoxyribonucleotides.

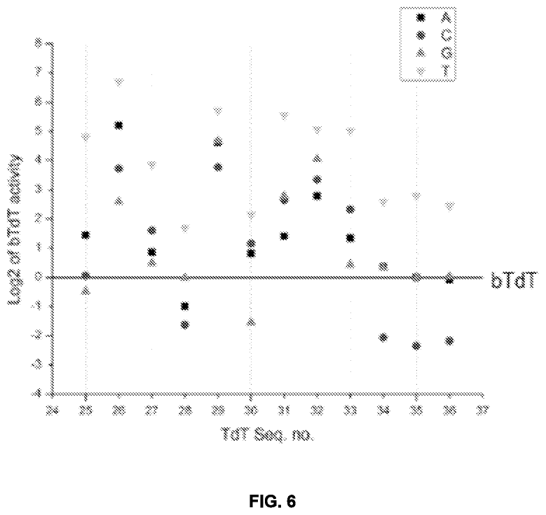

The results are shown on FIG. 6. The x-axis shows the sequence number of each TdT peptide, and the y-axis shows the number of turnovers for each deoxynucleotide tested. Results are shown in Log2 of bTdT activity. Most TdT peptides were better incorporating natural dNTPs than bTdT. Furthermore, every enzyme has a different activity regarding each nucleotide, which can be advantageous in certain applications, especially when the enzyme has good activity towards one specific nucleotide.

### Example 5: Evaluation of the Activity of TdT Peptides with Natural Ribonucleotides (NTPs)

### Activity Test

Activity of enzymes generated, expressed and purified according to example 1 (SEQ ID NO: 25 to 36) was evaluated through the following assay (Table 3). The activity of the TdT peptides was compared with a commercial (New England Biolabs) bovine TdT (bTdT).

TABLE 3
Activity test of TdT peptides with natural ribonucleotides (NTPs)

| | Stock | | Final | | To add | |
|---|---|---|---|---|---|---|
| Reagent | Concentration | | Concentration | | 1 reaction | |
| dH₂O above MM | | | | | 9.1 | μL |
| Buffer | 10x | | 1x | | 2 | μL |
| CoCl₂ (mM) | 100 | mM | 2 | mM | 0.4 | μL |
| Fluorescent primer DNA | 100 | μM | 2 | μM | 0.4 | μL |
| NTPs | 100 | μM | 4 | μM | 4 | μL |
| Pyrophosphatase (U/mL) | 100 | (U/mL) | 0.5 | (U/mL) | 0.1 | μL |
| TdT (uM) | 10 | uM | 0.2 | uM | 4 | μL |

The 10× buffer comprises Tris-Acetate 250 mM, K-acetate 2 M, pH 7.6. The NTPs used were ATP, GTP, CTP, and UTP. Reactions are incubated at 37° C. for 30 min and quenched by heating at 95° C. for 5 minutes. The fluorescent (FAM or Fluorescein amidites) primer DNA was 5′-TAATACGACTCACTTG-3′ (SEQ ID NO: 59).

### Analysis

The analysis involves polyacrylamide gel. Samples from activity tests were analyzed through a 7M urea, polyacrylamide 20% gel. Gels were made just before the analysis by pouring polyacrylamide inside glass plates and let it polymerize. The gel inside the glass plates was mounted on an adapted tank filled with TBE buffer for the electrophoresis step. The samples analyzed were loaded on the top of the gel. A tension of 300 V was applied between the top and bottom of the gel for 1 h at room temperature. Once migrated according to the sample target size, the system was dismounted, and gel fluorescence was scanned through with a gel documentation imaging system (Clinx Science Instruments). After image acquisition, GelAnalyzer software was used to analyze the number of turnovers of the ribonucleotides.

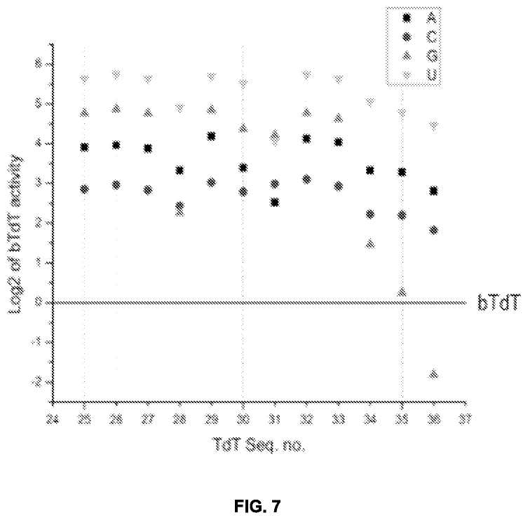

Results are shown on FIG. 7. The x-axis shows the sequence number of TdT peptides, and the y-axis shows the number of turnovers for each ribonucleotide tested. Results are shown in Log2 of bTdT activity. All TdT peptides were surprisingly better incorporating natural ribonucleotides than bTdT, which does not have activity for ribonucleotides.

### Example 6: Evaluation of the Activity of TdT Peptides with 3′-Blocked Nucleotides

### Activity Test

Activity of enzymes generated, expressed and purified according to example 1 (SEQ ID NO: 25 to 36) was evaluated through the following assay (Table 4). The activity of the TdT peptides was compared with a commercial (New England Biolabs) bovine TdT (bTdT).

TABLE 4
Activity test of TdT peptides with blocked
deoxyribonucleotides (NH₂-dNTPs)

| | Stock | | Final | | To add | |
|---|---|---|---|---|---|---|
| Reagent | Concentration | | Concentration | | 1 reaction | |
| dH₂O above MM | | | | | 9.1 | μL |
| Buffer | 10x | | 1x | | 2 | μL |
| CoCl₂ (mM) | 100 | mM | 2 | mM | 0.4 | μL |
| Fluorescent primer DNA | 100 | μM | 2 | μM | 0.4 | μL |
| NH₂-dNTPs | 100 | μM | 4 | μM | 4 | μL |
| Pyrophosphatase (U/mL) | 100 | (U/mL) | 0.5 | (U/mL) | 0.1 | μL |
| TdT (uM) | 10 | uM | 0.2 | uM | 4 | μL |

The 10× buffer comprises Tris-Acetate 250 mM, K-acetate 2 M, pH 7.6. The blocked NH₂-dNTPs used were NH₂-dATP, NH₂-dGTP, NH₂-dCTP, and NH₂-dTTP. Reactions were incubated at 37° C. for 30 min and quenched by heating at 95° C. for 5 minutes. The fluorescent (FAM or Fluorescein amidites) primer DNA was 5′-TAATACGACTCACTTG-3′ (SEQ ID NO: 59). The blocked deoxyribonucleotides were 3′-ONH₂ blocked.

### Analysis

The analysis involves polyacrylamide gel. Samples from activity tests were analyzed through a 7 M urea, polyacrylamide 20% gel. Gels were made just before the analysis by pouring polyacrylamide inside glass plates and let it polymerize. The gel inside the glass plates was mounted on an adapted tank filled with TBE buffer for the electrophoresis step. The samples analyzed were loaded on the top of the gel. A tension of 300 V was applied between the top and bottom of the gel for 1 h at room temperature. Once migrated according to the sample target size, the system was dismounted, and gel fluorescence was scanned through with a gel documentation imaging system (Clinx Science Instruments). After image acquisition, GelAnalyzer software was used to analyze the number of turnovers of the blocked nucleotides.

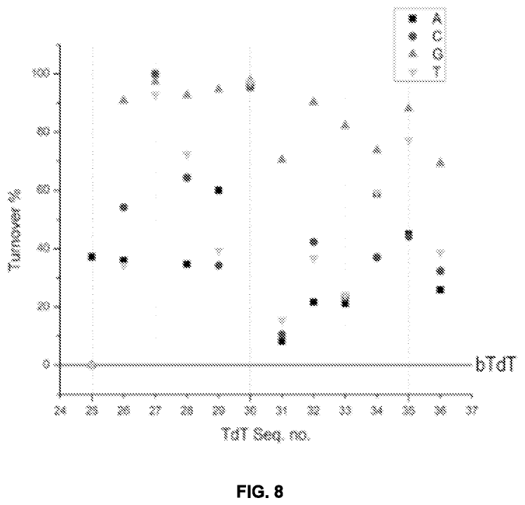

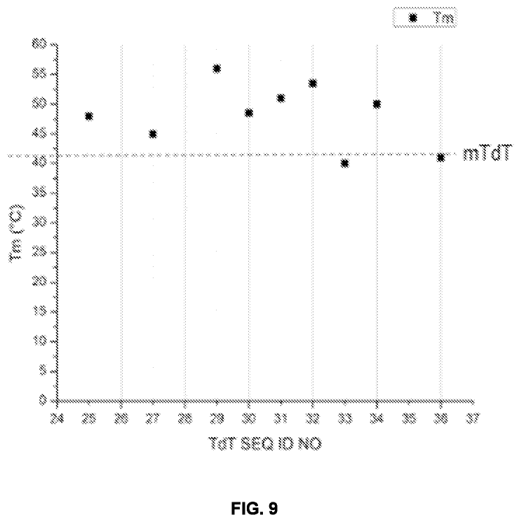

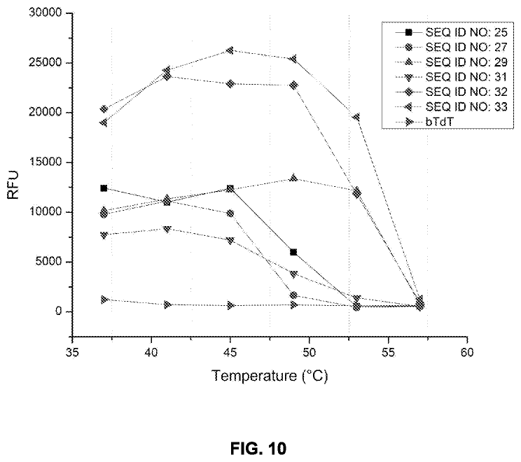

Results are shown on FIG. 8. The x-axis shows the sequence number of TdT peptides and the y-axis shows the number of turnovers for each blocked deoxynucleotide tested. The y-axis in the case of blocked nucleotides is expressed as a percentage of turnover due to the nature of the enzymatic synthesis. As each assay only yields one polymerization reaction, the data is better shown as the proportion of product given (length N) extended by the TdT (length N+1). The results show the activity of every TdT towards each blocked nucleotide. The analysis proves that the TdT peptides generated are capable of incorporating blocked nucleotides in a controlled way and are useful for nucleic acid synthesis applications.

### Example 7: Evaluation of Thermostability TdT Peptides

### Thermostability Test

The thermostability of the TdT peptides was tested by following FRET signal in a qPCR machine. Samples contained 10 μL of 10 μM TdT and 10 μL of SYPRO orange 40× prepared in water. Samples were incubated in the qPCR machine starting at 4° C. and increasing 0.5° C. every 30 seconds until reaching 95° C. FRET signal was measured every 30 seconds and the melting temperature corresponded to the temperature where the minimum of the derivative of the signal was detected.

Results are shown in FIG. 9. The melting temperature (Tm) of almost all TdT peptides are higher than the mTdT, showing desired features of thermostability for laboratory applications.

Table 5 shows a compilation of the results obtained for each TdT peptide. “Yes” means that the enzyme can incorporate at least 3 nucleotides. “n/a” means “not available”.

TABLE 5
Summary of the results obtained for each TdT peptide.

| SEQ ID NO: | RNA | DNA | Blocked DNA | Tm (°C) |
|---|---|---|---|---|
| 1/25 | Yes | No | No | 48 |
| 2/29 | Yes | Yes | Yes | n/a |
| 3/27 | Yes | Yes | Yes | 45 |
| 16/26 | Yes | No | Yes | n/a |
| 17/28 | Yes | Yes | Yes | 56 |
| 18/30 | Yes | Yes | Yes | 48.5 |
| 19/31 | Yes | Yes | Yes | 51 |
| 20/32 | Yes | Yes | Yes | 53.5 |
| 21/33 | Yes | Yes | Yes | 40 |
| 22/34 | Yes | Yes | Yes | 50 |
| 23/35 | Yes | No | Yes | n/a |
| 24/36 | Yes | No | Yes | 41 |

INFORMAL SEQUENCE LISTING

| SEQ ID | |
|---|---|
| NO: | Sequence |
| 1. | MDTSSPSPERSAPTPASPVATVSQYACQRRTTLENHNKIF |
| | TDAFEILAENYEFNESEGPCLAFMRAASVLKSLPHAISSL |
| | KDLEGLPCLGDQTKAVIEDILECGQSSKVQDVLCDERYKA |
| | FKLFTSVFGVGLKTAEKWYRRGFRSLEEVKADNSIHFTKM |
| | QKAGFLYYDDISRAVSKAEAEAIGQIVEETVHLIVPDAIV |
| | TLTGGFRRGKEFGHDVDFLITTPETGKEEGLLHKVINRLK |
| | NQGILLYYDIVESTFDKTKLPSRRFEAMDHFQKCFAILKL |
| | NKELVEGDGQEQSDERASKDWKAIRVDLVVPPVDNFAFAL |
| | LGWTGSRQFERDLRRYAHHEKKMLLDNHALYDKTKKVFLK |
| | AKTEEDIFAHLGLEYIEPWQRNA* |
| 2. | MEQQDQSPTLNTPESEVPSFTASKVSQYSCQRKTTLNNYN |
| | KKFTDAFEIMAENYEFKENEIFCLEFLRAASVLKFLPFPV |
| | VRMKDIQGLPCMGDQVRDVIEEIIEEGESSRAKDVLNDER |
| | YKSFKQFTSVFGVGVKTSEKWYRMGLRTLEEVKADKTLKL |
| | SKMQKAGFLYYEDLVSCVSKAEADAVSLIVKNTVCTFLPD |
| | ALVTITGGFRRGKKIGHDIDFLITNPGPREDDELLHKVVD |
| | LWKKQGLLLYCDIIESTFVKEQLPSRKVDAMDHFQKCFAI |
| | LKLYQPRVDNSSYNTSKEFDMAEVKDWKAIRVDLVISPFE |
| | QYAYALLGWTGSRQFGRDLRRYATHERKMMLDNHALYDKR |
| | KRIFLKAGSEEEIFAHLGLDYVEPWERNA* |
| 3. | MEQDQSPPLNTPESEVPSFIASKVSQYSCQRKTTLNNYNK |
| | KFTDAFEIMAENYEFKENEIFCLEFLRAASVLKFLPFPVT |
| | RMKDIQGLPCMGDRVRDVIEEIIEEGESSRAKEVLNDERY |
| | KSFKQFTSVFGVGVKTSEKWYRMGLRTLEEVKADKTLKLS |
| | KMQKAGFLYYEDLVSCVSKAEADAVSLIVKNTVCTFLPDA |
| | LVTITGGFRRGKKIGHDIDFLITNPGPREDDELLHKVVDL |
| | WKKQGLLLYCDIIESTFVKEQLPSRKVDAMDHFQKCFAIL |
| | KLYQPRVDNSSYNTSKKFDMAEVKDWKAIRVDLVITPFEQ |
| | YAYALLGWTGSRQFGRDLRRYATHERKMILDNHALYDRRK |
| | RIFLKAGSEEEIFAHLGLDYVEPWERNA* |
| 4. | SVFGVGLKTAEKWYRRGFRSLEEVKADNSIHFTKM |
| 5. | SVFGVGVKTSEKWYRMGLRTLEEVKADKTLKLSKM |
| 6. | SVFGVGVKTSEKWYRMGLRTLEEVKADKTLKLSKM |
| 7. | EAEAIGQIVEETVHLIVPDAIVTLTGGFRRGKEFGHDVDF |
| 8. | EADAVSLIVKNTVCTFLPDALVTITGGFRRGKKIGHDIDF |
| 9. | EADAVSLIVKNTVCTFLPDALVTITGGFRRGKKIGHDIDF |
| 10. | GILLYYDIVESTFDKTKLPSRRFEAMDHFQKCFAI |
| 11. | GLLLYCDIIESTFVKEQLPSRKVDAMDHFQKCFAI |
| 12. | GLLLYCDIIESTFVKEQLPSRKVDAMDHFQKCFAI |
| 13. | RVDLVVPPVDNFAFALLGWTGSRQFERDLRRYAHH |
| 14. | RVDLVISPFEQYAYALLGWTGSRQFGRDLRRYATH |
| 15. | RVDLVITPFEQYAYALLGWTGSRQFGRDLRRYATH |
| 16. | MEQDQSPPLNTPESEVPSFIASKVSQYSCQRKTTLNNYNK |
| | KFTDAFEIMAENYEFKENEIFCLEFLRAASVLKFLPFPVT |
| | RMKDIQGLPCMGDRVRDVIEEIIEEGESSRAKEVLNDERY |
| | KSFKQFTSVFGVGVKTSEKWYRMGLRTLEEVKADKTLKLS |
| | KMQKAGFLYYEDLVSCVSKAEADAVSLIVKNTVCTFLPDA |
| | LVTITGGFRLGGKIGHDIDFLITNPGPREDDELLHKVVDL |
| | WKKQGLLLYCDIIESTFVKEQLPSRKVDAMDHFQKCFAIL |
| | KLYQPRVDNSSYNTSKKFDMAEVKDWKAIRVDLVITPFEQ |
| | YAYALLGWTGSRQFGRDLRRYATHERKMILDNHALYDRRK |
| | RIFLKAGSEEEIFAHLGLDYVEPWERNA |
| 17. | MEQQDQSPTLNTPESEVPSFTASKVSQYSCQRKTTLNNYN |
| | KKFTDAFEIMAENYEFKENEIFCLEFLRAASVLKFLPFPV |
| | VRMKDIQGLPCMGDQVRDVIEEIIEEGESSRAKDVLNDER |
| | YKSFKQFTSVFGVGVKTSEKWYRMGLRTLEEVKADKTLKL |
| | SKMQKAGFLYYEDLVSCVSKAEADAVSLIVKNTVCTFLPD |
| | ALVTITGGFRLGGKIGHDIDFLITNPGPREDDELLHKVVD |
| | LWKKQGLLLYCDIIESTFVKEQLPSRKVDAMDHFQKCFAI |
| | LKLYQPRVDNSSYNTSKEFDMAEVKDWKAIRVDLVISPFE |
| | QYAYALLGWTGSRQFGRDLRRYATHERKMMLDNHALYDKR |
| | KRIFLKAGSEEEIFAHLGLDYVEPWERNA |
| 18. | MESRVASANPDPNAGMLKIQSPATKTISQYACQRRTTLNN |
| | HNQRFTDAFEILAKNYEFRENDGTCLTFMRATSVLKCLPF |
| | AIVSMKDTEGLPWIGDEVKGIMEEIIEDGESLEVQAVLND |
| | ERYQSFKLFTSVFGVGLKTAEKWYRMGFRTLSKIRSDKSL |
| | KLTKMQKAGFLYYEDLISCVSKAEADAVSLIVKEAVWTFL |
| | PDALVTITGGFRRGKEFGHDVDFLITSPGAEKEQEDQLLQ |
| | KVTNLWEKQGLLLYYDLIESTFEDLKLPSRKVDALDHFQK |
| | CFLILKLYHQRGDKSKWEGPEGSNELEAKNWKAIRVDLVV |
| | CPYDRYAYALLGWSGSRQFERDLRRYATHEKKMMLDNHAL |
| | YDKTKRIFLKAESEEEIFAHLGLEYIEPWERNA |
| 19. | MVRRDCSASPNPSTRMTKTPSLATKKISQYACQRRTTLNN |
| | CNQKFTDAFEILAENYEFRENEGTCLAFMRAASVLKCLPF |
| | TIVSMKDTEGLPWLGDQVKGIIEEIIEDGESSEVKAVLND |
| | ERYQSFKLFTSVFGVGLKTSEKWYRMGFRTLSKIRSDKSL |
| | KLTKMQKAGFLYYEDLISCVSKAEADAVSLIVKEAVWTFL |
| | PDALVTMTGGFRRGKKIGHDVDFLITSPGSGEEEEEQLLH |
| | KVINLWEKQGLLLYYDLIESTFEKLKLPSRKVDALDHFQK |
| | CFLILKLHHQRVDNSRYEGPEESNEQEAKNWKAIRVDLVM |
| | CPYERYAYALLGWTGSRQFERDLRRYATHEKKMMLDNHAL |
| | YDKTKRIFLKAESEEEIFAHLGLDYIEPWERNA |
| 20. | MVQQDCSASSSSSTRPTETPSLAAAKVSQYACQRRTTLNN |
| | CNKKFTDAFEILAENYEFRENEGPCLAFMRAASVLKSLPF |
| | TIVRMKDIEGLPWLGDQVKGVIEEIIEDGESSKVKDVLND |
| | ERYKSFKLFTSVFGVGLKTSEKWYRMGFRTLEEIKADKSL |
| | KLTKMQKAGFLYYEDLISCVSKAEADAVSLIVKDTVWTFL |
| | PDALVTLTGGFRRGKKIGHDVDFLITSPGPGEEEELLHKV |
| | INLWKKQGLLLYYDLIESTFEKTKLPSRKVDALDHFQKCF |
| | AILKLHKQRVDNSSSDGPEESDEQEAKNWKAIRVDLVISP |
| | FEQYAYALLGWTGSRQFERDLRRYATHEKKMMLDNHALYD |
| | KTKRIFLKAESEEEIFAHLGLDYIEPWERNA |
| 21. | MVQQDCSASYQSSSSTRPTETSSLAAAKVSQYACQRRTTL |
| | NNCNKKFTDAFEILAENYEFRENEGPCLAFMRAASVLKFL |
| | PFTIVRMKDIEGLPWMGDQVKGVIEEIIEEGESSRVKDVL |
| | NDERYKSFKLFTSVFGVGLKTSEKWYRMGFRTLEEVKADK |
| | SLKLTRMQKAGFLYYEDLISCVSKAEADAVSLIVKDTVWT |
| | FLPDALVTLTGGFRRGKKIGHDVDFLITSPGPREEEELLH |
| | KVIDLWKKQGLLLYYDLIESTFEKTKLPSRKVDALDHFQK |
| | CFAILKLHKQRVDNSSSDTSEESDEQEAKDWKAIRVDLVI |
| | SPFEQYAYALLGWTGSRQFERDLRRYATHEKKMMLDNHAL |
| | YDKTKRIFLKAESEEEIFAHLGLDYIEPWERNA |
| 22. | MVQQDCSASYQSSSSTRPTETSSLAAAKVSQYACQRRTTL |
| | NNCNKKFTDAFEILAENYEFRENEGPCLAFMRAASVLKFL |
| | PFTIVRMKDIEGLPWMGDQVKGVIEEIIEEGESSRVKDVL |
| | NDERYKSFKLFTSVFGVGLKTSEKWYRMGFRTLEEVKADK |
| | SLKLTRMQKAGFLYYEDLISCVSKAEADAVSLIVKDTVWT |
| | FLPDALVTLTGGFRLGGKIGHDVDFLITSPGPREEEELLH |
| | KVIDLWKKQGLLLYYDLIESTFEKTKLPSRKVDALDHFQK |
| | CFAILKLHKQRVDNSSSDTSEESDEQEAKDWKAIRVDLVI |
| | SPFEQYAYALLGWTGSRQFERDLRRYATHEKKMMLDNHAL |
| | YDKTKRIFLKAESEEEIFAHLGLDYIEPWERNA |
| 23. | MVQQDCSATSDTPESEASSFAAAKVSQYACQRRTTLNNCN |
| | KKFTDAFEILAENYEFRENEVPCLAFMRAASVLKFLPFTI |
| | VRMKDIEGLPCMGDQVRNVIEEIIEEGESSRAKDVLNDER |
| | YKSFKLFTSVFGVGLKTSEKWYRMGFRTLEEVKADKTLKL |
| | SRMQKAGFLYYEDLISCVSKAEADAVSLIVKDTVWTFLPD |
| | ALVTITGGFRLGGKIGHDVDFLITSPGPREEEELLHKVID |
| | LWKKQGLLLYYDLIESTFEKAKLPSRKVDALDHFQKCFAI |
| | LKLHQQRVDNSSSDTSKESDKAEVKDWKAIRVDLVISPFE |
| | QYAYALLGWTGSRQFERDLRRYATHERKMMLDNHALYDKT |
| | KRIFLKAESEEEIFAHLGLDYIEPWERNA |
| 24. | MVQQDCSATSDTPESEASSFAAAKVSQYACQRRTTLNNYN |
| | KKFTDAFEILAENYEFRENEVPCLEFLRAASVLKFLPFPI |
| | VRMKDIEGLPCMGDQVRNVIEEIIEEGESSRAKDVLNDER |
| | YKSFKLFTSVFGVGLKTSEKWYRMGFRTLEEVKADKTLKL |
| | SRMQKAGFLYYEDLVSCVSKAEADAVSLIVKDTVCTFLPD |
| | ALVTITGGFRLGGKIGHDVDFLITNPGPREEEELLHKVVD |
| | LWKKQGLLLYCDIIESTFEKEKLPSRKVDALDHFQKCFAI |
| | LKLHQQRVDNSSSNTSKESDKAEVKDWKAIRVDLVISPFE |
| | QYAYALLGWTGSRQFERDLRRYATHERKMMLDNHALYDKT |
| | KRIFLKAESEEEIFAHLGLDYIEPWERNA |
| 25. | MGHHHHHHHHHHSSGHIDDDDKHMMKIEEGKLVIWINGDK |
| | GYNGLAEVGKKFEKDTGIKVTVEHPDKLEEKFPQVAATGD |
| | GPDIIFWAHDRFGGYAQSGLLAEITPDKAFQDKLYPFTWD |
| | AVRYNGKLIAYPIAVEALSLIYNKDLLPNPPKTWEEIPAL |
| | DKELKAKGKSALMFNLQEPYFTWPLIAADGGYAFKYENGK |
| | YDIKDVGVDNAGAKAGLTFLVDLIKNKHMNADTDYSIAEA |
| | AFNKGETAMTINGPWAWSNIDTSKVNYGVTVLPTFKGQPS |
| | KPFVGVLSAGINAASPNKELAKEFLENYLLTDEGLEAVNK |
| | DKPLGAVALKSYEEELVKDPRIAATMENAQKGEIMPNIPQ |
| | MSAFWYAVRTAVINAASGRQTVDEALKDAQTNSSSNNNNN |
| | NNNNNLGSSGHMEQDQSPPLNTPESEVPSFIASKVSQYSC |
| | QRKTTLNNYNKKFTDAFEIMAENYEFKENEIFCLEFLRAA |
| | SVLKFLPFPVTRMKDIQGLPCMGDRVRDVIEEIIEEGESS |
| | RAKEVLNDERYKSFKQFTSVFGVGVKTSEKWYRMGLRTLE |
| | EVKADKTLKLSKMQKAGFLYYEDLVSCVSKAEADAVSLIV |
| | KNTVCTFLPDALVTITGGFRRGKKIGHDIDFLITNPGPRE |
| | DDELLHKVVDLWKKQGLLLYCDIIESTFVKEQLPSRKVDA |
| | MDHFQKCFAILKLYQPRVDNSSYNTSKKFDMAEVKDWKAI |
| | RVDLVITPFEQYAYALLGWTGSRQFGRDLRRYATHERKMI |
| | LDNHALYDRRKRIFLKAGSEEEIFAHLGLDYVEPWERNA |
| 26. | MGHHHHHHHHHHSSGHIDDDDKHMMKIEEGKLVIWINGDK |
| | GYNGLAEVGKKFEKDTGIKVTVEHPDKLEEKFPQVAATGD |
| | GPDIIFWAHDRFGGYAQSGLLAEITPDKAFQDKLYPFTWD |
| | AVRYNGKLIAYPIAVEALSLIYNKDLLPNPPKTWEEIPAL |
| | DKELKAKGKSALMFNLQEPYFTWPLIAADGGYAFKYENGK |
| | YDIKDVGVDNAGAKAGLTFLVDLIKNKHMNADTDYSIAEA |
| | AFNKGETAMTINGPWAWSNIDTSKVNYGVTVLPTFKGQPS |
| | KPFVGVLSAGINAASPNKELAKEFLENYLLTDEGLEAVNK |
| | DKPLGAVALKSYEEELVKDPRIAATMENAQKGEIMPNIPQ |
| | MSAFWYAVRTAVINAASGRQTVDEALKDAQTNSSSNNNNN |
| | NNNNNLGRDHMVLHEYVNAAGITDGGSGGGSTSHMEQDQS |
| | PPLNTPESEVPSFIASKVSQYSCQRKTTLNNYNKKFTDAF |
| | EIMAENYEFKENEIFCLEFLRAASVLKFLPFPVTRMKDIQ |
| | GLPCMGDRVRDVIEEIIEEGESSRAKEVLNDERYKSFKQF |
| | TSVFGVGVKTSEKWYRMGLRTLEEVKADKTLKLSKMQKAG |
| | FLYYEDLVSCVSKAEADAVSLIVKNTVCTFLPDALVTITG |
| | GFRLGGKIGHDIDFLITNPGPREDDELLHKVVDLWKKQGL |
| | LLYCDIIESTFVKEQLPSRKVDAMDHFQKCFAILKLYQPR |
| | VDNSSYNTSKKFDMAEVKDWKAIRVDLVITPFEQYAYALL |
| | GWTGSRQFGRDLRRYATHERKMILDNHALYDRRKRIFLKA |
| | GSEEEIFAHLGLDYVEPWERNA |
| 27. | MGHHHHHHHHHHSSGHIDDDDKHMMKIEEGKLVIWINGDK |
| | GYNGLAEVGKKFEKDTGIKVTVEHPDKLEEKFPQVAATGD |
| | GPDIIFWAHDRFGGYAQSGLLAEITPDKAFQDKLYPFTWD |
| | AVRYNGKLIAYPIAVEALSLIYNKDLLPNPPKTWEEIPAL |
| | DKELKAKGKSALMFNLQEPYFTWPLIAADGGYAFKYENGK |
| | YDIKDVGVDNAGAKAGLTFLVDLIKNKHMNADTDYSIAEA |
| | AFNKGETAMTINGPWAWSNIDTSKVNYGVTVLPTFKGQPS |
| | KPFVGVLSAGINAASPNKELAKEFLENYLLTDEGLEAVNK |
| | DKPLGAVALKSYEEELVKDPRIAATMENAQKGEIMPNIPQ |
| | MSAFWYAVRTAVINAASGRQTVDEALKDAQTNSSSNNNNN |
| | NNNNNLGSSGHMEQQDQSPTLNTPESEVPSFTASKVSQYS |
| | CQRKTTLNNYNKKFTDAFEIMAENYEFKENEIFCLEFLRA |
| | ASVLKFLPFPVVRMKDIQGLPCMGDQVRDVIEEIIEEGES |
| | SRAKDVLNDERYKSFKQFTSVFGVGVKTSEKWYRMGLRTL |
| | EEVKADKTLKLSKMQKAGFLYYEDLVSCVSKAEADAVSLI |
| | VKNTVCTFLPDALVTITGGFRRGKKIGHDIDFLITNPGPR |
| | EDDELLHKVVDLWKKQGLLLYCDIIESTFVKEQLPSRKVD |
| | AMDHFQKCFAILKLYQPRVDNSSYNTSKEFDMAEVKDWKA |
| | IRVDLVISPFEQYAYALLGWTGSRQFGRDLRRYATHERKM |
| | MLDNHALYDKRKRIFLKAGSEEEIFAHLGLDYVEPWERNA |
| 28. | MGHHHHHHHHHHSSGHIDDDDKHMMKIEEGKLVIWINGDK |
| | GYNGLAEVGKKFEKDTGIKVTVEHPDKLEEKFPQVAATGD |
| | GPDIIFWAHDRFGGYAQSGLLAEITPDKAFQDKLYPFTWD |
| | AVRYNGKLIAYPIAVEALSLIYNKDLLPNPPKTWEEIPAL |
| | DKELKAKGKSALMFNLQEPYFTWPLIAADGGYAFKYENGK |
| | YDIKDVGVDNAGAKAGLTFLVDLIKNKHMNADTDYSIAEA |
| | AFNKGETAMTINGPWAWSNIDTSKVNYGVTVLPTFKGQPS |
| | KPFVGVLSAGINAASPNKELAKEFLENYLLTDEGLEAVNK |
| | DKPLGAVALKSYEEELVKDPRIAATMENAQKGEIMPNIPQ |
| | MSAFWYAVRTAVINAASGRQTVDEALKDAQTNSSSNNNNN |
| | NNNNNLGRDHMVLHEYVNAAGITDGGSGGGSTSHMEQQDQ |
| | SPTLNTPESEVPSFTASKVSQYSCQRKTTLNNYNKKFTDA |
| | FEIMAENYEFKENEIFCLEFLRAASVLKFLPFPVVRMKDI |
| | QGLPCMGDQVRDVIEEIIEEGESSRAKDVLNDERYKSFKQ |
| | FTSVFGVGVKTSEKWYRMGLRTLEEVKADKTLKLSKMQKA |
| | GFLYYEDLVSCVSKAEADAVSLIVKNTVCTFLPDALVTIT |
| | GGFRLGGKIGHDIDFLITNPGPREDDELLHKVVDLWKKQG |
| | LLLYCDIIESTFVKEQLPSRKVDAMDHFQKCFAILKLYQP |
| | RVDNSSYNTSKEFDMAEVKDWKAIRVDLVISPFEQYAYAL |
| | LGWTGSRQFGRDLRRYATHERKMMLDNHALYDKRKRIFLK |
| | AGSEEEIFAHLGLDYVEPWERNA |
| 29. | MGHHHHHHHHHHSSGHIDDDDKHMMKIEEGKLVIWINGDK |
| | GYNGLAEVGKKFEKDTGIKVTVEHPDKLEEKFPQVAATGD |
| | GPDIIFWAHDRFGGYAQSGLLAEITPDKAFQDKLYPFTWD |
| | AVRYNGKLIAYPIAVEALSLIYNKDLLPNPPKTWEEIPAL |
| | DKELKAKGKSALMFNLQEPYFTWPLIAADGGYAFKYENGK |
| | YDIKDVGVDNAGAKAGLTFLVDLIKNKHMNADTDYSIAEA |
| | AFNKGETAMTINGPWAWSNIDTSKVNYGVTVLPTFKGQPS |
| | KPFVGVLSAGINAASPNKELAKEFLENYLLTDEGLEAVNK |
| | DKPLGAVALKSYEEELVKDPRIAATMENAQKGEIMPNIPQ |
| | MSAFWYAVRTAVINAASGRQTVDEALKDAQTNSSSNNNNN |
| | NNNNNLGSSGHMDTSSPSPERSAPTPASPVATVSQYACQR |
| | RTTLENHNKIFTDAFEILAENYEFNESEGPCLAFMRAASV |
| | LKSLPHAISSLKDLEGLPCLGDQTKAVIEDILECGQSSKV |
| | QDVLCDERYKAFKLFTSVFGVGLKTAEKWYRRGFRSLEEV |
| | KADNSIHFTKMQKAGFLYYDDISRAVSKAEAEAIGQIVEE |
| | TVHLIVPDAIVTLTGGFRRGKEFGHDVDFLITTPETGKEE |
| | GLLHKVINRLKNQGILLYYDIVESTFDKTKLPSRRFEAMD |
| | HFQKCFAILKLNKELVEGDGQEQSDERASKDWKAIRVDLV |
| | VPPVDNFAFALLGWTGSRQFERDLRRYAHHEKKMLLDNHA |
| | LYDKTKKVFLKAKTEEDIFAHLGLEYIEPWQRNA |
| 30. | MGHHHHHHHHHHSSGHIDDDDKHMMKIEEGKLVIWINGDK |
| | GYNGLAEVGKKFEKDTGIKVTVEHPDKLEEKFPQVAATGD |
| | GPDIIFWAHDRFGGYAQSGLLAEITPDKAFQDKLYPFTWD |
| | AVRYNGKLIAYPIAVEALSLIYNKDLLPNPPKTWEEIPAL |
| | DKELKAKGKSALMFNLQEPYFTWPLIAADGGYAFKYENGK |
| | YDIKDVGVDNAGAKAGLTFLVDLIKNKHMNADTDYSIAEA |
| | AFNKGETAMTINGPWAWSNIDTSKVNYGVTVLPTFKGQPS |
| | KPFVGVLSAGINAASPNKELAKEFLENYLLTDEGLEAVNK |
| | DKPLGAVALKSYEEELVKDPRIAATMENAQKGEIMPNIPQ |
| | MSAFWYAVRTAVINAASGRQTVDEALKDAQTNSSSNNNNN |
| | NNNNNLGSSGHMESRVASANPDPNAGMLKIQSPATKTISQ |
| | YACQRRTTLNNHNQRFTDAFEILAKNYEFRENDGTCLTFM |
| | RATSVLKCLPFAIVSMKDTEGLPWIGDEVKGIMEEIIEDG |
| | ESLEVQAVLNDERYQSFKLFTSVFGVGLKTAEKWYRMGFR |
| | TLSKIRSDKSLKLTKMQKAGFLYYEDLISCVSKAEADAVS |
| | LIVKEAVWTFLPDALVTITGGFRRGKEFGHDVDFLITSPG |
| | AEKEQEDQLLQKVTNLWEKQGLLLYYDLIESTFEDLKLPS |
| | RKVDALDHFQKCFLILKLYHQRGDKSKWEGPEGSNELEAK |
| | NWKAIRVDLVVCPYDRYAYALLGWSGSRQFERDLRRYATH |
| | EKKMMLDNHALYDKTKRIFLKAESEEEIFAHLGLEYIEPW |
| | ERNA |
| 31. | MGHHHHHHHHHHSSGHIDDDDKHMMKIEEGKLVIWINGDK |
| | GYNGLAEVGKKFEKDTGIKVTVEHPDKLEEKFPQVAATGD |
| | GPDIIFWAHDRFGGYAQSGLLAEITPDKAFQDKLYPFTWD |
| | AVRYNGKLIAYPIAVEALSLIYNKDLLPNPPKTWEEIPAL |
| | DKELKAKGKSALMFNLQEPYFTWPLIAADGGYAFKYENGK |
| | YDIKDVGVDNAGAKAGLTFLVDLIKNKHMNADTDYSIAEA |
| | AFNKGETAMTINGPWAWSNIDTSKVNYGVTVLPTFKGQPS |
| | KPFVGVLSAGINAASPNKELAKEFLENYLLTDEGLEAVNK |
| | DKPLGAVALKSYEEELVKDPRIAATMENAQKGEIMPNIPQ |
| | MSAFWYAVRTAVINAASGRQTVDEALKDAQTNSSSNNNNN |
| | NNNNNLGSSGHMVRRDCSASPNPSTRMTKTPSLATKKISQ |
| | YACQRRTTLNNCNQKFTDAFEILAENYEFRENEGTCLAFM |
| | RAASVLKCLPFTIVSMKDTEGLPWLGDQVKGIIEEIIEDG |
| | ESSEVKAVLNDERYQSFKLFTSVFGVGLKTSEKWYRMGFR |
| | TLSKIRSDKSLKLTKMQKAGFLYYEDLISCVSKAEADAVS |
| | LIVKEAVWTFLPDALVTMTGGFRRGKKIGHDVDFLITSPG |
| | SGEEEEEQLLHKVINLWEKQGLLLYYDLIESTFEKLKLPS |
| | RKVDALDHFQKCFLILKLHHQRVDNSRYEGPEESNEQEAK |
| | NWKAIRVDLVMCPYERYAYALLGWTGSRQFERDLRRYATH |
| | EKKMMLDNHALYDKTKRIFLKAESEEEIFAHLGLDYIEPW |
| | ERNA |
| 32. | MGHHHHHHHHHHSSGHIDDDDKHMMKIEEGKLVIWINGDK |
| | GYNGLAEVGKKFEKDTGIKVTVEHPDKLEEKFPQVAATGD |
| | GPDIIFWAHDRFGGYAQSGLLAEITPDKAFQDKLYPFTWD |
| | AVRYNGKLIAYPIAVEALSLIYNKDLLPNPPKTWEEIPAL |
| | DKELKAKGKSALMFNLQEPYFTWPLIAADGGYAFKYENGK |
| | YDIKDVGVDNAGAKAGLTFLVDLIKNKHMNADTDYSIAEA |
| | AFNKGETAMTINGPWAWSNIDTSKVNYGVTVLPTFKGQPS |
| | KPFVGVLSAGINAASPNKELAKEFLENYLLTDEGLEAVNK |
| | DKPLGAVALKSYEEELVKDPRIAATMENAQKGEIMPNIPQ |
| | MSAFWYAVRTAVINAASGRQTVDEALKDAQTNSSSNNNNN |
| | NNNNNLGSSGHMVQQDCSASSSSSTRPTETPSLAAAKVSQ |
| | YACQRRTTLNNCNKKFTDAFEILAENYEFRENEGPCLAFM |
| | RAASVLKSLPFTIVRMKDIEGLPWLGDQVKGVIEEIIEDG |
| | ESSKVKDVLNDERYKSFKLFTSVFGVGLKTSEKWYRMGFR |
| | TLEEIKADKSLKLTKMQKAGFLYYEDLISCVSKAEADAVS |
| | LIVKDTVWTFLPDALVTLTGGFRRGKKIGHDVDFLITSPG |
| | PGEEEELLHKVINLWKKQGLLLYYDLIESTFEKTKLPSRK |
| | VDALDHFQKCFAILKLHKQRVDNSSSDGPEESDEQEAKNW |
| | KAIRVDLVISPFEQYAYALLGWTGSRQFERDLRRYATHEK |
| | KMMLDNHALYDKTKRIFLKAESEEEIFAHLGLDYIEPWER |
| | NA |
| 33 | MGHHHHHHHHHHSSGHIDDDDKHMMKIEEGKLVIWINGDK |
| | GYNGLAEVGKKFEKDTGIKVTVEHPDKLEEKFPQVAATGD |
| | GPDIIFWAHDRFGGYAQSGLLAEITPDKAFQDKLYPFTWD |
| | AVRYNGKLIAYPIAVEALSLIYNKDLLPNPPKTWEEIPAL |
| | DKELKAKGKSALMFNLQEPYFTWPLIAADGGYAFKYENGK |
| | YDIKDVGVDNAGAKAGLTFLVDLIKNKHMNADTDYSIAEA |
| | AFNKGETAMTINGPWAWSNIDTSKVNYGVTVLPTFKGQPS |
| | KPFVGVLSAGINAASPNKELAKEFLENYLLTDEGLEAVNK |
| | DKPLGAVALKSYEEELVKDPRIAATMENAQKGEIMPNIPQ |
| | MSAFWYAVRTAVINAASGRQTVDEALKDAQTNSSSNNNNN |
| | NNNNNLGSSGHMVQQDCSASYQSSSSTRPTETSSLAAAKV |
| | SQYACQRRTTLNNCNKKFTDAFEILAENYEFRENEGPCLA |
| | FMRAASVLKFLPFTIVRMKDIEGLPWMGDQVKGVIEEIIE |
| | EGESSRVKDVLNDERYKSFKLFTSVFGVGLKTSEKWYRMG |
| | FRTLEEVKADKSLKLTRMQKAGFLYYEDLISCVSKAEADA |
| | VSLIVKDTVWTFLPDALVTLTGGFRRGKKIGHDVDFLITS |
| | PGPREEEELLHKVIDLWKKQGLLLYYDLIESTFEKTKLPS |
| | RKVDALDHFQKCFAILKLHKQRVDNSSSDTSEESDEQEAK |
| | DWKAIRVDLVISPFEQYAYALLGWTGSRQFERDLRRYATH |
| | EKKMMLDNHALYDKTKRIFLKAESEEEIFAHLGLDYIEPW |
| | ERNA |
| 34. | MGHHHHHHHHHHSSGHIDDDDKHMMKIEEGKLVIWINGDK |
| | GYNGLAEVGKKFEKDTGIKVTVEHPDKLEEKFPQVAATGD |
| | GPDIIFWAHDRFGGYAQSGLLAEITPDKAFQDKLYPFTWD |
| | AVRYNGKLIAYPIAVEALSLIYNKDLLPNPPKTWEEIPAL |
| | DKELKAKGKSALMFNLQEPYFTWPLIAADGGYAFKYENGK |
| | YDIKDVGVDNAGAKAGLTFLVDLIKNKHMNADTDYSIAEA |
| | AFNKGETAMTINGPWAWSNIDTSKVNYGVTVLPTFKGQPS |
| | KPFVGVLSAGINAASPNKELAKEFLENYLLTDEGLEAVNK |
| | DKPLGAVALKSYEEELVKDPRIAATMENAQKGEIMPNIPQ |
| | MSAFWYAVRTAVINAASGRQTVDEALKDAQTNSSSNNNNN |
| | NNNNNLGRDHMVLHEYVNAAGITDGGSGGGSTSHMVQQDC |
| | SASYQSSSSTRPTETSSLAAAKVSQYACQRRTTLNNCNKK |
| | FTDAFEILAENYEFRENEGPCLAFMRAASVLKFLPFTIVR |
| | MKDIEGLPWMGDQVKGVIEEIIEEGESSRVKDVLNDERYK |
| | SFKLFTSVFGVGLKTSEKWYRMGFRTLEEVKADKSLKLTR |
| | MQKAGFLYYEDLISCVSKAEADAVSLIVKDTVWTFLPDAL |
| | VTLTGGFRLGGKIGHDVDFLITSPGPREEEELLHKVIDLW |
| | KKQGLLLYYDLIESTFEKTKLPSRKVDALDHFQKCFAILK |
| | LHKQRVDNSSSDTSEESDEQEAKDWKAIRVDLVISPFEQY |
| | AYALLGWTGSRQFERDLRRYATHEKKMMLDNHALYDKTKR |
| | IFLKAESEEEIFAHLGLDYIEPWERNA |
| 35. | MGHHHHHHHHHHSSGHIDDDDKHMMKIEEGKLVIWINGDK |
| | GYNGLAEVGKKFEKDTGIKVTVEHPDKLEEKFPQVAATGD |
| | GPDIIFWAHDRFGGYAQSGLLAEITPDKAFQDKLYPFTWD |
| | AVRYNGKLIAYPIAVEALSLIYNKDLLPNPPKTWEEIPAL |
| | DKELKAKGKSALMFNLQEPYFTWPLIAADGGYAFKYENGK |
| | YDIKDVGVDNAGAKAGLTFLVDLIKNKHMNADTDYSIAEA |
| | AFNKGETAMTINGPWAWSNIDTSKVNYGVTVLPTFKGQPS |
| | KPFVGVLSAGINAASPNKELAKEFLENYLLTDEGLEAVNK |
| | DKPLGAVALKSYEEELVKDPRIAATMENAQKGEIMPNIPQ |
| | MSAFWYAVRTAVINAASGRQTVDEALKDAQTNSSSNNNNN |
| | NNNNNLGRDHMVLHEYVNAAGITDGGSGGGSTSHMVQQDC |
| | SATSDTPESEASSFAAAKVSQYACQRRTTLNNCNKKFTDA |
| | FEILAENYEFRENEVPCLAFMRAASVLKFLPFTIVRMKDI |
| | EGLPCMGDQVRNVIEEIIEEGESSRAKDVLNDERYKSFKL |
| | FTSVFGVGLKTSEKWYRMGFRTLEEVKADKTLKLSRMQKA |
| | GFLYYEDLISCVSKAEADAVSLIVKDTVWTFLPDALVTIT |
| | GGFRLGGKIGHDVDFLITSPGPREEEELLHKVIDLWKKQG |
| | LLLYYDLIESTFEKAKLPSRKVDALDHFQKCFAILKLHQQ |
| | RVDNSSSDTSKESDKAEVKDWKAIRVDLVISPFEQYAYAL |
| | LGWTGSRQFERDLRRYATHERKMMLDNHALYDKTKRIFLK |
| | AESEEEIFAHLGLDYIEPWERNA |
| 36 | MGHHHHHHHHHHSSGHIDDDDKHMMKIEEGKLVIWINGDK |
| | GYNGLAEVGKKFEKDTGIKVTVEHPDKLEEKFPQVAATGD |
| | GPDIIFWAHDRFGGYAQSGLLAEITPDKAFQDKLYPFTWD |
| | AVRYNGKLIAYPIAVEALSLIYNKDLLPNPPKTWEEIPAL |
| | DKELKAKGKSALMFNLQEPYFTWPLIAADGGYAFKYENGK |
| | YDIKDVGVDNAGAKAGLTFLVDLIKNKHMNADTDYSIAEA |
| | AFNKGETAMTINGPWAWSNIDTSKVNYGVTVLPTFKGQPS |
| | KPFVGVLSAGINAASPNKELAKEFLENYLLTDEGLEAVNK |
| | DKPLGAVALKSYEEELVKDPRIAATMENAQKGEIMPNIPQ |
| | MSAFWYAVRTAVINAASGRQTVDEALKDAQTNSSSNNNNN |
| | NNNNNLGRDHMVLHEYVNAAGITDGGSGGGSTSHMVQQDC |
| | SATSDTPESEASSFAAAKVSQYACQRRTTLNNYNKKFTDA |
| | FEILAENYEFRENEVPCLEFLRAASVLKFLPFPIVRMKDI |
| | EGLPCMGDQVRNVIEEIIEEGESSRAKDVLNDERYKSFKL |
| | FTSVFGVGLKTSEKWYRMGFRTLEEVKADKTLKLSRMQKA |
| | GFLYYEDLVSCVSKAEADAVSLIVKDTVCTFLPDALVTIT |
| | GGFRLGGKIGHDVDFLITNPGPREEEELLHKVVDLWKKQG |
| | LLLYCDIIESTFEKEKLPSRKVDALDHFQKCFAILKLHQQ |
| | RVDNSSSNTSKESDKAEVKDWKAIRVDLVISPFEQYAYAL |
| | LGWTGSRQFERDLRRYATHERKMMLDNHALYDKTKRIFLK |
| | AESEEEIFAHLGLDYIEPWERNA |
| 37 | RTDYSATPNPGFQKTPPLAVKKISQYACQRKTTLNNYNHI |
| | FTDAFEILAENSEFKENEVSYVTFMRAASVLKSLPFTIIS |
| | MKDTEGIPCLGDKVKCIIEEIIEDGESSEVKAVLNDERYQ |
| | SFKLFTSVFGVGLKTSEKWFRMGFRSLSKIMSDKTLKFTK |
| | MQKAGFLYYEDLVSCVTRAEAEAVGVLVKEAVWAFLPDAF |
| | VTMTGGFRRGKKIGHDVDFLITSPGSAEDEEQLLPKVINL |
| | WEKKGLLLYYDLVESTFEKFKLPSRQVDTLDHFQKCFLIL |
| | KLHHQRVDSSKSNQQEGKTWKAIRVDLVMCPYENRAFALL |
| | GWTGSRQFERDIRRYATHERKMMLDNHALYDKTKRVFLKA |
| | ESEEEIFAHLGLDYIEPWERNA |
| 38 | MGSSHHHHHHGSGLVPRGSASMSDSEVNQEAKPEVKPEVK |
| | PETHINLKVSDGSSEIFFKIKKTTPLRRLMEAFAKRQGKE |
| | MDSLRFLYDGIRIQADQTPEDLDMEDNDIIEAHREQIGGE |
| | LMRTDYSATPNPGFQKTPPLAVKKISQYACQRKTTLNNYN |
| | HIFTDAFEILAENSEFKENEVSYVTFMRAASVLKSLPFTI |
| | ISMKDTEGIPCLGDKVKCIIEEIIEDGESSEVKAVLNDER |
| | YQSFKLFTSVFGVGLKTSEKWFRMGFRSLSKIMSDKTLKF |
| | TKMQKAGFLYYEDLVSCVTRAEAEAVGVLVKEAVWAFLPD |
| | AFVTMTGGFRRGKKIGHDVDFLITSPGSAEDEEQLLPKVI |
| | NLWEKKGLLLYYDLVESTFEKFKLPSRQVDTLDHFQKCFL |
| | ILKLHHQRVDSSKSNQQEGKTWKAIRVDLVMCPYENRAFA |
| | LLGWTGSRQFERDIRRYATHERKMMLDNHALYDKTKRVFL |
| | KAESEEEIFAHLGLDYIEPWERNA |
| 39. | SPSPVPGSQNVPAPAVKKISQYACQRRTTLNNYNQLFTDA |
| | LDILAENDELRENEGSCLAFMRASSVLKSLPFPITSMKDT |
| | EGIPCLGDKVKSIIEGIIEDGESSEAKAVLNDERYKSFKL |
| | FTSVFGVGLKTAEKWFRMGFRTLSKIQSDKSLRFTQMQKA |
| | GFLYYEDLVSCVNRPEAEAVSMLVKEAVVTFLPDALVTMT |
| | GGFRRGKMTGHDVDFLITSPEATEDEEQQLLHKVTDFWKQ |
| | QGLLLYCDILESTFEKFKQPSRKVDALDHFQKCFLILKLD |
| | HGRVHSEKSGQQEGKGWKAIRVDLVMCPYDRRAFALLGWT |
| | GSRQFERDLRRYATHERKMMLDNHALYDRTKRVFLEAESE |
| | EEIFAHLGLDYIEPWERNA |
| 40. | MGHHHHHHHHHHSSGHIDDDDKHMMKIEEGKLVIWINGDK |
| | GYNGLAEVGKKFEKDTGIKVTVEHPDKLEEKFPQVAATGD |
| | GPDIIFWAHDRFGGYAQSGLLAEITPDKAFQDKLYPFTWD |
| | AVRYNGKLIAYPIAVEALSLIYNKDLLPNPPKTWEEIPAL |
| | DKELKAKGKSALMFNLQEPYFTWPLIAADGGYAFKYENGK |
| | YDIKDVGVDNAGAKAGLTFLVDLIKNKHMNADTDYSIAEA |
| | AFNKGETAMTINGPWAWSNIDTSKVNYGVTVLPTFKGQPS |
| | KPFVGVLSAGINAASPNKELAKEFLENYLLTDEGLEAVNK |
| | DKPLGAVALKSYEEELVKDPRIAATMENAQKGEIMPNIPQ |
| | MSAFWYAVRTAVINAASGRQTVDEALKDAQTNSSSNNNNN |
| | NNNNNLGIEGRISHMSMGGRDIVDGSEFSPSPVPGSQNVP |
| | APAVKKISQYACQRRTTLNNYNQLFTDALDILAENDELRE |
| | NEGSCLAFMRASSVLKSLPFPITSMKDTEGIPCLGDKVKS |
| | IIEGIIEDGESSEAKAVLNDERYKSFKLFTSVFGVGLKTA |
| | EKWFRMGFRTLSKIQSDKSLRFTQMQKAGFLYYEDLVSCV |
| | NRPEAEAVSMLVKEAVVTFLPDALVTMTGGFRRGKMTGHD |
| | VDFLITSPEATEDEEQQLLHKVTDFWKQQGLLLYCDILES |
| | TFEKFKQPSRKVDALDHFQKCFLILKLDHGRVHSEKSGQQ |
| | EGKGWKAIRVDLVMCPYDRRAFALLGWTGSRQFERDLRRY |
| | ATHERKMMLDNHALYDRTKRVFLEAESEEEIFAHLGLDYI |
| | EPWERNA |
| 41. | MGHHHHHHHHHHSSGHIDDDDKHMMKIEEGKLVIWINGDK |
| | GYNGLAEVGKKFEKDTGIKVTVEHPDKLEEKFPQVAATGD |
| | GPDIIFWAHDRFGGYAQSGLLAEITPDKAFQDKLYPFTWD |
| | AVRYNGKLIAYPIAVEALSLIYNKDLLPNPPKTWEEIPAL |
| | DKELKAKGKSALMFNLQEPYFTWPLIAADGGYAFKYENGK |
| | YDIKDVGVDNAGAKAGLTFLVDLIKNKHMNADTDYSIAEA |
| | AFNKGETAMTINGPWAWSNIDTSKVNYGVTVLPTFKGQPS |
| | KPFVGVLSAGINAASPNKELAKEFLENYLLTDEGLEAVNK |
| | DKPLGAVALKSYEEELVKDPRIAATMENAQKGEIMPNIPQ |
| | MSAFWYAVRTAVINAASGRQTVDEALKDAQTNSSS |
| 42. | NNNNNNNNNNLGSSGH |
| 43. | RDHMVLHEYVNAAGIT |
| 44. | NNNNNNNNNNLG |
| 45. | DGGSGGGSTSH |
| 46. | MGSSHHHHHHGSGLVPRGSASMSDSEVNQEAKPEVKPEVK |
| | PETHINLKVSDGSSEIFFKIKKTTPLRRLMEAFAKRQGKE |
| | MDSLRFLYDGIRIQADQTPEDLDMEDNDIIEAHREQIGG |
| 47. | ELM |
| 48 | MNKEILAVVEAVSNEKALPREKIFEALESALATATKKKYE |
| | QEIDVRVQIDRKSGDFDTFRRWLVVDEVTQPTKEITLEAA |
| | RYEDESLNLGDYVEDQIESVTFDRITTQTAKQVIVQKVRE |
| | AERAMVVDQFREHEGEIITGVVKKVNRDNISLDLGNNAEA |
| | VILREDMLPRENFRPGDRVRGVLYSVRPEARGAQLFVTRS |
| | KPEMLIELFRIEVPEIGEEVIEIKAAARDPGSRAKIAVKT |
| | NDKRIDPVGACVGMRGARVQAVSTELGGERIDIVLWDDNP |
| | AQFVINAMAPADVASIVVDEDKHTMDIAVEAGNLAQAIGR |
| | NGQNVRLASQLSGWELNVMTVDDLQAKHQAEAHAAIDTFT |
| | KYLDIDEDFATVLVEEGFSTLEELAYVPMKELLEIEGLDE |
| | PTVEALRERAKNALATIAQAQEESLGDNKPADDLLNLEGV |
| | DRDLAFKLAARGVCTLEDLAEQGIDDLADIEGLTDEKAGA |
| | LIMAARNICWFGDEA |
| 49. | MNKEILAVVEAVSNEKALPREKIFEALESALATATKKKYE |
| | QEIDVRVQIDRKSGDFDTFRRWLVVDEVTQPTKEITLEAA |
| | RYEDESLNLGDYVEDQIESVTFDRITTQTAKQVIVQKVRE |
| | AERAMVVDQFREHEGEIITGVVKKVNRDNISLDLGNNAEA |
| | VILREDMLPRENFRPGDRVRGVLYSVRPEARGAQLFVTRS |
| | KPEMLIELFRIEVPEIGEEVIEIKAAARDPGSRAKIAVKT |
| | NDKRIDPVGACVGMRGARVQAVSTELGGERIDIVLWDDNP |
| | AQFVINAMAPADVASIVVDEDKHTMDIAVEAGNLAQAIGR |
| | NGQNVRLASQLSGWELNVMTVDDLQAKHQAEAHAAIDTFT |
| | KYLDIDEDFATVLVEEGESTLEELAYVPMKELLEIEGLDE |
| | PTVEALRERAKNALATIAQAQEESLGDNKPADDLLNLEGV |
| | DRDLAFKLAARGVCTLEDLAEQGIDDLADIEGLTDEKAGA |
| | LIMAARNICWFGDEATSGSGHHHHHHHH |
| 50. | SDKIIHLTDDSFDTDVLKADGAILVDFWAEWCGPCKMIAP |
| | ILDEIADEYQGKLTVAKLNIDQNPGTAPKYGIRGIPTLLL |
| | FKNGEVAATKVGALSKGQLKEFLDANLA |
| 51. | MSDKIIHLTDDSFDTDVLKADGAILVDFWAEWCGPCKMIA |
| | PILDEIADEYQGKLTVAKLNIDQNPGTAPKYGIRGIPTLL |
| | LFKNGEVAATKVGALSKGQLKEFLDANLAGSSHHHHHHHH |
| 52. | MSPILGYWKIKGLVQPTRLLLEYLEEKYEEHLYERDEGDK |
| | WRNKKFELGLEFPNLPYYIDGDVKLTQSMAIIRYIADKHN |
| | MLGGCPKERAEISMLEGAVLDIRYGVSRIAYSKDFETLKV |
| | DFLSKLPEMLKMFEDRLCHKTYLNGDHVTHPDFMLYDALD |
| | VVLYMDPMCLDAFPKLVCFKKRIEAIPQIDKYLKSSKYIA |
| | WPLQGWQATFGGGDHPPK |
| 53. | HHHHHHHHHHAGMSPILGYWKIKGLVQPTRLLLEYLEEKY |
| | EEHLYERDEGDKWRNKKFELGLEFPNLPYYIDGDVKLTQS |
| | MAIIRYIADKHNMLGGCPKERAEISMLEGAVLDIRYGVSR |
| | IAYSKDFETLKVDFLSKLPEMLKMFEDRLCHKTYLNGDHV |
| | THPDFMLYDALDVVLYMDPMCLDAFPKLVCFKKRIEAIPQ |
| | IDKYLKSSKYIAWPLQGWQATFGGGDHPPK |
| 54. | EIGTGFPFDPHYVEVLGERMHYVDVGPRDGTPVLFLHGNP |
| | TSSYVWRNIIPHVAPTHRCIAPDLIGMGKSDKPDLGYFFD |
| | DHVRFMDAFIEALGLEEVVLVIHDWGSALGFHWAKRNPER |
| | VKGIAFMEFIRPIPTWDEWPEFARETFQAFRTTDVGRKLI |
| | IDQNVFIEGTLPMGVVRPLTEVEMDHYREPFLNPVDREPL |
| | WRFPNELPIAGEPANIVALVEEYMDWLHQSPVPKLLFWGT |
| | PGVLIPPAEAARLAKSLPNCKAVDIGPGLNLLQEDNPDLI |
| | GSEIARWLSTLEIS |
| 55. | EIGTGFPFDPHYVEVLGERMHYVDVGPRDGTPVLFLHGNP |
| | TSSYVWRNIIPHVAPTHRCIAPDLIGMGKSDKPDLGYFFD |
| | DHVRFMDAFIEALGLEEVVLVIHDWGSALGFHWAKRNPER |
| | VKGIAFMEFIRPIPTWDEWPEFARETFQAFRTTDVGRKLI |
| | IDQNVFIEGTLPMGVVRPLTEVEMDHYREPFLNPVDREPL |
| | WRFPNELPIAGEPANIVALVEEYMDWLHQSPVPKLLFWGT |
| | PGVLIPPAEAARLAKSLPNCKAVDIGPGLNLLQEDNPDLI |
| | GSEIARWLSTLEISGHHHHHHHHHH |
| 56. | MSDSEVNQEAKPEVKPEVKPETHINLKVSDGSSEIFFKIK |
| | KTTPLRRLMEAFAKRQGKEMDSLRFLYDGIRIQADQTPED |
| | LDMEDNDIIEAHREQIGG |
| 57. | MGHHHHHHHHHHSSGHIDDDDKHMMKIEEGKLVIWINGDK |
| | GYNGLAEVGKKFEKDTGIKVTVEHPDKLEEKFPQVAATGD |
| | GPDIIFWAHDRFGGYAQSGLLAEITPDKAFQDKLYPFTWD |
| | AVRYNGKLIAYPIAVEALSLIYNKDLLPNPPKTWEEIPAL |
| | DKELKAKGKSALMFNLQEPYFTWPLIAADGGYAFKYENGK |
| | YDIKDVGVDNAGAKAGLTFLVDLIKNKHMNADTDYSIAEA |
| | AFNKGETAMTINGPWAWSNIDTSKVNYGVTVLPTFKGQPS |
| | KPFVGVLSAGINAASPNKELAKEFLENYLLTDEGLEAVNK |
| | DKPLGAVALKSYEEELVKDPRIAATMENAQKGEIMPNIPQ |
| | MSAFWYAVRTAVINAASGRQTVDEALKDAQTRITK |
| 58. | MGHHHHHHHHHHSSGHIDDDDKHMM |
| 59. | TAATACGACTCACTTG |

### P Embodiments

P Embodiment 1. A recombinant terminal deoxynucleotidyl transferase (TdT) enzyme or a fragment thereof, capable of synthesizing DNA or RNA with high fidelity and efficiency, wherein the enzyme comprises a sequence with at most 10 amino acid units of difference with a sequence selected from the group consisting of: SEQ ID NO:4, SEQ ID NO:5, SEQ ID NO:6, SEQ ID NO:7, SEQ ID NO:8, SEQ ID NO:9, SEQ ID NO: 10, SEQ ID NO: 11, SEQ ID NO:12, SEQ ID NO:13, SEQ ID NO: 14, and SEQ ID NO:15.

P Embodiment 2. The recombinant terminal deoxynucleotidyl transferase (TdT) enzyme or a fragment thereof according to P embodiment 1, wherein the enzyme comprises a sequence with at most 6 amino acid units of difference with a sequence selected from the group consisting of: SEQ ID NO:4, SEQ ID NO:5, SEQ ID NO:6, SEQ ID NO: 7, SEQ ID NO:8, SEQ ID NO:9, SEQ ID NO: 10, SEQ ID NO:11, SEQ ID NO:12, SEQ ID NO: 13, SEQ ID NO:14, and SEQ ID NO:15.

P Embodiment 3. The recombinant terminal deoxynucleotidyl transferase (TdT) enzyme or a fragment thereof according to P embodiment 2, wherein the enzyme comprises a sequence with at most 3 amino acid units of difference with a sequence selected from the group consisting of: SEQ ID NO:4, SEQ ID NO:5, SEQ ID NO:6, SEQ ID NO:7, SEQ ID NO:8, SEQ ID NO: 9, SEQ ID NO:10, SEQ ID NO:11, SEQ ID NO: 12, SEQ ID NO:13, SEQ ID NO:14, and SEQ ID NO:15.

P Embodiment 4. The recombinant terminal deoxynucleotidyl transferase (TdT) enzyme or a fragment thereof according to P embodiment 3, wherein the enzyme comprises a group of 4 sequences, selected from the group consisting of: SEQ ID NO:4, SEQ ID NO:7, SEQ ID NO: 10, and SEQ ID NO:13; SEQ ID NO:5, SEQ ID NO:8, SEQ ID NO: 11, and SEQ ID NO: 14; and SEQ ID NO:6, SEQ ID NO:9, SEQ ID NO: 12, and SEQ ID NO:15.

P Embodiment 5. The recombinant terminal deoxynucleotidyl transferase (TdT) enzyme or a fragment thereof according to any of P embodiments 1 to 4, wherein the enzyme comprises a sequence with 85% identity to an amino acid sequence selected from the group consisting of: SEQ ID NO:1, SEQ ID NO:2, and SEQ ID NO:3.

P Embodiment 6. The recombinant terminal deoxynucleotidyl transferase (TdT) enzyme or a fragment thereof according to P embodiment 5, wherein the enzyme comprises a sequence with 90% identity to an amino acid sequence selected from the group consisting of: SEQ ID NO:1, SEQ ID NO:2, and SEQ ID NO:3.

P Embodiment 7. The recombinant terminal deoxynucleotidyl transferase (TdT) enzyme or a fragment thereof according to P embodiment 6, wherein the enzyme comprises a sequence with 95% identity to an amino acid sequence selected from the group consisting of: SEQ ID NO:1, SEQ ID NO:2, and SEQ ID NO:3.

P Embodiment 8. A method for synthesizing DNA or RNA sequences of interest using the recombinant terminal deoxynucleotidyl transferase enzyme or the fragment thereof according to any of P embodiments 1 to 7, wherein the method comprises the following steps: a) contacting the enzyme or the fragment thereof with a nucleotide chain immobilized in a solid support in the presence of nucleotides, therefore synthesizing a DNA or RNA sequence of interest by adding nucleotides to the growing nucleotide chain; and b) separating the synthesized DNA/RNA sequence of interest from said solid support.

P Embodiment 9. A composition comprising the recombinant terminal deoxynucleotidyl transferase enzyme or the fragment thereof according to any of P embodiments 1 to 7, and a suitable buffer.

P Embodiment 10. A kit for synthesizing DNA or RNA sequences of interest, wherein the kit comprises: a) a composition according to P embodiment 9; b) a solid support containing a growing chain of nucleotides; c) NTPs; d) a reducing agent; e) a detergent; and f) a cryopreservative.

### Embodiments

Embodiment 1. A terminal deoxynucleotidyl transferase (TdT) peptide, wherein the peptide comprises an amino acid sequence having at least 80% sequence identity to the amino acid sequence of SEQ ID NO:1.

Embodiment 2. The peptide of embodiment 1, wherein the peptide comprises an amino acid sequence having at least 95% sequence identity to the amino acid sequence of SEQ ID NO: 1.

Embodiment 3. The peptide of embodiment 1 or 2, wherein the peptide comprises the amino acid sequence of SEQ ID NO:1.

Embodiment 4. A terminal deoxynucleotidyl transferase (TdT) peptide, wherein the peptide comprises an amino acid sequence having at least 80% sequence identity to the amino acid sequence of SEQ ID NO:2.

Embodiment 5. The peptide of embodiment 4, wherein the peptide comprises an amino acid sequence having at least 95% sequence identity to the amino acid sequence of SEQ ID NO: 2.

Embodiment 6. The peptide of embodiment 4 or 5, wherein the peptide comprises the amino acid sequence of SEQ ID NO:2.

Embodiment 7. A terminal deoxynucleotidyl transferase (TdT) peptide, wherein the peptide comprises an amino acid sequence having at least 80% sequence identity to the amino acid sequence of SEQ ID NO:3.

Embodiment 8. The peptide of embodiment 7, wherein the peptide comprises an amino acid sequence having at least 95% sequence identity to the amino acid sequence of SEQ ID NO: 3.

Embodiment 9. The peptide of embodiment 7 or 8, wherein the peptide comprises the amino acid sequence of SEQ ID NO:3.

Embodiment 10. A terminal deoxynucleotidyl transferase (TdT) peptide, wherein the peptide comprises an amino acid sequence having at least 80% sequence identity to the amino acid sequence of SEQ ID NO:16.

Embodiment 11. The peptide of embodiment 10, wherein the peptide comprises an amino acid sequence having at least 95% sequence identity to the amino acid sequence of SEQ ID NO: 16.

Embodiment 12. The peptide of embodiment 10 or 11, wherein the peptide comprises the amino acid sequence of SEQ ID NO:16.

Embodiment 13. A terminal deoxynucleotidyl transferase (TdT) peptide, wherein the peptide comprises an amino acid sequence having at least 80% sequence identity to the amino acid sequence of SEQ ID NO:17.

Embodiment 14. The peptide of embodiment 13, wherein the peptide comprises an amino acid sequence having at least 95% sequence identity to the amino acid sequence of SEQ ID NO: 17.

Embodiment 15. The peptide of embodiment 13 or 14, wherein the peptide comprises the amino acid sequence of SEQ ID NO:17.

Embodiment 16. A terminal deoxynucleotidyl transferase (TdT) peptide, wherein the peptide comprises an amino acid sequence having at least 80% sequence identity to the amino acid sequence of SEQ ID NO:18.

Embodiment 17. The peptide of embodiment 16, wherein the peptide comprises an amino acid sequence having at least 95% sequence identity to the amino acid sequence of SEQ ID NO: 18.

Embodiment 18. The peptide of embodiment 16 or 17, wherein the peptide comprises the amino acid sequence of SEQ ID NO:18.

Embodiment 19. A terminal deoxynucleotidyl transferase (TdT) peptide, wherein the peptide comprises an amino acid sequence having at least 80% sequence identity to the amino acid sequence of SEQ ID NO:19.

Embodiment 20. The peptide of embodiment 19, wherein the peptide comprises an amino acid sequence having at least 95% sequence identity to the amino acid sequence of SEQ ID NO: 19.

Embodiment 21. The peptide of embodiment 19 or 20, wherein the peptide comprises the amino acid sequence of SEQ ID NO:19.

Embodiment 22. A terminal deoxynucleotidyl transferase (TdT) peptide, wherein the peptide comprises an amino acid sequence having at least 80% sequence identity to the amino acid sequence of SEQ ID NO:20.

Embodiment 23. The peptide of embodiment 22, wherein the peptide comprises an amino acid sequence having at least 95% sequence identity to the amino acid sequence of SEQ ID NO: 20.

Embodiment 24. The peptide of embodiment 22 or 23, wherein the peptide comprises the amino acid sequence of SEQ ID NO:20.

Embodiment 25. A terminal deoxynucleotidyl transferase (TdT) peptide, wherein the peptide comprises an amino acid sequence having at least 80% sequence identity to the amino acid sequence of SEQ ID NO:21.

Embodiment 26. The peptide of embodiment 25, wherein the peptide comprises an amino acid sequence having at least 95% sequence identity to the amino acid sequence of SEQ ID NO: 21.

Embodiment 27. The peptide of embodiment 25 or 26, wherein the peptide comprises the amino acid sequence of SEQ ID NO:21.

Embodiment 28. A terminal deoxynucleotidyl transferase (TdT) peptide, wherein the peptide comprises an amino acid sequence having at least 80% sequence identity to the amino acid sequence of SEQ ID NO:22.

Embodiment 29. The peptide of embodiment 28, wherein the peptide comprises an amino acid sequence having at least 95% sequence identity to the amino acid sequence of SEQ ID NO: 22.

Embodiment 30. The peptide of embodiment 28 or 29, wherein the peptide comprises the amino acid sequence of SEQ ID NO:22.

Embodiment 31. A terminal deoxynucleotidyl transferase (TdT) peptide, wherein the peptide comprises an amino acid sequence having at least 80% sequence identity to the amino acid sequence of SEQ ID NO:23.

Embodiment 32. The peptide of embodiment 31, wherein the peptide comprises an amino acid sequence having at least 95% sequence identity to the amino acid sequence of SEQ ID NO: 23.

Embodiment 33. The peptide of embodiment 31 or 32, wherein the peptide comprises the amino acid sequence of SEQ ID NO:23.

Embodiment 34. A terminal deoxynucleotidyl transferase (TdT) peptide, wherein the peptide comprises an amino acid sequence having at least 80% sequence identity to the amino acid sequence of SEQ ID NO:24.

Embodiment 35. The peptide of embodiment 34, wherein the peptide comprises an amino acid sequence having at least 95% sequence identity to the amino acid sequence of SEQ ID NO: 24.

Embodiment 36. The peptide of embodiment 34 or 35, wherein the peptide comprises the amino acid sequence of SEQ ID NO:24.

Embodiment 37. The peptide of any one of embodiments 1-36, further comprising a solubilizing amino acid sequence, wherein the solubility tag sequence is a maltose-binding protein (MBP) sequence, a small ubiquitin modified (SUMO) sequence, a thioredoxin (TrxA) sequence, a N-utilizing substance A (NusA) sequence, a Fasciola hepatica 8-kDa antigen (Fh8) sequence, a mutated dehalogenase (Halo) sequence, or a glutathione-S-transferase (GST) sequence.

Embodiment 38. The peptide of embodiment 37, wherein the solubilizing amino acid sequence comprises the amino acid sequence of SEQ ID NO:41, SEQ ID NO:46, SEQ ID NO: 48, SEQ ID NO:49, SEQ ID NO:50, SEQ ID NO:51, SEQ ID NO:52, SEQ ID NO:53, SEQ ID NO: 54, SEQ ID NO:55, SEQ ID NO:56, SEQ ID NO:57, or SEQ ID NO:58.

Embodiment 39. A method for synthesizing an oligonucleotide, the method comprises contacting the TdT peptide of any one of embodiments 1-38 with (i) an oligonucleotide primer, wherein the oligonucleotide primer comprises a free 3′-hydroxyl; and (ii) a nucleoside triphosphate or 3′-O-blocked nucleoside triphosphate under conditions that promote addition of the nucleotide triphosphate or 3′-O-blocked nucleoside triphosphate to the 3′ terminal end of the oligonucleotide primer, thereby forming an elongated oligonucleotide fragment.

Embodiment 40. The method of embodiment 39, wherein the method is repeated to add additional nucleotides, and wherein the additional nucleotide triphosphates are added to the 3′ terminal end of the elongated oligonucleotide fragment.

Embodiment 41. The method of embodiment 39, further comprising deblocking the elongated oligonucleotide fragment and allowing the addition of additional 3′-O-blocked nucleoside triphosphates to the 3′ terminal end of the elongated oligonucleotide fragment.

Embodiment 42. The method of any one of embodiments 39-41, wherein the oligonucleotide primer is immobilized on a solid support.

Embodiment 43. The method of any one of embodiments 39-42, wherein the elongated oligonucleotide fragment is isolated from the solid support.

Embodiment 44. A composition comprising the TdT peptide of any one of embodiments 1-38 and an appropriate buffer.

Embodiment 45. A kit for synthesizing a target oligonucleotide, the kit comprising: a) the composition of embodiment 44; b) a nucleotide triphosphate or a 3′-O-blocked nucleoside triphosphate; and c) an oligonucleotide primer comprising a free 3′-hydroxyl.

Embodiment 46. The kit of embodiment 45, wherein the oligonucleotide primer is immobilized on a solid support.

Embodiment 47. The kit of embodiment 45 or 46, further comprising a reducing agent, a detergent, and a cryopreservative.

Embodiment 48. The kit of any one of embodiments 45-47, wherein the TdT peptide comprises the amino acid sequence of SEQ ID NO:1, SEQ ID NO:2, SEQ ID NO:3, SEQ ID NO: 16, SEQ ID NO: 17, SEQ ID NO:18, SEQ ID NO: 19, SEQ ID NO:20, SEQ ID NO:21, SEQ ID NO: 22, SEQ ID NO:23, or SEQ ID NO:24.

What is claimed is:

1. A terminal deoxynucleotidyl transferase (TdT) peptide, wherein the peptide comprises an amino acid sequence having at least 80% sequence identity to the amino acid sequence of SEQ ID NO:1.

2. The peptide of claim 1, wherein the peptide comprises an amino acid sequence having at least 95% sequence identity to the amino acid sequence of SEQ ID NO:1.

3. The peptide of claim 1, wherein the peptide comprises the amino acid sequence of SEQ ID NO:1.

4. A terminal deoxynucleotidyl transferase (TdT) peptide, wherein the peptide comprises an amino acid sequence having at least 80% sequence identity to the amino acid sequence of SEQ ID NO:2.

5. The peptide of claim 4, wherein the peptide comprises an amino acid sequence having at least 95% sequence identity to the amino acid sequence of SEQ ID NO:2.

6. The peptide of claim 4, wherein the peptide comprises the amino acid sequence of SEQ ID NO:2.

7. A terminal deoxynucleotidyl transferase (TdT) peptide, wherein the peptide comprises an amino acid sequence having at least 80% sequence identity to the amino acid sequence of SEQ ID NO:3.

8. The peptide of claim 7, wherein the peptide comprises an amino acid sequence having at least 95% sequence identity to the amino acid sequence of SEQ ID NO:3.

9. The peptide of claim 7, wherein the peptide comprises the amino acid sequence of SEQ ID NO:3.

10. A terminal deoxynucleotidyl transferase (TdT) peptide, wherein the peptide comprises an amino acid sequence having at least 80% sequence identity to the amino acid sequence of SEQ ID NO:16.

11. The peptide of claim 10, wherein the peptide comprises an amino acid sequence having at least 95% sequence identity to the amino acid sequence of SEQ ID NO: 16.

12. The peptide of claim 10, wherein the peptide comprises the amino acid sequence of SEQ ID NO:16.

13. A terminal deoxynucleotidyl transferase (TdT) peptide, wherein the peptide comprises an amino acid sequence having at least 80% sequence identity to the amino acid sequence of SEQ ID NO:17.

14. The peptide of claim 13, wherein the peptide comprises an amino acid sequence having at least 95% sequence identity to the amino acid sequence of SEQ ID NO: 17.

15. The peptide of claim 13, wherein the peptide comprises the amino acid sequence of SEQ ID NO:17.

16. A terminal deoxynucleotidyl transferase (TdT) peptide, wherein the peptide comprises an amino acid sequence having at least 80% sequence identity to the amino acid sequence of SEQ ID NO: 18.

17. The peptide of claim 16, wherein the peptide comprises an amino acid sequence having at least 95% sequence identity to the amino acid sequence of SEQ ID NO: 18.

18. The peptide of claim 16, wherein the peptide comprises the amino acid sequence of SEQ ID NO:18.

19. A terminal deoxynucleotidyl transferase (TdT) peptide, wherein the peptide comprises an amino acid sequence having at least 80% sequence identity to the amino acid sequence of SEQ ID NO:19.

20. The peptide of claim 19, wherein the peptide comprises an amino acid sequence having at least 95% sequence identity to the amino acid sequence of SEQ ID NO: 19.

21. The peptide of claim 19, wherein the peptide comprises the amino acid sequence of SEQ ID NO:19.

22. A terminal deoxynucleotidyl transferase (TdT) peptide, wherein the peptide comprises an amino acid sequence having at least 80% sequence identity to the amino acid sequence of SEQ ID NO:20.

23. The peptide of claim 22, wherein the peptide comprises an amino acid sequence having at least 95% sequence identity to the amino acid sequence of SEQ ID NO: 20.

24. The peptide of claim 22, wherein the peptide comprises the amino acid sequence of SEQ ID NO:20.

25. A terminal deoxynucleotidyl transferase (TdT) peptide, wherein the peptide comprises an amino acid sequence having at least 80% sequence identity to the amino acid sequence of SEQ ID NO:21.

26. The peptide of claim 25, wherein the peptide comprises an amino acid sequence having at least 95% sequence identity to the amino acid sequence of SEQ ID NO: 21.

27. The peptide of claim 25, wherein the peptide comprises the amino acid sequence of SEQ ID NO:21.

28. A terminal deoxynucleotidyl transferase (TdT) peptide, wherein the peptide comprises an amino acid sequence having at least 80% sequence identity to the amino acid sequence of SEQ ID NO:22.

29. The peptide of claim 28, wherein the peptide comprises an amino acid sequence having at least 95% sequence identity to the amino acid sequence of SEQ ID NO: 22.

30. The peptide of claim 28, wherein the peptide comprises the amino acid sequence of SEQ ID NO:22.

31. A terminal deoxynucleotidyl transferase (TdT) peptide, wherein the peptide comprises an amino acid sequence having at least 80% sequence identity to the amino acid sequence of SEQ ID NO:23.

32. The peptide of claim 31, wherein the peptide comprises an amino acid sequence having at least 95% sequence identity to the amino acid sequence of SEQ ID NO: 23.

33. The peptide of claim 31, wherein the peptide comprises the amino acid sequence of SEQ ID NO:23.

34. A terminal deoxynucleotidyl transferase (TdT) peptide, wherein the peptide comprises an amino acid sequence having at least 80% sequence identity to the amino acid sequence of SEQ ID NO:24.

35. The peptide of claim 34, wherein the peptide comprises an amino acid sequence having at least 95% sequence identity to the amino acid sequence of SEQ ID NO: 24.

36. The peptide of claim 34, wherein the peptide comprises the amino acid sequence of SEQ ID NO:24.

37. The peptide of claim 1, further comprising a solubilizing amino acid sequence, wherein the solubility tag sequence is a maltose-binding protein (MBP) sequence, a small ubiquitin modified (SUMO) sequence, a thioredoxin (TrxA) sequence, a N-utilizing substance A (NusA) sequence, a Fasciola hepatica 8-kDa antigen (Fh8) sequence, a mutated dehalogenase (Halo) sequence, or a glutathione-S-transferase (GST) sequence.

38. The peptide of claim 37, wherein the solubilizing amino acid sequence comprises the amino acid sequence of SEQ ID NO:41, SEQ ID NO:46, SEQ ID NO:48, SEQ ID NO: 49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, or SEQ ID NO:58.

39. A method for synthesizing an oligonucleotide, the method comprises contacting the TdT peptide of claim 1 with (i) an oligonucleotide primer, wherein the oligonucleotide primer comprises a free 3′-hydroxyl; and (ii) a nucleoside triphosphate or 3′-O-blocked nucleoside triphosphate under conditions that promote addition of the nucleotide triphosphate or 3′-O-blocked nucleoside triphosphate to the 3′ terminal end of the oligonucleotide primer, thereby forming an elongated oligonucleotide fragment.

40. The method of claim 39, wherein the method is repeated to add additional nucleotides, and wherein the additional nucleotide triphosphates are added to the 3′ terminal end of the elongated oligonucleotide fragment.

41. The method of claim 39, further comprising deblocking the elongated oligonucleotide fragment and allowing the addition of additional 3′-O-blocked nucleoside triphosphates to the 3′ terminal end of the elongated oligonucleotide fragment.

42. The method of claim 39, wherein the oligonucleotide primer is immobilized on a solid support.

43. The method of claim 39, wherein the elongated oligonucleotide fragment is isolated from the solid support.

44. A composition comprising the TdT peptide of claim 1 and an appropriate buffer.

45. A kit for synthesizing a target oligonucleotide, the kit comprising:
a) the composition of claim 44;
b) a nucleotide triphosphate or a 3′-O-blocked nucleoside triphosphate; and
c) an oligonucleotide primer comprising a free 3′-hydroxyl.

46. The kit of claim 45, wherein the oligonucleotide primer is immobilized on a solid support.

47. The kit of claim 45, further comprising a reducing agent, a detergent, and a cryopreservative.

48. The kit of claim 45, wherein the TdT peptide comprises the amino acid sequence of SEQ ID NO:1, SEQ ID NO:2, SEQ ID NO:3, SEQ ID NO:16, SEQ ID NO:17, SEQ ID NO:18, SEQ ID NO:19, SEQ ID NO:20, SEQ ID NO:21, SEQ ID NO:22, SEQ ID NO: 23, or SEQ ID NO:24.
